SEQUENCE LISTING (54 sequences)

SEQ ID NO: 1 - protein, 135 aa, synthetic construct
MSDSNQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQGMSLNDFQKQQKQ

SEQ ID NO: 2 - protein, 85 aa, synthetic construct
QQQQQQQQQQQQQQQQQQQQQQQQQQQQQQQQQQQQQQQQQQQQQQQQQQQQQQQQQQQQQQQQQQQQQQQQQQQQQQQQQQQQQ

SEQ ID NO: 3 - protein, 20 aa, synthetic construct
QQQQQQQQQQQQQQQQQQQQ

SEQ ID NO: 4 - protein, 30 aa, synthetic construct
QQQQQQQQQQQQQQQQQQQQQQQQQQQQQQ

SEQ ID NO: 5 - protein, 70 aa, synthetic construct
QQQQQQQQQQQQQQQQQQQQQQQQQQQQQQQQQQQQQQQQQQQQQQQQQQQQQQQQQQQQQQQQQQQQQQ

SEQ ID NO: 6 - protein, 10 aa, synthetic construct
QQQQQQQQQQ

SEQ ID NO: 7 - protein, 55 aa, synthetic construct
QQQQQQQQQQQQQQQQQQQQQQQQQQQQQQQQQQQQQQQQQQQQQQQQQQQQQQQ

SEQ ID NO: 8 - protein, 140 aa, synthetic construct
QQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQ

SEQ ID NO: 9 - protein, 35 aa, synthetic construct
QQQQQQQQQQQQQQQQQQQQQQQQQQQQQQQQQQQ

SEQ ID NO: 10 - protein, 40 aa, synthetic construct
QQQQQQQQQQQQQQQQQQQQQQQQQQQQQQQQQQQQQQQQ

SEQ ID NO: 11 - protein, 45 aa, synthetic construct
QQQQQQQQQQQQQQQQQQQQQQQQQQQQQQQQQQQQQQQQQQQQQ

SEQ ID NO: 12 - protein, 50 aa, synthetic construct
QQQQQQQQQQQQQQQQQQQQQQQQQQQQQQQQQQQQQQQQQQQQQQQQQQ

SEQ ID NO: 13 - protein, 60 aa, synthetic construct
QQQNQQQNQQQNQQQNQQQNQQQNQQQNQQQNQQQNQQQNQQQNQQQNQQQNQQQNQQQN

SEQ ID NO: 14 - protein, 60 aa, synthetic construct
QQQQNQQQQNQQQQNQQQQNQQQQNQQQQNQQQQNQQQQNQQQQNQQQQNQQQQNQQQQN

SEQ ID NO: 15 - protein, 60 aa, synthetic construct
QQQQQNQQQQQNQQQQQNQQQQQNQQQQQNQQQQQNQQQQQNQQQQQNQQQQQNQQQQQN

SEQ ID NO: 16 - protein, 60 aa, synthetic construct
QQQQQQQNQQQQQQQNQQQQQQQNQQQQQQQNQQQQQQQNQQQQQQQNQQQQQQQNQQQQ

SEQ ID NO: 17 - protein, 60 aa, synthetic construct
QQQQQQQQQNQQQQQQQQQNQQQQQQQQQNQQQQQQQQQNQQQQQQQQQNQQQQQQQQQN

SEQ ID NO: 18 - protein, 60 aa, synthetic construct
NNNNNNNNNNNNNNNNNNNNNNNNNNNNNNNNNNNNNNNNNNNNNNNNNNNNNNNNNNNN

SEQ ID NO: 19 - protein, 60 aa, synthetic construct
QQQQQQQQQQQQQQQQQQQQQQQQQQQQQQQQQQQQQQQQQQQQQQQQQQQQQQQQQQQQ

SEQ ID NO: 20 - protein, 60 aa, synthetic construct
QNQNQNQNQNQNQNQNQNQNQNQNQNQNQNQNQNQNQNQNQNQNQNQNQNQNQNQNQNQN

SEQ ID NO: 21 - protein, 60 aa, synthetic construct
QQNQQNQQNQQNQQNQQNQQNQQNQQNQQNQQNQQNQQNQQNQQNQQNQQNQQNQQNQQN

SEQ ID NO: 22 - protein, 60 aa, synthetic construct
QQQQQQNQQQQQQNQQQQQQNQQQQQQNQQQQQQNQQQQQQNQQQQQQNQQQQQQNQQQQ

SEQ ID NO: 23 - protein, 60 aa, synthetic construct
NNNQNNNQNNNQNNNQNNNQNNNQNNNQNNNQNNNQNNNQNNNQNNNQNNNQNNNQNNNQ

SEQ ID NO: 24 - protein, 60 aa, synthetic construct
NNNNQNNNNQNNNNQNNNNQNNNNQNNNNQNNNNQNNNNQNNNNQNNNNQNNNNQNNNNQ

SEQ ID NO: 25 - protein, 60 aa, synthetic construct
QQQQQQQQNQQQQQQQQNQQQQQQQQNQQQQQQQQNQQQQQQQQNQQQQQQQQNQQQQQQ

SEQ ID NO: 26 - protein, 60 aa, synthetic construct
QQQSQQQSQQQSQQQSQQQSQQQSQQQSQQQSQQQSQQQSQQQSQQQSQQQSQQQSQQQS

SEQ ID NO: 27 - protein, 60 aa, synthetic construct
QQQQSQQQQSQQQQSQQQQSQQQQSQQQQSQQQQSQQQQSQQQQSQQQQSQQQQSQQQQS

SEQ ID NO: 28 - protein, 60 aa, synthetic construct
QQQQQSQQQQQSQQQQQSQQQQQSQQQQQSQQQQQSQQQQQSQQQQQSQQQQQSQQQQQS

SEQ ID NO: 29 - protein, 60 aa, synthetic construct
QQQAQQQAQQQAQQQAQQQAQQQAQQQAQQQAQQQAQQQAQQQAQQQAQQQAQQQAQQQA

SEQ ID NO: 30 - protein, 60 aa, synthetic construct
QQQQAQQQQAQQQQAQQQQAQQQQAQQQQAQQQQAQQQQAQQQQAQQQQAQQQQAQQQQA

SEQ ID NO: 31 - protein, 60 aa, synthetic construct
QQQQQAQQQQQAQQQQQAQQQQQAQQQQQAQQQQQAQQQQQAQQQQQAQQQQQAQQQQQA

SEQ ID NO: 32 - protein, 73 aa, synthetic construct
QQQHQQQHQQQHQQQHQQQHQQQHQQQHQQQHQQQHQQQHQQQHQQQHQQQHQQQHQQQHQQQHQQQHQQQHQ

SEQ ID NO: 33 - protein, 73 aa, synthetic construct
QQQQHQQQQHQQQQHQQQQHQQQQHQQQQHQQQQHQQQQHQQQQHQQQQHQQQQHQQQQHQQQQHQQQQHQQQ

SEQ ID NO: 34 - protein, 73 aa, synthetic construct
QQQQQHQQQQQHQQQQQHQQQQQHQQQQQHQQQQQHQQQQQHQQQQQHQQQQQHQQQQQHQQQQQHQQQQQHQ

SEQ ID NO: 35 - protein, 60 aa, synthetic construct
QQQGQQQGQQQGQQQGQQQGQQQGQQQGQQQGQQQGQQQGQQQGQQQGQQQGQQQGQQQG

SEQ ID NO: 36 - protein, 60 aa, synthetic construct
QQQQGQQQQGQQQQGQQQQGQQQQGQQQQGQQQQGQQQQGQQQQGQQQQGQQQQGQQQQG

SEQ ID NO: 37 - protein, 60 aa, synthetic construct
QQQQQGQQQQQGQQQQQGQQQQQGQQQQQGQQQQQGQQQQQGQQQQQGQQQQQGQQQQQG

SEQ ID NO: 38 - protein, 60 aa, synthetic construct
QQQNQQQNNQQQNQQQNNQQQNQQQNNQQQNQQQNNQQQNQQQNNQQQNQQQNNQQQNQQ

SEQ ID NO: 39 - protein, 60 aa, synthetic construct
QQQNQQQNQQQNNQQQNQQQNQQQNNQQQNQQQNQQQNNQQQNQQQNQQQNNQQQNQQQN

SEQ ID NO: 40 - protein, 60 aa, synthetic construct
QQQNQQQNQQQNQQQNNQQQNQQQNQQQNQQQNNQQQNQQQNQQQNQQQNNQQQNQQQNQ

SEQ ID NO: 41 - protein, 60 aa, synthetic construct
QQQQNNQQQQNNQQQQNNQQQQNNQQQQNNQQQQNNQQQQNNQQQQNNQQQQNNQQQQNN

SEQ ID NO: 42 - protein, 60 aa, synthetic construct
QQQQQQNNQQQQQQNNQQQQQQNNQQQQQQNNQQQQQQNNQQQQQQNNQQQQQQNNQQQQ

SEQ ID NO: 43 - protein, 60 aa, synthetic construct
QQQQQQQQNNQQQQQQQQNNQQQQQQQQNNQQQQQQQQNNQQQQQQQQNNQQQQQQQQNN

SEQ ID NO: 44 - protein, 60 aa, synthetic construct
NNQNNQNNQNNQNNQNNQNNQNNQNNQNNQNNQNNQNNQNNQNNQNNQNNQNNQNNQNNQ

SEQ ID NO: 45 - protein, 60 aa, synthetic construct
QQQNNQQQNNQQQNNQQQNNQQQNNQQQNNQQQNNQQQNNQQQNNQQQNNQQQNNQQQNN

SEQ ID NO: 46 - protein, 90 aa, synthetic construct
QQQQNQQQQNQQQQNQQQQNQQQQNQQQQNQQQNQQQNQQQNQQQNQQQNQQQNQQQNQQQNQQQNQQQNQQQNQQQNQQQNQQQNQQQN

SEQ ID NO: 47 - protein, 90 aa, synthetic construct
QQNQQNQQNQQNQQNQQNQQNQQNQQNQQNQQQNQQQNQQQNQQQNQQQNQQQNQQQNQQQNQQQNQQQNQQQNQQQNQQQNQQQNQQQN

SEQ ID NO: 48 - protein, 33 aa, synthetic construct
QQQQQQGGGQQQQQQGGGQQQQQQGGGQQQQQQ

SEQ ID NO: 49 - protein, 33 aa, synthetic construct
QQQQQQGGGQQQNQQGGGQQQQQQGGGQQQQQQ

SEQ ID NO: 50 - protein, 20 aa, synthetic construct
EAAAREAAAREAAAREAAAR

SEQ ID NO: 51 - protein, 20 aa, synthetic construct
GGGGSGGGGSGGGGSGGGGS

SEQ ID NO: 52 - protein, 39 aa, Homo sapiens
PEDEIAALKKEIAALKQENAALKQEIAALKKEIAALKQG

SEQ ID NO: 53 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 54 - protein, 39 aa, synthetic construct
PEDEIAALKKEIAALKQENAALKQEIAAKLEIKAAKLQG

(19) United States
(12) Patent Application Publication
(10) Pub. No.: US 2026/0036573 A1
(43) Pub. Date: Feb. 5, 2026

(54) METHODS FOR DIAGNOSING, SCREENING AND TREATING AN AMYLOID-ASSOCIATED CONDITION

(71) Applicant: STOWERS INSTITUTE FOR MEDICAL RESEARCH, Kansas City, MO (US)

(72) Inventors: Randall HALFMANN, Kansas City, MO (US); Jianzheng WU, Kansas City, MO (US)

(21) Appl. No.: 19/359,224

(22) Filed: Oct. 15, 2025

Related U.S. Application Data
(63) Continuation of application No. PCT/US2024/025718, filed on Apr. 22, 2024.
(60) Provisional application No. 63/497,652, filed on Apr. 21, 2023.
(60) Provisional application No. 63/513,658, filed on Jul. 14, 2023.

(51) Int. Cl. G01N 33/50; C12N 15/86

(52) U.S. Cl. CPC G01N 33/5091; C12N 15/86; C12N 2740/15043

(57) ABSTRACT

The present disclosure provides, inter alia, methods for preventing, treating or ameliorating the effects of an amyloid-associated condition including Huntington's disease in a subject. Also provided are methods for detecting amyloid nucleation of a protein of interest such as, e.g., the Huntingtin protein.

### CROSS REFERENCE TO RELATED APPLICATIONS

The present application is a continuation of PCT international application no. PCT/US2024/025718, filed on Apr. 22, 2024, which claims benefit of U.S. Provisional Patent Application Ser. No. 63/497,652, filed on Apr. 21, 2023, and U.S. Provisional Patent Application Ser. No. 63/513,658, filed on Jul. 14, 2023. The contents of above applications are incorporated by reference herein in their entireties.

### GOVERNMENT FUNDING

This invention was made with government support under grants no. R01GM130927 and R21AG080434, awarded by the National Institutes of Health. The government has certain rights in the invention.

### FIELD OF DISCLOSURE

The present disclosure provides, inter alia, methods for preventing, treating or ameliorating the effects of an amyloid-associated condition including Huntington's disease in a subject. Also provided are methods for detecting amyloid nucleation of a protein of interest such as, e.g., the Huntingtin protein.

### INCORPORATION BY REFERENCE OF SEQUENCE LISTING

This application contains references to amino acids and/or nucleic acid sequences that have been filed concurrently herewith as sequence listing XML file “1065334.000185-seq.xml”, file size of 50,152 bytes, created on Apr. 7, 2023. The aforementioned sequence listing is hereby incorporated by reference in its entirety pursuant to 37 C.F.R. § 1.52(e)(5).

### BACKGROUND OF THE DISCLOSURE

Amyloids are highly ordered protein aggregates with self-templating activity. This activity drives the progression of multiple incurable diseases of aging, such as Alzheimer's disease (Chiti and Dobson, 2017; Huang et al., 2019). Understanding how amyloids start, or nucleate, is therefore fundamental to preventing these diseases. However, despite decades of intense research, there still lacks a clear picture of even the gross anatomy of an amyloid nucleus.

Amyloid nuclei cannot be observed directly by any existing experimental approach. This is because unlike mature amyloid fibrils, which are stable and amenable to structural biology, nuclei are unstable by definition. Their structures do not necessarily propagate into or correspond with the structures of mature amyloids that arise from them (Auer et al., 2008; Buell, 2017; Hsieh et al., 2017; Levin et al., 2014; Liang et al., 2018; Li et al., 2010; Sil et al., 2018; Yamaguchi et al., 2005; Zanjani et al., 2020). Moreover, nucleation occurs far too infrequently, and involves far too many degrees of freedom, to simulate computationally from a naive state (Barrera et al., 2021; Kar et al., 2011; Strodel, 2021). Unlike phase separation, which primarily concerns a loss of intermolecular entropy, amyloid formation additionally involves a major loss of intramolecular entropy. That is, nucleation selects for a specific combination of backbone and side chain torsion angles (Khan et al., 2018; Vitalis and Pappu, 2011; Zhang and Schmit, 2016). As a consequence, amyloid-forming proteins can accumulate to supersaturating concentrations while remaining soluble, thereby storing potential energy that will subsequently drive their aggregation following a stochastic nucleating event (Buell, 2017; Khan et al., 2018).

Due to the improbability that the requisite increases in both density and conformational ordering will occur spontaneously at the same time (i.e. homogeneously), amyloid nucleation tends to occur heterogeneously. In other words, amyloids tend to emerge via a progression of relatively less ordered but more probable, metastable intermediates of varying stoichiometry and conformation (Auer et al., 2008; Buell, 2017; Hsieh et al., 2017; Levin et al., 2014; Liang et al., 2018; Li et al., 2010; Serio et al., 2000; Sil et al., 2018; Vekilov, 2012; Vitalis and Pappu, 2011; Yamaguchi et al., 2005; Zanjani et al., 2020). Heterogeneities divide the nucleation barrier into smaller, more probable steps, only one of which will be rate-limiting (FIG. 1A). Therefore, the occurrence of heterogeneities implies that the nature of the actual nucleus for a given amyloid can depend not only on the protein's sequence but also its concentration and cellular factors that influence its conformation (Bradley et al., 2002; Buell, 2017; Collinge and Clarke, 2007; Sanders et al., 2014; Tornquist et al., 2018).

Structural features of amyloid nucleation may be deduced by studying the effects on amyloid kinetics of rational mutations made to the polypeptide (Thakur and Wetzel, 2002). The occurrence of density heterogeneities confounds this approach, however, as they blunt the dependence of amyloid kinetics on concentration that could otherwise reveal nucleus stoichiometry (Vitalis and Pappu, 2011). Resolving this problem would require an assay that can detect nucleation events independently of amyloid growth kinetics, and which can scale to accommodate the large numbers of mutations necessary to identify sequence-structure relationships. Classic assays of in vitro amyloid assembly kinetics are poorly suited to this task, both because of their limited throughput, and because even the smallest experimentally tractable reaction volumes are too large to observe discrete aggregation events (Michaels et al., 2017).

Accordingly, there is a need for a novel technique to explore amyloid assembly kinetics, which facilitates the development of new methods for diagnosing, screening, and treating amyloid-associated conditions such as, e.g., Alzheimer's disease (AD) and Huntington's disease (HD). This disclosure is directed to meeting these and other needs.

### SUMMARY OF THE DISCLOSURE

A long-standing goal of amyloid research has been to characterize the structural basis of the rate-determining nucleating event. However, the ephemeral nature of nucleation has made this goal unachievable with existing biochemistry, structural biology, and computational approaches. The present disclosure addresses that limitation for polyglutamine (polyQ), a polypeptide sequence that causes Huntington's and other amyloid-associated neurodegenerative diseases when its length exceeds a characteristic threshold. To identify essential features of the polyQ amyloid nucleus, a direct intracellular reporter of self-association was used to quantify nucleation frequencies as a function of concentration, conformational templates, and rational polyQ sequence permutations. It was found that nucleation of pathologically expanded polyQ involves segments of three glutamine (Q) residues at every other position. The present disclosure demonstrates using molecular simulations that this pattern encodes a four-stranded steric zipper with interdigitated Q side chains. Once formed, the zipper poisoned its own growth by engaging naive polypeptides on orthogonal faces, in a fashion characteristic of polymer crystals with intramolecular nuclei. The present disclosure further shows that preemptive oligomerization of polyQ inhibits amyloid nucleation. By uncovering the physical nature of the rate-limiting event for polyQ aggregation in cells, the present disclosure elucidates the molecular etiology of polyQ diseases.

Accordingly, one aspect of the present disclosure is directed to a method for preventing, treating or ameliorating the effects of an amyloid-associated condition in a subject. This method comprises: (a) identifying a target protein whose aggregation causes the amyloid-associated condition in the subject; and (b) treating the subject by modifying the target protein to induce preemptive oligomerization thereof.

Another aspect of the present disclosure is directed to a method for preventing, treating or ameliorating the effects of a polyglutamine (polyQ) disease in a subject. This method comprises: (a) identifying a target protein whose aggregation causes the polyQ disease in the subject; (b) obtaining a biological sample from the subject and screening for expanded cytosine-adenine-guanine (CAG) repeats encoding a long polyQ tract in the target protein; (c) identifying the subject as having high risk to develop the polyQ disease, if the long polyQ tract in the target protein contains a number of glutamines exceeding a pathogenic threshold for the polyQ disease; and (d) treating the identified subject by modifying the target protein to induce preemptive oligomerization thereof.

Another aspect of the present disclosure is directed to a method for preventing, treating or ameliorating the effects of Huntington's disease (HD) in a subject. This method comprises: (a) obtaining a biological sample from the subject and screening for expanded cytosine-adenine-guanine (CAG) repeats encoding a long polyQ tract in the Huntingtin protein; (b) identifying the subject as having high risk to develop HD, if the long polyQ tract in the Huntingtin protein contains 36 or more glutamines; and (c) treating the identified subject by modifying the Huntingtin protein to induce preemptive oligomerization thereof.

Another aspect of the present disclosure is directed to a method for preventing, treating or ameliorating the effects of an amyloid-associated neurodegenerative disease in a subject. This method comprises: (a) identifying an amyloid-forming protein whose aggregation causes the amyloid-associated neurodegenerative disease in the subject; (b) obtaining a biological sample from the subject and screening for the amyloid-forming protein; (c) identifying the subject as having high risk to develop the amyloid-associated neurodegenerative disease, if the amyloid-forming protein exists in the subject; and (d) treating the identified subject by modifying the amyloid-forming protein to induce preemptive oligomerization thereof.

A further aspect of the present disclosure is directed to a method for preventing, treating or ameliorating the effects of a condition associated with TAR DNA-binding protein 43 (TDP-43) in a subject. This method comprises: (a) obtaining a biological sample from the subject and screening for TDP-43; (b) identifying the subject as having high risk to develop the condition, if TDP-43 exists in the subject; and (c) treating the identified subject by modifying TDP-43 to induce preemptive oligomerization thereof.

An additional aspect of the present disclosure relates to a method for detecting amyloid nucleation of a protein of interest. This method comprises: (a) generating a first yeast strain by (i) deleting PDR5 and ATG8 genes from a yeast strain rhy1713, and then (ii) integrating BDFP1.6:1.6 prior to the stop codon of chromosomal PGK1; (b) generating a second yeast strain by eliminating the amyloid form of Rnq1 from the first yeast strain; (c) transforming the first and second yeast strains with a plasmid encoding the protein of interest as a fusion to a photoconvertible tag; (d) incubating the transformed strains under suitable conditions and inducing them for a suitable time; (e) conducting high-throughput flow cytometry on these induced cells and collecting fluorescence signals; and (f) analyzing the collected signals to determine amyloid nucleation of the protein of interest.

Still another aspect of the present disclosure is directed to a method for constructing a biosensor cell that is used to detect aggregates of a specific protein in a biological sample. This method comprises: (a) constructing a reporter gene that encodes the specific protein fused with a fluorescent tag; (b) cloning the reporter gene onto a lentiviral transfer plasmid; (c) transfecting the lentviral transfer plasmid together with a packaging plasmid and an envelope plasmid into HEK293T cells; (d) incubating the transfected cells and collecting viral particles containing the reporter gene; (e) incubating the viral particles collected in step (d) with fresh HE293T cells; and (f) sorting for single cell clone containing the integrated reporter gene and expanding it as the biosensor cell.

In some embodiments, the fluorescent tag is one or more of a photoconvertible fluorescent tag and a self labeling fluorescent tag. In some embodiments, the fluorescent tag is one or more of mEos3.1, mEos3.2, HaloTag, SNAP-tag, TMP-tag, and fluorescence-activating and absorption shifting tag (FAST).

The present disclosure also provides biosensor cells prepared by the method disclosed herein, and the use of such biosensor cells in detecting and quantifying aggregates of a specific protein in a biological sample.

According to some aspects, the present disclosure provides a biosensor cell effective to detect aggregates of a specific protein in a biological sample, the biosensor cell comprising a reporter gene that encodes the specific protein fused with a fluorescent tag. In some embodiments, the fluorescent tag is one or more of a photoconvertible fluorescent tag and a self labeling fluorescent tag. In some embodiments, the fluorescent tag is one or more of mEos3.1, mEos3.2, HaloTag, SNAP-tag, TMP-tag, and fluorescence-activating and absorption shifting tag (FAST). In some embodiments, the biosensor cell is a mammalian cell. In some embodiments, the biosensor cell is a HEK293T cell. In some embodiments, the specific protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), and TAR DNA-binding protein 43 (TDP-43).

According to some aspects, the present disclosure provides a method for detecting aggregates of a specific protein in a biological sample comprising: contacting the biological sample with a biosensor cell as disclosed herein; and detecting and quantifying biosensor cells that have acquired increased FRET by flow cytometry to determine aggregates of the specific protein. In some embodiments, the specific protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), and TAR DNA-binding protein 43 (TDP-43).

According to some aspects, the present disclosure provides a method for constructing a biosensor cell that is used to detect aggregates of a specific protein in a biological sample, comprising: constructing a reporter gene that encodes the specific protein fused with a fluorescent tag; transporting the reporter gene inside a cell; and sorting and/or expanding cells containing the reporter gene as the biosensor cell. In some embodiments, the fluorescent tag is one or more of a photoconvertible fluorescent tag and a self labeling fluorescent tag. In some embodiments, the fluorescent tag is one or more of mEos3.1, mEos3.2, HaloTag, SNAP-tag, TMP-tag, and fluorescence-activating and absorption shifting tag (FAST). In some embodiments, the cell is a mammalian cell. In some embodiments, the cell is a HEK293T cell. In some embodiments, the transporting of the reporter gene comprises one or more of transfection, transduction, infection, or combinations thereof. In some embodiments, the transporting of the reporter gene comprises the use of a lentiviral vector. In some embodiments, the specific protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), and TAR DNA-binding protein 43 (TDP-43).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a reaction coordinate diagram schematizing the energy barriers for forming an amyloid nucleus homogeneously (left), facilitated by a density heterogeneity (middle), or facilitated by a conformational heterogeneity (right). Because amyloid involves a transition from disordered monomer to ordered multimer, the nucleation barrier (purple) results from a combination of high energy fluctuations in both density (blue) and conformation (red).

FIG. 1B shows that cellular volumes quantize amyloid nucleation. Amyloid nuclei occur at such low concentrations that fewer than one exists in the femtoliter volumes of cells. This causes amyloid formation to be rate-limited by stochastic nucleation in individual yeast cells (right) but not in the microliter volumes of conventional in vitro kinetic assays (left). Taking a population-level snapshot of the extent of protein self-assembly as a function of concentration in each cell reveals heterogeneity attributable to the nucleation barrier.

FIG. 1C provides DAmFRET plots, showing the extent of de novo self-assembly (AmFRET) as a function of protein concentration for polyglutamine (Q60) or polyasparagine (N60) in yeast cells, lacking endogenous amyloid ([pin⁻]). Cells expressing Q60 partition into distinct populations that either lack (no AmFRET) or contain (high AmFRET) amyloid. The bimodal distribution persists even among cells with the same concentration of protein, indicating that amyloid formation is rate-limited by nucleation. The nucleation barrier for N60 is so large that spontaneous amyloid formation occurs at undetectable frequencies. Insets show histograms of AmFRET values. AU, arbitrary units.

FIG. 1D shows a bar plot of the fraction of [pin⁻] cells in the AmFRET-positive population for the indicated length variants of polyQ, along with the pathologic length thresholds for polyQ tracts in the indicated proteins. Shown are means+/−SEM of biological triplicates. CACNA1A, Cav2.1; ATXN2, Ataxin-2; HTT, Huntingtin; AR, Androgen Receptor; ATXN7, Ataxin-7; ATXN1, Ataxin-1; TBP, TATA-binding protein; ATN1, Atrophin-1; ATXN3, Ataxin-3.

FIG. 1E shows a bar plot of the fraction of [pin⁻] cells in the AmFRET-positive population for the indicated sequences, showing that amyloid is inhibited when Q tracts are interrupted by a non-Q side chain at odd-numbered intervals. Shown are means+/−SEM of biological triplicates.

FIG. 2A shows a view down the axis of a local segment of all-glutamine steric zipper, or “Q zipper”, between two antiparallel two-stranded sheets. Residues with internally facing side-chains on the top layer are colored red to emphasize interdigitation and H-bonding (dashed lines) between the terminal amides and the opposing backbone.

FIG. 2B shows schema of the “odd dependency” for Q zipper formation, showing side chain arrangements along a continuous beta strand for sequences composed of tandem repeats of Qs (red) interrupted by single Ns (gray). Shading highlights contiguous stretches of Qs that would occur in a continuous beta strand. Note that the illustrated strands will not necessarily be continuous in the context of the nucleus; i.e. the nucleus may contain shorter strands connected by loops.

FIG. 2C shows schema of the tertiary contacts between two beta strands, as in a steric zipper. The zipper can be formed only when the single interrupting non-Q residue follows an odd number of Qs (e.g. Q₁N), but not when it follows an even number of Qs (e.g. Q₂N).

FIG. 2D shows molecular simulations of model Q zippers formed by a pair of two-stranded antiparallel beta sheets, wherein non-Q residues (in red) face either inward or outward. The schema are oriented so the viewer is looking down the axis between two sheets. The zipper is stable for pure polyQ (QQQQQQQ, top simulation), or when substitutions face outward (QQQNQQQ, second simulation; and QNQNQNQ, fourth simulation), but not when even a single substitution faces inward (QQQNQQQ, third simulation).

FIG. 2E shows a snapshot from the uninterrupted Q zipper simulation, showing H-bonds (black arrows) between internal extended Q side chains and the opposing backbones.

FIG. 2F shows a snapshot from the internally interrupted Q zipper simulation, illustrating that the side chain of N is too short to H-bond the opposing backbone. However, the N side chain is long enough to H-bond the opposing Q side chain (red arrow), thereby intercepting the side chain-backbone H-bond that would otherwise occur (dashed arrow) between that Q side chain and the backbone amide adjacent to the N. This leads to dissolution of the zipper.

FIG. 3A shows a schematic illustrating how sequences with bilaterally contiguous Qs (Q_{B}) can hypothetically allow for lateral growth (secondary nucleation) of Q zippers giving rise to lamellar amyloid fibers. In contrast, sequences with only unilateral contiguous Qs (Q_{U}) can form amyloids with only a single Q zipper.

FIG. 3B shows the maximum AmFRET values for the indicated sequences in [pin⁻] cells, suggesting that Q_{B} amyloids have a greater subunit density. Shown are means+/−SEM of the median AmFRET values of triplicates. ****p<0.0001; ANOVA and Dunnett's multiple comparison test.

FIG. 3C provides the densitometric analysis of SDD-AGE characterizing amyloid length distributions for the indicated Q_{U} and Q_{B} amyloids, showing that Q_{B} amyloid particles are larger. Data are representative of multiple experiments.

FIG. 3D shows the fraction of cells at intermediate AmFRET values for the indicated sequences in [pin⁻] cells, suggesting that Q_{B} amyloids grow slower. Shown are means+/−SEM of the percentage of cells between lower and upper populations, of triplicates. ****, ***, *p<0.0001, <0.001, <0.05; ANOVA.

FIG. 4A shows the fraction of [PIN⁺] cells in the AmFRET-positive population as a function of concentration for the indicated sequences. Arrows denote the population of cells with self-poisoned aggregation (inset), and the corresponding plateaus in the relationship of amyloid formation to concentration. The purple arrow highlights the sharp reduction in aggregation for Q₇N relative to Q₅N, which we attribute to enhanced poisoning as a result of intramolecular Q zipper formation. Shown are means+/−SEM of triplicates.

FIG. 4B shows the distribution of cytosolic concentrations (AFU/μm³) of Q60 in [pin⁻] cells either lacking or containing puncta, showing that the protein remains diffuse even when supersaturated relative to amyloid. Representative diffuse or punctate cells (N=31 and 26, respectively) of equivalent total concentration are shown. Scale bar: 5 μm.

FIG. 4C shows a schematic illustrating self-poisoned growth as a function of concentration for Q zippers of Q₅N and Q₇N. Conformational conversion of Q₅N to amyloid decelerates (becomes poisoned) at high concentrations, as a consequence of polypeptides interfering with each other's conversion on the templating surface. This is illustrated here by the red trace and inset showing entangled, partially ordered polypeptides on the axial surface. The presence of contralaterally contiguous Qs in Q₇N exacerbates poisoning at low concentrations, as illustrated here by the blue trace and inset showing partially-ordered species immobilized with bilateral zippers. Growth resumes at high concentrations through relatively disordered deposition with subsequent slow ordering.

FIG. 4D shows the graph of spline fits of AmFRET values for the indicated sequences in [PIN⁺] cells. The upper and lower populations of Q₃N and Q₅N were treated separately due to the extreme persistence of the low population for these sequences. The red dashed lines denote these are subpopulations of the same samples. The ability of amyloid to grow at low concentrations fell sharply with the onset of bilateral contiguity at Q₆N and then gradually increased with higher q values. Shown are means+/−SEM of triplicates.

FIG. 4E shows a histogram of AmFRET values for Q₇N-expressing cells transitioning from the low to high populations (boxed region from DAmFRET plot in FIG. 9D) upon translation inhibition for six hours following 18 hours of expression. Shown are means+/−95% CI of biological triplicates. Blocking new protein synthesis prior to analysis causes AmFRET to rise, whether by cycloheximide or lactimidomycin (p<0.01, <0.05, respectively, Dunnett's test).

FIG. 5A shows the fraction of cells in the AmFRET-positive population when expressing Q₃N with the indicated non-amyloidogenic fusions. Shown are means+/−SEM of triplicates. ***, **p<0.001, <0.01; ANOVA.

FIG. 5B shows the fraction of cells in the AmFRET-positive population (with higher AmFRET than that of the oligomer itself) when expressing the indicated protein fused to proteins with the indicated stoichiometry. Shown are means+/−SEM of triplicates. ***p<0.001; t-test.

FIG. 5C shows the schema, DAmFRET plots, and quantitation of amyloid formation by a synthetic minimal polyQ amyloid-forming sequence. Q side chains in the nucleating zipper are colored red, while those necessary for growth of the zipper—which requires lateral propagation due to its short length—are colored blue. The three G3 loops are represented by dashed gray lines; the actual topology of the loops may differ. Mutating a single Q to N blocks amyloid formation. Shown are means+/−SEM of triplicates. **p<0.01; t-test.

FIG. 6A shows DAmFRET plots of polyQ length variants. Shown are representative plots of biological triplicates.

FIG. 6B shows DAmFRET plots of polypeptides composed of tandem repeats of q (subscripted) Qs separated by an N for a total length of 60 residues. Plots are representative of biological triplicates.

FIG. 6C shows DAmFRET plots of polypeptides composed of tandem repeats of the indicated N-rich sequences, for a total length of 60 residues, showing negligible nucleation in the absence of a conformational template. Note that because the nominal pattern repeats, “Q₁N₂”, “Q₁N₃”, and “Q₁N₄” are synonymous to “N₂Q₁”, “N₃Q₁”, and “N₄Q₁”, respectively. Plots are representative of biological triplicates.

FIG. 6D shows DAmFRET plots of polypeptides composed of tandem repeats of the indicated sequences, for a total length of 60 residues, showing that Q₃X and Q₅X have a greater amyloid propensity than Q₄X regardless of the identity of X. Labels above the boxed regions of the [PIN⁺] Q₄N, Q₄G, and Q₄H plots indicate the percentage of cells in the high-FRET region, revealing rare but significant nucleation for the latter (p=0.046 versus Q₄N, one-tailed T-test). Plots are representative of biological triplicates.

FIG. 7A shows molecular simulations of model Q zippers formed by a pair of two-stranded antiparallel beta sheets, containing a single serine residue (QQQSQQQ) per strand. The structure is unstable when the S side chains face inward (top), but not when the S side chains face outward (bottom).

FIG. 7B shows simulations of model steric zippers formed by a pair of four-stranded antiparallel beta sheets, containing a single asparagine (top) or serine (bottom) residue per strand. The structure proved less stable in the case of asparagine.

FIG. 7C shows that as a consequence of the N side chain's interception of the opposing Q side chain's H-bond, the Q is no longer anchored in the outstretched configuration and sterically interferes with the ordering of adjacent Qs. This effect propagates through the zipper, resulting in its dissolution.

FIG. 7D shows that as for N, the side chain of S is too short to H-bond with the opposing backbone. Unlike for N, however, the S side chain is also too short to intercept the opposing Q side chain's H-bond, allowing the Q to H-bond (black arrow) the backbone amide adjacent to the S. Therefore, whereas Q zippers cannot accommodate internal N residues, they can accommodate sparse internal S residues.

FIG. 7E shows a schematic demonstrating how a polar clasp (red dashed line) would preclude Q zipper formation.

FIG. 7F shows schema and frequencies of polar clasps occurring between two unilaterally adjacent Q side chains (left) or a unilaterally adjacent N and Q side chain (right) within a QQQNQQQ peptide, simulated either with (top) or without (bottom) the backbone restrained in a beta conformation. The bar graphs show that polar clasps between Q and N occur less frequently than between Q and Q, indicating that the mechanism of Q zipper destabilization by N side chains cannot be attributed to polar clasps.

FIG. 7G shows schema and lifetimes of H-bonds between exterior stacked (axially adjacent) Q side chains (top) or N and Q side chains (bottom) in the Q zipper simulated in FIG. 2D, showing no difference in stabilities between axial H-bonds between Q and Q/N side chains.

FIG. 8A shows the DAmFRET plots of the indicated sequence variants with either a C-terminal (EAAAR)₄-mEos3.1 fusion, as used throughout this work, an N-terminal mEos3.1-(EAAAR)₄ fusion, or a C-terminal (GGGGS)₄-mEos3.1 fusion, showing that the sequence-specific differences in relative steady state AmFRET levels do not depend on the linker or terminus fused. Plots are representative of biological triplicates.

FIG. 8B shows fluorescence images of SDD-AGE gels showing the size distributions of SDS-resistant complexes of the indicated mEos3.1-tagged proteins. Left: raw data quantified in FIG. 3C. Right: Additional Q₃X, Q₄X, Q₅X proteins, showing that Q_{U} amyloids are consistently smaller than other amyloids, such as those of Q60 and Sup35 PrD (which only nucleates in [PIN⁺] cells). The solid line shows where the image was spliced, although all lanes are from the same gel. Lysates were normalized by fluorescence to within 50% of each other prior to loading.

FIG. 8C shows histograms of AmFRET values for the indicated gates (at the respective approximate EC50s) for the indicated sequences. The brown gate on the histograms shows the percentage of transitioning cells.

FIG. 9A shows DAmFRET plots of Q₅N, Q60, and Q₈N acquired using imaging flow cytometry, showing gates at high expression for both high- or low-AmFRET populations. Insets show from left to right the distribution of donor, FRET, and acceptor fluorescence, respectively, in representative cells from each gate.

FIG. 9B shows a schematic of the rate of polymer crystallization as a function of length, showing a sharp deceleration when the polymer length is equally compatible with either of two polymorphs. Adapted from (Ungar et al., 2005).

FIG. 9C shows DAmFRET plots of [pin⁻] cells expressing unilateral contiguity variants of the Q₃N base sequence, showing that at least five unilaterally contiguous glutamines (see schematic) are required for de novo nucleation of single long Q zipper amyloids. Plots are representative of biological triplicates.

FIG. 9D shows DAmFRET plots of bilateral contiguity variants (Q₄N₂, Q₆N₂, Q₈N₂), showing that at least six bilaterally contiguous Qs are required for de novo amyloid formation. Numbers indicate the percentage of cells in the high-FRET boxed region, revealing significant nucleation by Q₆N₂ (p=0.0004, T-test).

FIG. 9E shows DAmFRET plots of Q₇N in [pin⁻] cells treated as indicated for six hours prior to analysis. The boxed region was used to compute histograms of AmFRET in FIG. 4E.

FIG. 10A shows the DAmFRET plots of cells expressing Q₃N (length 60) with the indicated appendage (length 30). Plots are representative of biological triplicates.

FIG. 10B shows DAmFRET plots of [pin⁻] and [PIN⁻] cells expressing the indicated sequences either unfused (“monomer”) or fused to oDi (“dimer”) for FTH1 (24-mer). Plots are representative of biological triplicates.

FIG. 10C shows DAmFRET plots of cells expressing Q60 either with or without oDi and with the indicated linkers and termini of the fusion. Plots are representative of biological triplicates.

FIG. 10D shows quantification of the data in C), showing that oDi reduces Q60 nucleation irrespective of the linker and terminus it is fused to. Shown are means+/−SEM. **, ***, ****p<0.01, <0.001, <0.0001; t-test.

FIG. 10E shows DAmFRET plots of cells expressing Q60 either with or without oDi or a monomeric mutant of oDi (oDi{X}). Plots are representative of biological triplicates.

FIG. 10F shows quantification of the data in E), showing that the monomerizing mutation eliminates the amyloid-inhibiting effect of oDi. Shown are means+/−SEM. ****p<0.0001; t-test.

FIG. 10G shows DAmFRET plots of [pin⁻] cells expressing a synthetic minimal polyQ amyloid-forming sequence, or the same sequence with the tenth Q mutated to N. Quantitation is the same as in FIG. 5C. Plots are representative of biological triplicates.

FIG. 11 shows DAmFRET plots for the C-terminal prion-like domain of TDP-43 (“CTD”, residues 273-414) expressed in yeast with an N-terminal fusion to mEos3.1.

FIG. 12A shows the results of validation for the use of the biosensor cell lines to detect the presence of TDP-43 LCS (311-414) in a given sample using DAmFRET.

FIG. 12B shows the results of validation for the use of the biosensor cell lines to detect the presence of tau in a given sample using DAmFRET.

FIG. 13 shows the increased sensitivity of TDP-43 LCS (267-414) and (274-414) to other amyloids when mutated in the region from R361 to P363, as indicated by the increased fraction of cells in the high AmFRET population and an absence of overlapping low AmFRET population. [PIN⁺] denotes the presence of Rnq1 amyloids; [pin⁻] denotes the absence of amyloids.

FIG. 14 shows the use of fluorescence-activating and absorption shifting tag (FAST) to detect amyloid formation in cells. FAST is a small protein tag that specifically covalently binds fluorogen molecules. Fluorogens are dyes that only become fluorescent upon activation, in this case by FAST. Top: We constructed a DAmFRET plasmid expressing far red (fr)FAST in place of mEos3.1. Inclusion of the fluorogens TFLime and TFPoppy in the culture media allows for mixed labeling of the frFAST-tagged protein with a FRET donor and acceptor, respectively. Middle: DAmFRET plot of cells expressing frFAST alone, showing only a modest acquisition of FRET at high expression levels indicating the tag itself is largely monomeric. Bottom: DAmFRET plot of cells expressing the prion-like protein ASC fused to frFAST, revealing a bimodal distribution comprising both low- and high-FRET populations of cells qualitatively similar to that achieved by ASC fused to mEos3.1, confirming that self-labeling protein fusions allow for robust amyloid detection in cells.

### DETAILED DESCRIPTION OF THE DISCLOSURE

Polyglutamine (polyQ) is an amyloid-forming sequence common to eukaryotic proteomes (Mier et al., 2020). In humans, it is responsible for nine invariably fatal neurodegenerative diseases, the most prevalent of which is Huntington's disease. Cells exhibiting polyQ pathology, whether in Huntington's disease patients, tissue culture, organotypic brain slices, or animal models, die independently and stochastically with a constant frequency (Clarke et al., 2000; Linsley et al., 2019). Two observations suggest that this emerges directly from the amyloid nucleation barrier. First, polyQ aggregation itself occurs stochastically in cells (Colby et al., 2006; Kakkar et al., 2016; Sinnige et al., 2021). Second, polyQ disease onset and progression are determined almost entirely by an intramolecular change, specifically, a genetically encoded expansion in the number of sequential glutamines beyond a protein-specific threshold of approximately 36 residues (Lieberman et al., 2019). Unlike other amyloid-associated diseases (Book et al., 2018; Kim et al., 2020; Selkoe and Hardy, 2016), polyQ disease severity generally does not worsen with gene dosage (Cubo et al., 2019; Lee et al., 2019; Wexler et al., 1987), implying that the rate-determining step in neuronal death occurs in a fixed minor fraction of the polyglutamine molecules. It is unclear how, and why, those molecules differ from the bulk. Hence, therapeutic progress against polyQ diseases awaits detailed knowledge of the very earliest steps of amyloid formation.

Heterogeneities may be responsible for amyloid-associated proteotoxicity. Partially ordered species accumulate during the early stages of amyloid aggregation by all well-characterized pathological amyloids, but generally do not occur (or less so) during the formation of functional amyloids (Otzen and Riek, 2019). In the case of pathologically expanded polyQ, or the huntingtin protein containing pathologically expanded polyQ, amyloid-associated oligomers have been observed in vitro, in cultured cells, and in the brains of patients (Auer et al., 2008; Hsieh et al., 2017; Legleiter et al., 2010; Levin et al., 2014; Liang et al., 2018; Li et al., 2010; Olshina et al., 2010; Sathasivam et al., 2010; Sil et al., 2018; Takahashi et al., 2008; Vitalis and Pappu, 2011; Yamaguchi et al., 2005; Zanjani et al., 2020), and are likely culprits of proteotoxicity (Kim et al., 2016; Leitman et al., 2013; Lu and Palacino, 2013; Matlahov and van der Wel, 2019; Miller et al., 2011; Takahashi et al., 2008; Wetzel, 2020). In contrast, mature amyloid fibers are increasingly viewed as benign or even protective (Arrasate et al., 2004; Kim et al., 2016; Leitman et al., 2013; Lu and Palacino, 2013; Matlahov and van der Wel, 2019; Takahashi et al., 2008; Wetzel, 2020). The fact that polyQ disease kinetics is governed by amyloid nucleation, but the amyloids themselves appear not to be responsible, presents a paradox. A structural model of the polyQ amyloid nucleus can be expected to resolve this paradox by illuminating a path for the propagation of conformational order into distinct multimeric species that either cause or mitigate toxicity.

PolyQ is an ideal polypeptide with which to deduce the physical nature of a pathologic amyloid nucleus. It has zero complexity, which profoundly simplifies the design and interpretation of sequence variants. Unlike other pathogenic protein amyloids, which occur as different structural polymorphs each plausibly with their own structural nucleus, polyQ amyloids have an invariant core structure under different assembly conditions and in the context of different flanking domains (Boatz et al., 2020; Galaz-Montoya et al., 2021; Lin et al., 2017; Schneider et al., 2011). This core contains antiparallel beta-sheets (Buchanan et al., 2014; Matlahov and van der Wel, 2019; Schneider et al., 2011), while most other amyloids, including all other Q-rich amyloids, have a parallel beta-sheet core (Eisenberg and Jucker, 2012; Margittai and Langen, 2008). This suggests that amyloid nucleation and pathogenesis of polyQ may follow from an ability to spontaneously acquire a specific nucleating conformation. Any variant that decelerates nucleation can therefore be interpreted with respect to its effect on that conformation.

The present disclosure reveals that the polyQ amyloid nucleus is a steric zipper encoded by a pattern of approximately twelve Q residues in a single polypeptide molecule. It was found that the clinical length threshold for polyQ disease is the minimum length that can encompass the pattern. Consistent with a monomeric nucleus, amyloid formation occurred less frequently at high concentrations or when polyQ was expressed in oligomeric form. It was further found that the nucleus promotes its own kinetic arrest by templating competing dimensions of Q zipper ordering—both along the amyloid axis and orthogonally to it. This leads to the accumulation of partially ordered aggregates, which can inhibit amyloid nucleation through preemptive oligomerization of polyQ.

Accordingly, one aspect of the present disclosure is directed to a method for preventing, treating or ameliorating the effects of an amyloid-associated condition in a subject. This method comprises: (a) identifying a target protein whose aggregation causes the amyloid-associated condition in the subject; and (b) treating the subject by modifying the target protein to induce preemptive oligomerization thereof.

As used herein, the term “amyloid-associated condition” refers to a condition, disorder or disease characterized by a particular protein or polypeptide that aggregates in an ordered manner to form insoluble amyloid fibrils. In some embodiments, the amyloid-associated condition is a neurodegenerative disease selected from the group consisting of amyotrophic lateral sclerosis (ALS), frontotemporal dementia (FTD), dementia with Lewy bodies, familial British dementia, familial Danish dementia, Alzheimer's disease (AD), limbic predominant age-related TDP-43 encephalopathy (LATE), Parkinson's disease (PD), spongiform encephalopathies, and a polyglutamine (polyQ) disease. In some embodiments, the polyQ disease is selected from the group consisting of spinocerebellar ataxia type 1 (SCA1), SCA2, SCA6, SCA7, SCA17, Machado-Joseph disease (MJD/SCA3), Huntington's disease (HD), dentatorubral pallidoluysian atrophy (DRPLA), and spinal and bulbar muscular atrophy, X-linked 1 (SMAX1/SBMA).

In some embodiments, the target protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), TAR DNA-binding protein 43 (TDP-43), Cav2.1, ataxin-2, Huntingtin, androgen receptor, ataxin-7, ataxin-1, TATA-binding protein, atrophin-1, and ataxin-3.

In some embodiments, the amyloid-associated condition is a non-neurodegenerative disease selected from the group consisting of AL amyloidosis, AA amyloidosis, familial Mediterranean fever, senile systemic amyloidosis, familial amyloidotic polyneuropathy, hemodialysis-related amyloidosis, apoAI amyloidosis, apoAII amyloidosis, apoAIV amyloidosis, Finnish hereditary amyloidosis, lysozyme amyloidosis, fibrinogen amyloidosis, Icelandic hereditary cerebral amyloid angiopathy, Type II diabetes, medullary carcinoma of the thyroid, atrial amyloidosis, hereditary cerebral haemorrhage with amyloidosis, pituitary prolactinoma, injection-localized amyloidosis, aortic medial amyloidosis, hereditary lattice corneal dystrophy, corneal amylodosis associated with trichiasis, cataract, calcifying epithelial odontogenic tumors, pulmonary alveolar proteinosis, inclusion-body myositis, and cutaneous lichen amyloidosis.

In some embodiments, the target protein is selected from the group consisting of immunoglobulin light chains or fragments, serum amyloid A protein, transthyretin, β2-microglobulin, apolipoprotein AI, apolipoprotein AII, apolipoprotein AIV, gelsolin, lysozyme, fibrinogen α-chain, cystatin C, amylin, calcitonin, atrial natriuretic factor, amyloid β peptide, prolactin, insulin, medin, kerato-epithelin, lactoferrin, γ-crystallins, lung surfactant protein C, heterogeneous nuclear ribonucleoprotein D like (hnRNPDL), receptor-interacting serine/threonine-protein kinase 1 (RIPK1), receptor-interacting serine/threonine-protein kinase 3 (RIPK3), galectin-7, S100 calcium-binding protein A8 (S100A8), S100 calcium-binding protein A9 (S100A9), semenogelin 1 (SEM1), leukocyte chemotactic factor 2 (LECT-2), and keratins.

In some embodiments, the treatment is carried out in vivo or ex vivo. As used herein, “in vivo treatment” refers to direct delivery of therapeutics such as genetic material either intravenously (e.g., through an IV) or locally to a specific organ (e.g., direct injection) to target cells that remain inside a person's body, while “ex vivo treatment” refers to a process of removing specific cells from a person, treating them (e.g., genetically altering) in a laboratory, and then transplanting them back into the person. A therapeutic of the present disclosure may be administered to a subject via oral, parenteral or other administration in any appropriate manner such as, e.g., intraperitoneal, subcutaneous, topical, intradermal, sublingual, intramuscular, intravenous, intraarterial, intrathecal, or intralymphatic. A therapeutic of the present disclosure may be encapsulated or otherwise protected against gastric or other secretions, if desired. Further, such agent may be administered in conjunction with other treatments.

In some embodiments, the modification of the target protein is pre- or post-translational. As used herein, “pre-translational modification” of a protein refers to modifications that affect expression of the protein's encoding gene. Modifications that affect gene expression can occur at any point along the path from packaged DNA to protein expression. For example, alterations to chromosome structure or changes in chromosome copy number can affect expression at a chromosomal level. Epigenetic chromatin modifications, including histone acetylation and histone methylation, can also alter gene expression levels. At the DNA level, mutations in DNA sequence or DNA methylation can change the nature of the genes expressed as well as their level of expression. RNA transcript copy number also affects protein expression. The term “post-translational modification” or “PTM” refers to amino acid side chain modification in some proteins after their biosynthesis. There are more than 400 different types of PTMs affecting many aspects of protein functions. Some major PTMs include, e.g., phosphorylation, acetylation ubiquitination, methylation, and amidation.

In some embodiments, the modification of the target protein is carried out by modifying its encoding gene. In some embodiments, for example, the encoding gene of the target protein is modified by mutagenesis or gene fusion. As used herein, the term “mutagenesis” refers to a process by which the genetic information of an organism is changed by the production of a mutation. It can be achieved experimentally using laboratory procedures such as, e.g., site-directed mutagenesis (also called site-specific mutagenesis or oligonucleotide-directed mutagenesis), which is a molecular biology method that is used to make specific and interntional mutating changes to the DNA sequence of a gene and any gene products. Examples of specific DNA alterations include insertions, deletions and substitutions. The term “gene fusion”, as used herein, refers to a process to generate fusion gene, which is a hybrid gene formed from two previously independent genes. It can be achieved by, e.g., translocation, interstitial deletion, or chromosomal inversion.

In some embodiments, the modification of the target protein is carried out by introducing into the subject a transgene expressing a homo-oligomerizing moiety that is fused to a binding protein against the target protein. The transgene can be introduced by any techniques known or to be developed in the art. For example, in some embodiments, the transgene is introduced by an adeno-associated virus (AAV). As used herein, “adeno-associated viruses” or “AAV” are small (approximately 26 nm in diameter) replication-defective, nonenveloped viruses and have linear single-stranded DNA (ssDNA) genome of approximately 4.8 kilobases (kb). They belong to the genus Dependoparvovirus in the family of Parvoviridae. They are widely used for creating viral vectors for gene therapy, and for the creation of isogenic human disease models. Gene therapy vectors using AAV can infect both dividing and quiescent cells and persist in an extrachromosomal state without integrating into the genome of the host cell.

In some embodiments, the binding protein is an intrabody. As used herein, an “intrabody” is an antibody that works within the cell to bind to an intracellular protein. It is modified for intracellular localization.

In some embodiments, the modification of the target protein is carried out by administering to the subject an effective amount of a small molecule binder against the target protein. In some embodiments, the small molecule binder comprise a self-reactive moiety that makes the small molecule binder multivalent.

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc. In some embodiments, the subject is a mammal selected from the group consisting of humans, primates, farm animals, and domestic animals. In some embodiments, the subject is a human. In some embodiments of the present disclosure, the phrase “a subject” means a subject having an amyloid-associated neurodegenerative disease such as, e.g., Alzheimer's disease, or having a high risk to develop such disease.

Another aspect of the present disclosure is directed to a method for preventing, treating or ameliorating the effects of a polyglutamine (polyQ) disease in a subject. This method comprises: (a) identifying a target protein whose aggregation causes the polyQ disease in the subject; (b) obtaining a biological sample from the subject and screening for expanded cytosine-adenine-guanine (CAG) repeats encoding a long polyQ tract in the target protein; (c) identifying the subject as having high risk to develop the polyQ disease, if the long polyQ tract in the target protein contains a number of glutamines exceeding a pathogenic threshold for the polyQ disease; and (d) treating the identified subject by modifying the target protein to induce preemptive oligomerization thereof.

As used herein, a “polyglutamine disease” or “polyQ disease” refers to a group of neurodegenerative disorders caused by expanded cytosine-adenine-guanine (CAG) repeats encoding a long polyQ tract in the respective proteins. Polyglutamine (PolyQ)-related diseases are dominant late-onset genetic disorders that are manifested by progressive neurodegeneration, leading to behavioral and physical impairments. Polyglutamine disease family encompasses at least nine heritable disorders, including, e.g., Huntington disease (HD) and the spinocerebellar ataxias SCA1, SCA2, SCA3, SCA6, SCA7 and SCA17. In some embodiments, the polyQ disease is selected from the group consisting of spinocerebellar ataxia type 1 (SCA1), SCA2, SCA6, SCA7, SCA17, Machado-Joseph disease (MJD/SCA3), Huntington's disease (HD), dentatorubral pallidoluysian atrophy (DRPLA), and spinal and bulbar muscular atrophy, X-linked 1 (SMAX1/SBMA). In some embodiments, the target protein is selected from the group consisting of Cav2.1, ataxin-2, Huntingtin, androgen receptor, ataxin-7, ataxin-1, TATA-binding protein, atrophin-1, and ataxin-3.

In some embodiments, the treatment is carried out in vivo or ex vivo. In some embodiments, the modification of the target protein is pre- or post-translational. In some embodiments, the modification of the target protein is carried out by modifying its encoding gene. In some embodiments, the encoding gene of the target protein is modified by mutagenesis or gene fusion.

As defined above, in the context of the present disclosure, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc. In some embodiments, the subject is a mammal selected from the group consisting of humans, primates, farm animals, and domestic animals. In some embodiments, the subject is a human. In some embodiments of the present disclosure, the phrase “a subject” means a subject having a polyglutamine (polyQ) disease such as, e.g., Huntington's disease, or having a high risk to develop such disease.

Except SCA6, the polyQ expansion of which occurs in the context of a membrane protein, for the other eight ployQ described above, the polyQ expansion occurs in the context of soluble proteins localized to the cytosol or the nucleus of neuronal cells. Interestingly, each of these disorders is characterized by a unique pathogenic polyQ-expansion threshold. Once that threshold is exceeded, the age of disease onset is inversely proportional to polyQ expansion length. Pathogenic polyQ-expansion threshold of soluble proteins in the polyglutamine disease family varies from 32Q (for SCA2) to 54Q (for SCA3). In some embodiments, the pathogenic threshold of the polyQ disease is in the range of 32 to 54 glutamines in the long polyQ tract of the target protein.

In some embodiments, the modification of the target protein is carried out by mutating its encoding gene that results in a mutant target protein having the amino acid pattern of Q_{A}X_{B} in its long polyQ tract, wherein X is any amino acid other than Q and wherein A and B are independently positive integers. In some embodiments, A is an even number. In some embodiments, A=2 or 4 and B=1. In some embodiments, A=3 or 5 and B=1.

Another aspect of the present disclosure is directed to a method for preventing, treating or ameliorating the effects of Huntington's disease (HD) in a subject. This method comprises: (a) obtaining a biological sample from the subject and screening for expanded cytosine-adenine-guanine (CAG) repeats encoding a long polyQ tract in the Huntingtin protein; (b) identifying the subject as having high risk to develop HD, if the long polyQ tract in the Huntingtin protein contains 36 or more glutamines; and (c) treating the identified subject by modifying the Huntingtin protein to induce preemptive oligomerization thereof.

In some embodiments, the treatment is carried out in vivo or ex vivo. In some embodiments, the modification of the Huntingtin protein is pre- or post-translational. In some embodiments, the modification of the Huntingtin protein is carried out by modifying the HTT gene. In some embodiments, the HTT gene is modified by mutagenesis or gene fusion.

In some embodiments, the subject is a mammal selected from the group consisting of humans, primates, farm animals, and domestic animals. In some embodiments, the subject is a human. In some embodiments of the present disclosure, the phrase “a subject” means a subject having Huntington's disease, or having a high risk to develop Huntington's disease.

In some embodiments, the modification of the Huntingtin protein is carried out by mutating the HTT gene that results in a mutant Huntingtin protein having the amino acid pattern of Q_{A}X_{B} in its long polyQ tract, wherein X is any amino acid other than Q and wherein A and B are independently positive integers. In some embodiments, A is an even number. In some embodiments, A=2 or 4 and B=1. In some embodiments, A=3 or 5 and B=1. In some embodiments, X is an amino acid selected from the group consisting of asparagine (N), glycine (G), alanine (A), serine(S), and histidine (H). In some embodiments, X is asparagine (N). In some embodiments, the amino acid pattern of Q_{A}X_{B} is Q₅N₁.

In some embodiments, modification of the Huntingtin protein is carried out by fusing the HTT gene with a sequence encoding a homo-oligomeric protein. As used herein, a “homo-oligomeric protein” refers to proteins that have a natural tendency to self-associate into homo-oligomeric protein complexes, also termed homomers, which are composed of two or more identical subunits. In some embodiments, the homo-oligomeric protein is a coiled coil dimer or human FTH1. As used herein, a “coiled coil” refers to a structure formed when two or more α-helices self-assemble by winding around each other to form a left-handed supercoil. Although dimers, trimers, and tetramers are the most common structures, larger coiled-coils of up to seven helices can now be prescriptively designed. Human FTH1 protein (ferritin heavy chain) is a ferroxidase enzyme that is encoded by the human Fth1 gene. It is composed of 24 subunits of the heavy ferritin chains.

Another aspect of the present disclosure is directed to a method for preventing, treating or ameliorating the effects of an amyloid-associated neurodegenerative disease in a subject. This method comprises: (a) identifying an amyloid-forming protein whose aggregation causes the amyloid-associated neurodegenerative disease in the subject; (b) obtaining a biological sample from the subject and screening for the amyloid-forming protein; (c) identifying the subject as having high risk to develop the amyloid-associated neurodegenerative disease, if the amyloid-forming protein exists in the subject; and (d) treating the identified subject by modifying the amyloid-forming protein to induce preemptive oligomerization thereof.

In some embodiments, the amyloid-forming protein is wild type. In some embodiments, the amyloid-forming protein comprises at least one amyloid-promoting mutation.

In some embodiments, the amyloid-associated neurodegenerative disease is a selected from the group consisting of amyotrophic lateral sclerosis (ALS), frontotemporal dementia (FTD), dementia with Lewy bodies, familial British dementia, familial Danish dementia, Alzheimer's disease (AD), limbic predominant age-related TDP-43 encephalopathy (LATE), spongiform encephalopathies, and Parkinson's disease (PD).

In some embodiments, the amyloid-forming protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), and TAR DNA-binding protein 43 (TDP-43).

As used herein, an “amyloid-promoting mutation” refers to a mutation in a target protein that results in irreversible amyloid aggregation and disease. All mutation sites described below in this disclosure correspond to their locations in respective human gene. In some embodiments, the amyloid-forming protein is β-amyloid precursor protein (APP) and the at least one amyloid-promoting mutation is selected from the group consisting of K670_M671delinsNL, A673T, A673V, D678N, D678H, E682K, K687Q, L688V, A692G, E693K, E693Q, E693G, E693del, D694N, L705V, T714A, T714I, V715M, V715A, I716F, V717L, V717I, V717F, V717G, T719N, M722K, L723P, and combinations thereof.

In some embodiments, the amyloid-forming protein is tau and the at least one amyloid-promoting mutation is selected from the group consisting of R5L, G55R, K257T, I260V, L266V, G272V, G273R, N279K, L284R, N296D, N296del, N296H, P301T, P301S, P301L, G303V, S305I, S305N, K317M, K317N, S320F, P332S, G335S, G335A, Q336R, Q336H, V337M, E342V, S352L, S356T, P364S, G366R, K369I, E372G, G389R, P397S, R406W, N410H, T427M, S320Y, S385R, and combinations thereof.

In some embodiments, the amyloid-forming protein is α-synuclein and the at least one amyloid-promoting mutation is selected from the group consisting of A30P, E46K, H50Q, G51D, A53E, A53T, and combinations thereof.

In some embodiments, the amyloid-forming protein is TDP-43 and the at least one amyloid-promoting mutation is selected from the group consisting of G294A, G294V, G295S, A315T, Q331K, M337V, M337P, Q343R, N345K, R361S, R361RT, E362S, P363A, P363V, P363G, P363H, N390D, N390S, and combinations thereof.

In some embodiments, the treatment is carried out in vivo or ex vivo. In some embodiments, the modification of the amyloid-forming protein is pre- or post-translational. In some embodiments, the modification of the amyloid-forming protein is carried out by modifying its encoding gene. In some embodiments, the encoding gene of the amyloid-forming protein is modified by mutagenesis or gene fusion. In some embodiments, the gene fusion is carried out by fusing the encoding gene of the amyloid-forming protein with a sequence encoding a homo-oligomeric protein. In some embodiments, the homo-oligomeric protein is a coiled coil dimer or human FTH1.

In some embodiments, the subject is a mammal selected from the group consisting of humans, primates, farm animals, and domestic animals. In some embodiments, the subject is a human. In some embodiments of the present disclosure, the phrase “a subject” means a subject having an amyloid-associated neurodegenerative disease such as, e.g., Alzheimer's disease, or having a high risk to develop such disease.

A further aspect of the present disclosure is directed to a method for preventing, treating or ameliorating the effects of a condition associated with TAR DNA-binding protein 43 (TDP-43) in a subject. This method comprises: (a) obtaining a biological sample from the subject and screening for TDP-43; (b) identifying the subject as having high risk to develop the condition, if TDP-43 exists in the subject; and (c) treating the identified subject by modifying TDP-43 to induce preemptive oligomerization thereof.

In some embodiments, the condition associated with TDP-43 is selected from the group consisting of amyotrophic lateral sclerosis (ALS), ALS related cognitive/behavioural impairment (ALS-ci/bi), multiple system proteinopathy-A familial disorder (MSP), frontotemporal lobar degeneration (FTLD), limbic-predominant agerelated TDP-43 encephalopathy (LATE), cerebral age-related TDP-43 with sclerosis (CARTS), Perry disease, facial onset sensory and motor neuronopathy (FOSMN), sporadic inclusion body myositis (sIBM), Alzheimer's disease (AD), dementia with Lewy bodies, Parkinson's disease (PD), Huntington's disease (HD), chronic traumatic encephalopathy (CTE), primary progressive aphasia (PSP), corticobasal degeneration (CBD), argyrophilic grain disease (AGD), and combinations thereof.

In some embodiments, the treatment is carried out in vivo or ex vivo. In some embodiments, the modification of TDP-43 is pre- or post-translational.

In some embodiments, the modification of TDP-43 is carried out by modifying the TARDBP gene. In some embodiments, the TARDBP gene is modified by gene fusion. In some embodiments, the modification of TDP-43 is carried out by fusing the TARDBP gene with a sequence encoding a homo-oligomeric protein. In some embodiments, the homo-oligomeric protein is a coiled coil dimer or human FTH1.

In some embodiments, TDP-43 is wild type. In some embodiments, TDP-43 comprises at least one amyloid-promoting mutation. In some embodiments, the at least one amyloid-promoting mutation is selected from the group consisting of G294A, G294V, G295S, A315T, Q331K, M337V, M337P, Q343R, N345K, R361S, R361RT, E362S, P363A, P363V, P363G, P363H, N390D, N390S, and combinations thereof.

In some embodiments, the subject is a mammal selected from the group consisting of humans, primates, farm animals, and domestic animals. In some embodiments, the subject is a human. In some embodiments of the present disclosure, the phrase “a subject” means a subject having amyotrophic lateral sclerosis (ALS), or having a high risk to develop ALS.

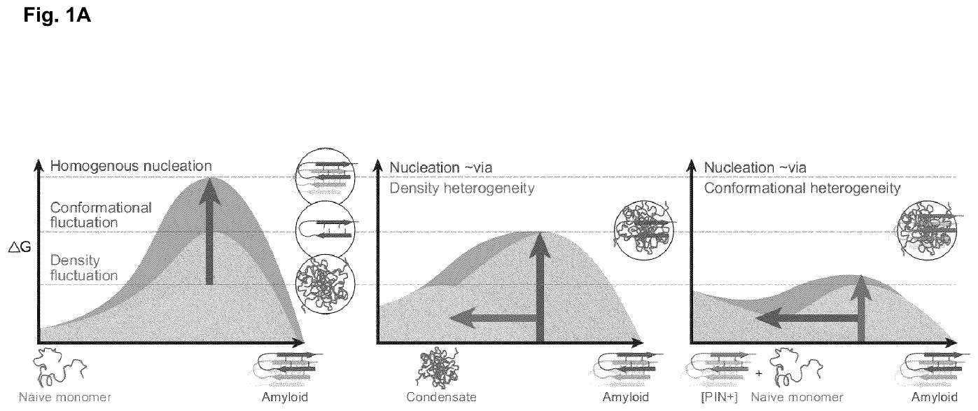

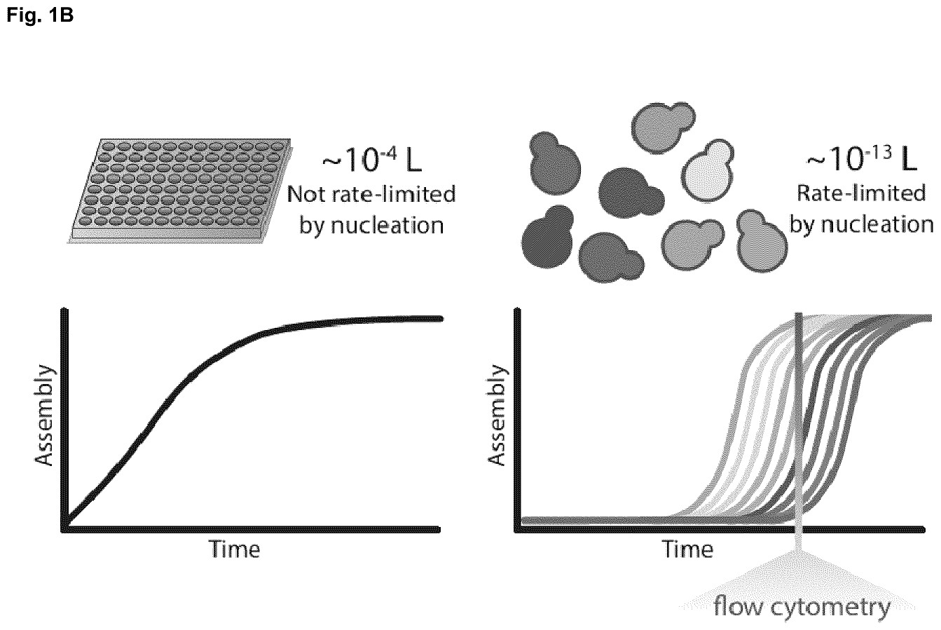

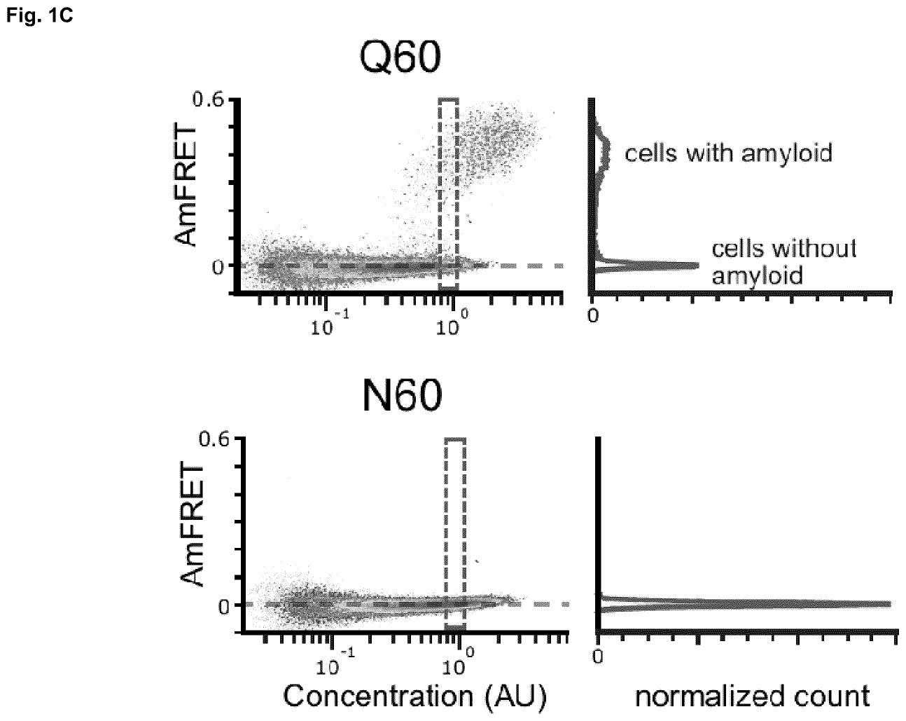

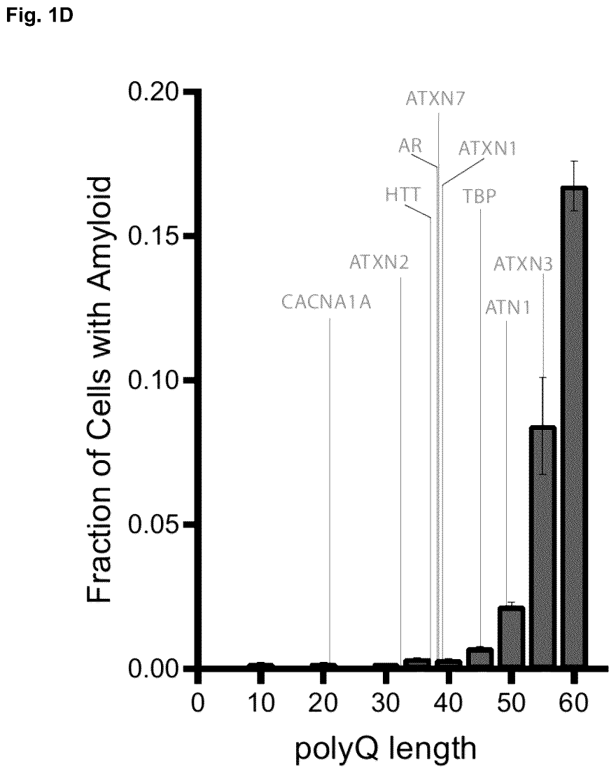

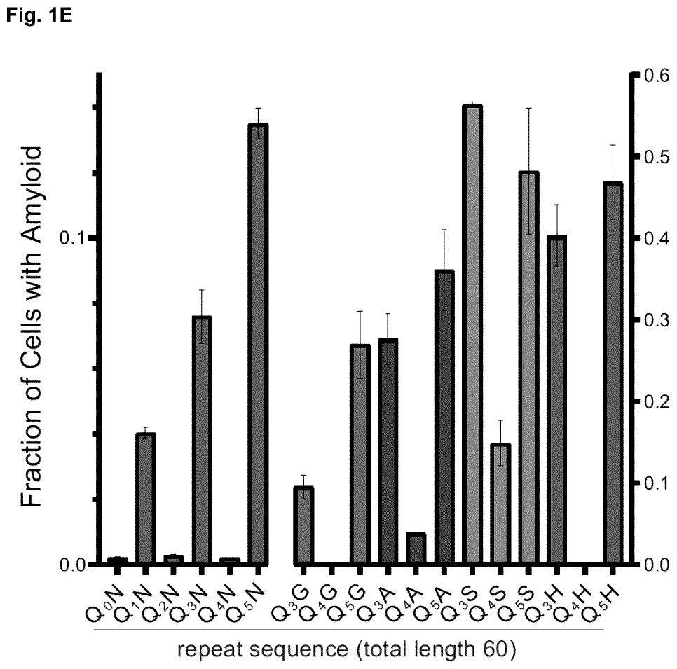

The present disclosure also provides an assay to circumvent the limitations in classic assays of in vitro amyloid assembly kinetics (Khan et al., 2018; Posey et al., 2021; Venkatesan et al., 2019). This novel assay, named Distributed Amphifluoric FRET (DAmFRET), uses a photoconvertible fusion tag and high throughput flow cytometry to treat living cells as femtoliter-volume test tubes, thereby providing the large numbers of independent reaction vessels of exceptionally small volume that are required to discriminate independent nucleation events under physiological conditions (FIG. 1B). Compared to conventional reaction volumes for protein self-assembly assays, the budding yeast cells employed in DAmFRET increase the dependence of amyloid formation on nucleation by nine orders of magnitude (the difference in volumes), allowing amyloid to form in the same nucleation-limited fashion as it does in afflicted neurons (Colby et al., 2006; Wetzel, 2006). DAmFRET employs a high-variance expression system to induce a hundred-fold range of concentrations of the protein of interest. By taking a single snapshot of the protein's extent of self-association in each cell, at each concentration, at a point in time appropriate for the kinetics of amyloid formation (hours), DAmFRET probes the existence and magnitude of critical density fluctuations that may govern nucleation.

The yeast system additionally allows for orthogonal experimental control over the critical conformational fluctuation. This is because yeast cells normally contain exactly one cytosolic amyloid species—a prion state formed by the low abundance Q-rich endogenous protein, Rnq1 (Kryndushkin et al., 2013; Nizhnikov et al., 2014). The prion state can be gained or eliminated experimentally to produce cells whose sole difference is whether the Rnq1 protein does ([PIN⁺]) or does not ([pin⁻]) populate an amyloid conformation (Derkatch et al., 2001). [PIN⁺] serves as a partial template for amyloid formation by compositionally similar proteins including polyQ (Alexandrov et al., 2008; Duennwald et al., 2006a; Meriin et al., 2002; Serpionov et al., 2015). Known as cross-seeding, this phenomenon is analogous to, but much less efficient than, amyloid elongation by molecularly identical species (Keefer et al., 2017; Khan et al., 2018; Serio, 2018). By evaluating nucleation frequencies as a function of concentration in both [pin⁻] and [PIN⁺] cells, this new assay can uncouple the two components of the nucleation barrier and thereby relate specific sequence features to the nucleating conformation. Sequence changes that are specific to the sequence-encoded nucleus will more strongly influence nucleation in [pin⁻] cells than in [PIN⁺] cells.

Accordingly, an additional aspect of the present disclosure relates to a method for detecting amyloid nucleation of a protein of interest. This method comprises: (a) generating a first yeast strain by (i) deleting PDR5 and ATG8 genes from a yeast strain rhy1713, and then (ii) integrating BDFP1.6:1.6 prior to the stop codon of chromosomal PGK1; (b) generating a second yeast strain by eliminating the amyloid form of Rnq1 from the first yeast strain; (c) transforming the first and second yeast strains with a plasmid encoding the protein of interest as a fusion to a photoconvertible tag; (d) incubating the transformed strains under suitable conditions and inducing them for a suitable time; (e) conducting high-throughput flow cytometry on these induced cells and collecting fluorescence signals; and (f) analyzing the collected signals to determine amyloid nucleation of the protein of interest.

A similar system has also been implemented in human cell lines, e.g., the HEK293T cells. Accordingly, still another aspect of the present disclosure is directed to a method for constructing a biosensor cell that is used to detect aggregates of a specific protein in a biological sample. This method comprises: (a) constructing a reporter gene that encodes the specific protein fused with a fluorescent tag; (b) cloning the reporter gene onto a lentiviral transfer plasmid; (c) transfecting the lentviral transfer plasmid together with a packaging plasmid and an envelope plasmid into HEK293T cells; (d) incubating the transfected cells and collecting viral particles containing the reporter gene; (e) incubating the viral particles collected in step (d) with fresh HE293T cells; and (f) sorting for single cell clone containing the integrated reporter gene and expanding it as the biosensor cell. In some embodiments, the fluorescent tag is one or more of a photoconvertible fluorescent tag and a self labeling fluorescent tag. In some embodiments, the fluorescent tag is one or more of mEos3.1, mEos3.2, HaloTag, SNAP-tag, TMP-tag, and fluorescence-activating and absorption shifting tag (FAST).

In some embodiments, the specific protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), and TAR DNA-binding protein 43 (TDP-43).

The present disclosure also provides a biosensor cell prepared by the method disclosed herein. Also provided is a method for detecting aggregates of a specific protein in a biological sample, comprising: incudating the sample with a biosensor cell disclosed herein; and detecting and quantifying biosensor cells that have acquired increased FRET by flow cytometry to determine aggregates of the specific protein.

In some embodiments, the specific protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), and TAR DNA-binding protein 43 (TDP-43).

The present disclosure further provides a method for diagnosing or screening a subject for risks of developing an amyloid-associated condition. This method comprises: (a) obtaining a biological sample from the subject; (b) identifying a protein from the sample that is related to the condition and transforming its encoding gene into the yeast or cell culture systems disclosed herein; and (c) determining the risks of developing the condition in the subject based on the amyloid nucleation kinetics of the protein. In some embodiments, the condition-related protein can be modified and tested for its amyloid nucleation kinetics to determine the effect of a personalized treatment.

The DAmFRET technique may be performed directly in cells derived from a subject, which allows for determining probability of developing certain diseases in the subject's own genetic context. Accordingly, the present disclosure also provides a method for diagnosing or screening a subject for risks of developing an amyloid-associated condition. This method comprises: (a) obtaining a biological sample from the subject; (b) deriving a cell line from the subject; (c) transforming a gene encoding an aggregation-prone protein into the cells; and (d) determining the risks of developing the condition in the subject based on the amyloid nucleation kinetics of the protein. In some embodiments, the condition-related protein can be modified and tested for its amyloid nucleation kinetics to determine the effect of a personalized treatment.

### Additional Definitions

The term “amino acid” means naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. An “amino acid analog” means compounds that have the same basic chemical structure as a naturally occurring amino acid, e.g., a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Imino acids such as, e.g., proline, are also within the scope of “amino acid” as used here. An “amino acid mimetic” means a chemical compound that has a structure that is different from the general chemical structure of an amino acid, but that functions similarly to a naturally occurring amino acid.

As used herein, the terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymers.

“Nucleic acid” or “oligonucleotide” or “polynucleotide” used herein means at least two nucleotides covalently linked together. Many variants of a nucleic acid may be used for the same purpose as a given nucleic acid. Thus, a nucleic acid also encompasses substantially identical nucleic acids and complements thereof.

Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be synthesized as a single stranded molecule or expressed in a cell (in vitro or in vivo) using a synthetic gene. Nucleic acids may be obtained by chemical synthesis methods or by recombinant methods.

The nucleic acid may also be an RNA such as an mRNA, tRNA, short hairpin RNA (shRNA), short interfering RNA (siRNA), double-stranded RNA (dsRNA), transcriptional gene silencing RNA (ptgsRNA), Piwi-interacting RNA, pri-miRNA, pre-miRNA, micro-RNA (miRNA), or anti-miRNA.

As used herein, a “biological sample” means a biological specimen, which may be a bodily fluid or a tissue. Biological samples include, for example, whole blood, serum, plasma, cerebro-spinal fluid, leukocytes or leukocyte subtype cells (e.g. neutrophils, basophils, and eosinophils, lymphocytes, monocytes, macrophages), fibroblast sample, olfactory neuron sample, and tissues from the central nervous system, such as the cortex (e.g., dorsolateral PFC) and hippocampus, and cells previously exposed to the CNS environment, such as dendritic cells trafficked from the brain, or other immune or other cell types (Mohamed-M G et al., 2014). Examples of preferred biological samples include, e.g., a blood sample, a biopsy sample, a plasma sample, a saliva sample, a tissue sample, a serum sample, a tear sample, a sweat sample, a skin sample, a cell sample, a hair sample, an excretion sample, a waste sample, a bodily fluid sample, a nail sample, a cheek swab, a cheek cell sample, or a mucous sample. In some embodiments, the biological sample can be a tissue section or a biopsy from dorsolateral PFC, blood, or other appropriate bodily fluid.

As used herein, the terms “prevent,” “preventing,” or “prevention,” and grammatical variations thereof refer to the prophylactic treatment of a subject in need thereof.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

In the present disclosure, an “effective amount” or “therapeutically effective amount” of an agent is an amount of such an agent that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of an agent according to the disclosure will be that amount of the agent, which is the lowest dose effective to produce the desired effect with no or minimal side effects. The effective dose of an agent according to the present disclosure may be administered as two, three, four, five, six or more sub-doses, administered separately at appropriate intervals throughout the day.

The terminology used herein is for the purpose of describing particular embodiments only and is not intended to be limiting. As used in the specification and the appended claims, the singular forms “a,” “an,” and “the” include plural referents unless the context clearly dictates otherwise.

The following examples are provided to further illustrate the methods of the present disclosure. These examples are illustrative only and are not intended to limit the scope of the disclosure in any way.

### EXAMPLES

### Example 1

### Materials and Methods

### Plasmid and Strain Construction

ORFs were codon optimized for expression in S. cerevisiae, synthesized, and cloned into vector V08 (Khan et al., 2018) by Genscript (Piscataway, NJ, USA). See Table 1 for all full-length protein sequences.

TABLE 1
List of protein sequences.

| | | Extra | | | | Insertd | | | | |
|---|---|---|---|---|---|---|---|---|---|---|
| Plasmid | Annotated | N-Term | | N-Term | N-Term | Peptide | | C-Term | | Extra |
| Name | Sequence | Tag | Linker | Tag | Linker | cSequene | Linker | Tag | Linker | Tag |
| rhx0 | mEos3. | | | | | | | | | |
| 935 | 1 | | | | | | | | | |
| rhx0 | Sup35 | | | | | MSDSN | EAAARE | mEos3. | | |
| 974 | PrD | | | | | QGNNQ | AAAR | 1 | | |
| | | | | | | QNYQQ | EAAARE | | | |
| | | | | | | YSQNG | AAAR | | | |
| | | | | | | NQQQ | (SEQ ID | | | |
| | | | | | | GNNRY | NO: 50) | | | |
| | | | | | | Q | | | | |
| | | | | | | GYQAY | | | | |
| | | | | | | NAQAQ | | | | |
| | | | | | | PAGGY | | | | |
| | | | | | | YQNYQ | | | | |
| | | | | | | GYSGY | | | | |
| | | | | | | QQGGY | | | | |
| | | | | | | QQYNP | | | | |
| | | | | | | DAGYQ | | | | |
| | | | | | | QQYNP | | | | |
| | | | | | | QGGYQ | | | | |
| | | | | | | QYNPQ | | | | |
| | | | | | | GGYQQ | | | | |
| | | | | | | QFNPQ | | | | |
| | | | | | | GGRGN | | | | |
| | | | | | | YKNFN | | | | |
| | | | | | | YNNNL | | | | |
| | | | | | | QGYQA | | | | |
| | | | | | | GFQPQ | | | | |
| | | | | | | SQGMS | | | | |
| | | | | | | LNDFQ | | | | |
| | | | | | | KQQKQ | | | | |
| | | | | | | (SEQ ID | | | | |
| | | | | | | NO: 1) | | | | |
| rhx1 | Q85 | | | | | QQQQ | EAAARE | mEos3. | | |
| 177a | | | | | | QQQQ | AAAR | 1 | | |
| | | | | | | QQ | EAAARE | | | |
| | | | | | | QQQQ | AAAR | | | |
| | | | | | | QQQQ | (SEQ ID | | | |
| | | | | | | QQ | NO: 50) | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | Q (SEQ | | | | |
| | | | | | | ID NO: | | | | |
| | | | | | | 2) | | | | |
| rhx1 | Q20 | | | | | QQQQ | EAAARE | mEos3. | | |
| 177d | | | | | | QQQQ | AAAR | 1 | | |
| | | | | | | QQ | EAAARE | | | |
| | | | | | | QQQQ | AAAR | | | |
| | | | | | | QQQQ | (SEQ ID | | | |
| | | | | | | QQ | NO: 50) | | | |
| | | | | | | (SEQ ID | | | | |
| | | | | | | NO: 3) | | | | |
| rhx1e | Q30 | | | | | QQQQ | EAAARE | mEos3. | | |
| 177 | | | | | | QQQQ | AAAR | 1 | | |
| | | | | | | QQ | EAAARE | | | |
| | | | | | | QQQQ | AAAR | | | |
| | | | | | | QQQQ | (SEQ ID | | | |
| | | | | | | QQ | NO: 50) | | | |
| | | | | | | QQQQ | | | | |
| | | | | | | QQQQ | | | | |
| | | | | | | QQ | | | | |
| | | | | | | (SEQ ID | | | | |
| | | | | | | NO: 4) | | | | |
| rhx1 | Q70 | | | | | QQQQ | EAAARE | mEos3. | | |
| 177f | | | | | | QQQQ | AAAR | 1 | | |
| | | | | | | QQ | EAAARE | | | |
| | | | | | | QQQQ | AAAR | | | |
| | | | | | | QQQQ | (SEQ ID | | | |
| | | | | | | QQ | NO: 50) | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | (SEQ ID | | | | |
| | | | | | | NO: 5) | | | | |
| rhx1 | Q10 | | | | | QQQQ | EAAARE | mEos3. | | |
| 177g | | | | | | QQQQ | AAAR | 1 | | |
| | | | | | | QQ | EAAARE | | | |
| | | | | | | (SEQ ID | AAAR | | | |
| | | | | | | NO: 6) | (SEQ ID | | | |
| | | | | | | | NO: 50) | | | |
| rhx1 | Q55 | | | | | QQQQ | EAAARE | mEos3. | | |
| 177h | | | | | | QQQQ | AAAR | 1 | | |
| | | | | | | QQ | EAAARE | | | |
| | | | | | | QQQQ | AAAR | | | |
| | | | | | | QQQQ | (SEQ ID | | | |
| | | | | | | QQ | NO: 50) | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | Q (SEQ | | | | |
| | | | | | | NO: | | | | |
| | | | | | | 7) | | | | |
| rhx1 | Q140 | | | | | QQQQ | EAAARE | mEos3. | | |
| 177j | | | | | | QQQQ | AAAR | 1 | | |
| | | | | | | QQ | EAAARE | | | |
| | | | | | | QQQQ | AAAR | | | |
| | | | | | | QQQQ | (SEQ ID | | | |
| | | | | | | QQ | NO: 50) | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | (SEQ ID | | | | |
| | | | | | | NO: 8) | | | | |
| rhx1 | Q35 | | | | | QQQQ | EAAARE | mEos3. | | |
| 177l | | | | | | QQQQ | AAAR | 1 | | |
| | | | | | | QQ | EAAARE | | | |
| | | | | | | QQQQ | AAAR | | | |
| | | | | | | QQQQ | (SEQ ID | | | |
| | | | | | | QQ | NO: 50) | | | |
| | | | | | | QQQQ | | | | |
| | | | | | | QQQQ | | | | |
| | | | | | | QQ | | | | |
| | | | | | | QQQQ | | | | |
| | | | | | | Q (SEQ | | | | |
| | | | | | | ID NO: | | | | |
| | | | | | | 9) | | | | |
| rhx1 | Q40 | | | | | QQQQ | EAAARE | mEos3. | | |
| 177m | | | | | | QQQQ | AAAR | 1 | | |
| | | | | | | QQ | EAAARE | | | |
| | | | | | | QQQQ | AAAR | | | |
| | | | | | | QQQQ | (SEQ ID | | | |
| | | | | | | QQ | NO: 50) | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | (SEQ ID | | | | |
| | | | | | | NO: 10) | | | | |
| rhx1 | Q45 | | | | | QQQQ | EAAARE | mEos3. | | |
| 177n | | | | | | QQQQ | AAAR | 1 | | |
| | | | | | | QQ | EAAARE | | | |
| | | | | | | QQQQ | AAAR | | | |
| | | | | | | QQQQ | (SEQ ID | | | |
| | | | | | | QQ | NO: 50) | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | Q (SEQ | | | | |
| | | | | | | ID NO: | | | | |
| | | | | | | 11) | | | | |
| rhx1 | Q50 | | | | | QQQQ | EAAARE | mEos3. | | |
| 177o | | | | | | QQQQ | AAAR | 1 | | |
| | | | | | | QQ | EAAARE | | | |
| | | | | | | QQQQ | AAAR | | | |
| | | | | | | QQQQ | (SEQ ID | | | |
| | | | | | | QQ | NO: 50) | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | (SEQ ID | | | | |
| | | | | | | NO: 12) | | | | |
| rhx2 | Q₃N | | | | | QQQN | EAAARE | mEos3. | | |
| 602 | | | | | | QQQN | AAAR | 1 | | |
| | | | | | | QQ | EAAARE | | | |
| | | | | | | QNQQ | AAAR | | | |
| | | | | | | QNQQ | (SEQ ID | | | |
| | | | | | | QN | NO: 50) | | | |
| | | | | | | QQQN | | | | |
| | | | | | | QQQN | | | | |
| | | | | | | QQ | | | | |
| | | | | | | QNQQ | | | | |
| | | | | | | QNQQ | | | | |
| | | | | | | QN | | | | |
| | | | | | | QQQN | | | | |
| | | | | | | QQQN | | | | |
| | | | | | | QQ | | | | |
| | | | | | | QNQQ | | | | |
| | | | | | | QNQQ | | | | |
| | | | | | | QN | | | | |
| | | | | | | (SEQ ID | | | | |
| | | | | | | NO: 13) | | | | |
| rhx2 | Q₄N | | | | | QQQQ | EAAARE | mEos3. | | |
| 603 | | | | | | NQQQ | AAAR | 1 | | |
| | | | | | | QN | EAAARE | | | |
| | | | | | | QQQQ | AAAR | | | |
| | | | | | | NQQQ | (SEQ ID | | | |
| | | | | | | QN | NO: 50) | | | |
| | | | | | | QQQQ | | | | |
| | | | | | | NQQQ | | | | |
| | | | | | | QN | | | | |
| | | | | | | QQQQ | | | | |
| | | | | | | NQQQ | | | | |
| | | | | | | QN | | | | |
| | | | | | | QQQQ | | | | |
| | | | | | | NQQQ | | | | |
| | | | | | | QN | | | | |
| | | | | | | QQQQ | | | | |
| | | | | | | NQQQ | | | | |
| | | | | | | QN | | | | |
| | | | | | | (SEQ ID | | | | |
| | | | | | | NO: 14) | | | | |
| rhx2 | Q₅N | | | | | QQQQ | EAAARE | mEos3. | | |
| 604 | | | | | | QNQQ | AAAR | 1 | | |
| | | | | | | QQ | EAAARE | | | |
| | | | | | | QNQQ | AAAR | | | |
| | | | | | | QQQN | (SEQ ID | | | |
| | | | | | | QQ | NO: 50) | | | |
| | | | | | | QQQN | | | | |
| | | | | | | QQQQ | | | | |
| | | | | | | QN | | | | |
| | | | | | | QQQQ | | | | |
| | | | | | | QNQQ | | | | |
| | | | | | | QQ | | | | |
| | | | | | | QNQQ | | | | |
| | | | | | | QQQN | | | | |
| | | | | | | QQ | | | | |
| | | | | | | QQQN | | | | |
| | | | | | | QQQQ | | | | |
| | | | | | | QN | | | | |
| | | | | | | (SEQ ID | | | | |
| | | | | | | NO: 15) | | | | |
| rhx2 | Q₇N | | | | | QQQQ | EAAARE | mEos3. | | |
| 605 | | | | | | QQQN | AAAR | 1 | | |
| | | | | | | QQ | EAAARE | | | |
| | | | | | | QQQQ | AAAR | | | |
| | | | | | | QNQQ | (SEQ ID | | | |
| | | | | | | QQ | NO: 50) | | | |
| | | | | | | QQQN | | | | |
| | | | | | | QQQQ | | | | |
| | | | | | | QQ | | | | |
| | | | | | | QNQQ | | | | |
| | | | | | | QQQQ | | | | |
| | | | | | | QN | | | | |
| | | | | | | QQQQ | | | | |
| | | | | | | QQQN | | | | |
| | | | | | | QQ | | | | |
| | | | | | | QQQQ | | | | |
| | | | | | | QNQQ | | | | |
| | | | | | | QQ | | | | |
| | | | | | | (SEQ ID | | | | |
| | | | | | | NO: 16) | | | | |
| rhx2 | Q₉N | | | | | QQQQ | EAAARE | mEos3. | | |
| 606 | | | | | | QQQQ | AAAR | 1 | | |
| | | | | | | QN | EAAARE | | | |
| | | | | | | QQQQ | AAAR | | | |
| | | | | | | QQQQ | (SEQ ID | | | |
| | | | | | | QN | NO: 50) | | | |
| | | | | | | QQQQ | | | | |
| | | | | | | QQQQ | | | | |
| | | | | | | QN | | | | |
| | | | | | | QQQQ | | | | |
| | | | | | | QQQQ | | | | |
| | | | | | | QN | | | | |
| | | | | | | QQQQ | | | | |
| | | | | | | QQQQ | | | | |
| | | | | | | QN | | | | |
| | | | | | | QQQQ | | | | |
| | | | | | | QQQQ | | | | |
| | | | | | | QN | | | | |
| | | | | | | (SEQ ID | | | | |
| | | | | | | NO: 17) | | | | |
| rhx2 | N60/Q₀N | | | | | NNNNN | EAAARE | mEos3. | | |
| 681 | | | | | | NNNNN | AAAR | 1 | | |
| | | | | | | NNNNN | EAAARE | | | |
| | | | | | | NNNNN | AAAR | | | |
| | | | | | | NNNNN | (SEQ ID | | | |
| | | | | | | NNNNN | NO: 50) | | | |
| | | | | | | NNNNN | | | | |
| | | | | | | NNNNN | | | | |
| | | | | | | NNNNN | | | | |
| | | | | | | NNNNN | | | | |
| | | | | | | NNNNN | | | | |
| | | | | | | NNNNN | | | | |
| | | | | | | (SEQ ID | | | | |
| | | | | | | NO: 18) | | | | |
| rhx2 | Q60 | | | | | QQQQ | EAAARE | mEos3. | | |
| 682 | | | | | | QQQQ | AAAR | 1 | | |
| | | | | | | QQ | EAAARE | | | |
| | | | | | | QQQQ | AAAR | | | |
| | | | | | | QQQQ | (SEQ ID | | | |
| | | | | | | QQ | NO: 50) | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | (SEQ ID | | | | |
| | | | | | | NO: 19) | | | | |
| rhx2 | Q₁N | | | | | QNQNQ | EAAARE | mEos3. | | |
| 683 | | | | | | NQNQN | AAAR | 1 | | |
| | | | | | | QNQNQ | EAAARE | | | |
| | | | | | | NQNQN | AAAR | | | |
| | | | | | | QNQNQ | (SEQ ID | | | |
| | | | | | | NQNQN | NO: 50) | | | |
| | | | | | | QNQNQ | | | | |
| | | | | | | NQNQN | | | | |
| | | | | | | QNQNQ | | | | |
| | | | | | | NONQN | | | | |
| | | | | | | QNQNQ | | | | |
| | | | | | | NQNQN | | | | |
| | | | | | | (SEQ ID | | | | |
| | | | | | | NO: 20) | | | | |
| rhx2 | Q₂N | | | | | QQNQ | EAAARE | mEos3. | | |
| 684 | | | | | | QNQQN | AAAR | 1 | | |
| | | | | | | Q | EAAARE | | | |
| | | | | | | QNQQN | AAAR | | | |
| | | | | | | QQNQ | (SEQ ID | | | |
| | | | | | | Q | NO: 50) | | | |
| | | | | | | NQQNQ | | | | |
| | | | | | | QNQQN | | | | |
| | | | | | | QQNQ | | | | |
| | | | | | | QNQQN | | | | |
| | | | | | | Q | | | | |
| | | | | | | QNQQN | | | | |
| | | | | | | QQNQ | | | | |
| | | | | | | Q | | | | |
| | | | | | | NQQNQ | | | | |
| | | | | | | QNQQN | | | | |
| | | | | | | (SEQ ID | | | | |
| | | | | | | NO: 21) | | | | |
| rhx2 | Q₆N | | | | | QQQQ | EAAARE | mEos3. | | |
| 685 | | | | | | QQNQ | AAAR | 1 | | |
| | | | | | | QQ | EAAARE | | | |
| | | | | | | QQQN | AAAR | | | |
| | | | | | | QQQQ | (SEQ ID | | | |
| | | | | | | QQ | NO: 50) | | | |
| | | | | | | NQQQ | | | | |
| | | | | | | QQQN | | | | |
| | | | | | | QQ | | | | |
| | | | | | | QQQQ | | | | |
| | | | | | | NQQQ | | | | |
| | | | | | | QQ | | | | |
| | | | | | | QNQQ | | | | |
| | | | | | | QQQQ | | | | |
| | | | | | | NQ | | | | |
| | | | | | | QQQQ | | | | |
| | | | | | | QNQQ | | | | |
| | | | | | | QQ | | | | |
| | | | | | | (SEQ ID | | | | |
| | | | | | | NO: 22) | | | | |
| rhx2 | Q₁N₃ | | | | | NNNQN | EAAARE | mEos3. | | |
| 686 | | | | | | NNQNN | AAAR | 1 | | |
| | | | | | | NQNNN | EAAARE | | | |
| | | | | | | QNNNQ | AAAR | | | |
| | | | | | | NNNQN | (SEQ ID | | | |
| | | | | | | NNQNN | NO: 50) | | | |
| | | | | | | NQNNN | | | | |
| | | | | | | QNNNQ | | | | |
| | | | | | | NNNQN | | | | |
| | | | | | | NNQNN | | | | |
| | | | | | | NQNNN | | | | |
| | | | | | | QNNNQ | | | | |
| | | | | | | (SEQ ID | | | | |
| | | | | | | NO: 23) | | | | |
| rhx2 | Q₁N₄ | | | | | NNNNQ | EAAARE | mEos3. | | |
| 687 | | | | | | NNNNQ | AAAR | 1 | | |
| | | | | | | NNNNQ | EAAARE | | | |
| | | | | | | NNNNQ | AAAR | | | |
| | | | | | | NNNNQ | (SEQ ID | | | |
| | | | | | | NNNNQ | NO: 50) | | | |
| | | | | | | NNNNQ | | | | |
| | | | | | | NNNNQ | | | | |
| | | | | | | NNNNQ | | | | |
| | | | | | | NNNNQ | | | | |
| | | | | | | NNNNQ | | | | |
| | | | | | | NNNNQ | | | | |
| | | | | | | (SEQ ID | | | | |
| | | | | | | NO: 24) | | | | |
| rhx3 | Q₈N | | | | | QQQQ | EAAARE | mEos3. | | |
| 071 | | | | | | QQQQ | AAAR | 1 | | |
| | | | | | | NQ | EAAARE | | | |
| | | | | | | QQQQ | AAAR | | | |
| | | | | | | QQQN | (SEQ ID | | | |
| | | | | | | QQ | NO: 50) | | | |
| | | | | | | QQQQ | | | | |
| | | | | | | QQNQ | | | | |
| | | | | | | QQ | | | | |
| | | | | | | QQQQ | | | | |
| | | | | | | QNQQ | | | | |
| | | | | | | QQ | | | | |
| | | | | | | QQQQ | | | | |
| | | | | | | NQQQ | | | | |
| | | | | | | QQ | | | | |
| | | | | | | QQQN | | | | |
| | | | | | | QQQQ | | | | |
| | | | | | | QQ | | | | |
| | | | | | | (SEQ ID | | | | |
| | | | | | | NO: 25) | | | | |
| rhx3 | Q₃S | | | | | QQQSQ | EAAARE | mEos3. | | |
| 117 | | | | | | QQSQQ | AAAR | 1 | | |
| | | | | | | QSQQQ | EAAARE | | | |
| | | | | | | SQQQS | AAAR | | | |
| | | | | | | QQQSQ | (SEQ | | | |
| | | | | | | QQSQQ | ID NO: | | | |
| | | | | | | QSQQQ | 50) | | | |
| | | | | | | SQQQS | | | | |
| | | | | | | QQQSQ | | | | |
| | | | | | | QQSQQ | | | | |
| | | | | | | QSQQQ | | | | |
| | | | | | | SQQQS | | | | |
| | | | | | | (SEQ ID | | | | |
| | | | | | | NO: 26) | | | | |
| rhx3 | Q₄S | | | | | QQQQS | EAAARE | mEos3. | | |
| 118 | | | | | | QQQQS | AAAR | 1 | | |
| | | | | | | QQQQS | EAAARE | | | |
| | | | | | | QQQQS | AAAR | | | |
| | | | | | | QQQQS | (SEQ ID | | | |
| | | | | | | QQQQS | NO: 50) | | | |
| | | | | | | QQQQS | | | | |
| | | | | | | QQQQS | | | | |
| | | | | | | QQQQS | | | | |
| | | | | | | QQQQS | | | | |
| | | | | | | QQQQS | | | | |
| | | | | | | QQQQS | | | | |
| | | | | | | (SEQ ID | | | | |
| | | | | | | NO: 27) | | | | |
| rhx3 | Q₅S | | | | | QQQQ | EAAARE | mEos3. | | |
| 119 | | | | | | QSQQQ | AAAR | 1 | | |
| | | | | | | Q | EAAAR | | | |
| | | | | | | QSQQQ | EAAAR | | | |
| | | | | | | QQSQQ | (SEQ ID | | | |
| | | | | | | QQQSQ | NO: 50) | | | |
| | | | | | | QQQQS | | | | |
| | | | | | | QQQQ | | | | |
| | | | | | | QSQQQ | | | | |
| | | | | | | Q | | | | |
| | | | | | | QSQQQ | | | | |
| | | | | | | QQSQQ | | | | |
| | | | | | | QQQSQ | | | | |
| | | | | | | QQQS | | | | |
| | | | | | | (SEQ ID | | | | |
| | | | | | | NO: 28) | | | | |
| rhx3 | Q₃A | | | | | QQQAQ | EAAARE | mEos3. | | |
| 265 | | | | | | QQAQQ | AAAR | 1 | | |
| | | | | | | QAQQQ | EAAARE | | | |
| | | | | | | AQQQA | AAAR | | | |
| | | | | | | QQQAQ | (SEQ ID | | | |
| | | | | | | QQAQQ | NO: 50) | | | |
| | | | | | | QAQQQ | | | | |
| | | | | | | AQQQA | | | | |
| | | | | | | QQQAQ | | | | |
| | | | | | | QQAQQ | | | | |
| | | | | | | QAQQQ | | | | |
| | | | | | | AQQQA | | | | |
| | | | | | | (SEQ ID | | | | |
| | | | | | | NO: 29) | | | | |
| rhx3 | Q₄A | | | | | QQQQA | EAAARE | mEos3. | | |
| 266 | | | | | | QQQQA | AAAR | 1 | | |
| | | | | | | QQQQA | EAAARE | | | |
| | | | | | | QQQQA | AAAR | | | |
| | | | | | | QQQQA | (SEQ ID | | | |
| | | | | | | QQQQA | NO: 50) | | | |
| | | | | | | QQQQA | | | | |
| | | | | | | QQQQA | | | | |
| | | | | | | QQQQA | | | | |
| | | | | | | QQQQA | | | | |
| | | | | | | QQQQA | | | | |
| | | | | | | QQQQA | | | | |
| | | | | | | (SEQ ID | | | | |
| | | | | | | NO: 30) | | | | |
| rhx3 | Q₅A | | | | | QQQQ | EAAARE | mEos3. | | |
| 267 | | | | | | QAQQQ | AAAR | 1 | | |
| | | | | | | Q | EAAARE | | | |
| | | | | | | QAQQQ | AAAR | | | |
| | | | | | | QQAQQ | (SEQ ID | | | |
| | | | | | | QQQAQ | NO: 50) | | | |
| | | | | | | QQQQA | | | | |
| | | | | | | QQQQ | | | | |
| | | | | | | QAQQQ | | | | |
| | | | | | | Q | | | | |
| | | | | | | QAQQQ | | | | |
| | | | | | | QQAQQ | | | | |
| | | | | | | QQQAQ | | | | |
| | | | | | | QQQQA | | | | |
| | | | | | | (SEQ ID | | | | |
| | | | | | | NO: 31) | | | | |
| rhx4 | Q₃H | | | | | QQQH | EAAARE | mEos3. | | |
| 276 | | | | | | QQQH | AAAR | 1 | | |
| | | | | | | QQ | EAAARE | | | |
| | | | | | | QHQQ | AAAR | | | |
| | | | | | | QHQQ | (SEQ ID | | | |
| | | | | | | QH | NO: 50) | | | |
| | | | | | | QQQH | | | | |
| | | | | | | QQQH | | | | |
| | | | | | | QQ | | | | |
| | | | | | | QHQQ | | | | |
| | | | | | | QHQQ | | | | |
| | | | | | | QH | | | | |
| | | | | | | QQQH | | | | |
| | | | | | | QQQH | | | | |
| | | | | | | QQ | | | | |
| | | | | | | QHQQ | | | | |
| | | | | | | QHQQ | | | | |
| | | | | | | QH | | | | |
| | | | | | | QQQH | | | | |
| | | | | | | QQQH | | | | |
| | | | | | | Q | | | | |
| | | | | | | QHQ | | | | |
| | | | | | | (SEQ ID | | | | |
| | | | | | | NO: 32) | | | | |
| rhx4 | Q₄H | | | | | QQQQ | EAAARE | mEos3. | | |
| 277 | | | | | | HQQQ | AAAR | 1 | | |
| | | | | | | QH | EAAARE | | | |
| | | | | | | QQQQ | AAAR | | | |
| | | | | | | HQQQ | (SEQ ID | | | |
| | | | | | | QH | NO: 50) | | | |
| | | | | | | QQQQ | | | | |
| | | | | | | HQQQ | | | | |
| | | | | | | QH | | | | |
| | | | | | | QQQQ | | | | |
| | | | | | | HQQQ | | | | |
| | | | | | | QH | | | | |
| | | | | | | QQQQ | | | | |
| | | | | | | HQQQ | | | | |
| | | | | | | QH | | | | |
| | | | | | | QQQQ | | | | |
| | | | | | | HQQQ | | | | |
| | | | | | | QH | | | | |
| | | | | | | QQQQ | | | | |
| | | | | | | HQQQ | | | | |
| | | | | | | QH | | | | |
| | | | | | | QQQ | | | | |
| | | | | | | (SEQ ID | | | | |
| | | | | | | NO: 33) | | | | |
| rhx4 | Q₅H | | | | | QQQQ | EAAARE | mEos3. | | |
| 278 | | | | | | QHQQ | AAAR | 1 | | |
| | | | | | | QQ | EAAARE | | | |
| | | | | | | QHQQ | AAAR | | | |
| | | | | | | QQQH | (SEQ ID | | | |
| | | | | | | QQ | NO: 50) | | | |
| | | | | | | QQQH | | | | |
| | | | | | | QQQQ | | | | |
| | | | | | | QH | | | | |
| | | | | | | QQQQ | | | | |
| | | | | | | QHQQ | | | | |
| | | | | | | QQ | | | | |
| | | | | | | QHQQ | | | | |
| | | | | | | QQQH | | | | |
| | | | | | | QQ | | | | |
| | | | | | | QQQH | | | | |
| | | | | | | QQQQ | | | | |
| | | | | | | QH | | | | |
| | | | | | | QQQQ | | | | |
| | | | | | | QHQQ | | | | |
| | | | | | | QQ | | | | |
| | | | | | | QHQ | | | | |
| | | | | | | (SEQ ID | | | | |
| | | | | | | NO: 34) | | | | |
| rhx3 | Q₃G | | | | | QQQG | EAAARE | mEos3. | | |
| 453 | | | | | | QQQG | AAAR | 1 | | |
| | | | | | | QQ | EAAARE | | | |
| | | | | | | QGQQ | AAAR | | | |
| | | | | | | QGQQ | (SEQ ID | | | |
| | | | | | | QG | NO: 50) | | | |
| | | | | | | QQQG | | | | |
| | | | | | | QQQG | | | | |
| | | | | | | QQ | | | | |
| | | | | | | QGQQ | | | | |
| | | | | | | QGQQ | | | | |
| | | | | | | QG | | | | |
| | | | | | | QQQG | | | | |
| | | | | | | QQQG | | | | |
| | | | | | | 2Q | | | | |
| | | | | | | QGQQ | | | | |
| | | | | | | QGQQ | | | | |
| | | | | | | QG | | | | |
| | | | | | | (SEQ ID | | | | |
| | | | | | | NO: 35) | | | | |
| rhx3 | Q₄G | | | | | QQQQ | EAAARE | mEos3. | | |
| 454 | | | | | | GQQQ | AAAR | 1 | | |
| | | | | | | QG | EAAARE | | | |
| | | | | | | QQQQ | AAAR | | | |
| | | | | | | GQQQ | (SEQ ID | | | |
| | | | | | | QG | NO: 50) | | | |
| | | | | | | QQQQ | | | | |
| | | | | | | GQQQ | | | | |
| | | | | | | QG | | | | |
| | | | | | | QQQQ | | | | |
| | | | | | | GQQQ | | | | |
| | | | | | | 2G | | | | |
| | | | | | | QQQQ | | | | |
| | | | | | | GQQQ | | | | |
| | | | | | | QG | | | | |
| | | | | | | QQQQ | | | | |
| | | | | | | GQQQ | | | | |
| | | | | | | QG | | | | |
| | | | | | | (SEQ ID | | | | |
| | | | | | | NO: 36) | | | | |
| rhx3 | Q₅G | | | | | QQQQ | EAAARE | mEos3. | | |
| 455 | | | | | | QGQQ | AAAR | 1 | | |
| | | | | | | QQ | EAAARE | | | |
| | | | | | | QGQQ | AAAR | | | |
| | | | | | | QQQG | (SEQ ID | | | |
| | | | | | | QQ | NO: 50) | | | |
| | | | | | | QQQG | | | | |
| | | | | | | QQQQ | | | | |
| | | | | | | QG | | | | |
| | | | | | | QQQQ | | | | |
| | | | | | | QGQQ | | | | |
| | | | | | | QQ | | | | |
| | | | | | | QGQQ | | | | |
| | | | | | | QQQG | | | | |
| | | | | | | QQ | | | | |
| | | | | | | QQQG | | | | |
| | | | | | | QQQQ | | | | |
| | | | | | | QG | | | | |
| | | | | | | (SEQ ID | | | | |
| | | | | | | NO: 37) | | | | |
| rhx3 | Q60 | | mEos3. | EAAA | QQQQ | | | | | |
| 989 | | | 1 | REAA | QQQQ | | | | | |
| | | | | AR | QQ | | | | | |
| | | | | EAAA | QQQQ | | | | | |
| | | | | REAA | QQQQ | | | | | |
| | | | | AR | QQ | | | | | |
| | | | | (SEQ | QQQQ | | | | | |
| | | | | ID NO: | QQQQ | | | | | |
| | | | | 50) | QQ | | | | | |
| | | | | | QQQQ | | | | | |
| | | | | | QQQQ | | | | | |
| | | | | | QQ | | | | | |
| | | | | | QQQQ | | | | | |
| | | | | | QQQQ | | | | | |
| | | | | | QQ | | | | | |
| | | | | | QQQQ | | | | | |
| | | | | | QQQQ | | | | | |
| | | | | | 2Q | | | | | |
| | | | | | (SEQ | | | | | |
| | | | | | ID | | | | | |
| | | | | | NO: 19) | | | | | |
| rhx4 | Q₃N | | mEos3. | EAAA | QQQN | | | | | |
| 710 | | | 1 | REAA | QQQN | | | | | |
| | | | | AR | QQ | | | | | |
| | | | | EAAA | QNQQ | | | | | |
| | | | | REAA | QNQQ | | | | | |
| | | | | AR | QN | | | | | |
| | | | | (SEQ | QQQN | | | | | |
| | | | | ID NO: | QQQN | | | | | |
| | | | | 50) | QQ | | | | | |
| | | | | | QNQQ | | | | | |
| | | | | | QNQQ | | | | | |
| | | | | | QN | | | | | |
| | | | | | QQQN | | | | | |
| | | | | | QQQN | | | | | |
| | | | | | QQ | | | | | |
| | | | | | QNQQ | | | | | |
| | | | | | QNQQ | | | | | |
| | | | | | QN | | | | | |
| | | | | | (SEQ | | | | | |
| | | | | | ID | | | | | |
| | | | | | NO: 13) | | | | | |
| rhx4 | Q₇N | | mEos3. | EAAA | QQQQ | | | | | |
| 068 | | | 1 | REAA | QQQN | | | | | |
| | | | | AR | QQ | | | | | |
| | | | | EAAA | QQQQ | | | | | |
| | | | | REAA | QNQQ | | | | | |
| | | | | AR | QQ | | | | | |
| | | | | (SEQ | QQQN | | | | | |
| | | | | ID NO: | QQQQ | | | | | |
| | | | | 50) | QQ | | | | | |
| | | | | | QNQQ | | | | | |
| | | | | | QQQQ | | | | | |
| | | | | | QN | | | | | |
| | | | | | QQQQ | | | | | |
| | | | | | QQQN | | | | | |
| | | | | | QQ | | | | | |
| | | | | | QQQQ | | | | | |
| | | | | | QNQQ | | | | | |
| | | | | | QQ | | | | | |
| | | | | | (SEQ | | | | | |
| | | | | | ID | | | | | |
| | | | | | NO: 16) | | | | | |
| rhx4 | Q60 | | | | | QQQQ | GGGGS | mEos3. | | |
| 584 | | | | | | QQQQ | GGGGS | 1 | | |
| | | | | | | QQ | GGGGS | | | |
| | | | | | | QQQQ | GGGGS | | | |
| | | | | | | QQQQ | (SEQ ID | | | |
| | | | | | | QQ | NO: 51) | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | (SEQ ID | | | | |
| | | | | | | NO: 19) | | | | |
| rhx4 | Q₃N | | | | | QQQN | GGGGS | mEos3. | | |
| 585 | | | | | | QQQN | GGGGS | 1 | | |
| | | | | | | QQ | GGGGS | | | |
| | | | | | | QNQQ | GGGGS | | | |
| | | | | | | QNQQ | (SEQ ID | | | |
| | | | | | | QN | NO: 51) | | | |
| | | | | | | QQQN | | | | |
| | | | | | | QQQN | | | | |
| | | | | | | QQ | | | | |
| | | | | | | QNQQ | | | | |
| | | | | | | QNQQ | | | | |
| | | | | | | QN | | | | |
| | | | | | | QQQN | | | | |
| | | | | | | QQQN | | | | |
| | | | | | | QQ | | | | |
| | | | | | | QNQQ | | | | |
| | | | | | | QNQQ | | | | |
| | | | | | | QN | | | | |
| | | | | | | (SEQ ID | | | | |
| | | | | | | NO: 13) | | | | |
| rhx4 | Q₇N | | | | | QQQQ | GGGGS | mEos3. | | |
| 586 | | | | | | QQQN | GGGGS | 1 | | |
| | | | | | | QQ | GGGGS | | | |
| | | | | | | QQQQ | GGGGS | | | |
| | | | | | | QNQQ | (SEQ ID | | | |
| | | | | | | QQ | NO: 51) | | | |
| | | | | | | QQQN | | | | |
| | | | | | | QQQQ | | | | |
| | | | | | | QQ | | | | |
| | | | | | | QNQQ | | | | |
| | | | | | | QQQQ | | | | |
| | | | | | | QN | | | | |
| | | | | | | QQQQ | | | | |
| | | | | | | QQQN | | | | |
| | | | | | | QQ | | | | |
| | | | | | | QQQQ | | | | |
| | | | | | | QNQQ | | | | |
| | | | | | | QQ | | | | |
| | | | | | | (SEQ ID | | | | |
| | | | | | | NO: 16) | | | | |
| rhx3 | Q₃NQ₃N₂ | | | | | QQQN | | | | |
| 450 | | | | | | QQQNN | | | | |
| | | | | | | Q | | | | |
| | | | | | | QQNQ | | | | |
| | | | | | | QQNNQ | | | | |
| | | | | | | Q | | | | |
| | | | | | | QNQQ | | | | |
| | | | | | | QNNQQ | | | | |
| | | | | | | Q | | | | |
| | | | | | | NQQQN | | | | |
| | | | | | | NQQQN | | | | |
| | | | | | | QQQNN | | | | |
| | | | | | | QQQN | | | | |
| | | | | | | Q | | | | |
| | | | | | | QQNNQ | | | | |
| | | | | | | QQNQ | | | | |
| | | | | | | Q (SEQ | | | | |
| | | | | | | ID NO: | | | | |
| | | | | | | 38) | | | | |
| rhx3 | Q₃NQ₃N | | | | | QQQN | | | | |
| 451 | Q₃N₂ | | | | | QQQN | | | | |
| | | | | | | QQ | | | | |
| | | | | | | QNNQQ | | | | |
| | | | | | | QNQQ | | | | |
| | | | | | | Q | | | | |
| | | | | | | NQQQN | | | | |
| | | | | | | NQQQN | | | | |
| | | | | | | QQQN | | | | |
| | | | | | | QQQNN | | | | |
| | | | | | | Q | | | | |
| | | | | | | QQNQ | | | | |
| | | | | | | QQNQ | | | | |
| | | | | | | QQ | | | | |
| | | | | | | NNQQQ | | | | |
| | | | | | | NQQQN | | | | |
| | | | | | | (SEQ ID | | | | |
| | | | | | | NO: 39) | | | | |
| rhx3 | Q₃NQ₃N | | | | | QQQN | | | | |
| 452 | Q₃NQ₃N₂ | | | | | QQQN | | | | |
| | | | | | | QQ | | | | |
| | | | | | | QNQQ | | | | |
| | | | | | | QNNQQ | | | | |
| | | | | | | Q | | | | |
| | | | | | | NQQQN | | | | |
| | | | | | | QQQN | | | | |
| | | | | | | Q | | | | |
| | | | | | | QQNNQ | | | | |
| | | | | | | QQNQ | | | | |
| | | | | | | Q | | | | |
| | | | | | | QNQQ | | | | |
| | | | | | | QNQQ | | | | |
| | | | | | | QN | | | | |
| | | | | | | NQQQN | | | | |
| | | | | | | QQQN | | | | |
| | | | | | | Q (SEQ | | | | |
| | | | | | | ID NO: | | | | |
| | | | | | | 40) | | | | |
| rhx2 | Q₄N₂ | | | | | QQQQ | EAAARE | mEos3. | | |
| 608 | | | | | | NNQQQ | AAAR | 1 | | |
| | | | | | | Q | EAAARE | | | |
| | | | | | | NNQQQ | AAAR | | | |
| | | | | | | QNNQQ | (SEQ ID | | | |
| | | | | | | QQNNQ | NO: 50) | | | |
| | | | | | | QQQNN | | | | |
| | | | | | | QQQQ | | | | |
| | | | | | | NNQQQ | | | | |
| | | | | | | Q | | | | |
| | | | | | | NNQQQ | | | | |
| | | | | | | QNNQQ | | | | |
| | | | | | | QQNNQ | | | | |
| | | | | | | QQQNN | | | | |
| | | | | | | (SEQ ID | | | | |
| | | | | | | NO: 41) | | | | |
| rhx3 | Q₆N₂ | | | | | QQQQ | EAAARE | mEos3. | | |
| 789 | | | | | | QQNNQ | AAAR | 1 | | |
| | | | | | | Q | EAAARE | | | |
| | | | | | | QQQQ | AAAR | | | |
| | | | | | | NNQQQ | (SEQ ID | | | |
| | | | | | | Q | NO: 50) | | | |
| | | | | | | QQNNQ | | | | |
| | | | | | | QQQQ | | | | |
| | | | | | | Q | | | | |
| | | | | | | NNQQQ | | | | |
| | | | | | | QQQNN | | | | |
| | | | | | | QQQQ | | | | |
| | | | | | | QQNNQ | | | | |
| | | | | | | Q | | | | |
| | | | | | | QQQQ | | | | |
| | | | | | | NNQQQ | | | | |
| | | | | | | Q (SEQ | | | | |
| | | | | | | ID NO: | | | | |
| | | | | | | 42) | | | | |
| rhx2 | Q₈N₂ | | | | | QQQQ | EAAARE | mEos3. | | |
| 609 | | | | | | QQQQ | AAAR | 1 | | |
| | | | | | | NN | EAAARE | | | |
| | | | | | | QQQQ | AAAR | | | |
| | | | | | | QQQQ | (SEQ ID | | | |
| | | | | | | NN | NO: 50) | | | |
| | | | | | | QQQQ | | | | |
| | | | | | | QQQQ | | | | |
| | | | | | | NN | | | | |
| | | | | | | QQQQ | | | | |
| | | | | | | QQQQ | | | | |
| | | | | | | NN | | | | |
| | | | | | | QQQQ | | | | |
| | | | | | | QQQQ | | | | |
| | | | | | | NN | | | | |
| | | | | | | QQQQ | | | | |
| | | | | | | QQQQ | | | | |
| | | | | | | NN | | | | |
| | | | | | | (SEQ ID | | | | |
| | | | | | | NO: 43) | | | | |
| rhx3 | Q₁N₂ | | | | | NNQNN | EAAARE | mE | | |
| 947 | | | | | | QNNQN | AAAR | os3. | | |
| | | | | | | NQNNQ | EAAARE | 1 | | |
| | | | | | | NNQNN | AAAR | | | |
| | | | | | | QNNQN | (SEQ ID | | | |
| | | | | | | NQNNQ | NO: 50) | | | |
| | | | | | | NNQNN | | | | |
| | | | | | | QNNQN | | | | |
| | | | | | | NQNNQ | | | | |
| | | | | | | NNQNN | | | | |
| | | | | | | QNNQN | | | | |
| | | | | | | NQNNQ | | | | |
| | | | | | | (SEQ ID | | | | |
| | | | | | | NO: 44) | | | | |
| rhx4 | Q₃N₂ | | | | | QQQNN | EAAARE | mEos3. | | |
| 709 | | | | | | QQQNN | AAAR | 1 | | |
| | | | | | | QQQNN | EAAARE | | | |
| | | | | | | QQQNN | AAAR | | | |
| | | | | | | QQQNN | (SEQ ID | | | |
| | | | | | | QQQNN | NO: 50) | | | |
| | | | | | | QQQNN | | | | |
| | | | | | | QQQNN | | | | |
| | | | | | | QQQNN | | | | |
| | | | | | | QQQNN | | | | |
| | | | | | | QQQNN | | | | |
| | | | | | | QQQNN | | | | |
| | | | | | | (SEQ ID | | | | |
| | | | | | | NO: 45) | | | | |
| rhx3 | (Q₄N)₆ | | | | | QQQQ | EAAARE | mEos3. | | |
| 458 | (Q₃N)₁₅ | | | | | NQQQ | AAAR | 1 | | |
| | | | | | | QN | EAAARE | | | |
| | | | | | | QQQQ | AAAR | | | |
| | | | | | | NQQQ | (SEQ ID | | | |
| | | | | | | QN | NO: 50) | | | |
| | | | | | | QQQQ | | | | |
| | | | | | | NQQQ | | | | |
| | | | | | | QN | | | | |
| | | | | | | QQQN | | | | |
| | | | | | | QQQN | | | | |
| | | | | | | QQ | | | | |
| | | | | | | QNQQ | | | | |
| | | | | | | QNQQ | | | | |
| | | | | | | QN | | | | |
| | | | | | | QQQN | | | | |
| | | | | | | QQQN | | | | |
| | | | | | | QQ | | | | |
| | | | | | | QNQQ | | | | |
| | | | | | | QNQQ | | | | |
| | | | | | | QN | | | | |
| | | | | | | QQQN | | | | |
| | | | | | | QQQN | | | | |
| | | | | | | QQ | | | | |
| | | | | | | QNQQ | | | | |
| | | | | | | QNQQ | | | | |
| | | | | | | QN | | | | |
| | | | | | | (SEQ ID | | | | |
| | | | | | | NO: 46) | | | | |
| rhx4 | (Q₂N)₁₀ | | | | | QQNQ | EAAARE | mEos3. | | |
| 612 | (Q₃N)₁₅ | | | | | QNQQN | AAAR | 1 | | |
| | | | | | | Q | EAAARE | | | |
| | | | | | | QNQQN | AAAR | | | |
| | | | | | | QQNQ | (SEQ ID | | | |
| | | | | | | Q | NO: 50) | | | |
| | | | | | | NQQNQ | | | | |
| | | | | | | QNQQN | | | | |
| | | | | | | QQQN | | | | |
| | | | | | | QQQN | | | | |
| | | | | | | QQ | | | | |
| | | | | | | QNQQ | | | | |
| | | | | | | QNQQ | | | | |
| | | | | | | QN | | | | |
| | | | | | | QQQN | | | | |
| | | | | | | QQQN | | | | |
| | | | | | | QQ | | | | |
| | | | | | | QNQQ | | | | |
| | | | | | | QNQQ | | | | |
| | | | | | | QN | | | | |
| | | | | | | QQQN | | | | |
| | | | | | | QQQN | | | | |
| | | | | | | QQ | | | | |
| | | | | | | QNQQ | | | | |
| | | | | | | QNQQ | | | | |
| | | | | | | QN | | | | |
| | | | | | | (SEQ ID | | | | |
| | | | | | | NO: 47) | | | | |
| rhx4 | Q₃N-oDi | | | | | QQQN | EAAARE | mEos3. | GGGG | PEDEIA |
| 294 | | | | | | QQQN | AAAR | 1 | SGGG | ALKK |
| | | | | | | QQ | EAAARE | | GS | EIAALK |
| | | | | | | QNQQ | AAAR | | GGGG | QENA |
| | | | | | | QNQQ | (SEQ ID | | SGGG | ALKQEI |
| | | | | | | QN | NO: 50) | | GS | AALK |
| | | | | | | QQQN | | | (SEQ | KEIAAL |
| | | | | | | QQQN | | | ID NO: | KQG |
| | | | | | | QQ | | | 51) | (SEQ ID |
| | | | | | | QNQQ | | | | NO: 52) |
| | | | | | | QNQQ | | | | |
| | | | | | | QN | | | | |
| | | | | | | QQQN | | | | |
| | | | | | | QQQN | | | | |
| | | | | | | QQ | | | | |
| | | | | | | QNQQ | | | | |
| | | | | | | QNQQ | | | | |
| | | | | | | QN | | | | |
| | | | | | | (SEQ ID | | | | |
| | | | | | | NO: 13) | | | | |
| rhx4 | Q₃N-FT | | | | | QQQN | EAAARE | mEos3. | GGGG | MTTAS |
| 633 | H1 | | | | | QQQN | AAAR | 1 | SGGG | TSQVR |
| | | | | | | QQ | EAAARE | | GS | QNYHQ |
| | | | | | | QNQQ | AAAR | | GGGG | DSEAA |
| | | | | | | QNQQ | (SEQ ID | | SGGG | INRQIN |
| | | | | | | QN | NO: 50) | | GS | LELY |
| | | | | | | QQQN | | | (SEQ | ASYVYL |
| | | | | | | QQQN | | | ID NO: | SMSY |
| | | | | | | QQ | | | 51) | YFDRD |
| | | | | | | QNQQ | | | | DVALK |
| | | | | | | QNQQ | | | | NFAKYF |
| | | | | | | QN | | | | LHQS |
| | | | | | | QQQN | | | | HEERE |
| | | | | | | QQQN | | | | HAEKL |
| | | | | | | QQ | | | | MKLQN |
| | | | | | | QNQQ | | | | QRGGR |
| | | | | | | QNQQ | | | | IFLQDIK |
| | | | | | | QN | | | | KPD |
| | | | | | | (SEQ ID | | | | CDDWE |
| | | | | | | NO: 13) | | | | SGLNA |
| | | | | | | | | | | MECAL |
| | | | | | | | | | | HLEKN |
| | | | | | | | | | | VNQSLL |
| | | | | | | | | | | ELHK |
| | | | | | | | | | | LATDKN |
| | | | | | | | | | | DPHL |
| | | | | | | | | | | CDFIET |
| | | | | | | | | | | HYLN |
| | | | | | | | | | | EQVKAI |
| | | | | | | | | | | KELG |
| | | | | | | | | | | DHVTN |
| | | | | | | | | | | LRKMG |
| | | | | | | | | | | APESG |
| | | | | | | | | | | LAEYL |
| | | | | | | | | | | FDKHTL |
| | | | | | | | | | | GDSD |
| | | | | | | | | | | NES |
| | | | | | | | | | | (SEQ ID |
| | | | | | | | | | | NO: 53) |
| rhx4 | Q₇N-oDi | | | | | QQQQ | EAAARE | mEos3. | GGGG | PEDEIA |
| 293 | | | | | | QQQN | AAAR | 1 | SGGG | ALKK |
| | | | | | | QQ | EAAARE | | GS | EIAALK |
| | | | | | | QQQQ | AAAR | | GGGG | QENA |
| | | | | | | QNQQ | (SEQ ID | | SGGG | ALKQEI |
| | | | | | | QQ | NO: 50) | | GS | AALK |
| | | | | | | QQQN | | | (SEQ | KEIAAL |
| | | | | | | QQQQ | | | ID NO: | KQG |
| | | | | | | QQ | | | 51) | (SEQ ID |
| | | | | | | QNQQ | | | | NO: 52) |
| | | | | | | QQQQ | | | | |
| | | | | | | QN | | | | |
| | | | | | | QQQQ | | | | |
| | | | | | | QQQN | | | | |
| | | | | | | QQ | | | | |
| | | | | | | QQQQ | | | | |
| | | | | | | QNQQ | | | | |
| | | | | | | QQ | | | | |
| | | | | | | (SEQ ID | | | | |
| | | | | | | NO: 16) | | | | |
| rhx4 | Q₇N-FT | | | | | QQQQ | EAAARE | mEos3. | GGGG | MTTAS |
| 631 | H1 | | | | | QQQN | AAAR | 1 | SGGG | TSQVR |
| | | | | | | QQ | EAAARE | | GS | QNYHQ |
| | | | | | | QQQQ | AAAR | | GGGG | DSEAA |
| | | | | | | QNQQ | (SEQ ID | | SGGG | INRQIN |
| | | | | | | QQ | NO: 50) | | GS | LELY |
| | | | | | | QQQN | | | (SEQ | ASYVYL |
| | | | | | | QQQQ | | | ID NO: | SMSY |
| | | | | | | QQ | | | 51) | YFDRD |
| | | | | | | QNQQ | | | | DVALK |
| | | | | | | QQQQ | | | | NFAKYF |
| | | | | | | QN | | | | LHQS |
| | | | | | | QQQQ | | | | HEERE |
| | | | | | | QQQN | | | | HAEKL |
| | | | | | | QQ | | | | MKLQN |
| | | | | | | QQQQ | | | | QRGGR |
| | | | | | | QNQQ | | | | IFLQDIK |
| | | | | | | QQ | | | | KPD |
| | | | | | | (SEQ ID | | | | CDDWE |
| | | | | | | NO: 16) | | | | SGLNA |
| | | | | | | | | | | MECAL |
| | | | | | | | | | | HLEKN |
| | | | | | | | | | | VNQSLL |
| | | | | | | | | | | ELHK |
| | | | | | | | | | | LATDKN |
| | | | | | | | | | | DPHL |
| | | | | | | | | | | CDFIET |
| | | | | | | | | | | HYLN |
| | | | | | | | | | | EQVKAI |
| | | | | | | | | | | KELG |
| | | | | | | | | | | DHVTN |
| | | | | | | | | | | LRKMG |
| | | | | | | | | | | APESG |
| | | | | | | | | | | LAEYL |
| | | | | | | | | | | FDKHTL |
| | | | | | | | | | | GDSD |
| | | | | | | | | | | NES |
| | | | | | | | | | | (SEQ ID |
| | | | | | | | | | | NO: 53) |
| rhx4 | Q60-oDi | | | | | QQQQ | EAAARE | mEos3. | GGGG | PEDEIA |
| 242 | | | | | | QQQQ | AAAR | 1 | SGGG | ALKK |
| | | | | | | QQ | EAAARE | | GS | EIAALK |
| | | | | | | QQQQ | AAAR | | GGGG | QENA |
| | | | | | | QQQQ | (SEQ ID | | SGGG | ALKQEI |
| | | | | | | QQ | NO: 50) | | GS | AALK |
| | | | | | | QQQQ | | | (SEQ | KEIAAL |
| | | | | | | QQQQ | | | ID NO: | KQG |
| | | | | | | QQ | | | 51) | (SEQ ID |
| | | | | | | QQQQ | | | | NO: 52) |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | (SEQ ID | | | | |
| | | | | | | NO: 19) | | | | |
| rhx4 | Q60- | | | | | QQQQ | EAAARE | mEos3. | GGGG | MTTAS |
| 632 | FTH1 | | | | | QQQQ | AAAR | 1 | SGGG | TSQVR |
| | | | | | | QQ | EAAARE | | GS | QNYHQ |
| | | | | | | QQQQ | AAAR | | GGGG | DSEAA |
| | | | | | | QQQQ | (SEQ ID | | SGGG | INRQIN |
| | | | | | | QQ | NO: 50) | | GS | LELY |
| | | | | | | QQQQ | | | (SEQ | ASYVYL |
| | | | | | | QQQQ | | | ID NO: | SMSY |
| | | | | | | QQ | | | 51) | YFDRD |
| | | | | | | QQQQ | | | | DVALK |
| | | | | | | QQQQ | | | | NFAKYF |
| | | | | | | QQ | | | | LHQS |
| | | | | | | QQQQ | | | | HEERE |
| | | | | | | QQQQ | | | | HAEKL |
| | | | | | | QQ | | | | MKLQN |
| | | | | | | QQQQ | | | | QRGGR |
| | | | | | | QQQQ | | | | IFLQDIK |
| | | | | | | QQ | | | | KPD |
| | | | | | | (SEQ ID | | | | CDDWE |
| | | | | | | NO: 19) | | | | SGLNA |
| | | | | | | | | | | MECAL |
| | | | | | | | | | | HLEKN |
| | | | | | | | | | | VNQSLL |
| | | | | | | | | | | ELHK |
| | | | | | | | | | | LATDKN |
| | | | | | | | | | | DPHL |
| | | | | | | | | | | CDFIET |
| | | | | | | | | | | HYLN |
| | | | | | | | | | | EQVKAI |
| | | | | | | | | | | KELG |
| | | | | | | | | | | DHVTN |
| | | | | | | | | | | LRKMG |
| | | | | | | | | | | APESG |
| | | | | | | | | | | LAEYL |
| | | | | | | | | | | FDKHTL |
| | | | | | | | | | | GDSD |
| | | | | | | | | | | NES |
| | | | | | | | | | | (SEQ ID |
| | | | | | | | | | | NO: 53) |
| rhx4 | Q60 | | | | | QQQQ | GGGGS | mEos3. | | |
| 584 | | | | | | QQQQ | GGGGS | 1 | | |
| | | | | | | QQ | GGGGS | | | |
| | | | | | | QQQQ | GGGGS | | | |
| | | | | | | QQQQ | (SEQ ID | | | |
| | | | | | | QQ | NO: 51) | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | (SEQ ID | | | | |
| | | | | | | NO: 19) | | | | |
| rhx4 | oDi-Q60 | PEDEI | GGGG | mEos3. | EAAA | QQQQ | | | | |
| 345b | | AALK | SGGG | 1 | REAA | QQQQ | | | | |
| | | K | GS | | AR | QQ | | | | |
| | | EIAAL | GGGG | | EAAA | QQQQ | | | | |
| | | KQEN | SGGG | | REAA | QQQQ | | | | |
| | | A | | | AR | QQ | | | | |
| | | ALKQ | | | | QQQQ | | | | |
| | | EIAAL | | | | QQQQ | | | | |
| | | K | GS | | | QQ | | | | |
| | | KEIAA | | | | QQQQ | | | | |
| | | LKQG | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | (SEQ ID | | | | |
| | | | | | | NO: 19) | | | | |
| rhx4 | Q60-oDi | | | | | QQQQ | GGGGS | mEos3. | GGGG | PEDEIA |
| 615 | | | | | | QQQQ | GGGGS | 1 | SGGG | ALKK |
| | | | | | | QQ | GGGGS | | GS | EIAALK |
| | | | | | | QQQQ | GGGGS | | GGGG | QENA |
| | | | | | | QQQQ | (SEQ ID | | SGGG | ALKQEI |
| | | | | | | QQ | NO: 51) | | GS | AALK |
| | | | | | | QQQQ | | | | KEIAAL |
| | | | | | | QQQQ | | | | KQG |
| | | | | | | QQ | | | | (SEQ ID |
| | | | | | | QQQQ | | | | NO: 52) |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | (SEQ ID | | | | |
| | | | | | | NO: 19) | | | | |
| rhx4 | Q60- | | | | | QQQQ | GGGGS | mE | GGGG | PEDEIA |
| 619 | oDi{X} | | | | | QQQQ | GGGGS | os3. | SGGG | ALKK |
| | | | | | | QQ | GGGGS | 1 | GS | EIAALK |
| | | | | | | QQQQ | GGGGS | | GGGG | QENA |
| | | | | | | QQQQ | (SEQ ID | | SGGG | ALKQEI |
| | | | | | | QQ | NO: 51) | | GS | AAKL |
| | | | | | | QQQQ | | | | EIKAAK |
| | | | | | | QQQQ | | | | LQG |
| | | | | | | QQ | | | | (SEQ ID |
| | | | | | | QQQQ | | | | NO: 54) |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | QQQQ | | | | |
| | | | | | | QQQQ | | | | |
| | | | | | | QQ | | | | |
| | | | | | | (SEQ ID | | | | |
| | | | | | | NO: 19) | | | | |
| rhx4 | (Q₆)4 | | | | | QQQQ | EAAARE | mEos3. | | |
| 071 | | | | | | QQGG | AAAR | 1 | | |
| | | | | | | GQ | EAAARE | | | |
| | | | | | | QQQQ | AAAR | | | |
| | | | | | | QGGG | (SEQ ID | | | |
| | | | | | | QQ | NO: 50) | | | |
| | | | | | | QQQQ | | | | |
| | | | | | | GGGQ | | | | |
| | | | | | | QQ | | | | |
| | | | | | | QQQ | | | | |
| | | | | | | (SEQ ID | | | | |
| | | | | | | NO: 48) | | | | |
| rhx4 | (Q₆)4 | | | | | QQQQ | EAAARE | mEos3. | | |
| 134 | ″Q10N″ | | | | | QQGG | AAAR | 1 | | |
| | | | | | | GQ | EAAARE | | | |
| | | | | | | QQNQ | AAAR | | | |
| | | | | | | QGGG | (SEQ ID | | | |
| | | | | | | QQ | NO: 50) | | | |
| | | | | | | QQQQ | | | | |
| | | | | | | GGGQ | | | | |
| | | | | | | QQ | | | | |
| | | | | | | QQQ | | | | |
| | | | | | | (SEQ ID | | | | |
| | | | | | | NO: 49) | | | | |

Yeast strain rhy3078a ([PIN⁺]) was constructed as follows. We first deleted PDR5 and ATG8 from strain rhy1713 (Khan et al., 2018) by sequentially mating and sporulating it with the respective strains from the MATa deletion collection (Open Biosystems). PCR-based mutagenesis (Goldstein and McCusker, 1999) was then used with template vector CX (Miller et al. submitted) to integrate BDFP1.6:1.6 prior to the stop codon of chromosomal PGK1. Yeast strain rhy3082 ([pin⁻]) is rhy3078a with the amyloid form of Rnq1 eliminated by passaging it four times on YPD plates containing 3 mM GdHCl, a prion-curing agent (Ferreira et al., 2001).

### DAmFRET

The yeast strains were transformed using a standard lithium acetate protocol with plasmids encoding the sequence to be tested as a fusion to the indicated tags (Table 1) under the control of the GAL1 promoter.

Individual colonies were picked and incubated in 200 μL of a standard synthetic media containing 2% dextrose (SD-ura) overnight while shaking on a Heidolph Titramax-1000 at 1000 rpm at 30° C. Following overnight growth, cells were spun down and resuspended in a synthetic induction media containing 2% galactose (SGal-ura). Cells were induced for 16 hours while shaking before being resuspended in fresh 2% SGal-ura for 4 hours to reduce autofluorescence. 75 μL of cells were then re-arrayed from a 96 well plate to a 384 well plate and photoconverted, while shaking at 800 rpm, for 25 minutes using an OmniCure S1000 fitted with a 320-500 nm (violet) filter and a beam collimator (Exfo), positioned 45 cm above the plate.

High-throughput flow cytometry (10 uL/well, flow speed 1.8) was performed on the Bio-Rad ZE5 with a Propel automation setup and a GX robot (PAA Inc). Donor and FRET signals were collected from a 488 nm laser set to 100 mW, with voltages 351 and 370 respectively, into 525/35 and 593/52 detectors. Acceptor signal was collected from a 561 nm laser set to 50 mW with voltage at 525, into a 589/15 detector. Autofluorescence was collected from a 405 nm laser at 100 mW and voltage 450, into a 460/22 detector.

Compensation was performed manually, collecting files for non-photoconverted mEos3.1 for pure donor fluorescence and dsRed2 to represent acceptor signal as it has a similar spectrum to the red form of mEos3.1. FRET is compensated only in the direction of donor and acceptor fluorescence out of FRET channels, as there is not a pure FRET control. Acceptor fluorescence intensities were divided by side scatter (SSC), a proxy for cell volume (Miller et al. submitted), to convert them to concentration (in arbitrary units).

Imaging flow cytometry was conducted as in previous work (Khan et al., 2018; Venkatesan et al., 2019).

### DAmFRET Automated Analysis

FCS 3.1 files resulting from assay were gated using an automated R-script running in flowCore. Prior to gating, the forward scatter (FS00.A, FS00.W, FS00.H), side scatter (SS02.A), donor fluorescence (FL03.A) and autofluorescence (FL17.A) channels were transformed using a logicle transform in R. Gating was then done using flowCore by sequentially gating for cells using FS00.A vs SS02.A then selecting for single cells using FS00.H vs FS00.W and finally selecting for expressing cells using FL03.A vs FL17.A.

Gating for cells was done using a rectangular gate with values of Xmin=2.7, Xmax=4.8, Ymin=2.7, Ymax=4.7). Gating for single cells was done using a rectangular gate with values of Xmin=4.45, Xmax=4.58, Ymin=2.5, Ymax=4.4. Gating of expressing cells was done using a polygon gate with x/y vertices of (1, 0.1), (1.8, 2), (5, 2), (5, 0.1). Cells falling within all of these gates were then exported as FCS3.0 files for further analysis.

The FCS files resulting from the autonomous gating in Step 1 were then utilized for the JAVA-based quantification of a curve similar to the analysis found in the original assay (Khan et al 2018). Specifically:

The quantification procedure first divides a defined negative control DAmFRET histogram into 64 logarithmically spaced bins across a pre-determined range large enough to accommodate all potential data sets. Then upper gate values were determined for each bin as the 99^{th} percentile of the DamFRET distribution in that bin. For bins at very low and very high acceptor intensities, there are not enough cells to accurately calculate this gate value. As a result, for bins above the 99^{th} acceptor percentile and bins below 2 million acceptor intensity units, the upper gate value was set to the value of the nearest valid bin. The upper gate profile was then smoothed by boxcar smoothing with a width of 5 bins and shifted upwards by 0.028 DAmFRET units to ensure that the negative control signal lies completely within the negative FRET gate. The lower gate values for all bins were set equal to −0.2 DamFRET units. For all samples, then, cells falling above this negative FRET gate can be said to contain assembled (FRET-positive) protein. A metric reporting the gross percentage of the expressing cells containing assembled proteins is therefore reported as fgate which is a unitless statistic between 0 and 1.

This gate is then applied to all DAmFRET plots to define cells containing proteins that are either positive (self-assembled) or negative (monomeric). In each of the 64 gates, the fraction of cells in the assembled population were plotted as a ratio to total cells in the gate.

### Microscopy

Cells were imaged using a CSU-W1 spinning disc Ti2 microscope (Nikon) and visualized through a 100× Plan Apochromat objective (NA 1.45). mEos3.1 was excited at 488 nm and emission was collected for 50 ms per frame through a ET525/36M bandpass filter on to a Flash 4 camera (Hamamatsu). Full z stacks of all cells were acquired over ˜15 μm total distance with z spacing of 0.2 μm. Transmitted light was collected at the middle of the z stack for reference. To quantify the total intensity of each Fiji cell, the z stacks were processed using F (https://imagej.net/software/fiji/). Images were first converted to 32-bit and sum projected in Z. Regions of interest (ROIs) were hand drawn around each cell using the ellipse tool in Fiji on the transmitted light image. These ROIs were then used to measure the area, mean, standard deviation, and integrated density of each cell on the fluorescence channel. Each cell was then classified as being diffuse or punctate by calculating the coefficient of variance (CV, standard deviation divided by the square root of the mean intensity) of the fluorescence. Cells were only considered punctate if their CV was greater than 30. Cells from each category that had equivalent integrated densities were directly compared and the images were plotted on the same intensity scale. The volume of each cell was calculated by fitting the transmitted light hand drawn ROI to an ellipse. The cell was then assumed to be a symmetric ellipsoid with the parameters of the fit ellipse from Fiji. The volume was calculated by 4/3*pi*major*minor*minor, where major and minor are the major and minor axes of the Fiji ellipse fit to the hand drawn ellipse ROI. The volumes reported are the volumes of the 3D ellipsoids. Concentrations were calculated by dividing the integrated densities by the calculated volume in μm³, yielding units of fluorescence per μm³.

### SDD-AGE

Semi-denaturing detergent agarose gel electrophoresis (SDD-AGE) was done as in (Khan et al., 2018). The gel was imaged directly using a GE Typhoon Imaging System using a 488 laser and 525(40) BP filter. Images were then loaded into ImageJ for contrast adjustment. Images were gaussian blurred with a radius of 1 and then background subtracted with a 200 pixel rolling ball. Representative samples were then cropped from the original image for emphasis. Dotted or solid lines denote where different regions of the same gel were aligned for comparison. Line profiles of the protein smear were quantified using the following procedure. All images were processed in Fiji (https://imagej.net/software/fiji/). Gel images were first background subtracted using a rolling ball with a radius of 200 pixels. The images were then rotated so that the orientation was perfectly aligned. A user-defined line was drawn on the image down the first lane. A 10 pixel wide line profile was generated using an in house written plugin “polyline kymograph jru v1”. This line was then programmatically shifted to every other lane and line profiles were generated for each. Each line profile was then normalized to the integral under the line profile using “normalize trajectories jru v1”. From these line profiles, csv sheets were generated and these were imported to python to make line profile plots.

### Amyloid Prediction

Comparative analyses were performed using default settings at the listed, public web servers as were available on Apr. 1, 2021. See Table 2 for further details.

TABLE 2
Summary of results from publically available APR and aggregation propensity predictor webservers.

| | | Amino Acid Sequence | | | | | |
|---|---|---|---|---|---|---|---|
| Application | | | | Q₃N | Q₄N | Q₅N | |
| Name | Link | Q(60) | N(60) | (60) | (60) | (60) | Reference |
| AGGRESCAN | http://bioinf.uab.es/aggrescan | non- | non- | non- | non- | non- | Chonchillo- |
| | | amyloid | amyloid | amyloid | amyloid | amyloid | Solï¿½ |
| | | | | | | | et al. |
| | | | | | | | (2007) |
| WALTZ | https://walktz.switchlab.org | non- | non- | non- | non- | non- | Maurer-Stroh |
| | | amyloid | amyloid | amyloid | amyloid | amyloid | et al. |
| | | | | | | | (2010) |
| FoldAmyloid | http://bioinfo.protres.ru/fold-amyloid/ | amyloid | amyloid | amyloid | amyloid | amyloid | Garbuzynskiy |
| | | | | | | | et al. |
| | | | | | | | (2010) |
| 3D | http://services.mbi.ucla.edu/zipperdb/ | non- | non- | non- | non- | non- | Thompson |
| Profile | | amyloid | amyloid | amyloid | amyloid | amyloid | et al. |
| (ZipperDB) | | | | | | | (2006) |
| Amylogram | http://smorfland.uni.wroc.pl/shiny/ | non- | non- | non- | non- | non- | Burdukiewicz |
| | AmyloGramhttp://smorfland.uni.wroc.pl/ | amyloid | amyloid | amyloid | amyloid | amyloid | et al. |
| | shiny/AmyloGram | | | | | | (2017) |
| ANuPP | https://web.iitm.ac.in/bioinfo2/ANuPP/ | non- | non- | non- | non- | non- | Prabakaran |
| | | amyloid | amyloid | amyloid | amyloid | amyloid | et al. |
| | | | | | | | (2020) |
| TANGO | http://tango.crg.es/ | non- | non- | non- | non- | non- | Fernandez- |
| | | amyloid | amyloid | amyloid | amyloid | amyloid | Escamilla |
| | | | | | | | et al. |
| | | | | | | | (2004) |
| SecStr | http://biophysics.biol.uoa.gr/SecStr/ | non- | non- | non- | non- | non- | Hamodrakas |
| | | amyloid | amyloid | amyloid | amyloid | amyloid | et al. |
| | | | | | | | (2007) |
| BETASCAN | http://betascan.csail.mit.edu/ | non- | non- | non- | non- | non- | Bryan |
| | | amyloid | amyloid | amyloid | amyloid | amyloid | et al. |
| | | | | | | | (2009) |
| Net- | http://cssp2.sookmyung.ac.kr/index.html | non- | non- | non- | non- | non- | Kim |
| CSSP | | amyloid | amyloid | amyloid | amyloid | amyloid | et al. |
| | | | | | | | (2009) |
| Amyloid | http://amyloid.csail.mit.edu/ | amyloid | non- | non- | non- | non- | O'Donnell |
| Mutants | | | amyloid | amyloid | amyloid | amyloid | et al. |
| | | | | | | | (2011) |
| PASTA2 | http://old.protein.bio.unipd.it/pasta2/ | non- | non- | non- | non- | non- | Walsh |
| | | amyloid | amyloid | amyloid | amyloid | amyloid | et al. |
| | | | | | | | (2014) |
| GAP | http://www.iitm.ac.in/bioinfo/GAP/ | amyloid | amyloid | amyloid | amyloid | amyloid | Thangakani |
| | | | | | | | et al. |
| | | | | | | | (2014) |
| MetAmyl | http://metamyl.genouest.org/ | non- | non- | non- | non- | non- | Emily |
| | | amyloid | amyloid | amyloid | amyloid | amyloid | et al. |
| | | | | | | | (2013) |
| Budapest | https://pitgroup.org/bap/ | non- | non- | non- | non- | non- | Keresztes, |
| Amyloid | | amyloid | amyloid | amyloid | amyloid | amyloid | et al. |
| Predictor | | | | | | | (2020) |
| iAMY-SCM | http://camt.pythonanywhere.com/ | amyloid | amyloid | amyloid | amyloid | amyloid | Charoenkwan, |
| | iAMY-SCM | | | | | | et al. |
| | | | | | | | (2020) |

### Molecular Simulations

The simulations were carried out using the AMBER 20 package (Case et al., 2020) with ff14SB force field (Maier et al., 2015) and explicit TIP3P water model (Price and Brooks, 2004). The aggregates were placed in a cubic solvation box. In each aggregate, the minimum distance from the aggregate to the box boundary was set to be 12 nm to avoid self-interactions. An 8 Å cutoff was applied for the nonbonded interactions, and the particle mesh Ewald (PME) method (Essmann et al., 1995) was used to calculate the electrostatic interaction with cubic-spline interpolation and a grid spacing of approximately 1 Å. Once the box was set up, we performed a structural optimization with aggregates fixed, and on a second step allowed them to move, followed by a graduate heating procedure (NVT) with individual steps of 200 ps from 0 K to 300 K. Finally, the production runs were carried out using NPT Langevin dynamics with constant pressure of 1 atm at 300 K.

### Example 2

### Amyloid Nucleation is Governed by a Hidden Pattern of Glutamines

To validate the use of DAmFRET for pathologic polyQ, we first surveyed the length-dependence for amyloid formation by polyQ tracts expressed as fusions to mEos3.1 in nondividing [pin⁻] yeast cells. We genetically compromised protein degradation in these cells to prevent differential turnover of potential polyQ heterogeneities, which could otherwise obscure the relationship of aggregation to concentration. The data recapitulated the pathologic threshold—Q lengths 35 and shorter lacked AmFRET, indicating a failure to aggregate or even appreciably oligomerize, while Q lengths 40 and longer did acquire AmFRET in a length and concentration-dependent manner (FIGS. 1C top, 1D, 6A). Specifically, the cells partitioned into two discontinuous populations: one with high AmFRET and the other with none (top and bottom populations in FIG. 1C). The two populations occurred with overlapping concentration ranges (red dashed box in FIG. 1C top), indicating a kinetic barrier to forming the high-FRET state. The two populations were well-resolved at high concentrations, where relatively few cells populated intermediate values of AmFRET. These features together indicate that aggregation of polyQ is rate-limited by a nucleation barrier associated with a rare conformational fluctuation.

Sixty residues proved to be the optimum length to observe both the pre- and post-nucleated states of polyQ in single experiments, and corresponds clinically to disease onset in early adulthood (Kuiper et al., 2017). The frequency of polyQ nucleation greatly exceeds that of other Q-rich amyloid-forming proteins of comparable length (Khan et al., 2018; Posey et al., 2021). To determine if the protein's exceptional amyloid propensity can be attributed to a property of the Q residues themselves, as opposed to its extremely low sequence complexity, next tested was a 60-residue homopolymer of asparagine (polyN), whose side chain is one methylene shorter but otherwise identical to that of glutamine. PolyN populated the high FRET state at much lower frequencies (typically undetectable) than polyQ even at the highest concentrations (FIG. 1C bottom, approximately 200 UM (Khan et al., 2018)).

The much larger nucleation barrier for polyN—despite its physicochemical similarity to polyQ—led us to consider whether substituting Qs for Ns at specific positions in polyQ might reveal patterns that uniquely encode the structure of the polyQ nucleus. It was reasoned that a random screen of Q/N sequence space would be unlikely to yield informative patterns, however, given that there are more than one trillion combinations of Q and N for even a 40-residue peptide. A systematic approach was devised to rationally sample Q/N sequence space. Specifically, DAmFRET was used to characterize a series of related sequences with a single N residue inserted after every q Q residues over a total length of 60, for all values of q from 0 through 9 (designated Q₁N, Q₂N, etc. with the value of q subscripted; Table 1). The resulting dataset revealed a shockingly strong dependence of amyloid propensity on the exact sequence of Q residues, and specifically the following two determinants.

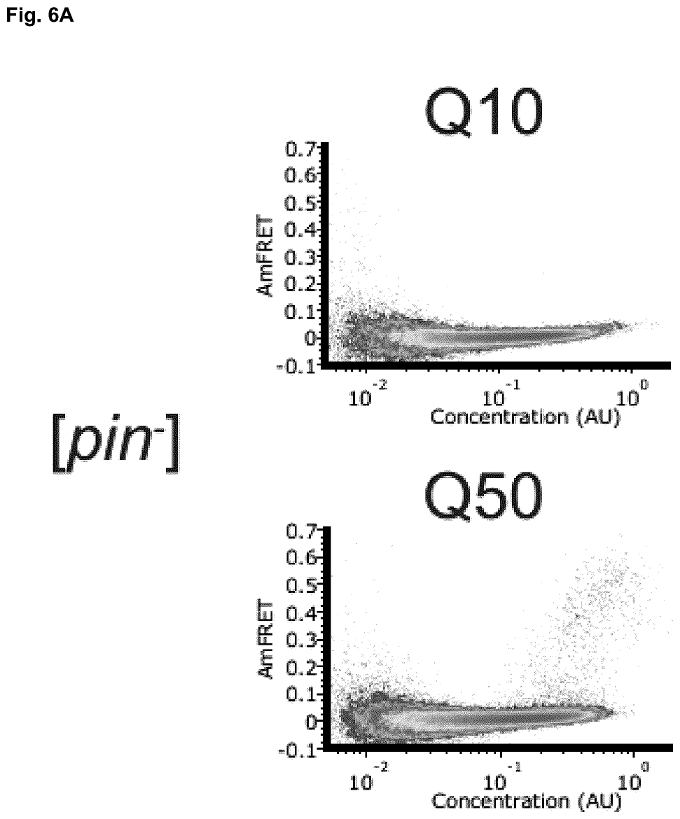

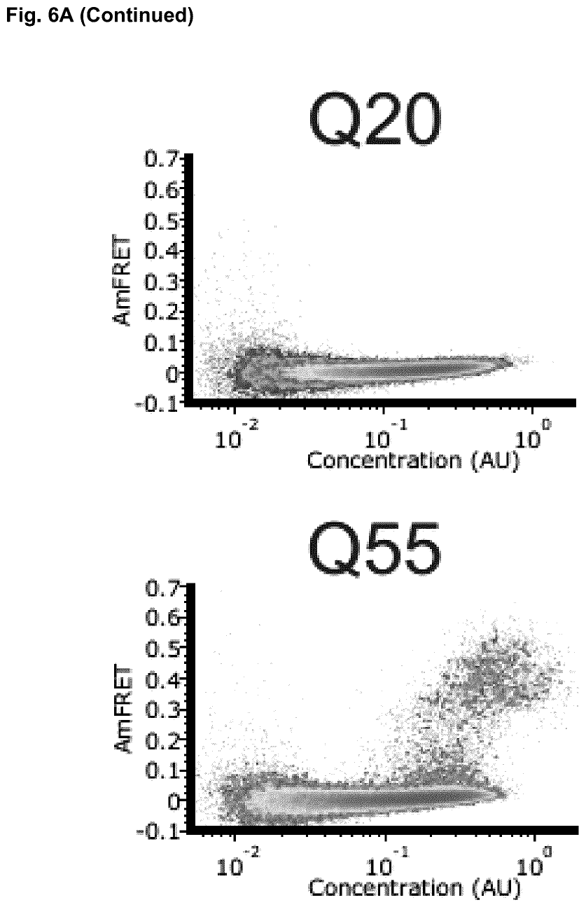

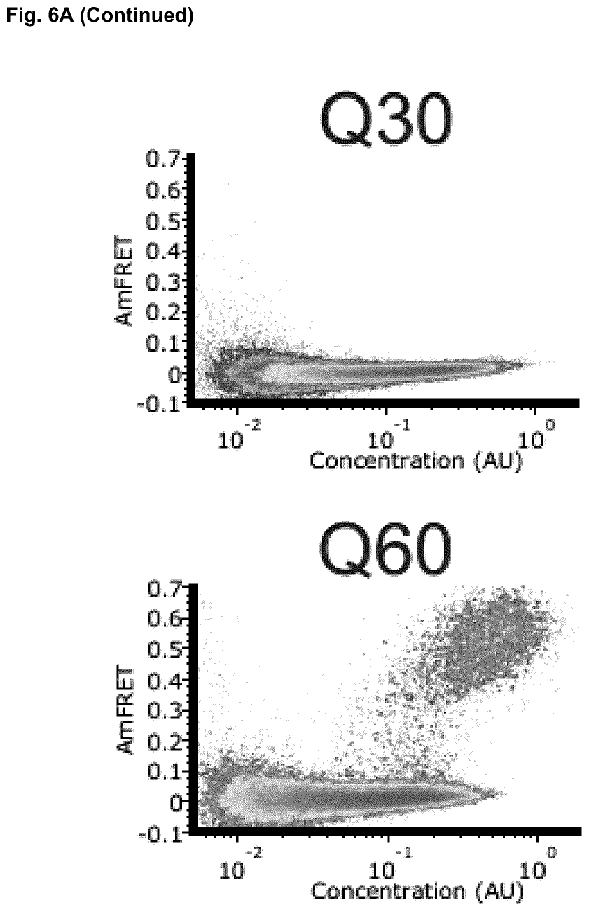

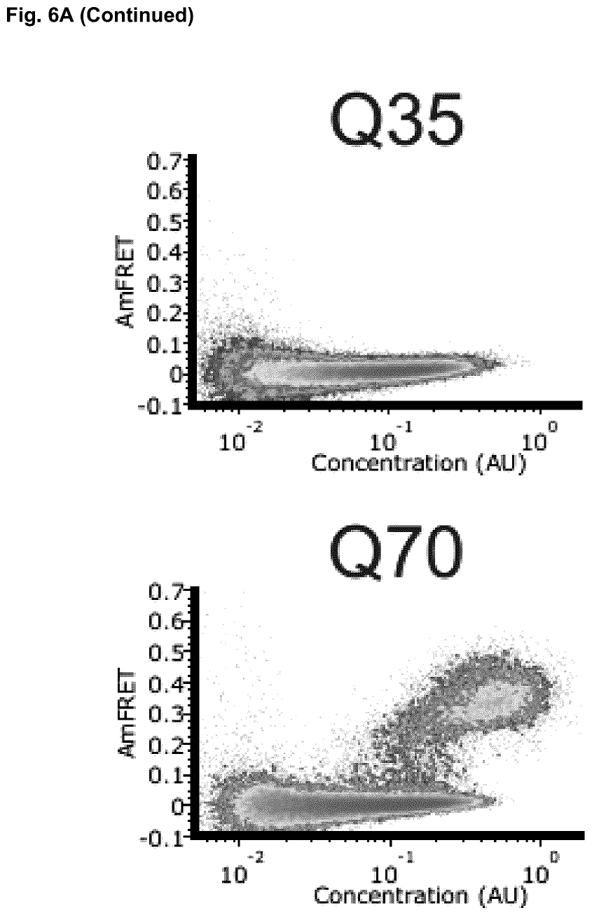

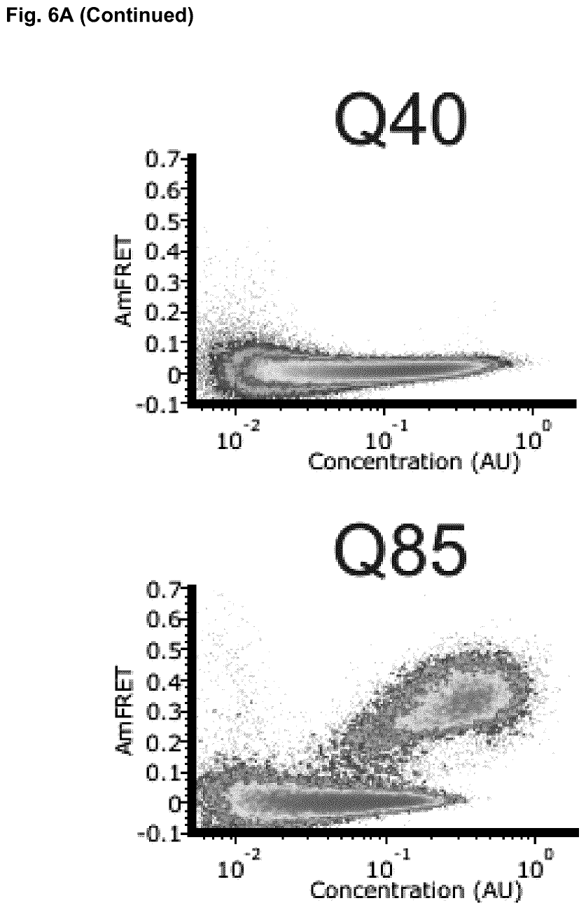

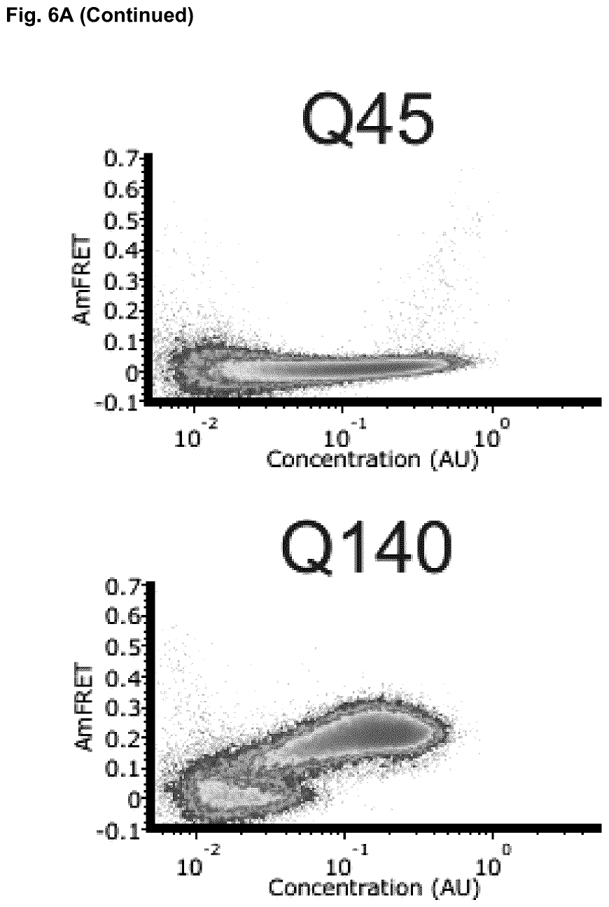

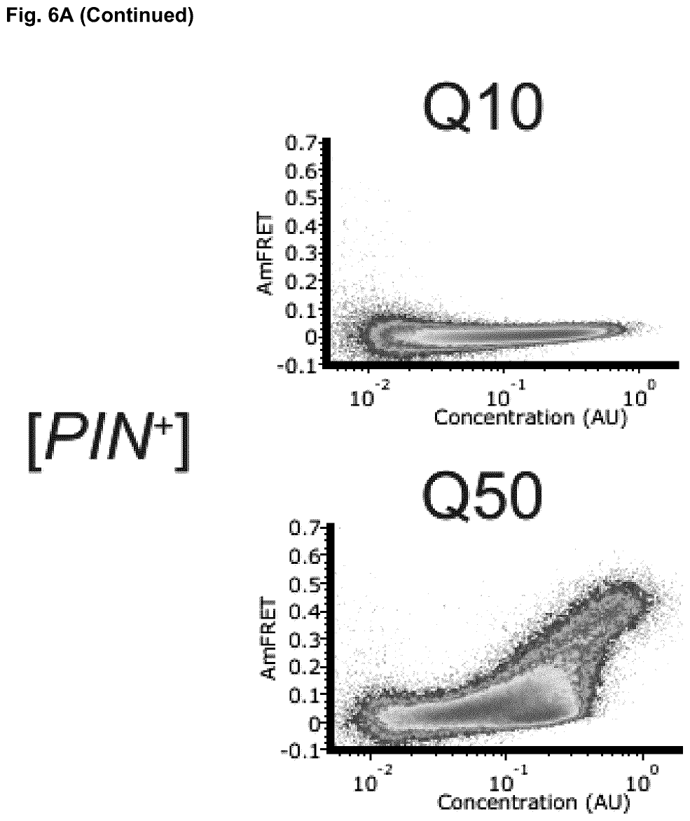

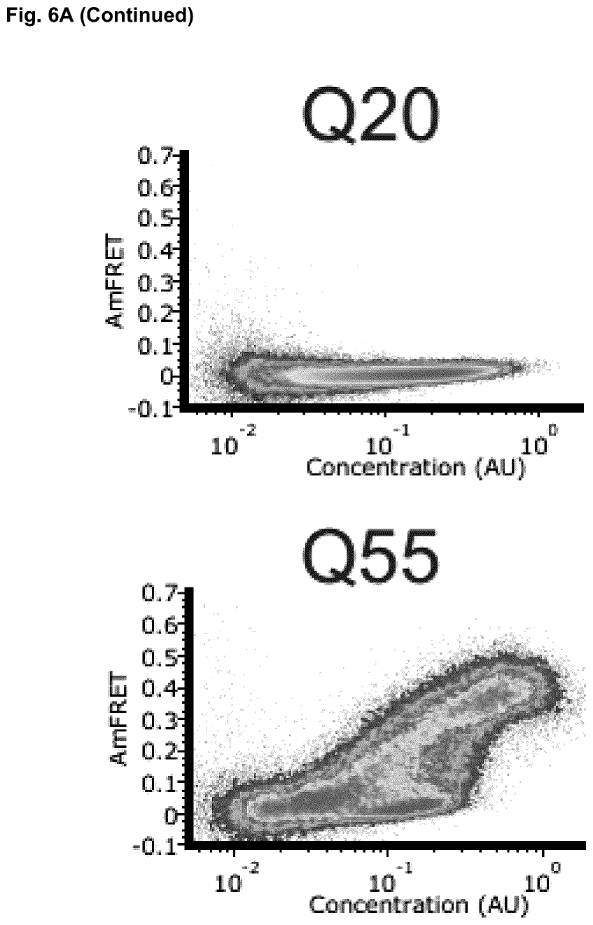

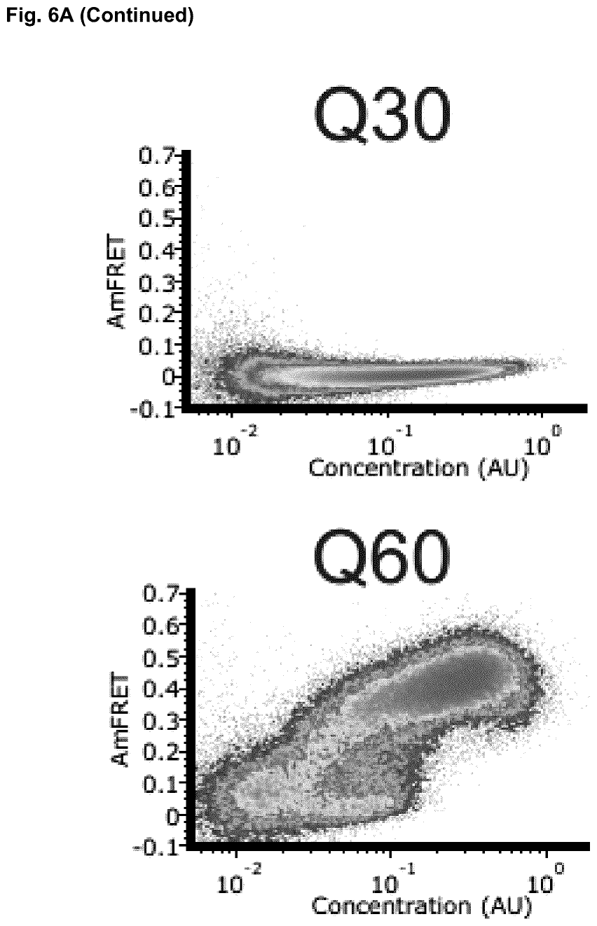

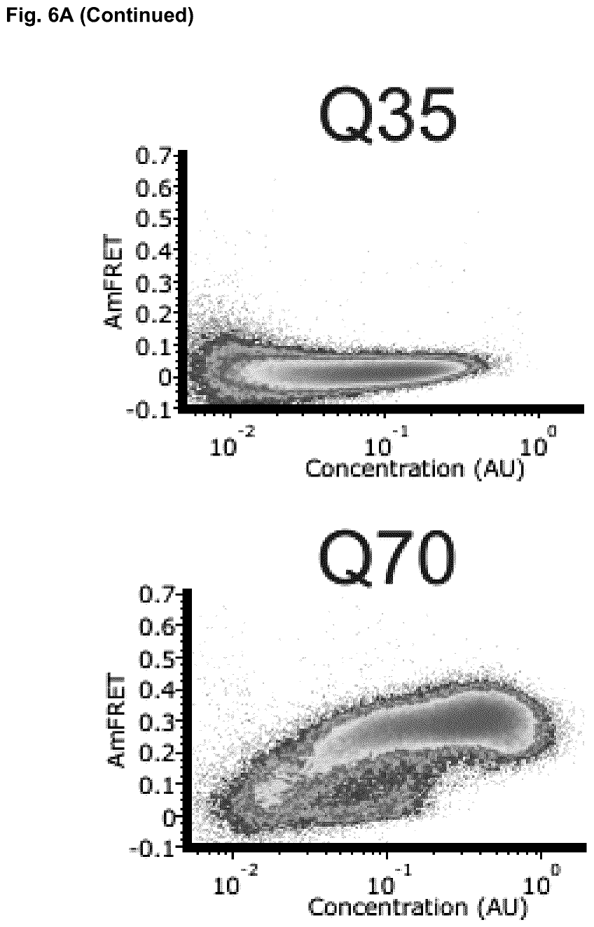

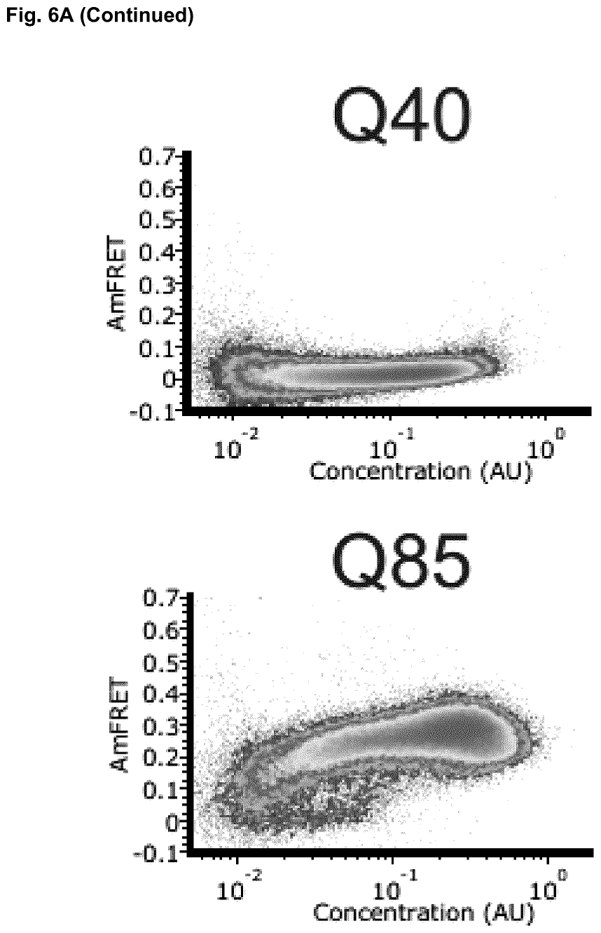

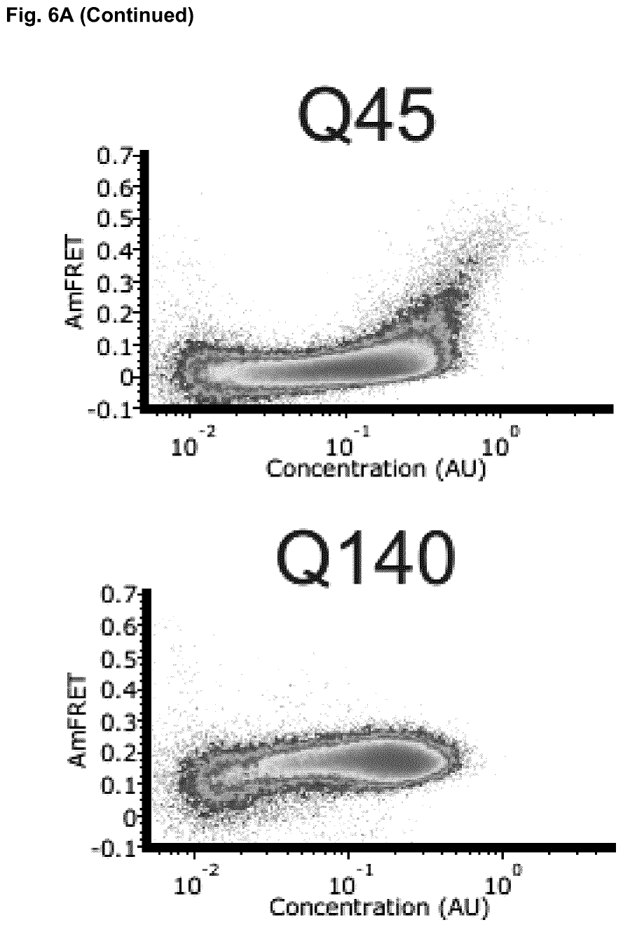

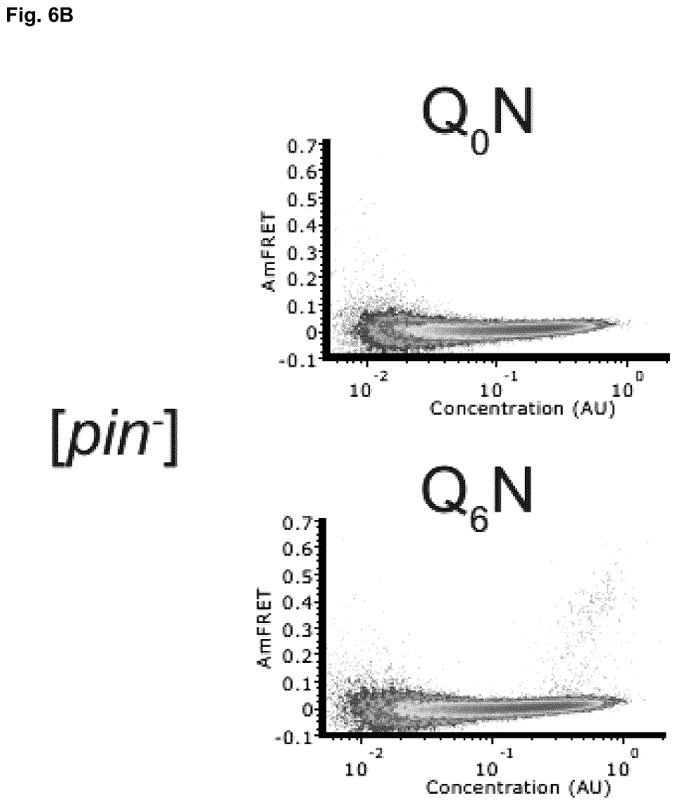

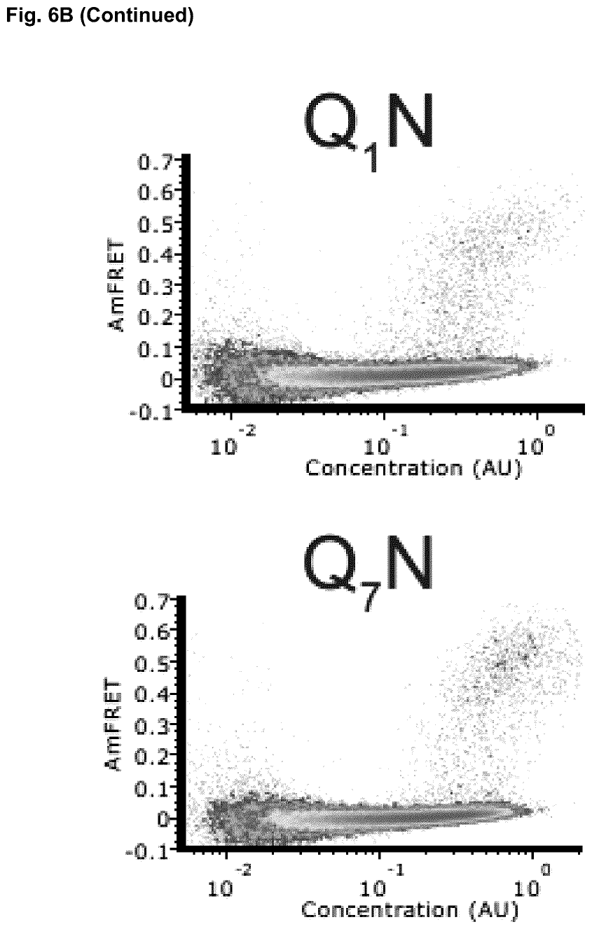

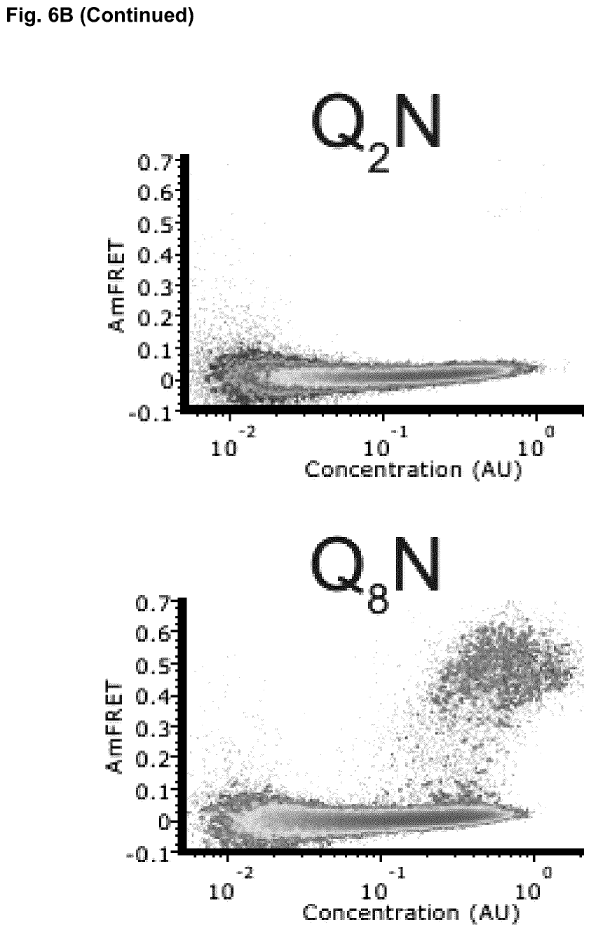

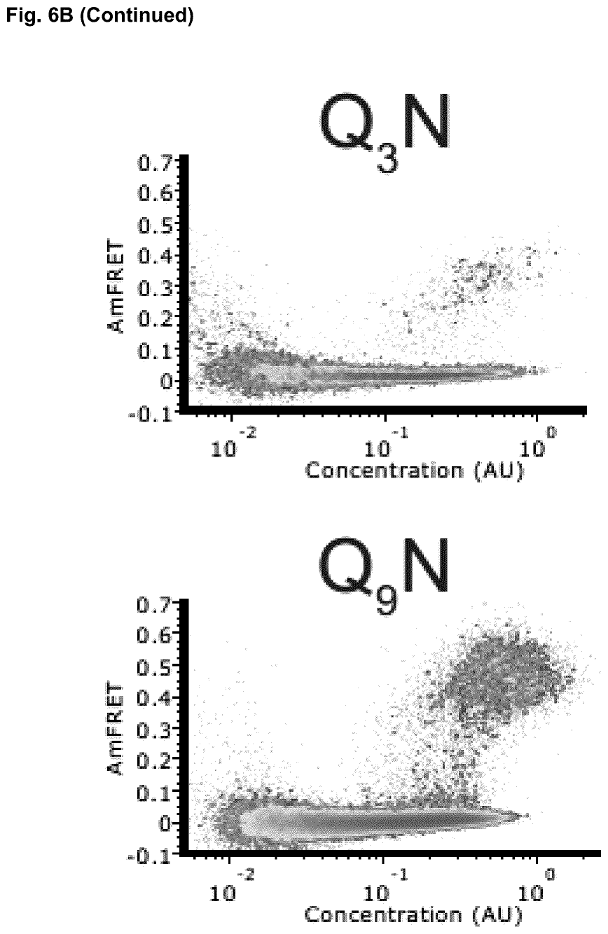

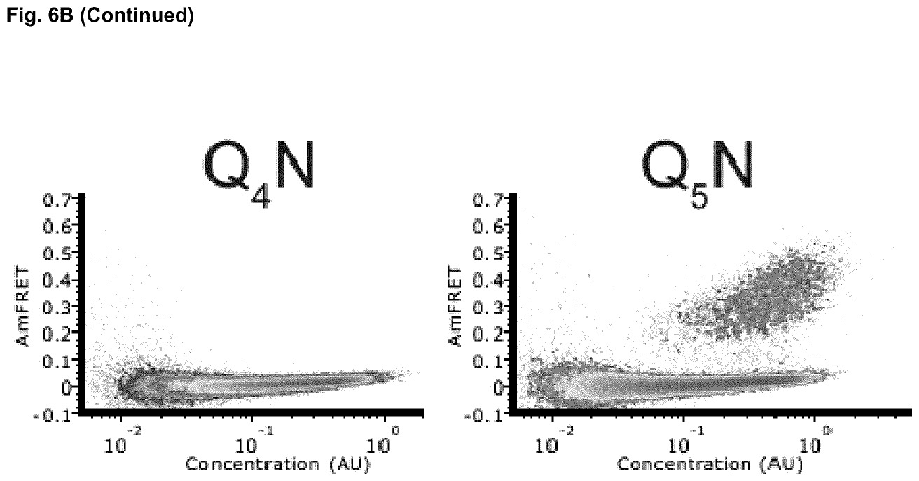

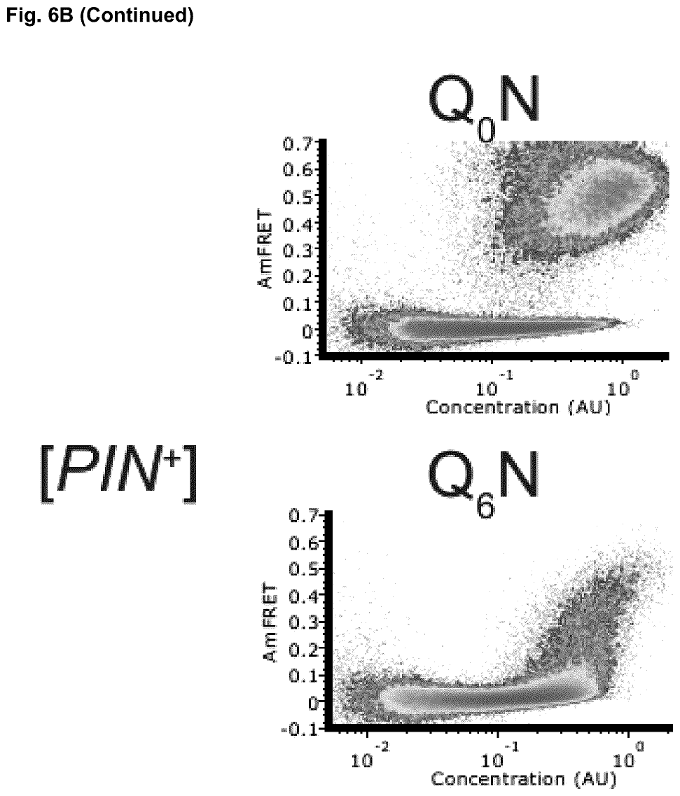

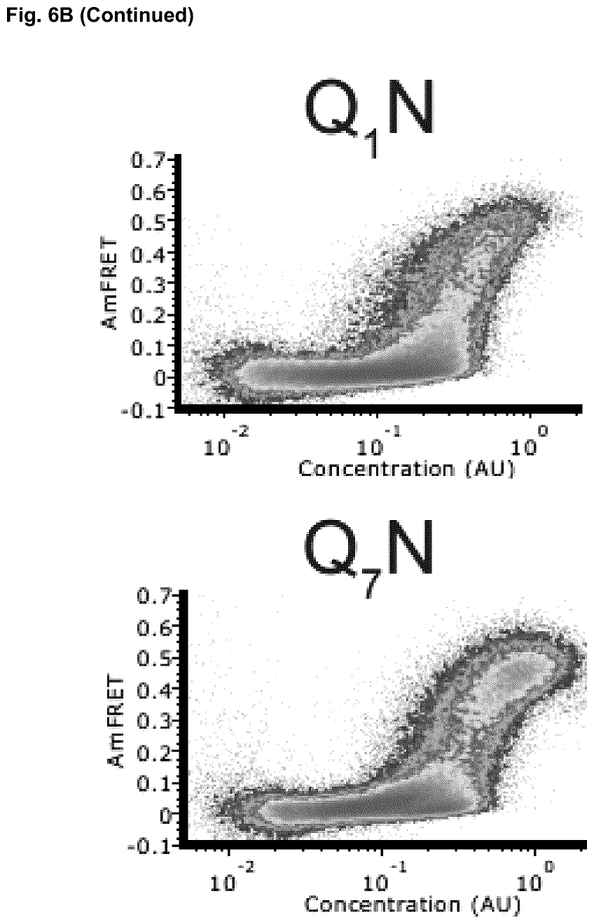

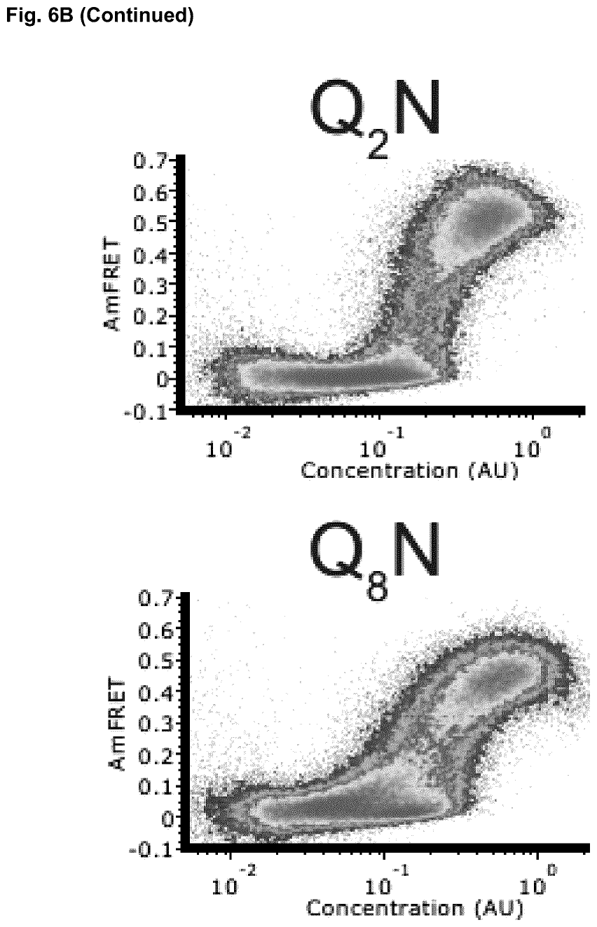

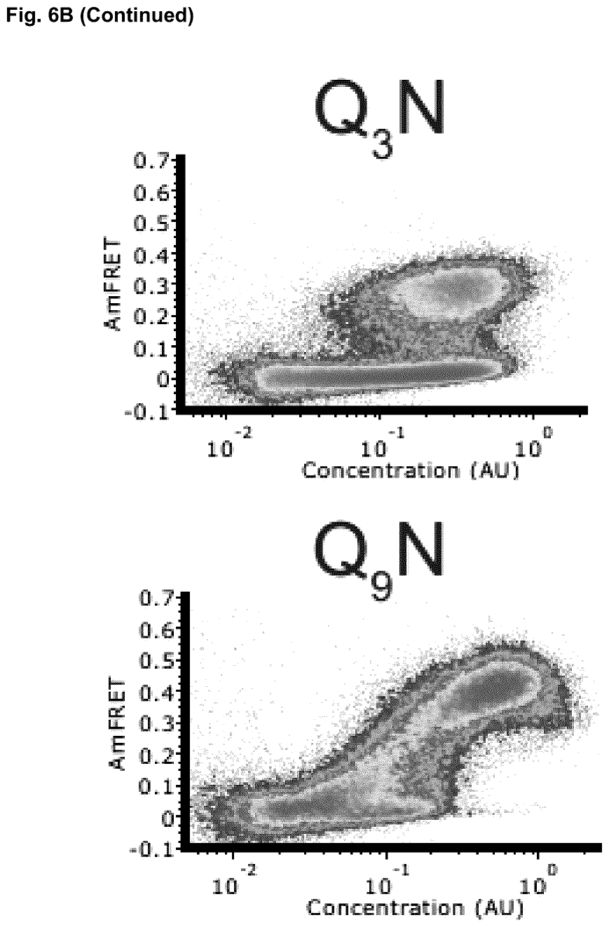

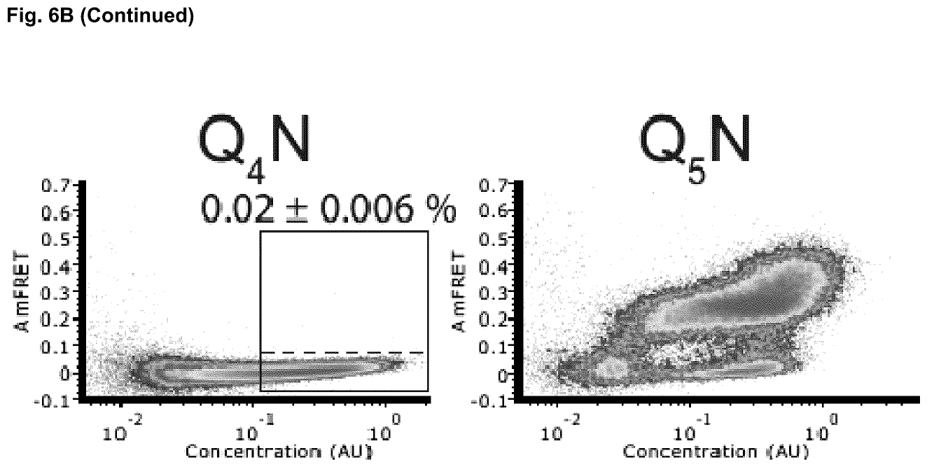

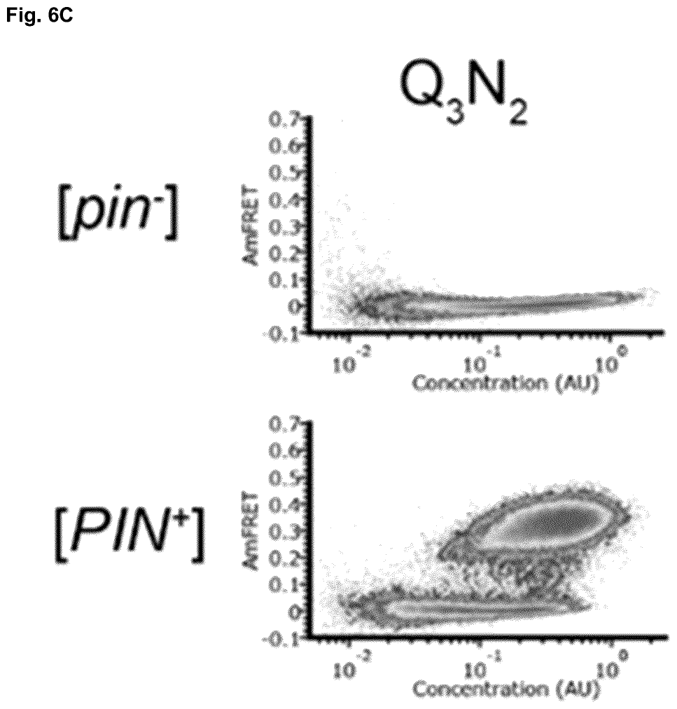

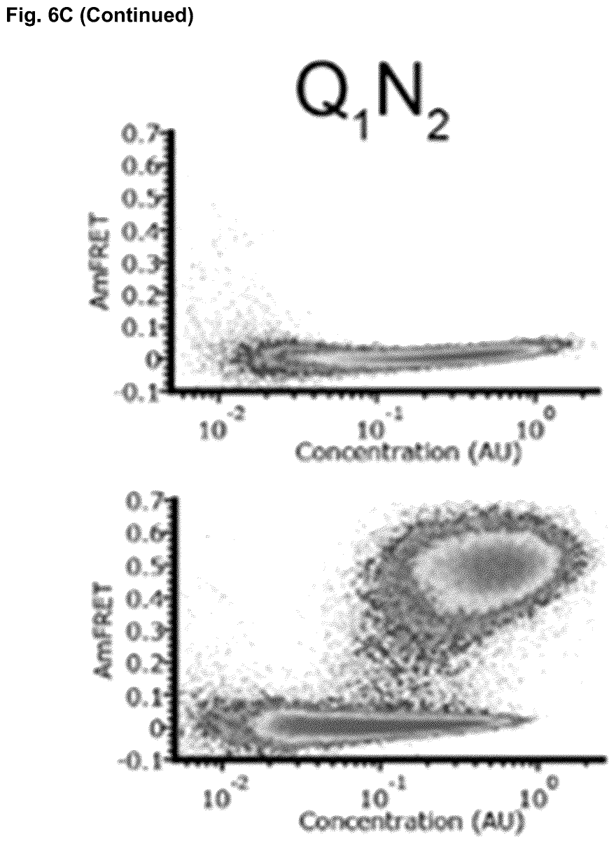

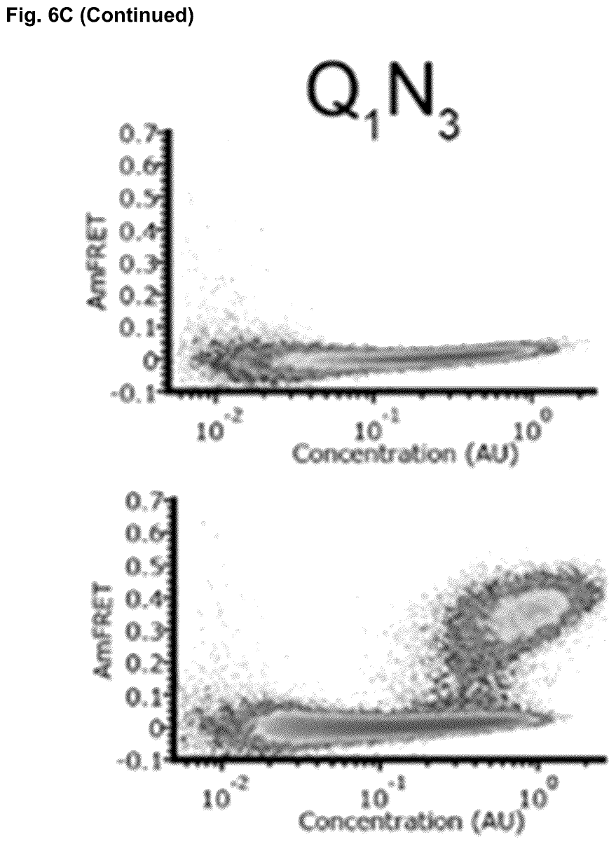

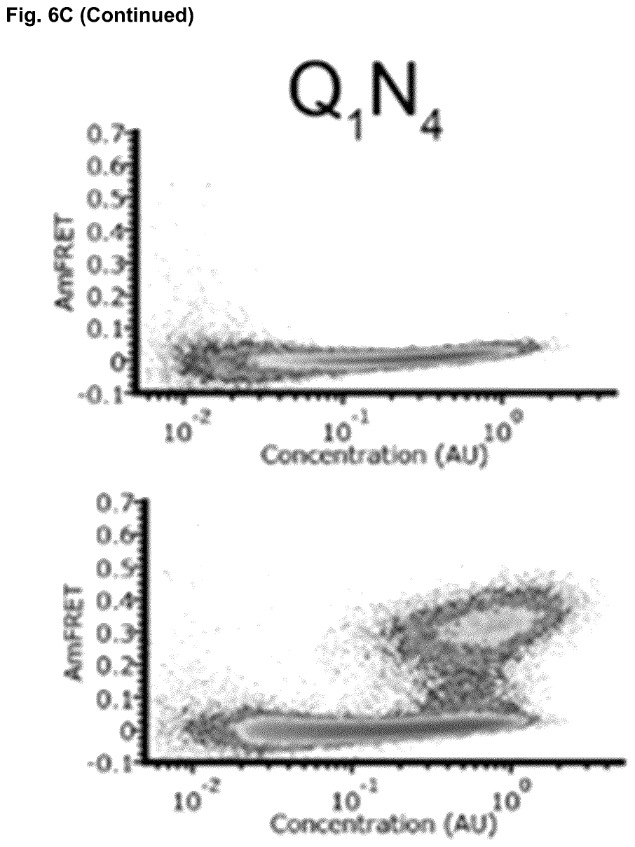

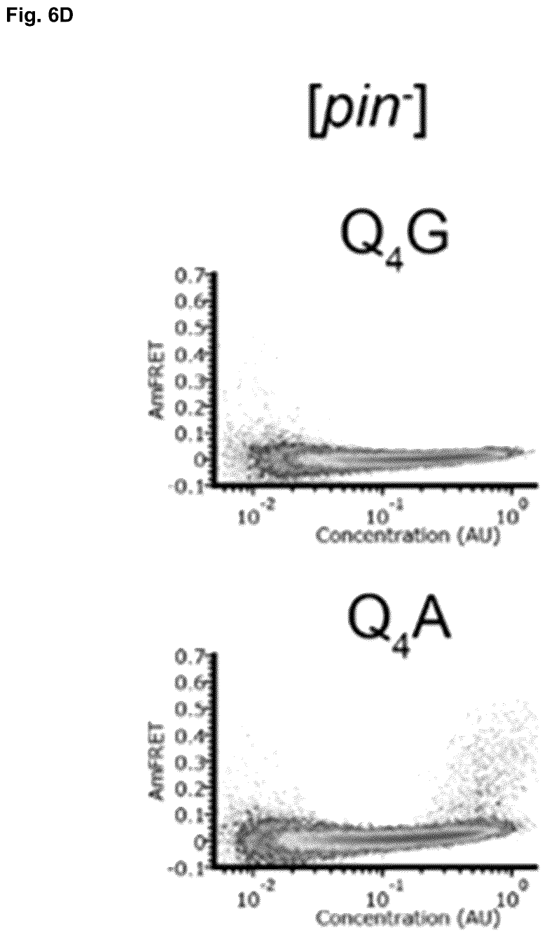

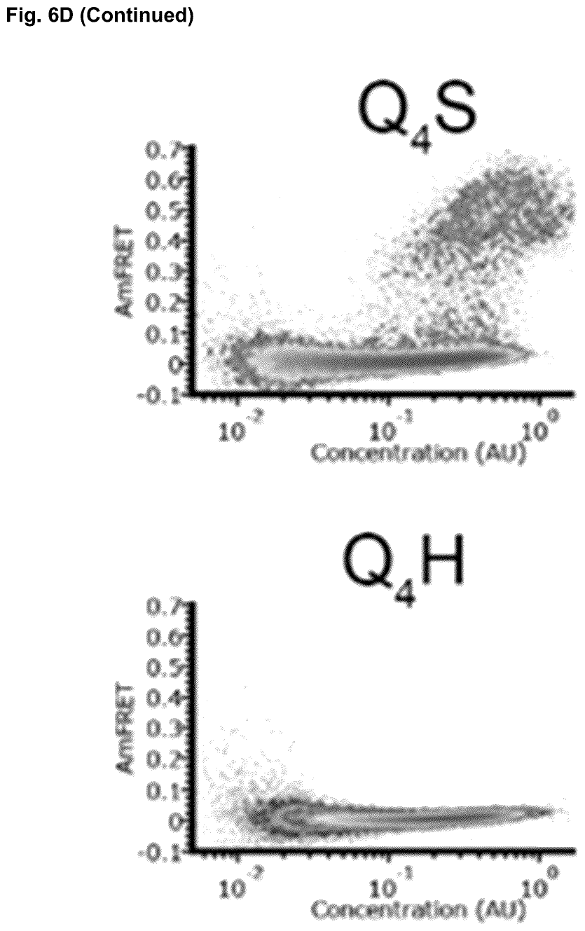

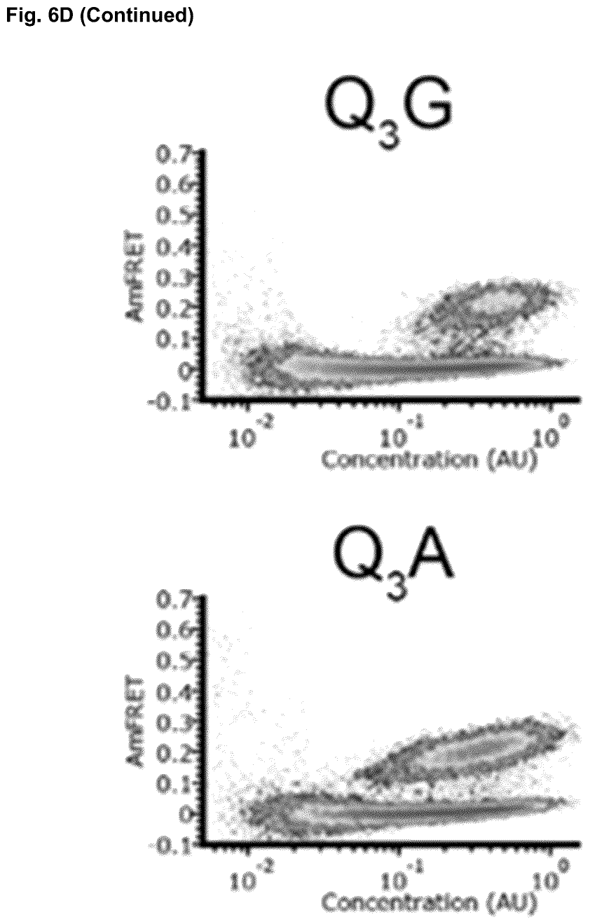

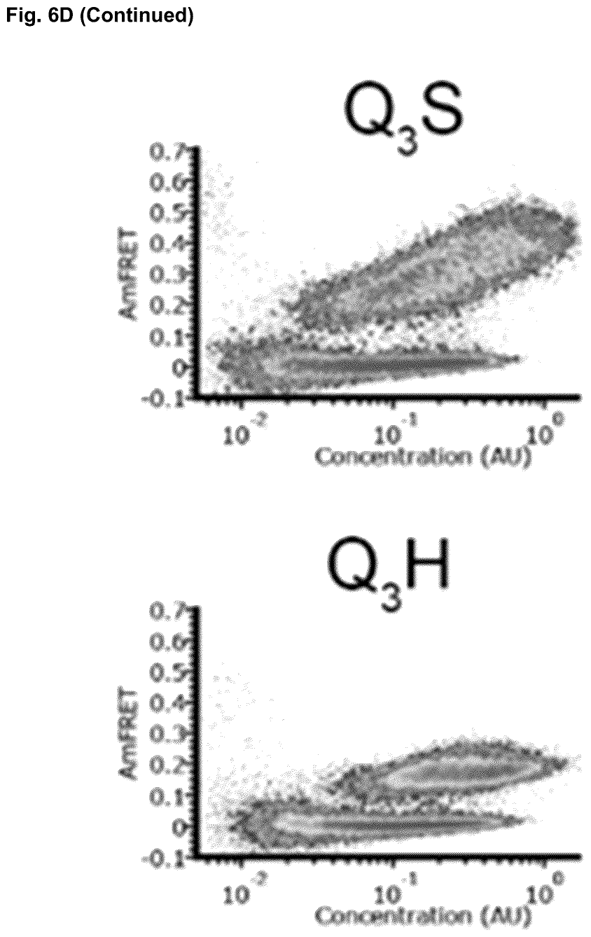

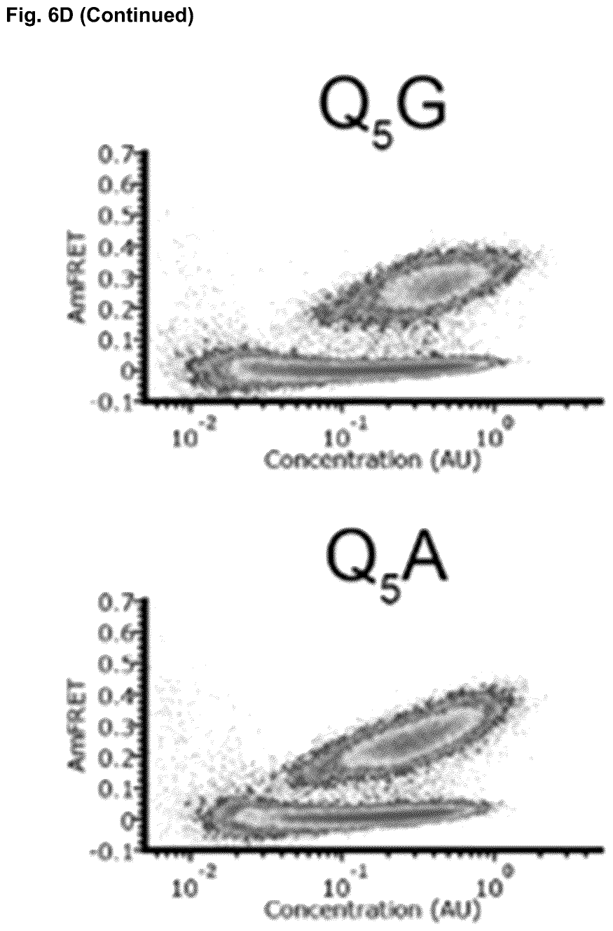

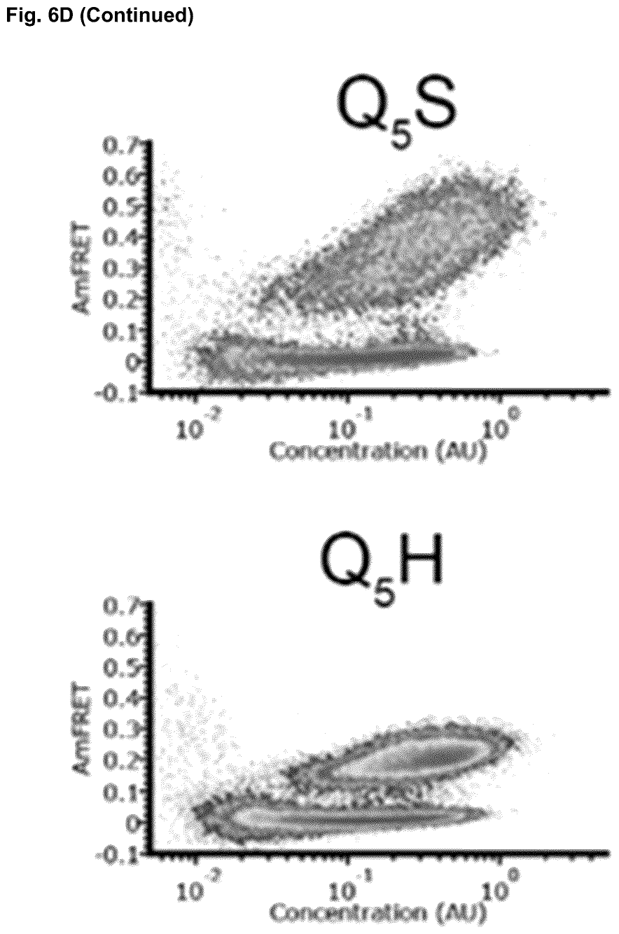

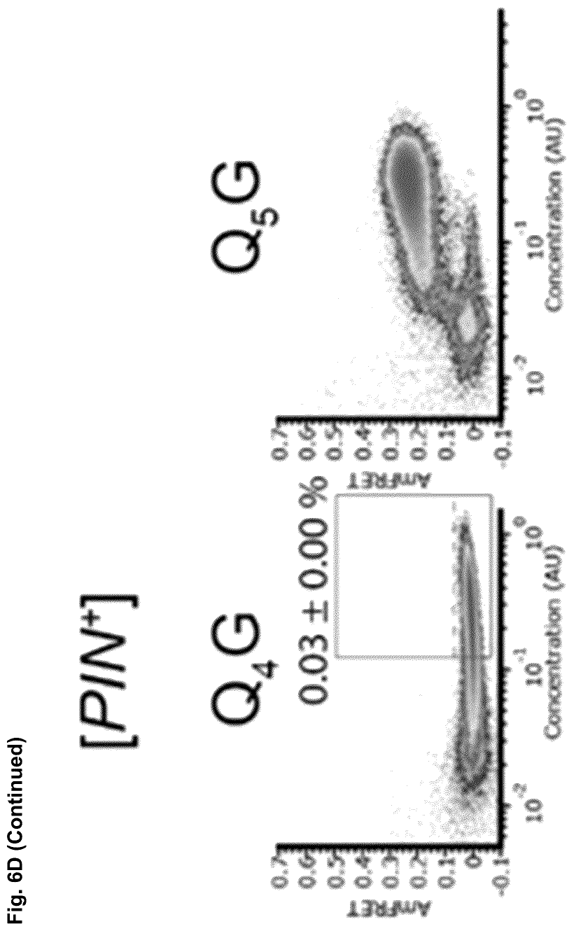

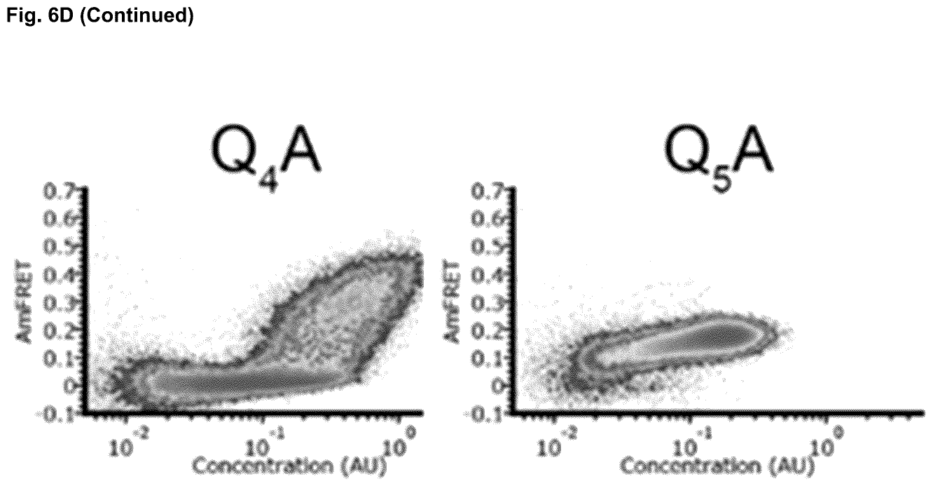

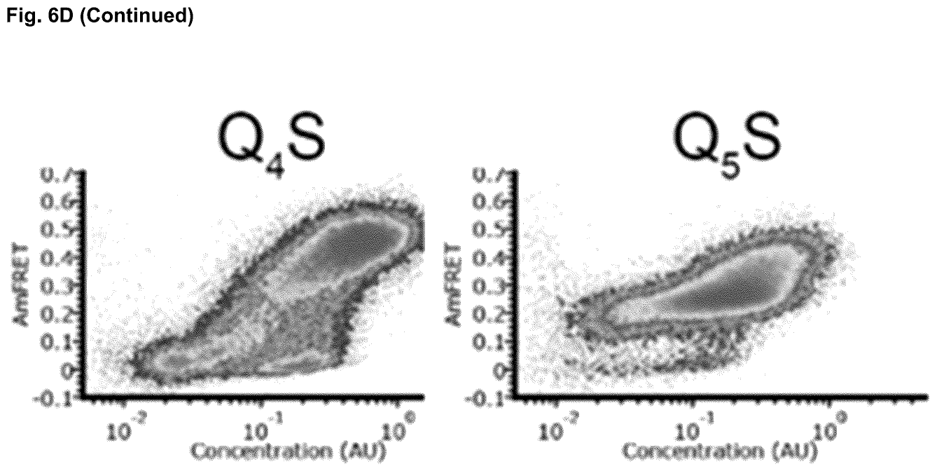

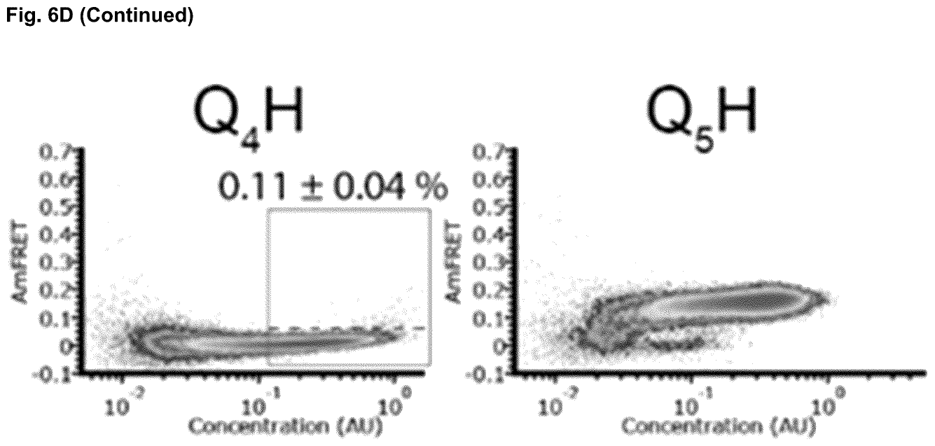

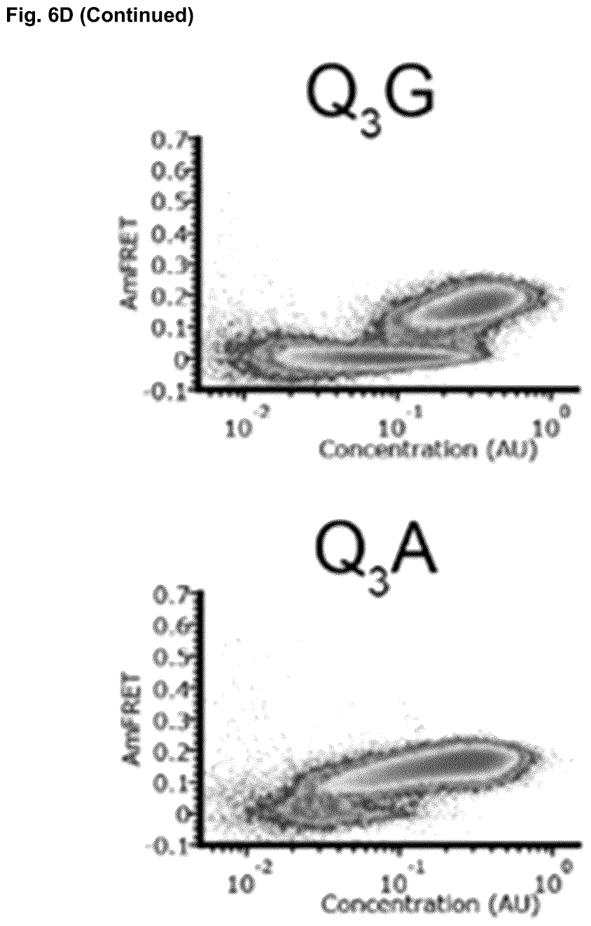

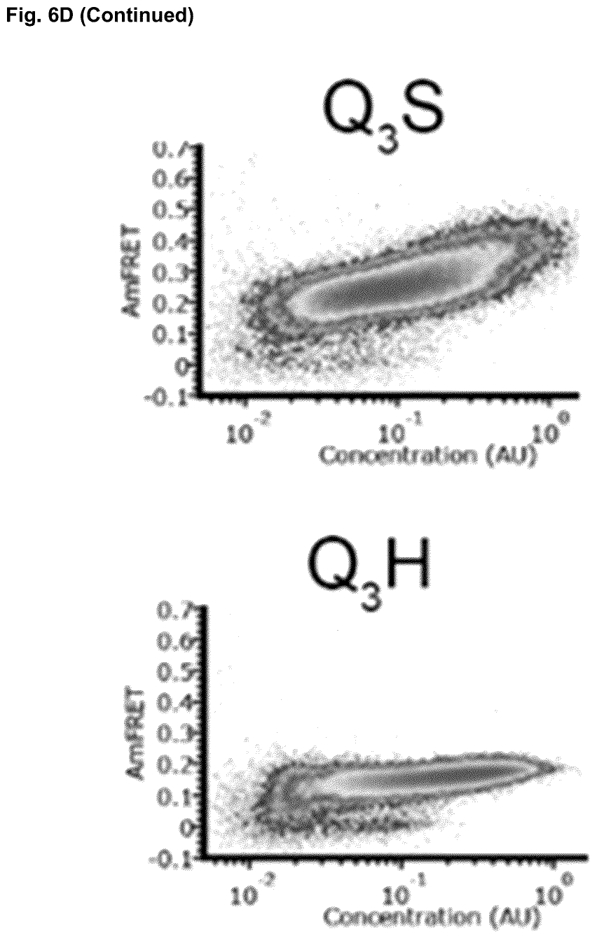

First, it was observed that amyloid formation for all values of q≥6 (FIG. 6B). Second, amyloid formation for values of q<6 was limited to odd numbers 1, 3, and 5 (FIG. 1E). This simple pattern could be explained by a local requirement for Q at every other position along the sequence (i.e. i, i+2, i+4, etc.). To test this, a second N residue was inserted into the repeats for two such sequences. Indeed, these failed to aggregate (FIG. 6C).

To determine if this “odd” dependency results from a general preference of amyloid for homogeneity at every other position, irrespective of their amino acid identity, an analogous “polyN” series was created with a single Q placed after every first, second, third, or fourth N. This series showed no preference for even or odd values (FIG. 6C), confirming that Qs, specifically, are required at every other position. We next asked if the odd dependency concerns the Q side chains themselves, rather than their interaction with Ns, by replacing the Ns with residues of diverse physicochemistry—either glycine, alanine, serine, or histidine. Again, nucleation was much more frequent for Q₃X and Q₅X than for Q₄X (FIGS. 1E, 6D), confirming that the odd dependency resulted from the Q side chains interacting specifically with other Q side chains. The identity of X did, however, influence nucleation frequency particularly for Q₄X. Glycine proved just as detrimental as asparagine; histidine slightly less so; and alanine lesser still. Serine was relatively permissive. The relative indifference of Q₃X and Q₅X to the identity of X suggests that these sequences form a pure Q core that excludes every other side chain. In other words, the nucleus is encoded by segments of sequence with Qs at every other position.

To reduce the contribution of the conformational fluctuation to nucleation, we next performed DAmFRET experiments for all sequences in [PIN⁺] cells. Amyloid formation broadly increased (FIGS. 6A-D), with polyQ now nucleating in most [PIN⁺] cells even at the lowest concentrations sampled by DAmFRET (˜1 μM, (Khan et al., 2018)). Importantly, amyloid formation again occurred only for polyQ lengths exceeding the clinical threshold. Additionally, nucleation in [PIN⁺] retained the structural constraints of de novo nucleation, as indicated by a) the relative impacts of different X substitutions, which again increased in the order serine<alanine<histidine<glycine/asparagine; and b) the persistence of an odd preference, with q=1, 3, and 5 nucleating more frequently than q=4. The latter failed entirely to form amyloid in the cases of asparagine and glycine. All N-predominant sequences and only two Q-predominant sequences (Q₂N and (Q₃N)₂N; introduced below) nucleated robustly in [PIN⁺], even though they failed entirely to do so in [pin⁻] cells. Their behavior resembles that of typical “prion-like” sequences (Khan et al., 2018; Posey et al., 2021), consistent with our deduction that these amyloids necessarily have a different nucleus structure than that of uninterrupted polyQ.

### Example 3

### A Single, Singular Steric Zipper was Encoded by the Pattern

What structure does the pattern encode, and why is it so sensitive to Ns? We sought to uncover a physical basis for the different nucleation propensities between Q and N using state-of-the-art amyloid predictors (Charoenkwan et al., 2021; Keresztes et al., 2021; Prabakaran et al., 2021). Unfortunately, none of the predictors were able to distinguish Q₃N and Q₅N from Q₄N (Table 2), despite their very dissimilar experimentally-determined amyloid propensities. In fact, most predictors failed outright to detect amyloid propensity in this class of sequences. Apparently, polyQ is exceptional among the known amyloid-forming sequences on which these predictors were trained, again hinting at a specific nucleus structure.

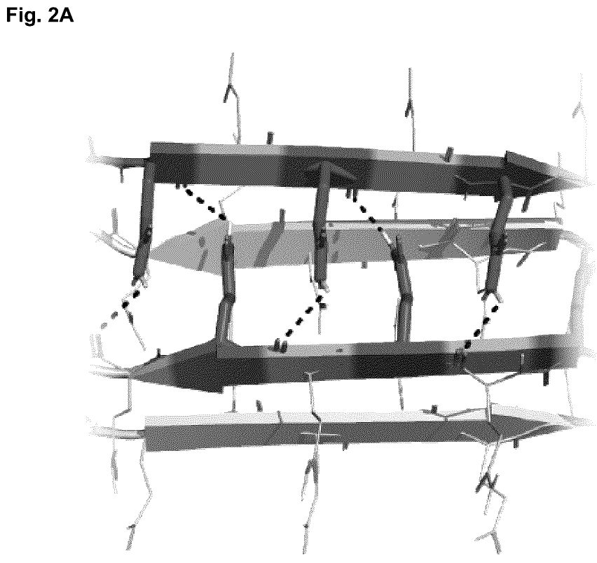

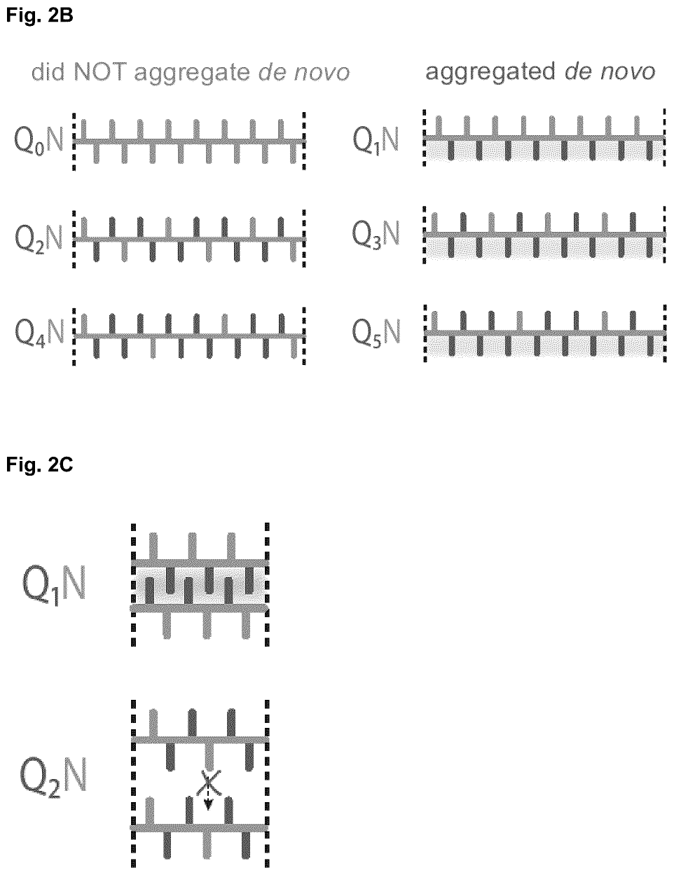

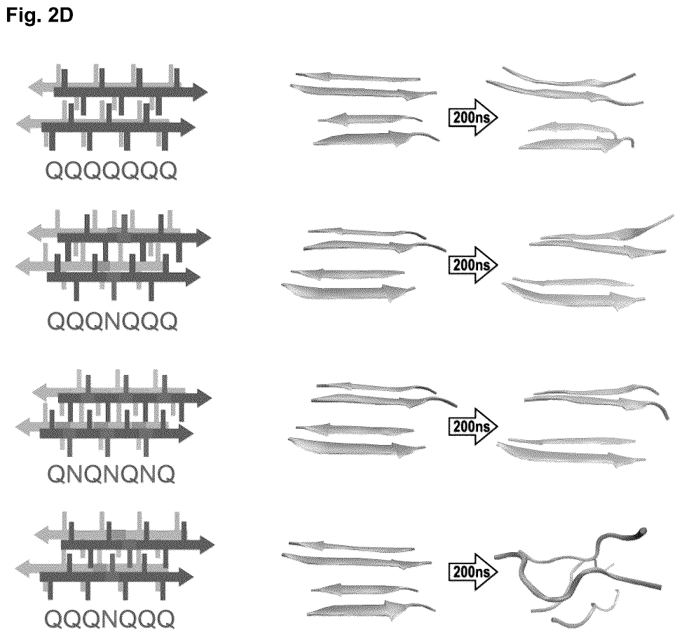

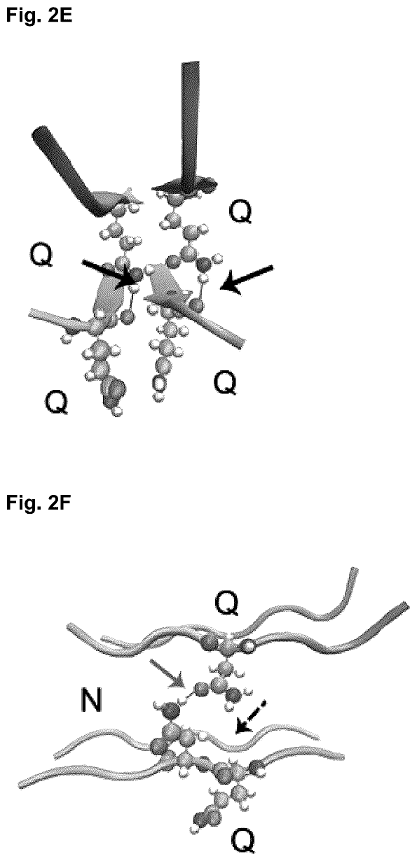

Most pathogenic amyloids feature residues that are hydrophobic and/or have a high propensity for beta strands. Glutamine falls into neither of these categories (Fujiwara et al., 2012; Nacar, 2020; Simm et al., 2016). We reasoned that nucleation of polyQ therefore corresponds to the formation of a specific tertiary structure unique to Q residues. Structural investigations of predominantly Q-containing amyloid cores reveal an exquisitely ordered tertiary motif wherein columns of Q side chains from each sheet fully extend and interdigitate with those of the opposing sheet (Hoop et al., 2016; Schneider et al., 2011; Sikorski and Atkins, 2005). The resulting structural element exhibits exceptional shape complementarity (even among amyloids) and is stabilized by the multitude of resulting van der Waals interactions. Additionally, the terminal amides of Q side chains form regular hydrogen bonds to the backbones of the opposing beta-sheet (Esposito et al., 2008; Hervas et al., 2020; Man et al., 2015; Schneider et al., 2011; Y. Zhang et al., 2016). To minimize confusion while referring to these distinguishing tertiary features of the all-Q amyloid core, relative to the similarly packed cores of virtually all other amyloids, which lack regular side chain-to-backbone H bonds (Eisenberg and Sawaya, 2017; Sawaya et al., 2007), we will henceforth refer to them as “Q zippers” (FIG. 2A).

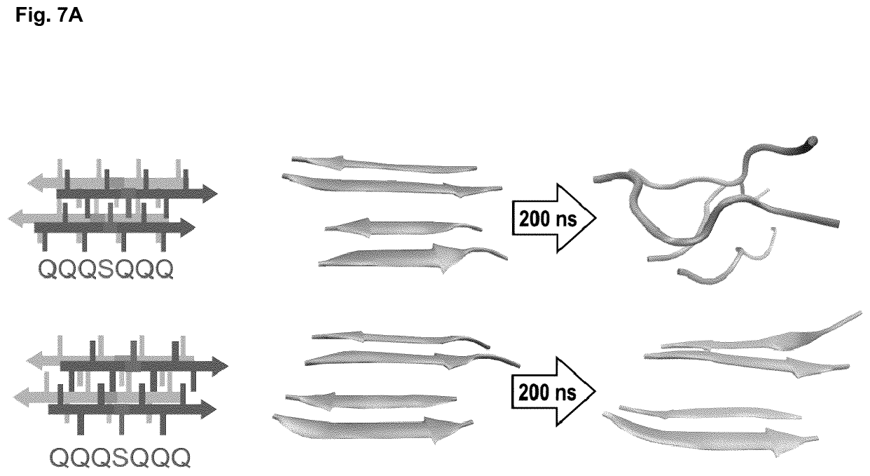

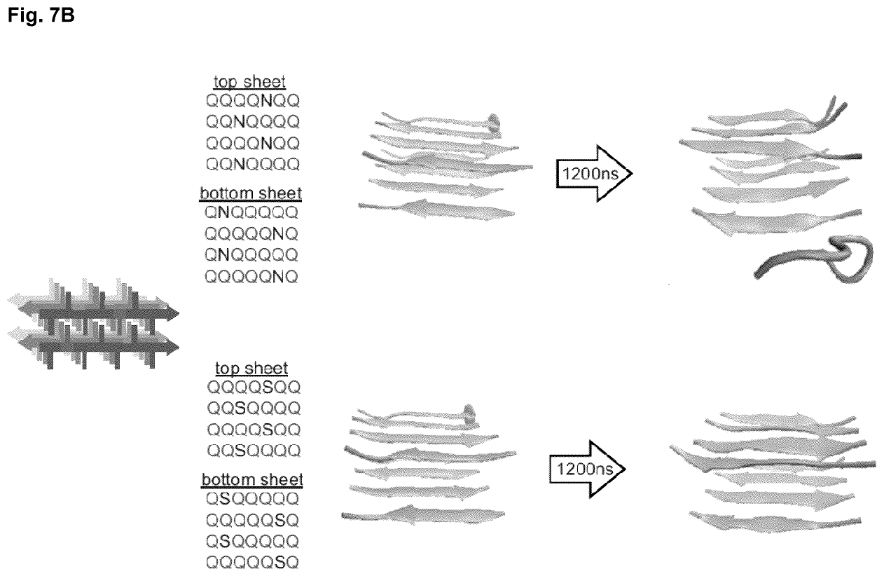

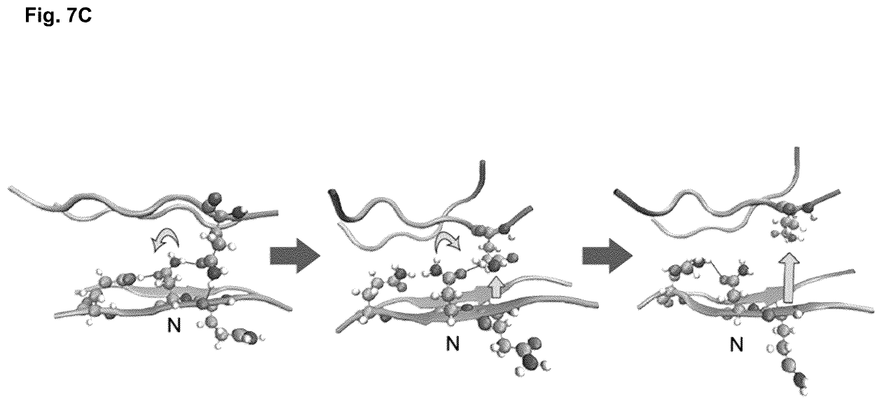

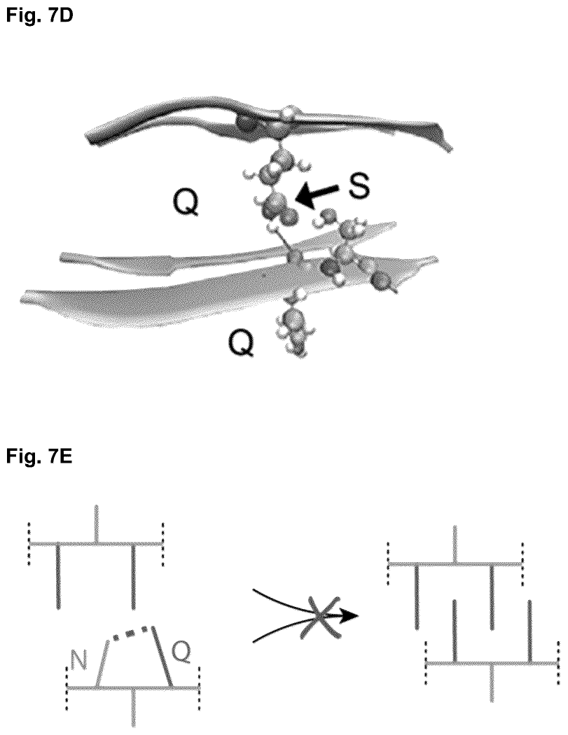

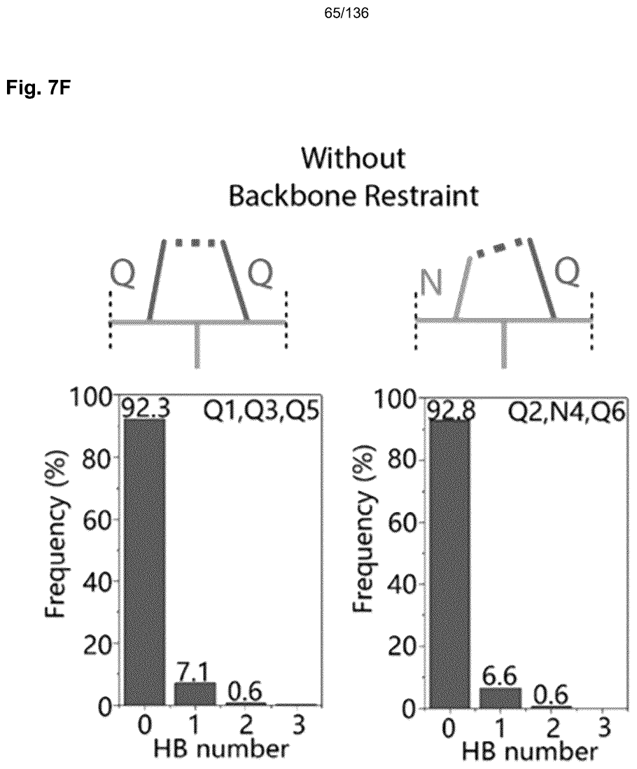

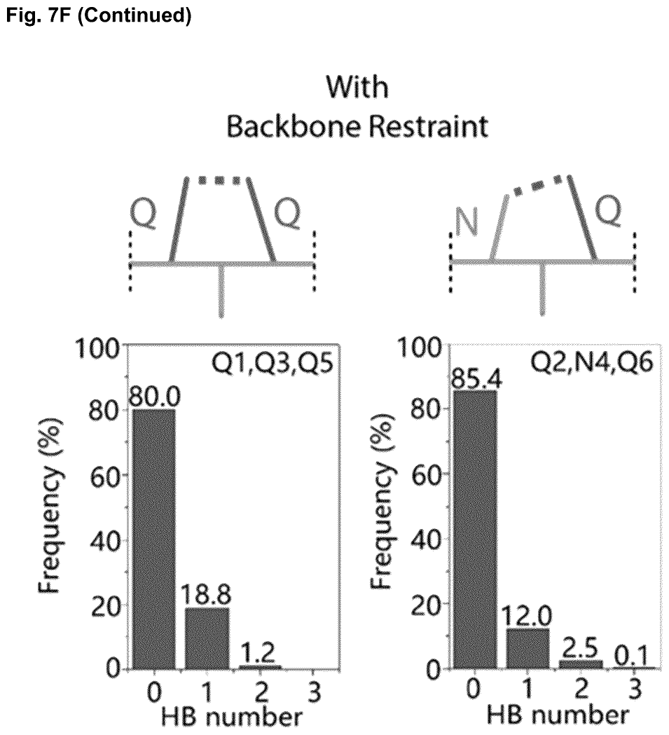

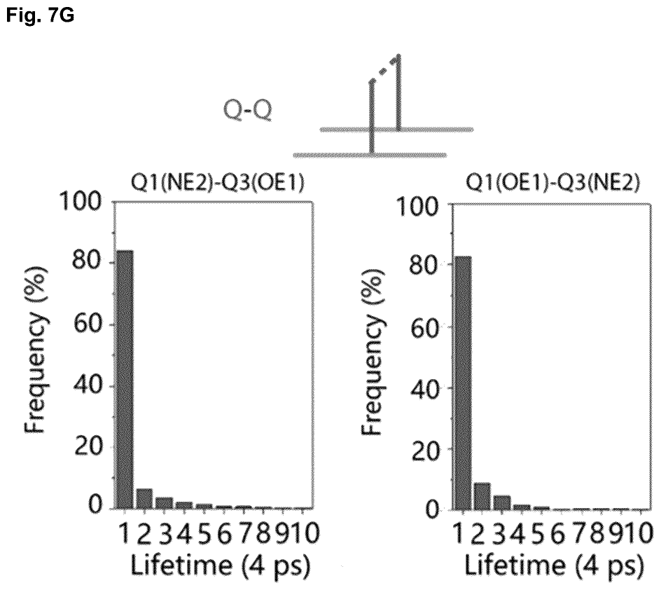

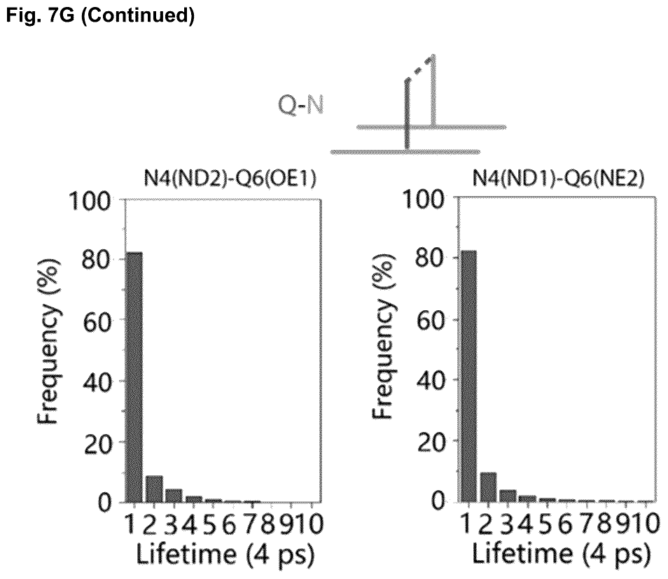

It was reasoned that the entropic cost of acquiring such an exquisitely ordered structure by the entirely disordered ensemble of naive polyQ (Chen et al., 2001; Moradi et al., 2012; Newcombe et al., 2018; Vitalis et al., 2007; Wang et al., 2006) may very well underlie the nucleation barrier. If so, then the odd dependency could relate to the fact that successive side chains alternate by 180° along a beta strand (FIGS. 2B-C). This would force non-Q side chains into the zipper for all even values of q. Our experimental results imply that the intruding residue is most disruptive when it is an N, and much less so when it is an S. To determine if the Q zipper has such selectivity, we carried out fully atomistic molecular dynamics simulations on Q-substituted variants of a minimal Q zipper model (Y. Zhang et al., 2016). Specifically, four Q7 peptides were aligned into a pair of interdigitated two-stranded antiparallel β-sheets with interdigitated Q side chains. We then substituted Q residues in different positions with either N or S and allowed the structures to evolve for 200 ns. In agreement with the experimental data, the Q zipper was highly specific for Q side chains: it rapidly dissolved when any proximal pair of inward pointing Qs were substituted, as would occur for unilaterally discontinuous sequences such as Q₄X (FIGS. 2D, 7A). In contrast, it remained intact when any number of outward-pointing Q residues were substituted, as would occur for Q₃X and Q₅X. Given the sensitivity of this minimal zipper to substitutions, we next constructed a zipper with twice the number of strands, where each strand contained a single inward pointing N or S substitution, and allowed the structures to evolve for 1200 ns. In this case, the S-containing zipper remained intact while the N-containing zipper dissolved (FIG. 7B), consistent with our experimental results.

A close examination of the simulation trajectories revealed how N disrupts Q zippers. When inside a Q zipper, the N side chain is too short to H-bond the opposing backbone. In our simulations, the terminal amide of N competed against the adjacent backbone amides for H-bonding with the terminal amide of an opposing Q side-chain, blocking that side chain from fully extending as required for Q zipper stability (FIGS. 2E-F). The now detached Q side chain collided with adjacent Q side chains, causing them to also detach, ultimately unzipping the zipper (FIG. 7C). It was found that the side chain of S, which is slightly shorter and less bulky than that of N, does not intercept H-bonds from the side chains of opposing Q residues (FIG. 7D). As a result, order is maintained so long as multiple S side chains do not occur in close proximity inside the zipper.

We also considered an alternative mechanism whereby N side chains H-bonded to adjacent Q side chains on the same side of the strand (i.e. i+2). This structure—termed a “polar clasp” (Gallagher-Jones et al., 2018)—would be incompatible with interdigitation by opposing Q side chains (FIG. 7E). We therefore quantified the frequencies and durations of side chain-side chain H-bonds in simulated singly N-substituted polyQ strands in explicit solvent, both in the presence and absence of restraints locking the backbone into a beta strand. This analysis showed no evidence that Q side chains preferentially H-bond adjacent N side chains (FIGS. 7F-G), ruling out the polar clasp mechanism of inhibition.

In sum, our simulations are consistent with polyQ amyloid nucleating from a single Q zipper comprising two β-sheets engaged across an interface of fully interdigitated Q side chains

### Example 4

### The Q Zipper Grows in Two Dimensions

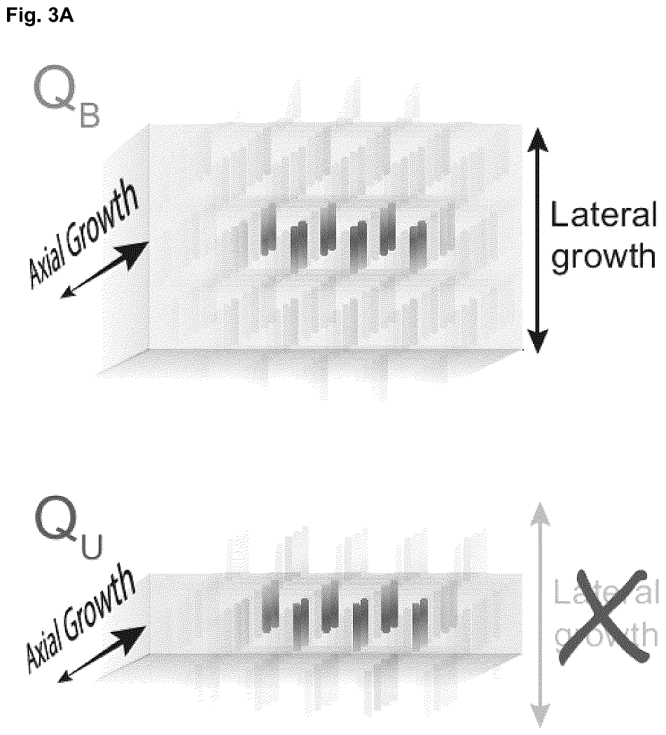

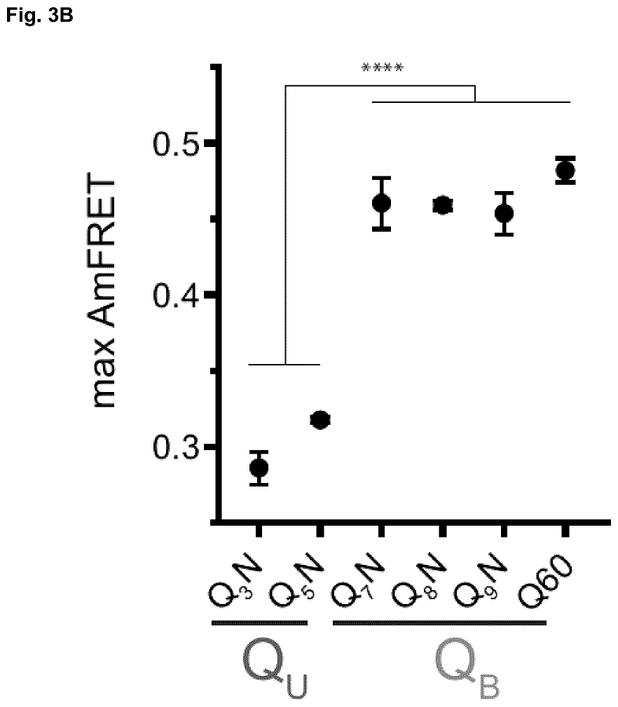

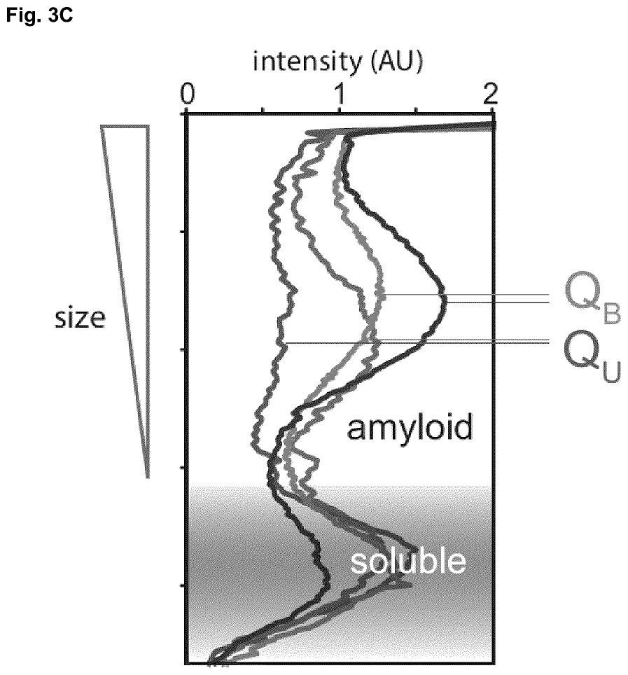

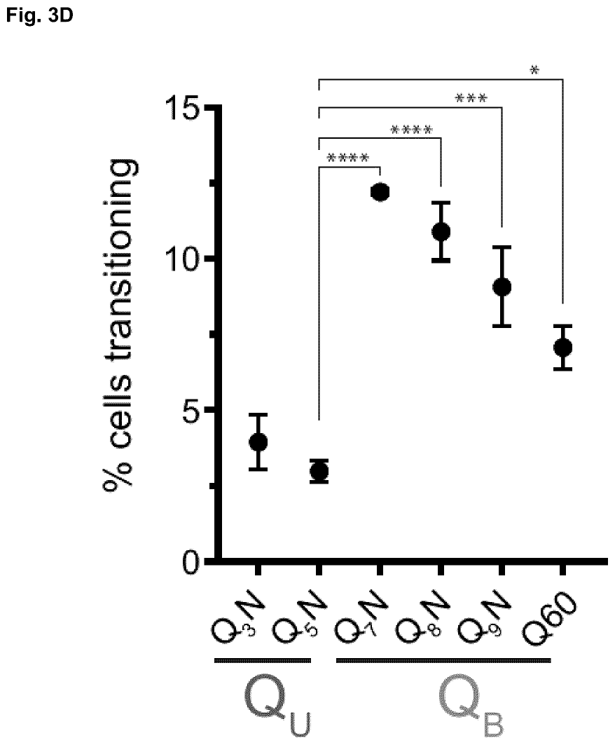

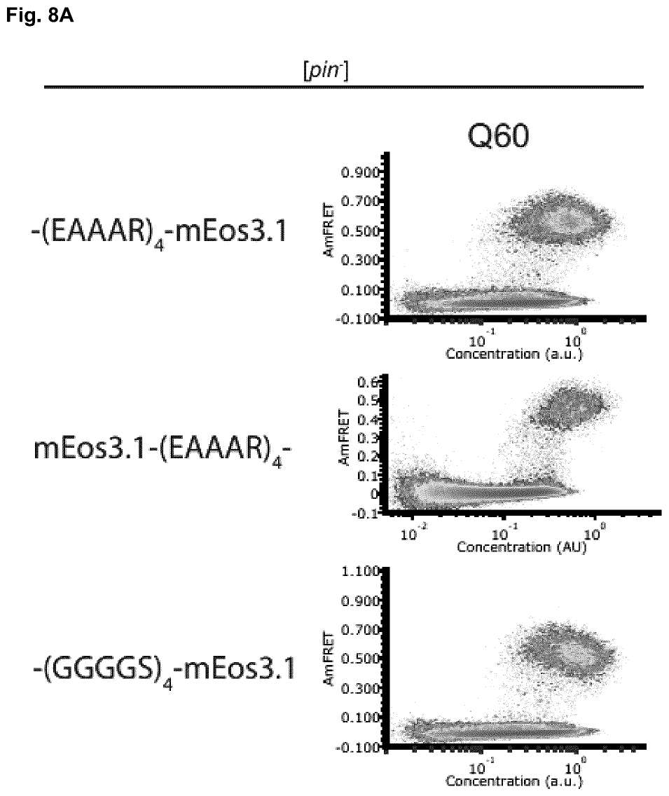

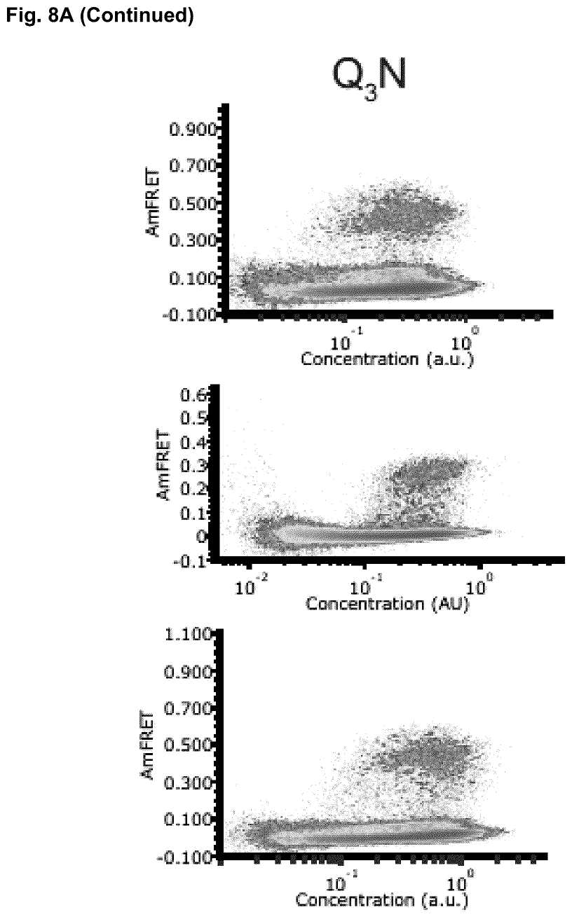

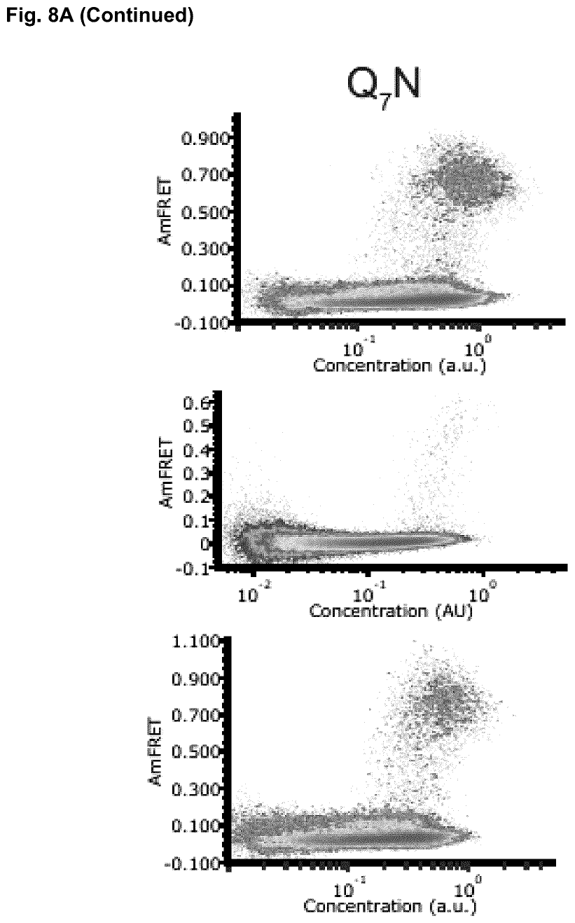

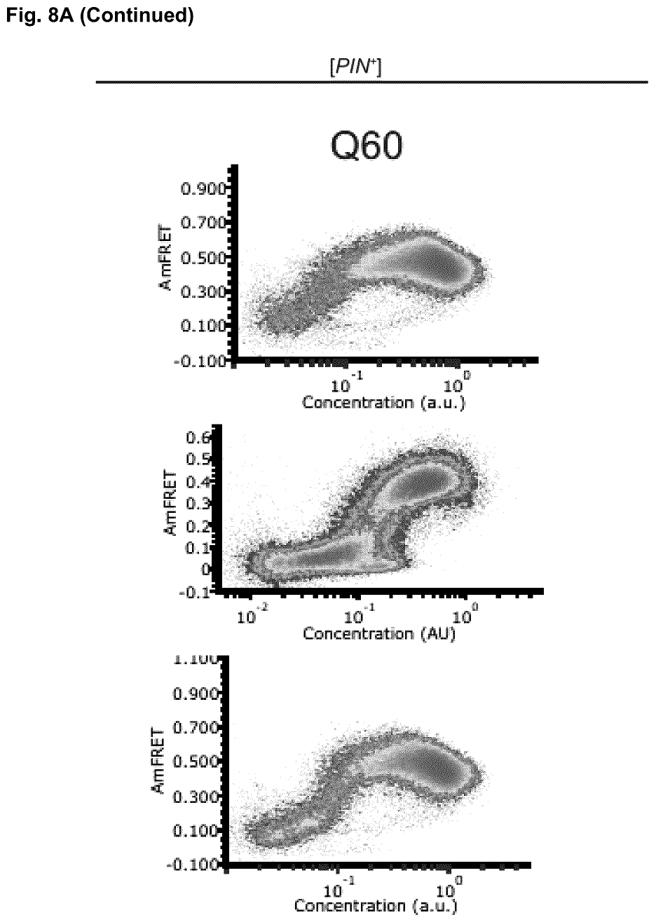

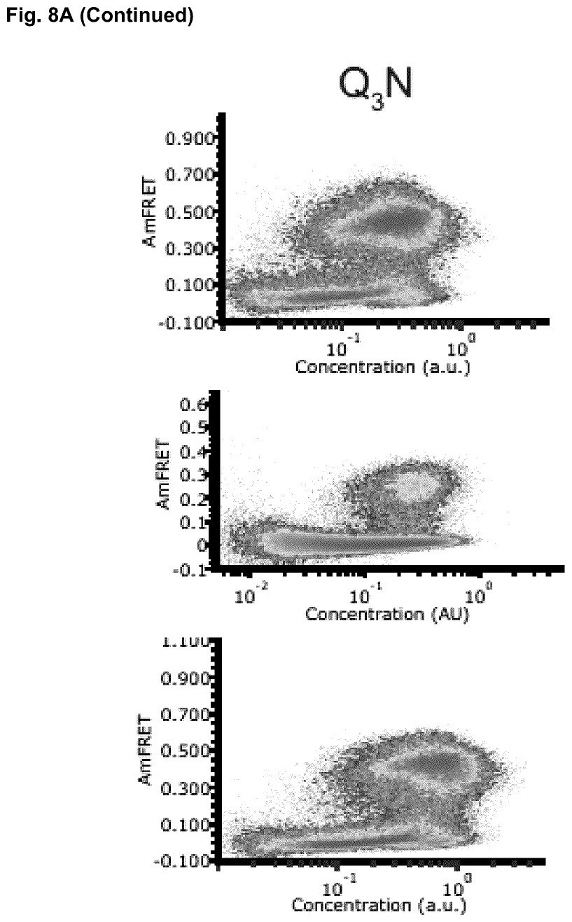

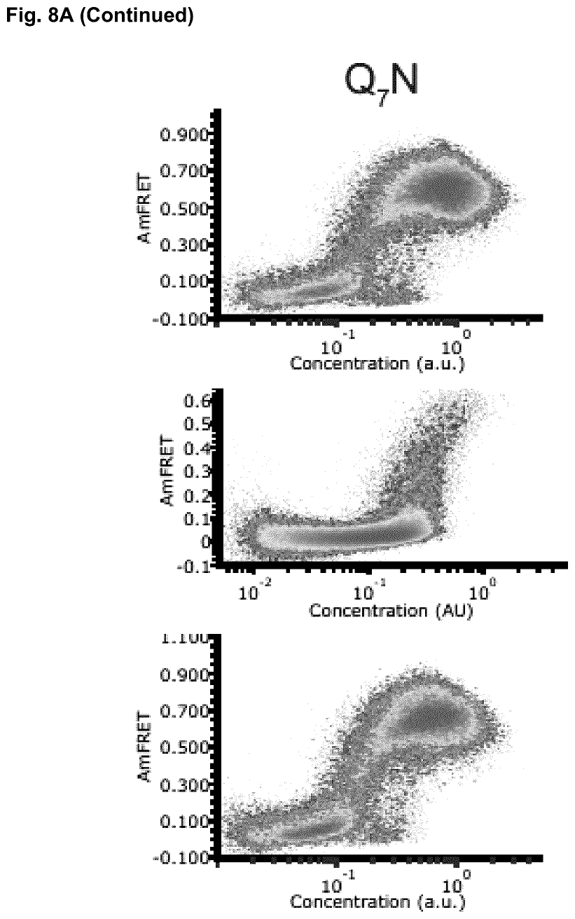

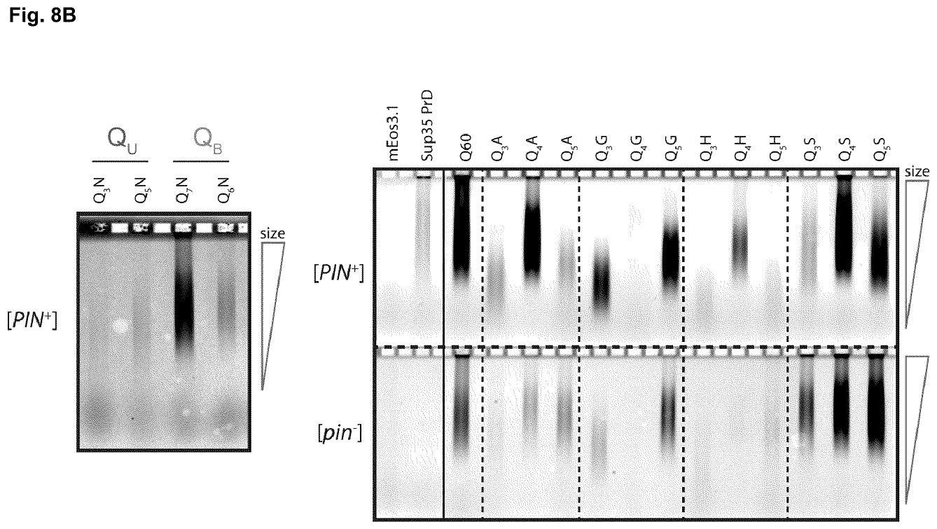

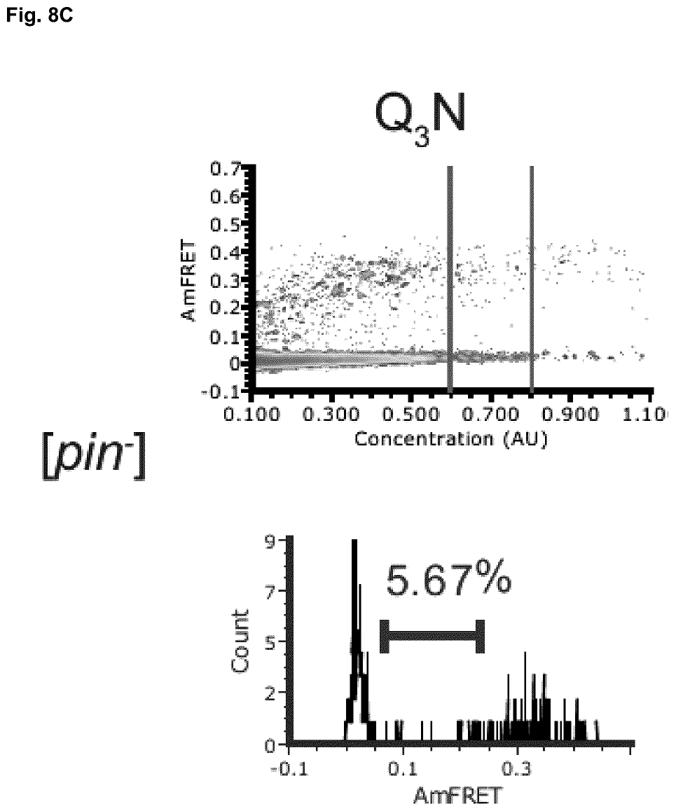

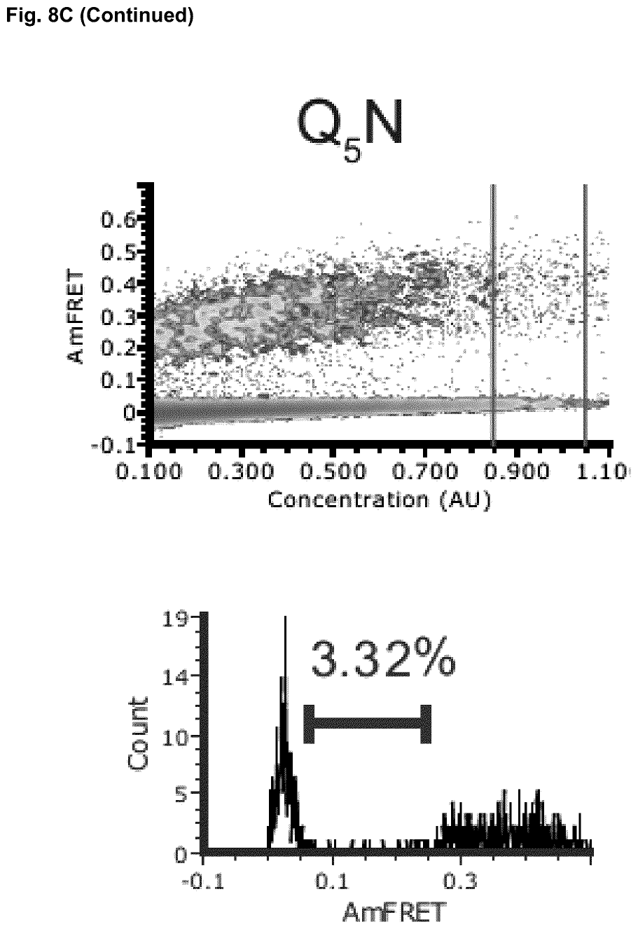

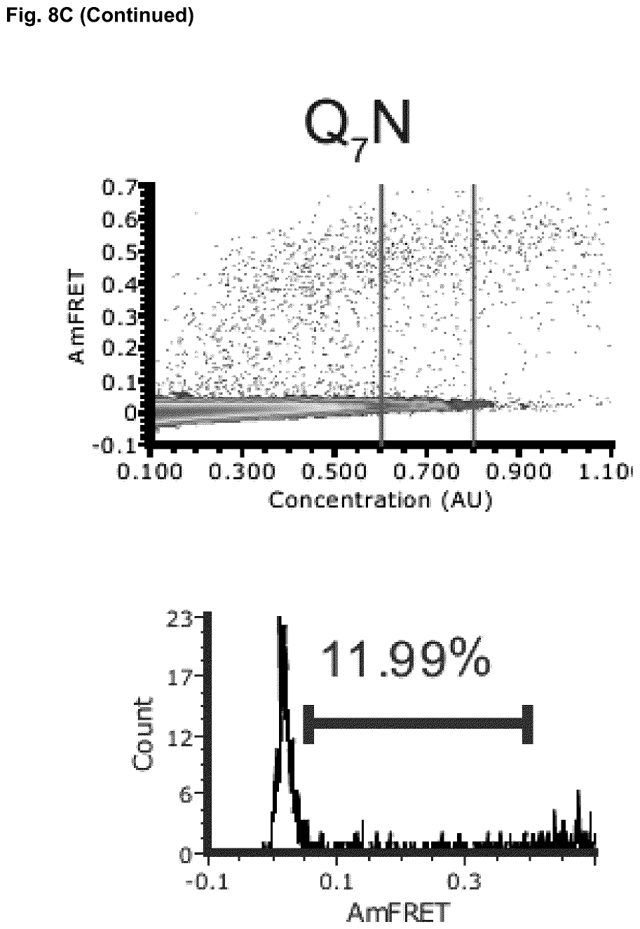

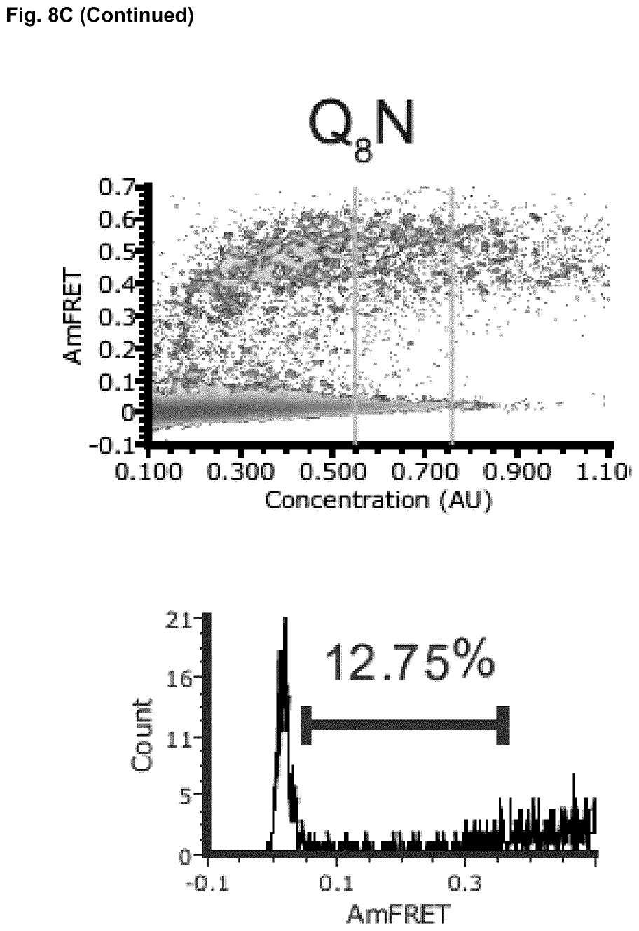

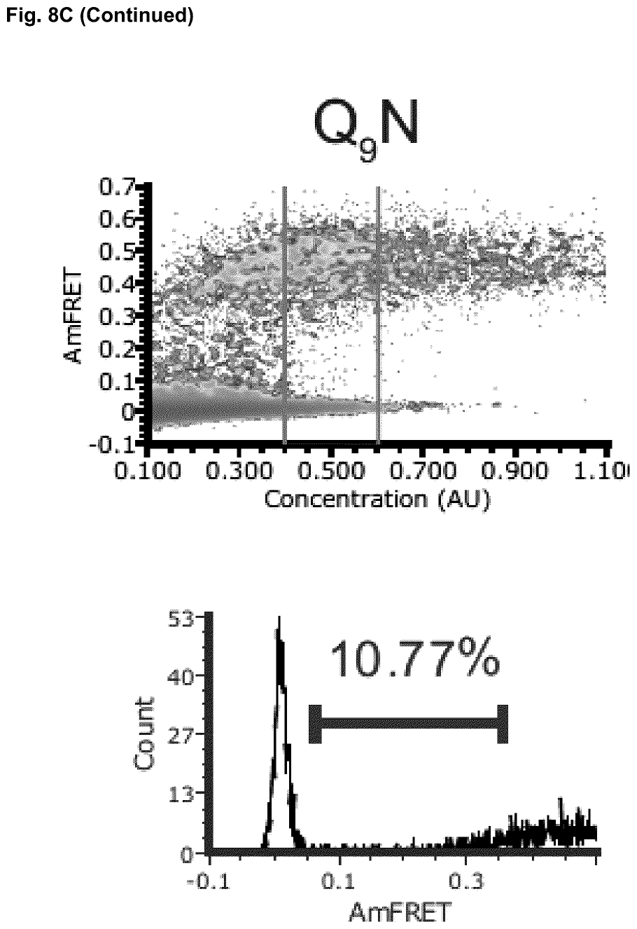

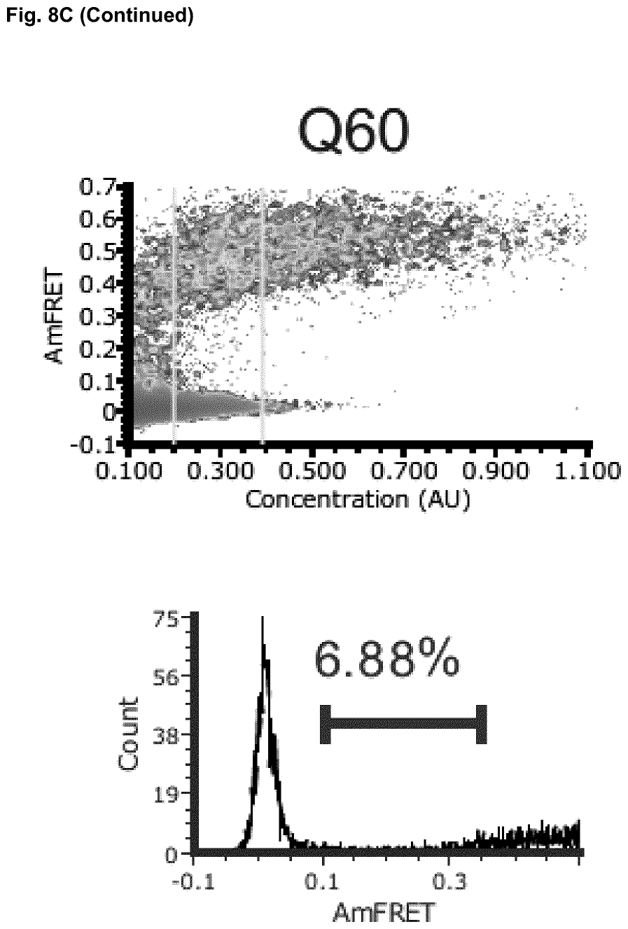

Prior structural studies show that polyQ amyloids have a lamellar or “slab-like” architecture (Boatz et al., 2020; Galaz-Montoya et al., 2021; Nazarov et al., 2022; Sathasivam et al., 2010; Sharma et al., 2005). This indicates that a nucleus comprising a single Q zipper would have to propagate not only axially but also laterally as it matures toward amyloid, provided that both sides of each strand can form a Q zipper (FIG. 3A). It was observed that when expressed to high concentrations, sequences with q values larger than 5 generally reached higher AmFRET values than those with q=3 or 5 (FIG. 3B). This suggests that the former has a higher subunit density consistent with a multilamellar structure. We will therefore refer to such sequences as bilaterally contiguous or Q_{B} and sequences that are only capable of Q zipper formation on one side of a strand, such as Q₃X and Q₅X, as unilaterally contiguous or Q_{U}. AmFRET is a measure of total cellular FRET normalized by concentration. In a two phase regime, AmFRET scales with the fraction of protein in the assembled phase (Posey et al., 2021). At very high expression levels where approximately all the protein is assembled, AmFRET should theoretically approach a maximum value determined only by the proximity of fluorophores to one another in the assembled phase. This proximity in turn reflects the density of subunits in the phase as well as the orientation of the fluorophores on the subunits. To control for the latter, we varied the terminus and type of linker used to attach mEos3.1 to polyQ, Q₃N, and Q₇N. It was found that polyQ and Q₇N achieved higher AmFRET than Q₃N regardless of linker terminus and identity (FIG. 8A). The high AmFRET level achieved by polyQ amyloids therefore results from the subunits occurring in closer proximity than in amyloids that lack the ability to form lamella.

To more directly investigate structural differences between amyloids of these sequences, we analyzed the size distributions of SDS-insoluble multimers using semi-denaturing detergent-agarose gel electrophoresis (SDD-AGE) (Halfmann and Lindquist, 2008; Kryndushkin et al., 2003). It was found that Q_{U} amyloid particles were smaller than those of Q_{B} (FIGS. 3C, 8B). This difference necessarily means that they either nucleate more frequently (resulting in more but smaller multimers at steady state), grow slower, and/or fragment more than polyQ amyloids (Knowles et al., 2009). The DAmFRET data do not support the first possibility (FIG. 6D). To compare growth rates, we examined AmFRET histograms through the bimodal region of DAmFRET plots for [pin⁻] cells. Cells with intermediate AmFRET values (i.e. cells in which amyloid had nucleated but not yet reached steady state) were less frequent for Q_{U} (FIGS. 3D, 8C), which is inconsistent with the second possibility. Therefore, the SDD-AGE data are most consistent with the third possibility—increased fragmentation. We attribute this increase to a difference in the structures rather than sequences of the two types of amyloids, because Q and N residues are not directly recognized by protein disaggregases (Alexandrov et al., 2012, 2008; Osherovich et al., 2004). Based on the fact that larger amyloid cores, such as those with multiple steric zippers, oppose fragmentation (Tanaka et al., 2006; Verges et al., 2011; Zanjani et al., 2020), these data are therefore consistent with a lamellar (FIG. 3A) architecture for polyQ.

### Example 5

### Q Zippers Poison Themselves

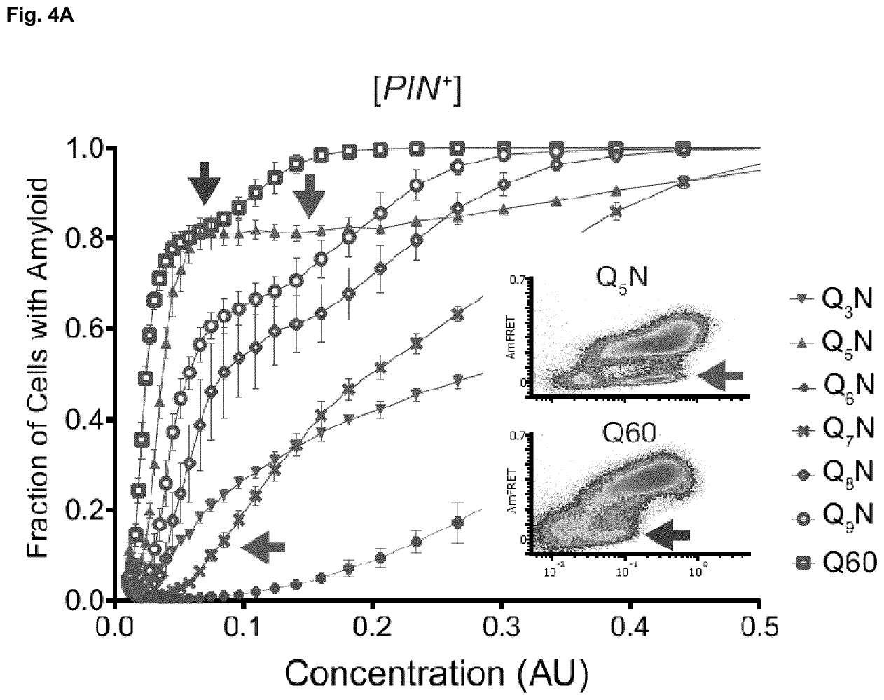

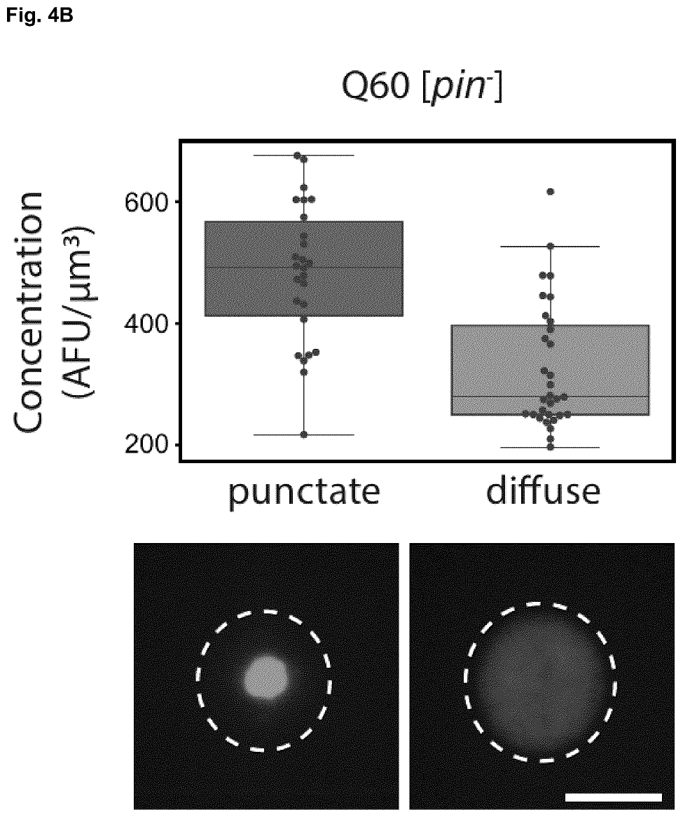

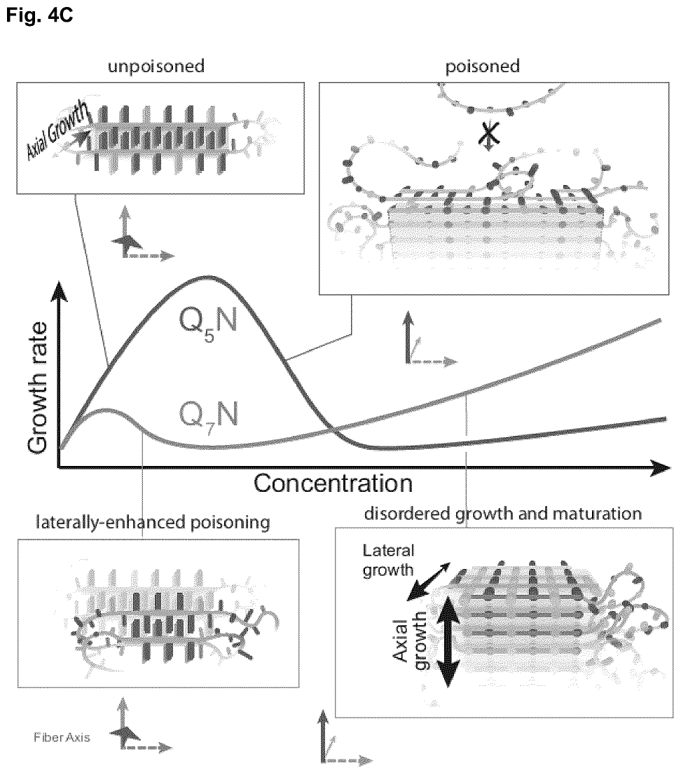

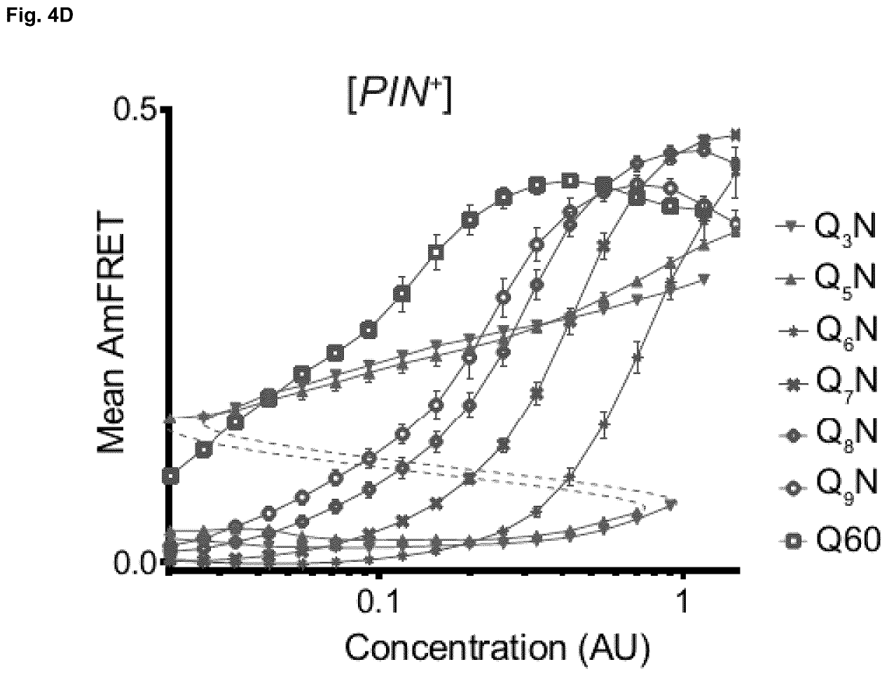

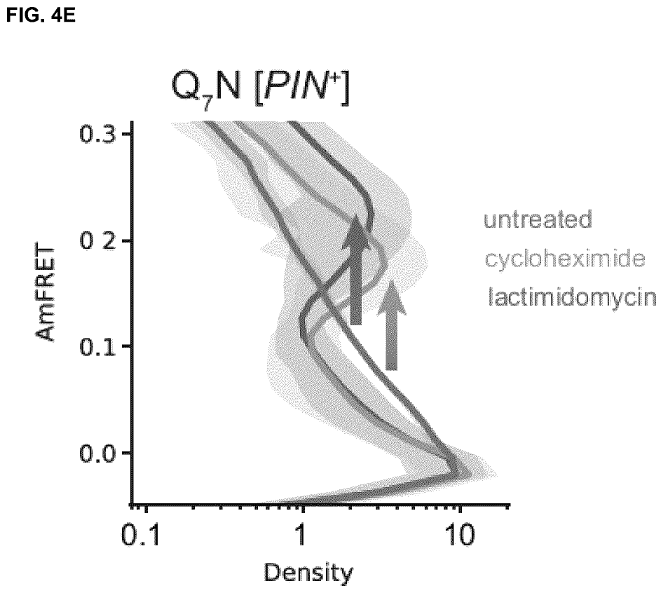

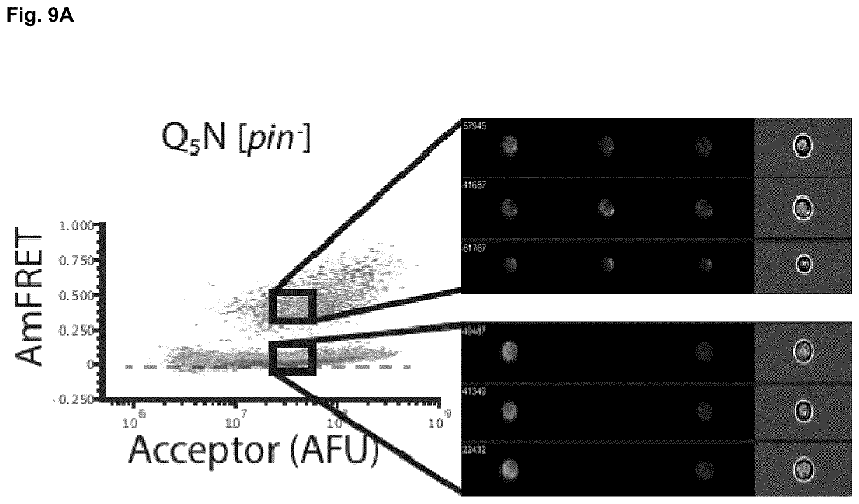

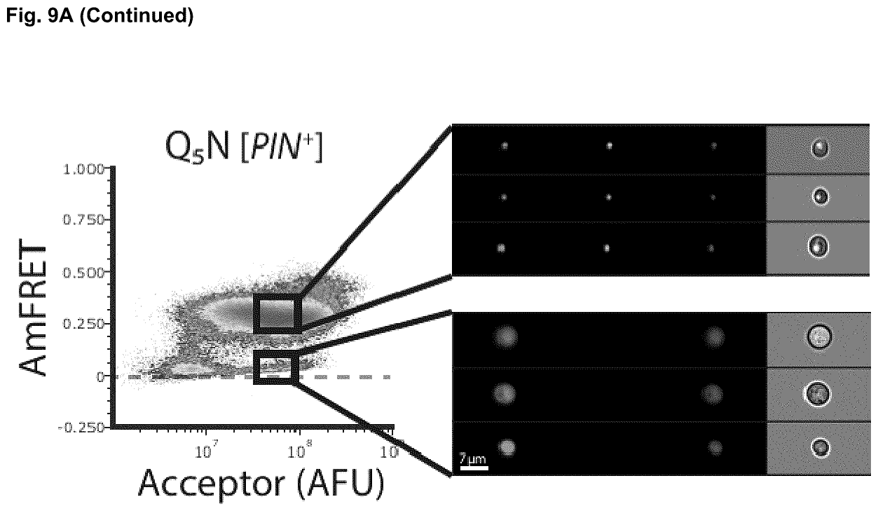

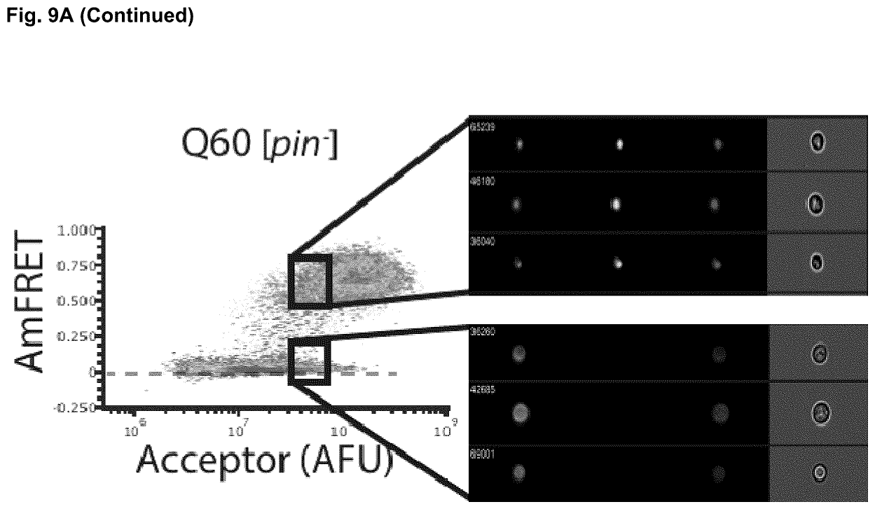

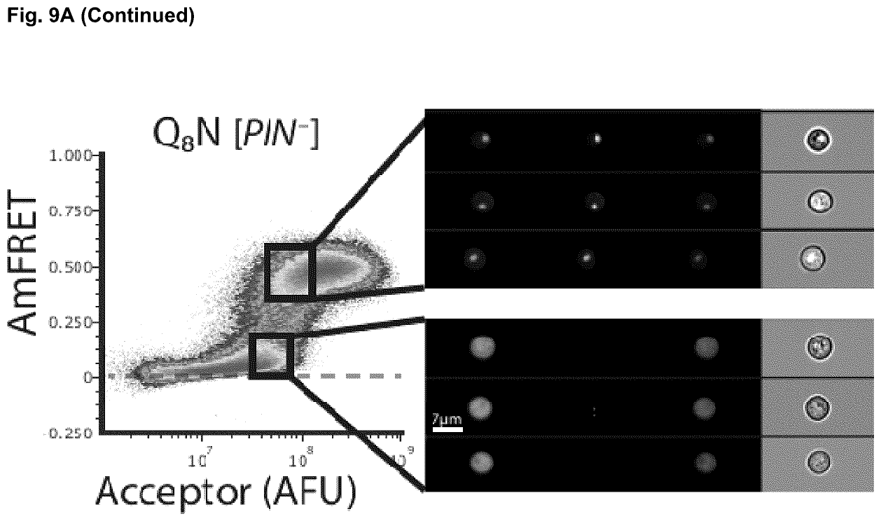

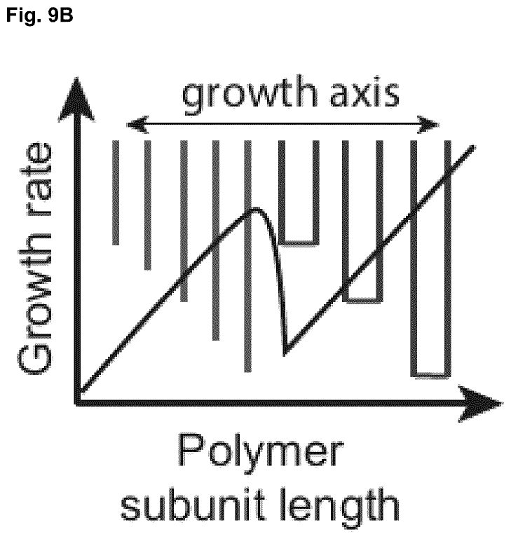

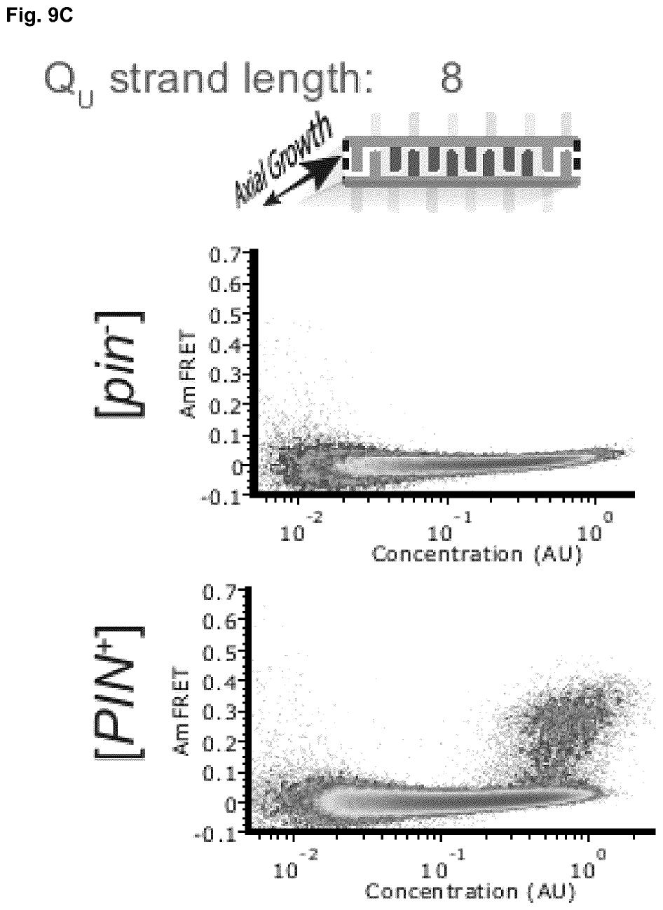

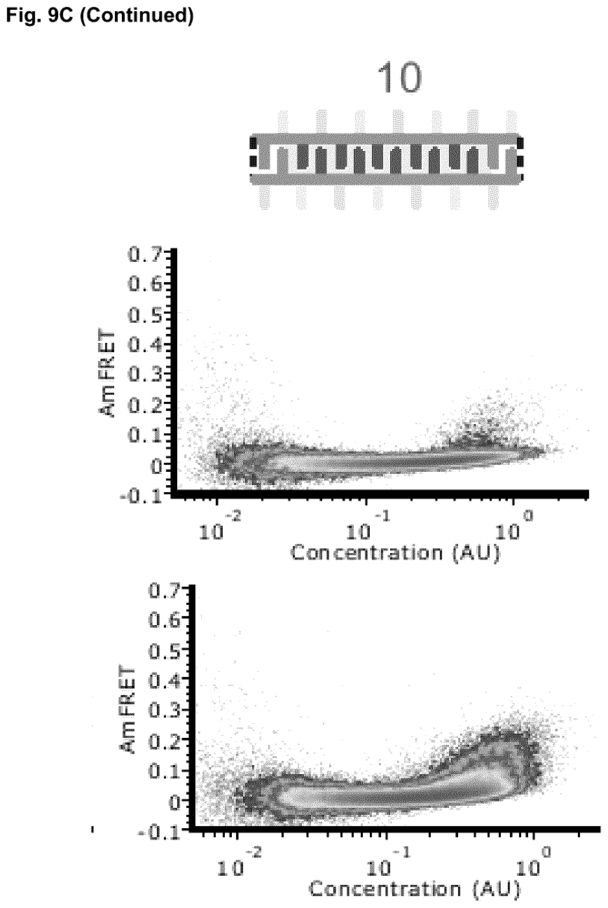

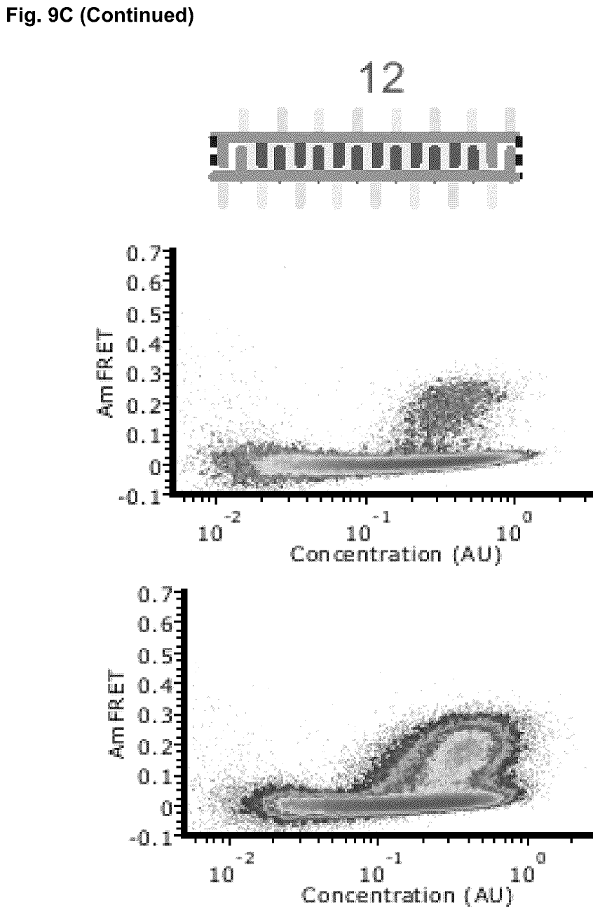

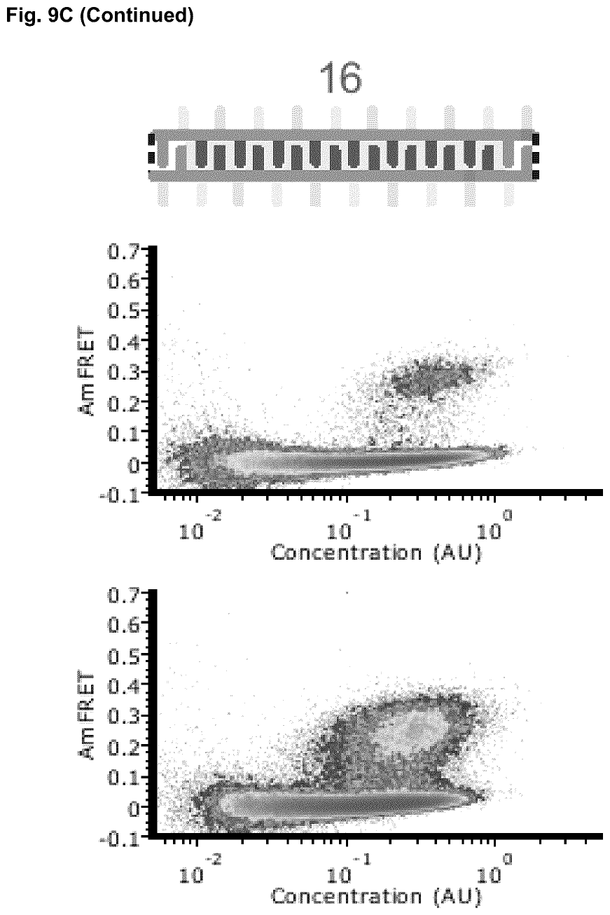

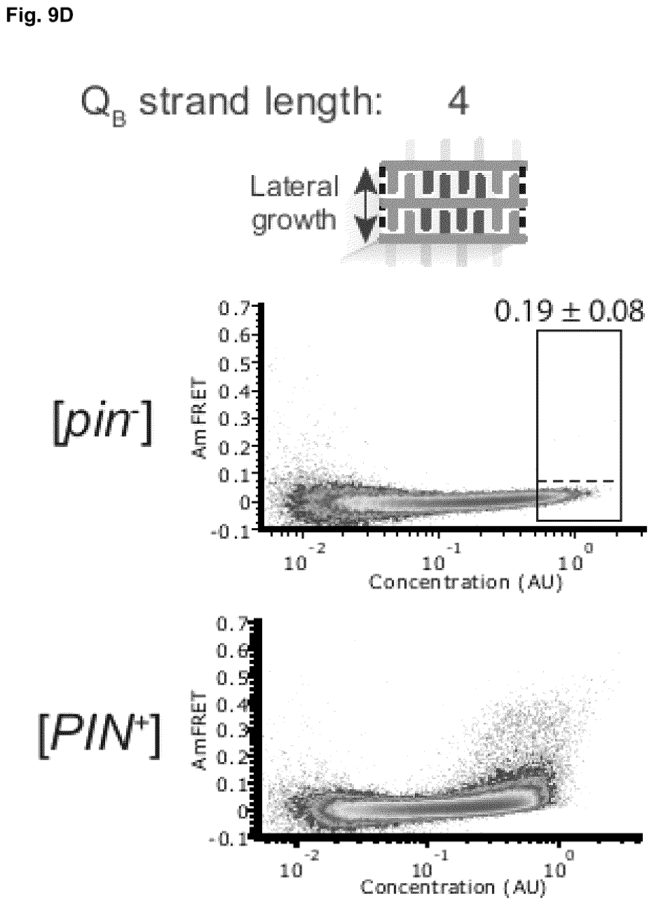

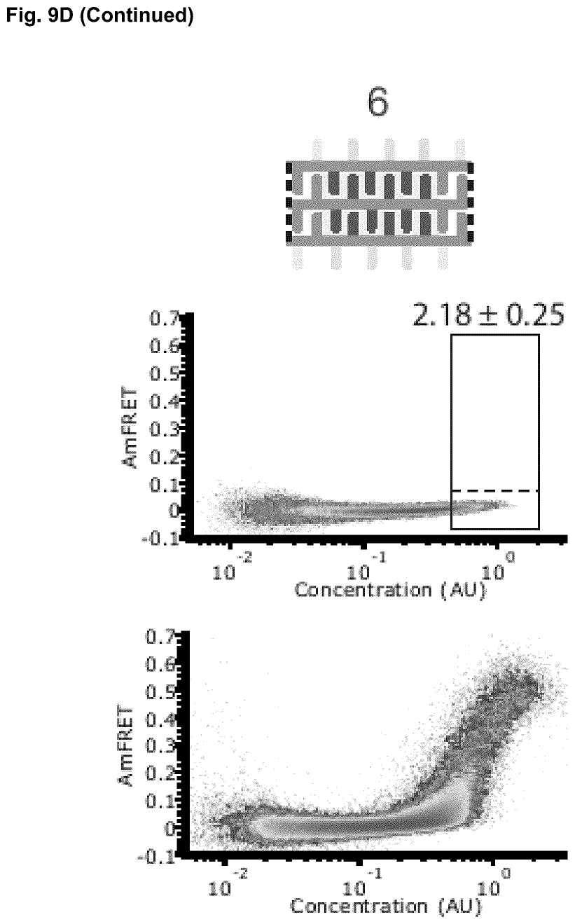

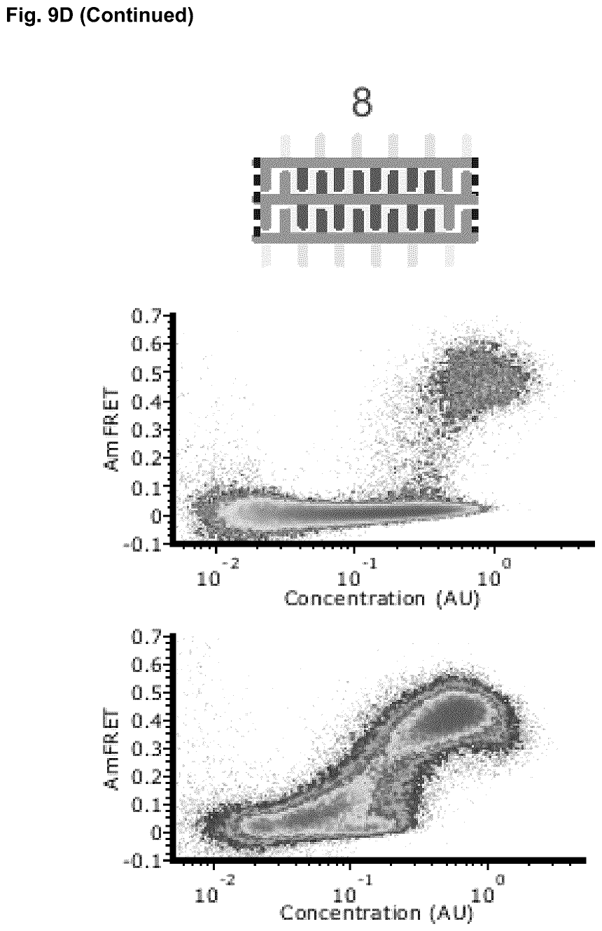

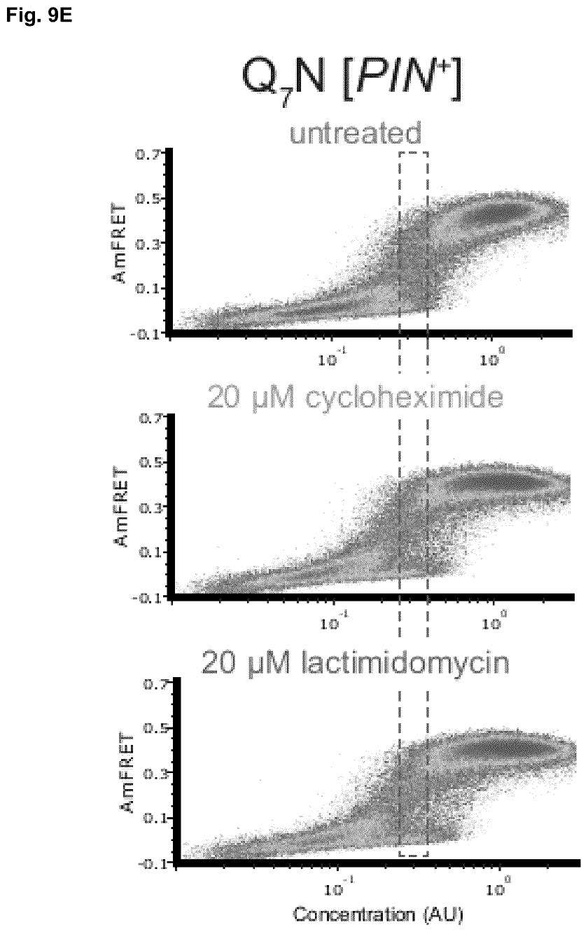

A close look at the DAmFRET data reveals a complex relationship of amyloid formation to concentration. While all sequences formed amyloid more frequently with concentration in the low regime, as concentrations increased further, the frequency of amyloid formation plateaued somewhat before increasing again at higher concentrations (FIG. 4A). A multiphasic dependence of amyloid formation on concentration implies that some higher-order species inhibits nucleation and/or growth (Vitalis and Pappu, 2011). The non-amyloid containing cells in the lower population lacked AmFRET altogether, suggesting that the inhibitory species either have very low densities of the fluorophore (e.g. due to co-assembly with other proteins), and/or do not accumulate to detectable concentrations. Liquid-liquid phase separation by low-complexity sequences can give rise to condensates with low densities and heterogeneous compositions (Wei et al., 2017), and condensation has been shown to inhibit amyloid formation in some contexts (Gabryelczyk et al., 2022; Küffner et al., 2021; Lipiński et al., 2022). We therefore inspected the subcellular distribution of representative proteins in different regions of their respective DAmFRET plots. The amyloid-containing cells had large round or stellate puncta, as expected. To our surprise, however, cells lacking AmFRET contained exclusively diffuse protein (no detectable puncta), even at high expression (FIGS. 4B, 9A). This means that naive polyQ does not itself phase separate under normal cellular conditions. The inhibitory species are therefore some form of soluble oligomer. That these oligomers are too sparse to detect by AmFRET, even at high total protein concentration, further suggests that they are dead-end (kinetically trapped) products of the Q zipper nuclei themselves. In other words, high concentrations of soluble polyQ stall the growth of nascent Q zippers.

This phenomenon was strikingly similar to “self-poisoning” in the polymer crystal literature (Sadler, 1983; Ungar et al., 2005; Whitelam et al., 2016; M. Zhang et al., 2016; Zhang et al., 2021). Self-poisoning is a deceleration of crystal growth with increasing driving force. In short, when multiple molecules simultaneously engage the templating crystal surface, they tend to “trap” each other in partially ordered configurations that block the recruitment of subsequent molecules (FIG. 4C, red). This phenomenon requires conformational conversion on the templating surface to be slow relative to the arrival of new molecules. Indeed, disordered polyQ is an extremely viscous globule (Crick et al., 2006; Dougan et al., 2009; Kang et al., 2017; Walters and Murphy, 2009) whose slow conformational fluctuations limit the rate of amyloid elongation (Walters et al., 2012).

Self-poisoning is most extreme where phase boundaries converge, as when a polymer is equally compatible with either of two crystal polymorphs (Hu, 2018; Ungar et al., 2005). For example, the rate of polymer crystallization rises, falls, and then rises again as a function of polymer length, where the second minimum results from the polymer heterogeneously conforming to either a single-long-strand polymorph or a hairpin polymorph (FIG. 9B). This is a consequence of secondary nucleation within the molecule (Hu, 2018; Ungar et al., 2005; M. Zhang et al., 2016; Zhang et al., 2021). Remarkably, an analogous phenomenon manifested for Q zippers. Specifically, we noticed that while Q₅N formed amyloid robustly at low concentrations (i.e. prior to self-poisoning at higher concentrations), Q₇N did not (FIG. 4A, purple arrow). We were initially perplexed by this because all odd q sequences have a fully contiguous pattern of Qs at every other position that should allow for Q zipper nucleation. We then realized, however, that the anomalous solubility of Q₇N coincides with the expected enhancement of self-poisoning at the transition between single long Q zippers (favored by Q_{U} sequences) and short lamellar Q zippers (favored by Q_{B} sequences). In other words, the striking difference between Q₅N and Q₇N appears to result from the formation of intramolecular Q zippers with strands only six residues long, which form contralaterally to the intermolecular Q zipper interface (FIG. 4C, blue). As for other polymers, Q_{B} sequences increasingly escaped the poisoned state as the potential length of lamellar strands increased beyond the minimum, i.e. with increasing q values beyond 6 (FIG. 4D).

In our experimental system, ongoing protein translation will cause the polypeptide to become increasingly supersaturated with respect to the self-poisoned amyloid phase. However, as concentrations increase, the contributions of unstructured low affinity interactions (primarily H-bonds between Q side chains (Kang et al., 2018; Punihaole et al., 2018)) to intermolecular associations will increase relative to the contributions of Q zipper elements. Disordered polyQ has a greater affinity for itself than does disordered polyN (Halfmann et al., 2011). The ability of Q_{B} sequences to escape poisoning with increasing concentrations may therefore be a simple consequence of their greater content of Qs. Indeed, polyQ amyloid (Walters et al., 2012), as for other polymer crystals (Zhang et al., 2021), has a relatively disordered growth front at high polymer concentrations.

Our finding that polyQ does not phase-separate prior to Q zipper nucleation implies that intermolecular Q zippers will be less stable than intramolecular Q zippers of the same length, because the effective concentration of Q zipper-forming segments will always be lower outside the intramolecular globule than inside it. Therefore, the critical strand length for growth of a single (non-lamellar) Q zipper must be longer than six, whereas strands of length six will only grow in the context of lamella. To test this prediction, we designed a series of sequences with variably restricted unilateral or bilateral contiguity (FIGS. 9C-D). It was found that, while all sequences formed amyloid with a detectable frequency in [PIN⁺] cells, only those with at least five unilaterally contiguous Qs, or at least six bilaterally contiguous Qs, did so in [pin⁻] cells. Given that the ability to nucleate in the absence of a pre-existing conformational template is characteristic of Q zippers, this result is consistent with a threshold strand length of nine or ten residues for single Q zippers to propagate, and six residues for lamellar Q zippers to propagate.

One consequence of self-poisoning is that crystallization rates accelerate with monomer depletion (Ungar et al., 2005). In our system, poisoning should be most severe in the early stages of amyloid formation when templates are too few for polypeptide deposition to outpace polypeptide synthesis, thereby maintaining concentrations at poisoning levels. For sufficiently fast nucleation rates and slow growth rates, this property predicts a bifurcation in AmFRET values as concentrations enter the poisoning regime, as we see for Q_{B} in [PIN⁺] cells. To determine if the mid-AmFRET cells in the bifurcated regime indeed have not yet reached steady state, we used translation inhibitors to stop the influx of new polypeptides—and thereby relax self-poisoning—for six hours prior to analysis. As predicted, treated AmFRET-positive cells in the bifurcated regime achieved higher AmFRET values than cells whose translation was not arrested (FIGS. 4E, 9E).

### Example 6

The Nucleus Forms within a Single Molecule

Because amyloid formation is a transition in both conformational ordering and density, and the latter is not rate-limiting over our experimental time-scales (Khan et al., 2018), the critical conformational fluctuation for most amyloid-forming sequences is generally presumed to occur within disordered multimers (Auer et al., 2008; Buell, 2017; Serio et al., 2000; Vekilov, 2012; Vitalis and Pappu, 2011). In contrast, homopolymer crystals nucleate preferentially within monomers when the chain exceeds a threshold length (Hu, 2018; Xu et al., 2021). The possibility that polyQ amyloid may nucleate as a monomer has long been suspected to underlie the length threshold for polyQ pathology (Chen et al., 2002). A minimal intramolecular Q zipper would comprise a pair of two-stranded beta sheets connected by loops of approximately four residues (Chou and Fasman, 1977; Hennetin et al., 2006). If we take the clinical threshold of approximately 36 residues as the length required to form this structure, then the strands must be approximately six residues with three unilaterally contiguous Qs—exactly the length we deduced for intramolecular strand formation in the previous section.

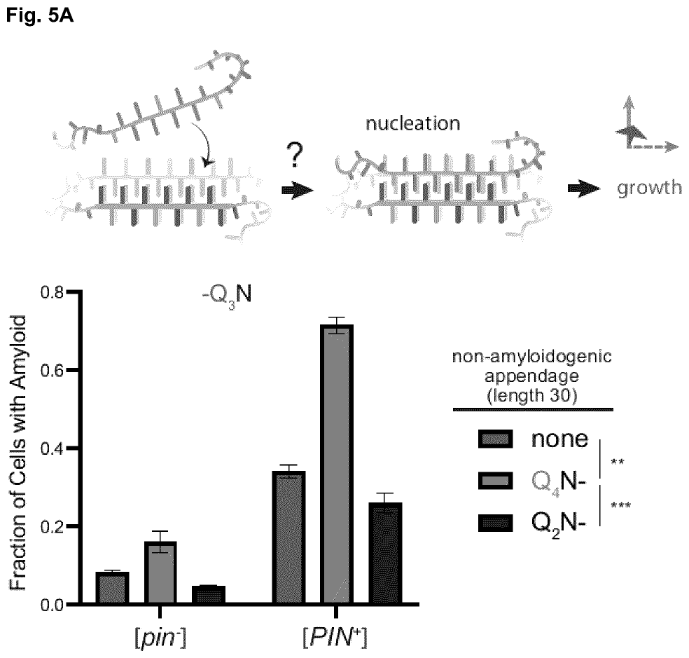

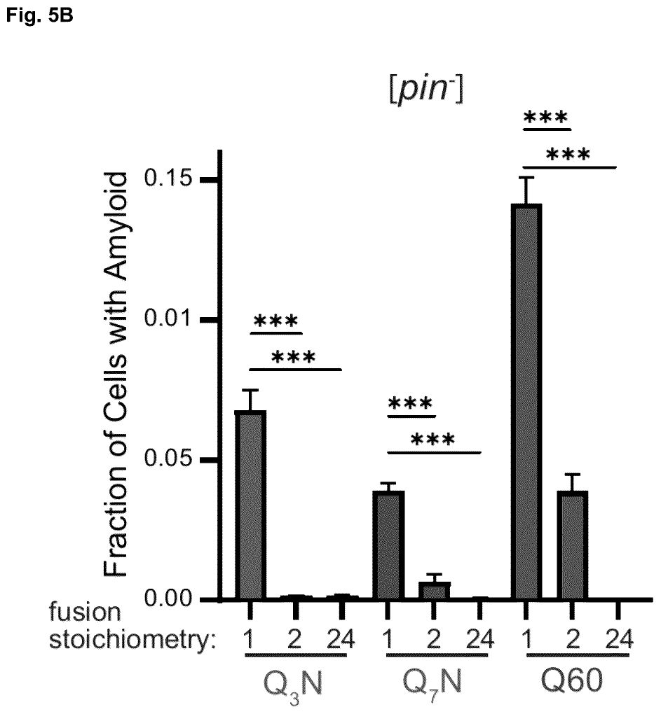

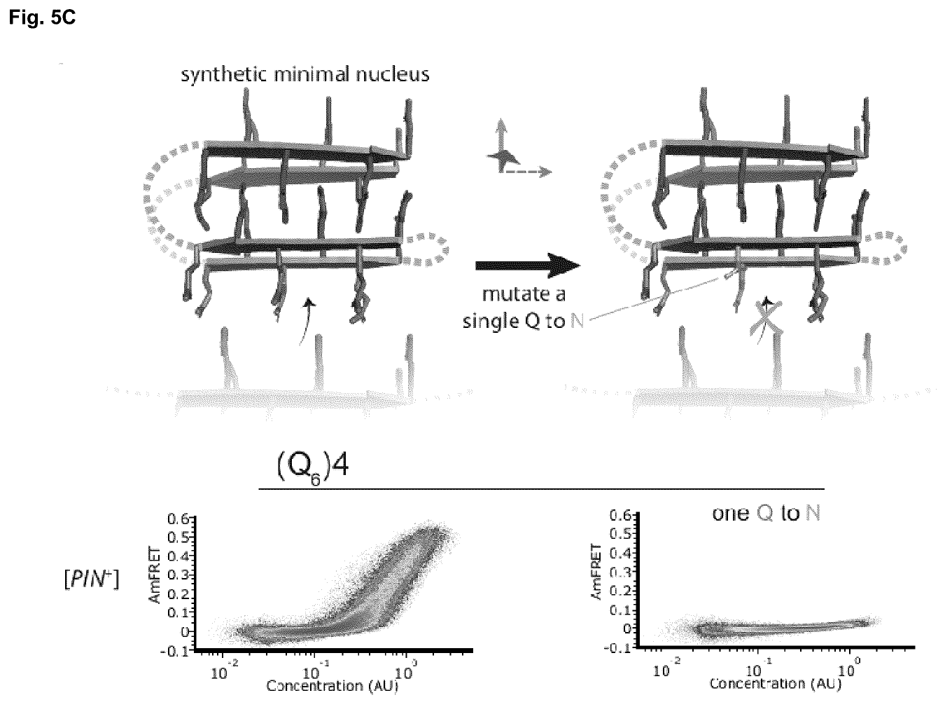

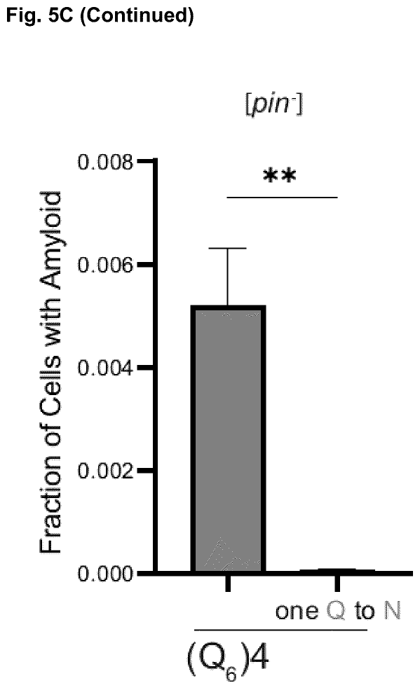

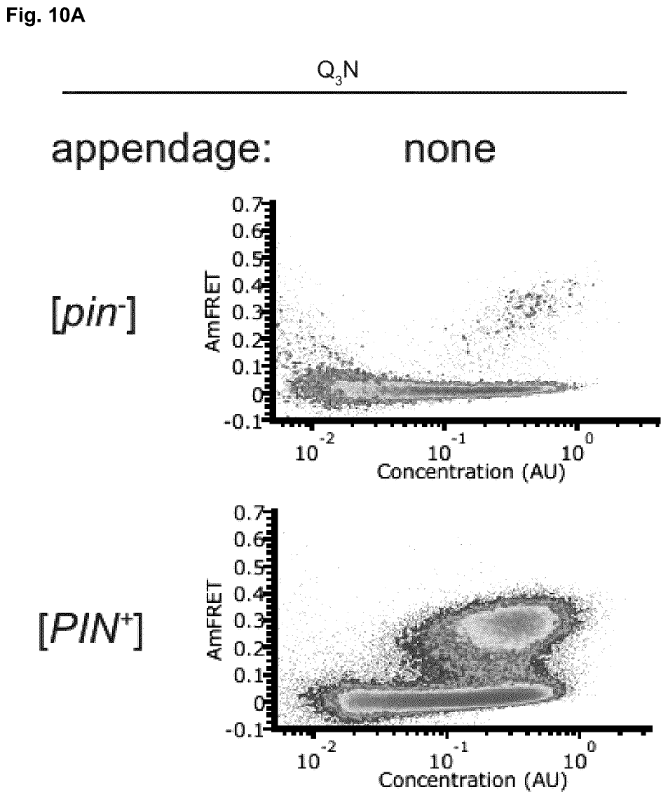

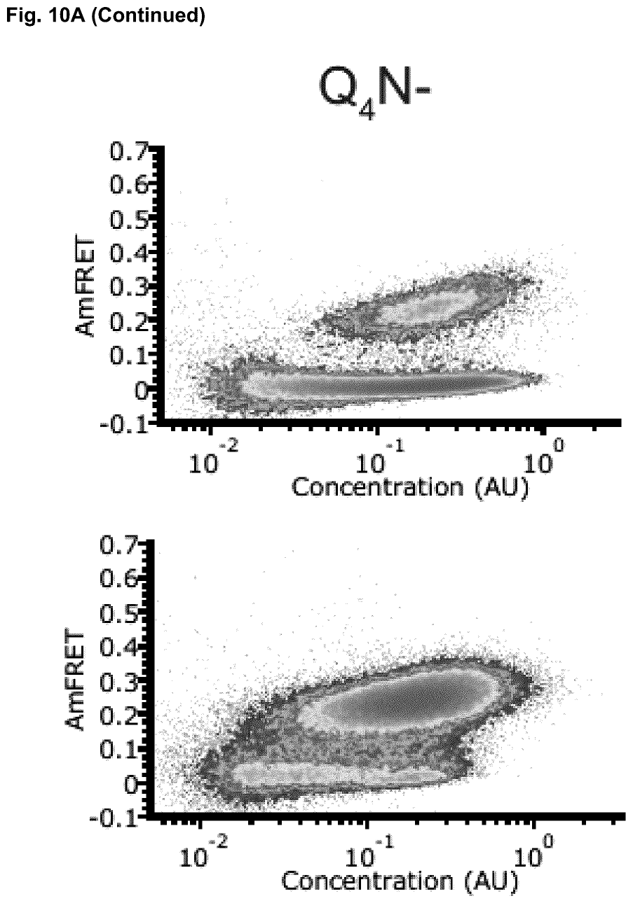

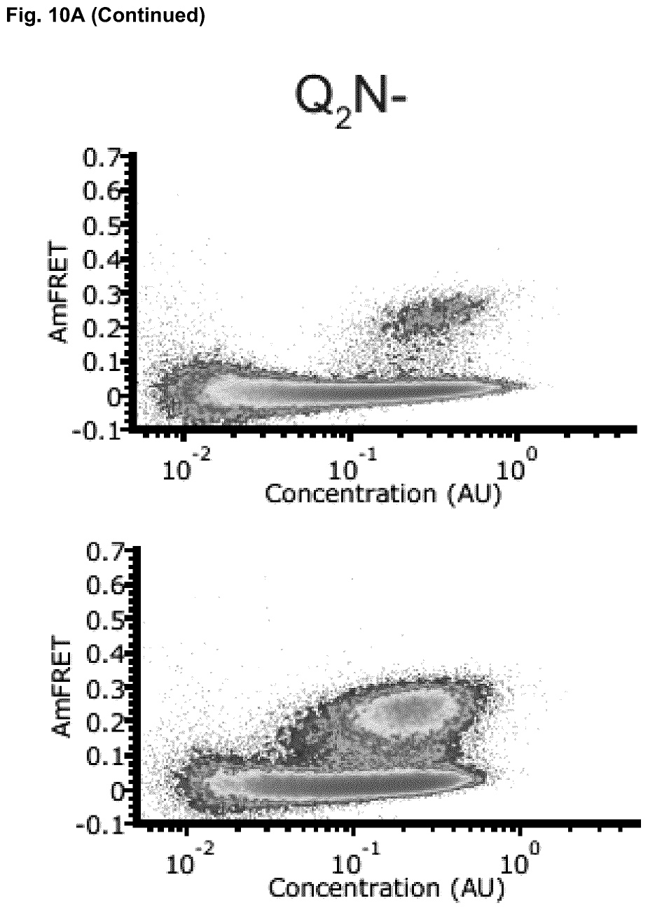

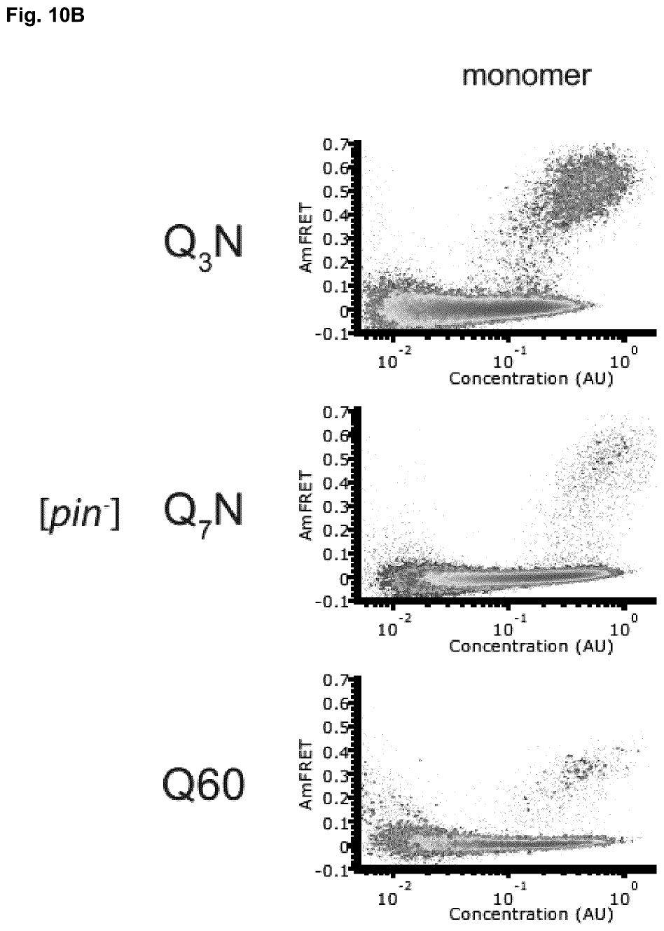

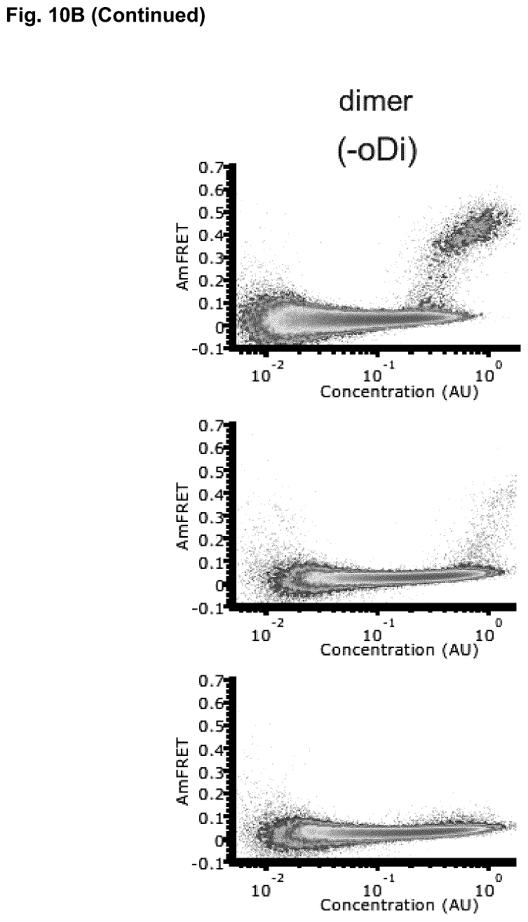

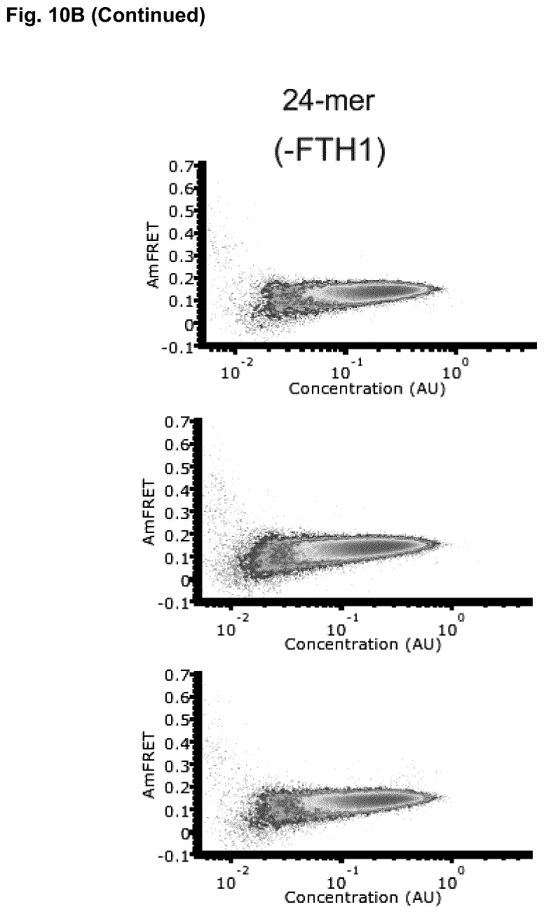

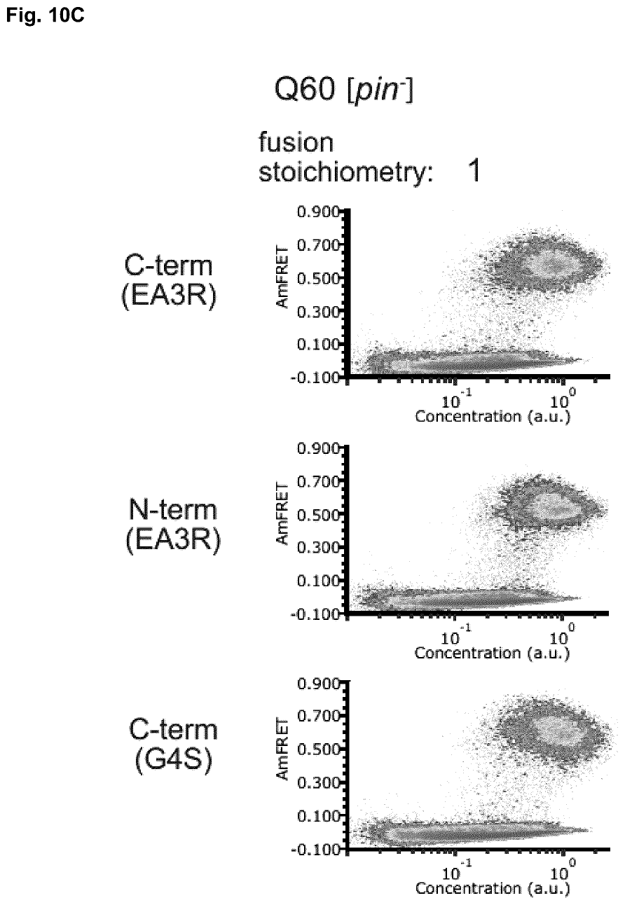

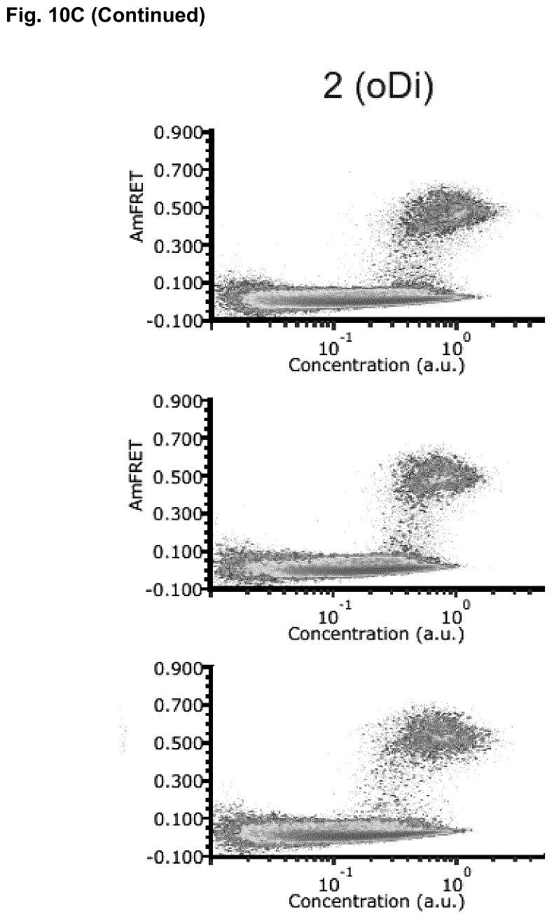

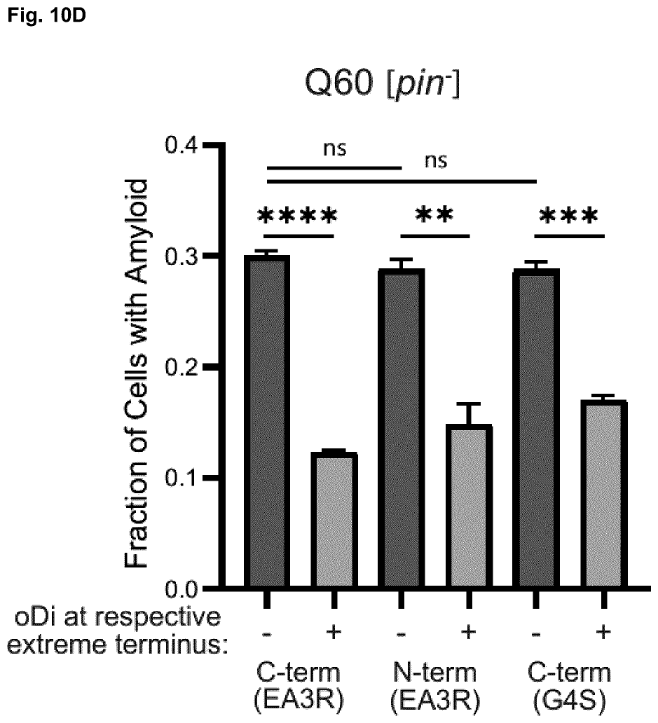

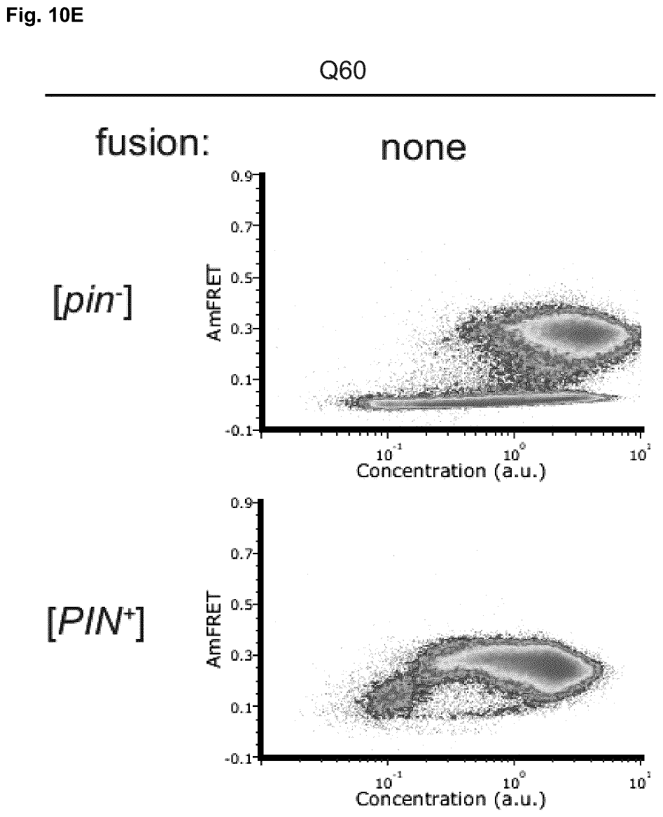

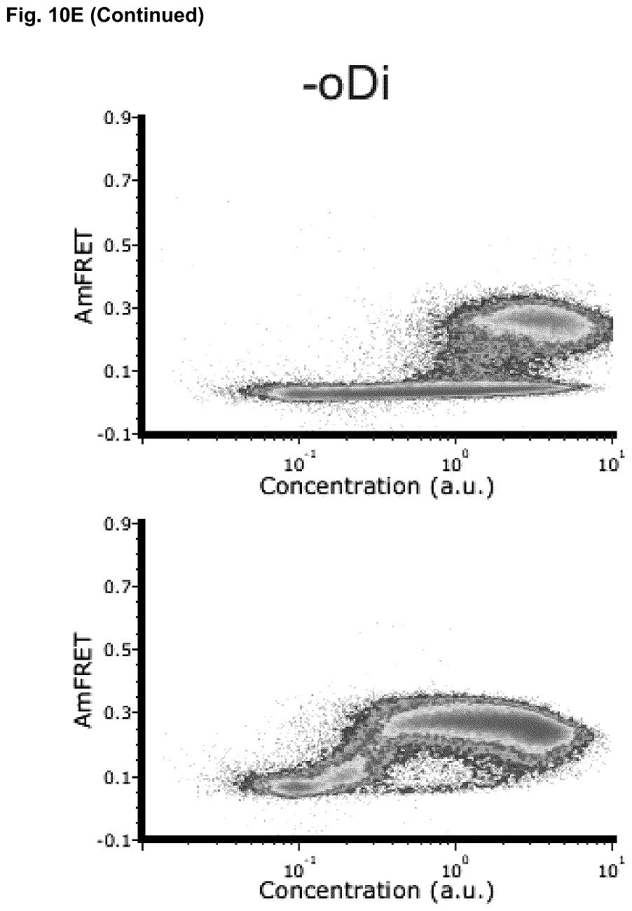

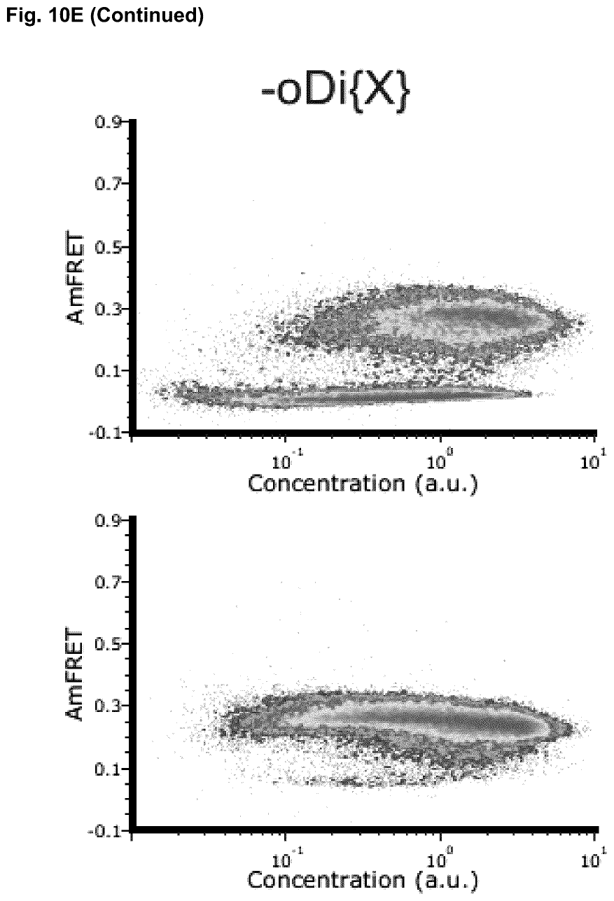

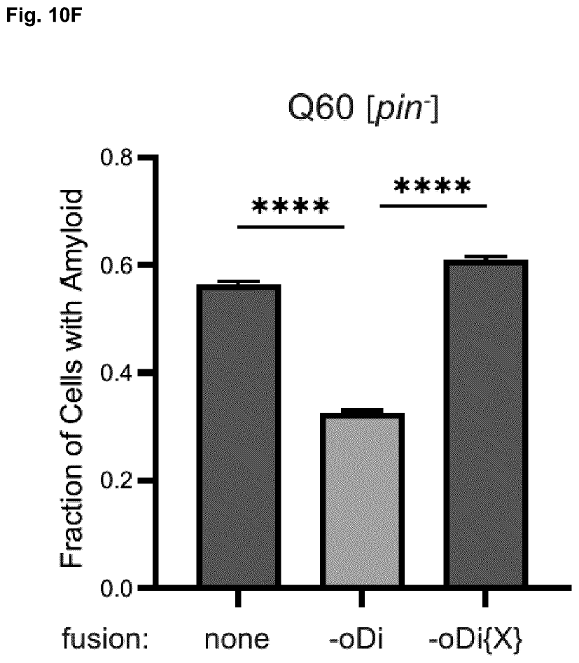

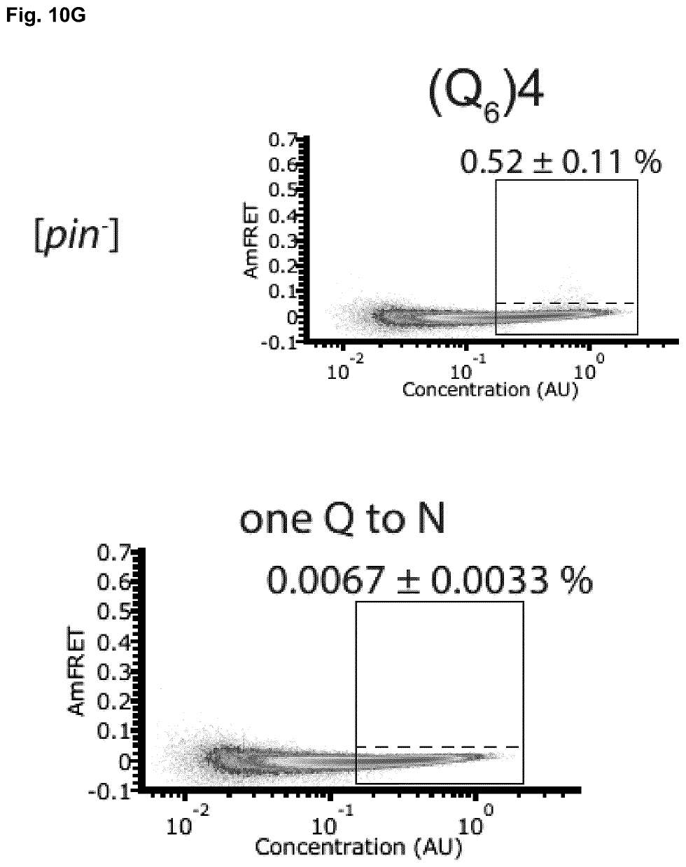

This interpretation leads to an important prediction concerning the inability of Q₄N to form amyloid. With segments of four unilaterally and bilaterally contiguous Qs, this sequence should be able to form the hypothetical intramolecular Q zipper, but fail to propagate it either axially or laterally as doing so requires at least five unilaterally or six bilaterally contiguous Qs, respectively. If the rate-limiting step for amyloid is the formation of a single short Q zipper, then appending Q₄N to another Q zipper amyloid-forming sequence will facilitate its nucleation. If nucleation instead requires longer or lamellar zippers, then appending Q₄N will inhibit nucleation. To test this prediction, we employed Q₃N as a non-lamellar “sensor” of otherwise cryptic Q zipper strands donated by a 30 residue Q₄N tract appended to it. To control for the increased total length of the polypeptide, we separately appended a non Q zipper-compatible tract: Q₂N. As predicted, the Q₄N appendage increased the fraction of cells in the high-AmFRET population relative to those expressing Q₃N alone, and even more so relative to those expressing the Q₂N-appended protein (FIGS. 5A, 10A). These data are consistent with a single short Q zipper as the amyloid nucleus.

To now determine if the nucleating zipper occurs preferentially in a single polypeptide chain, we genetically fused Q_{U} and Q_{B} to either of two homo-oligomeric modules: a coiled coil dimer (oDi, (Fletcher et al., 2012)), or a 24-mer (human FTH1, (Bracha et al., 2018)). Importantly, neither of these fusion partners contain β-structure that could conceivably template Q zippers. Remarkably, the oDi fusion reduced amyloid formation, and the FTH1 fusion all but eliminated it, for both Q_{U} and Q_{B} sequences (FIGS. 5B, 10B). To exclude trivial explanations for this result, we additionally tested fusions to oDi on the opposite terminus, with a different linker, or with a mutation designed to block dimerization (designated Odi{X}). The inhibitory effect of oDi manifested regardless of the terminus tagged or the linker used, and the monomerizing mutation rescued amyloid formation (FIGS. 10C-F), altogether confirming that preemptive oligomerization prevents Q zipper formation.

Finally, a sequence representing the minimal “polyQ” amyloid nucleus was designed. This sequence has the precise number and placement of Qs necessary for amyloid to form via intramolecular Q zipper nucleation: four tracts of six Qs linked by minimal loops of three glycines. The sequence itself is too short—at 33 residues—to form amyloid unless the glycine loops are correctly positioned (recall from FIGS. 1D and 6A that pure polyQ shorter than 40 residues does not detectably aggregate). We found that the protein indeed formed amyloid robustly, and with a concentration-dependence and [PIN⁺]-independence that is characteristic of Q_{B} (FIGS. 5C, 10G). If the nucleus is indeed a monomer, then a defect in even one of the four strands should severely restrict amyloid formation. We therefore mutated a single Q residue to an N. Remarkably, this tiny change—removing just one carbon atom from the polypeptide—completely eliminated amyloid formation (FIGS. 5C, 10G). We conclude that the polyQ amyloid nucleus is an intramolecular Q zipper.

### Example 7

### Discussion

PolyQ Amyloid Begins within a Molecule

The initiating molecular event leading to pathogenic aggregates is the most important yet intractable step in the progression of age-associated neurodegenerative diseases such as Huntington's disease. Here, a newly developed assay was employed to identify sequence features that govern the dependence of nucleation frequency on concentration and conformational templates, to deduce that amyloid formation by pathologically expanded polyQ begins with the formation of a minimal Q zipper within a single polypeptide molecule.

Kinetic studies performed both with synthetic peptides in vitro (Bhattacharyya et al., 2005; Chen et al., 2002; Kar et al., 2011) and recombinant protein in animals (Sinnige et al., 2021) and neuronal cell culture (Colby et al., 2006) have found that polyQ aggregates with a reaction order of approximately one. Under the assumption of homogeneous nucleation, this implies that pathogenic nucleation occurs within a monomer (Bhattacharyya et al., 2005; Chen et al., 2002; Kar et al., 2011). However, that conclusion, along with the assumption it rests on, contrasts with observations that polyQ can form apparently non-amyloid multimers prior to amyloids in vitro (Crick et al., 2006; Vitalis and Pappu, 2011), and that phase separation can promote amyloid formation (Babinchak and Surewicz, 2020; Camino et al., 2021; Shin and Brangwynne, 2017; Sprunger and Jackrel, 2021). By explicitly manipulating concentration, length, density and conformational heterogeneities, we found that, indeed, the rate-limiting step for pathologic polyQ amyloid formation occurs within a monomer. Importantly, it does so even in the dynamic, intermingled, living cellular environment.

Once formed, this minimal Q zipper germinates in all three dimensions to ultimately produce amyloid fibers with lamellar zippers longer than that of the nucleus proposed here. Both features have been confirmed by structural studies (Boatz et al., 2020; Galaz-Montoya et al., 2021; Hoop et al., 2016; Nazarov et al., 2022). The difference in critical lengths between intra- and intermolecular Q zippers follows from the fact that intramolecular segments are covalently constrained to much higher effective concentrations. In the case of polyQ, the short-stranded nucleus is merely a catalyst for longer, lamellar Q zippers that make up the amyloid core. That the nascent amyloid should become progressively more ordered is in keeping with decades of experimental and theoretical work by polymer physicists (Keller and O'Connor, 1957; Xu et al., 2021; M. Zhang et al., 2016). The minimally competent zipper identified here provides lower bounds on the widths of fibers—they can be as thin as two sheets (1.6 nm) and as short as six residues. This hypothetical restriction is thinner and shorter than the cores of all known amyloid structures of full length polypeptides (Sawaya et al., 2021). Remarkably, observations from recent cryoEM tomography studies closely match this prediction: pathologic polyQ or huntingtin amyloids, whether in cells or in vitro, feature a slab-like architecture with restrictions as thin as 2 nm (Galaz-Montoya et al., 2021) and as short as five residues (Nazarov et al., 2022).

Multidimensional polymer crystal growth offers a simple explanation for the characteristically antiparallel arrangement of strands in polyQ amyloid. Each lamellum first nucleates then propagates along the templating lamellum by the back-and-forth folding of monomers (M. Zhang et al., 2016). In the context of polyQ, the end result is a stack of beta hairpins. If lamella are responsible for antiparallel strands, then Q_{U} sequences should form typical amyloid fibers with parallel beta strands. The only atomic resolution structure of a Q zipper amyloid happens to be of a protein with striking unilateral (but not bilateral) contiguity, and the strands are indeed arranged in parallel (Hervas et al., 2020).

Seven of the nine polyQ diseases have length thresholds at or above (Lieberman et al., 2019) the minimum length for an intramolecular Q zipper as deduced here. The two polyQ diseases with length thresholds below that of the intramolecular Q zipper, SCA2 (32 residues) and SCA6 (21 residues), are also atypical for this class of diseases in that disease onset is accelerated in homozygous individuals (Laffita-Mesa et al., 2012; Mariotti et al., 2001; Soga et al., 2017; Spadafora et al., 2007; Tojima et al., 2018). This is consistent with nucleation occurring in oligomers, as would necessarily be the case for such short polyQ tracts.

It is expected that the monomeric nucleus of polyQ will prove to be unusual among pathologic amyloid-forming proteins. Among the hundreds of amyloid-forming proteins we have now characterized by DAmFRET (Khan et al., 2018; Posey et al., 2021), and unpublished), only Cyc8 PrD has a similar concentration-dependence. We now attribute this to self-poisoning due to its exceptionally long tract of 50 unilaterally contiguous Qs, primarily in the form of QA dipeptide repeats.

### Proteotoxic Multimers are Likely to be Self-Poisoned Polymer Crystals

Soluble oligomers accumulate during the aggregation of pathologically lengthened polyQ and/or Htt in vitro (Auer et al., 2008; Hsieh et al., 2017; Levin et al., 2014; Liang et al., 2018; Li et al., 2010; Sil et al., 2018; Vitalis and Pappu, 2011; Yamaguchi et al., 2005; Zanjani et al., 2020), in cultured cells (Olshina et al., 2010; Takahashi et al., 2008), and in the brains of patients (Legleiter et al., 2010; Sathasivam et al., 2010). They are likely culprits of proteotoxicity (Kim et al., 2016; Leitman et al., 2013; Lu and Palacino, 2013; Matlahov and van der Wel, 2019; Takahashi et al., 2008; Wetzel, 2020). We found that Q zipper nucleation precedes the accumulation of multimers, rather than the other way around. Notwithstanding one report to the contrary (Peskett et al., 2018)—which we respectfully attribute to a known artifact of the FLAG tag when fused to polyQ (Duennwald et al., 2006a, 2006b; Jiang et al., 2017)—our findings corroborate prior demonstrations that polyQ (absent flanking domains) does not phase separate prior to amyloid formation, whether expressed in human neuronal cells (Colby et al., 2006; Kakkar et al., 2016), C. elegans body wall muscle cells (Sinnige et al., 2021), or [pin−] yeast cells (Duennwald et al., 2006a, 2006b; Jiang et al., 2017).

Our findings that kinetically arrested aggregates emerge from the same nucleating event responsible for amyloid formation suggests a resolution to the paradox that polyQ diseases are rate-limited by amyloid nucleation despite the implication of non-amyloid species (Kim et al., 2016; Leitman et al., 2013; Lu and Palacino, 2013; Matlahov and van der Wel, 2019; Takahashi et al., 2008; Wetzel, 2020). Likewise, our discovery that polyQ amyloid formation is blocked by oligomerization or phase separation, together with demonstrations that full length Huntingtin protein with a pathologically lengthened polyQ tract can phase separate in cells (Aktar et al., 2019; de Mattos et al., 2022; Peskett et al., 2018; Wan et al., 2021), suggests a simple explanation for the otherwise paradoxical fact that Huntington's Disease onset and severity do not increase in homozygous individuals (Cubo et al., 2019; Lee et al., 2019; Wexler et al., 1987). Specifically, if mutant Huntingtin forms an endogenous condensate (if only in some microcompartment of the cell) when expressed from just one allele, then the buffering effect of phase separation (Klosin et al., 2020) will prevent a second allele from increasing the concentration of nucleation-competent monomers.

Our data indirectly illuminate the nature of these arrested multimers. For Q_{U}, the multimers failed to accumulate to a level detectable by AmFRET, and failed to coalesce to microscopic puncta. They therefore seem to involve only a small fraction of the total protein, presumably limited to soluble species that have acquired a nascent Q zipper. The disordered polyQ globule is extremely viscous and this causes very slow conformational conversion on the tips of growing fibers (Bhattacharyya et al., 2005; Walters et al., 2012). It should therefore be highly susceptible to self-poisoning as has been widely observed and studied for polymer crystals in vitro (Jiang et al., 2016; Ungar and Keller, 1987; Whitelam et al., 2016; Zhang et al., 2020, 2018). In short, the multimers are nascent amyloids that templated their own arrest in the earliest stages of growth following nucleation. We do not yet know if these aborted amyloids contribute to pathology. However, the role of contralateral zippers in their formation is consistent with the apparent protective effect of CAT trinucleotide insertions that preserve unilateral contiguity (QHQHQH) in the polyQ disease protein, SCA1 (Menon et al., 2013; Nethisinghe et al., 2018; Sen et al., 2003), and the absence of bilateral contiguity in the only known functional Q zipper, formed by the neuronal translational regulator, Orb2 (Hervas et al., 2020). We speculate that the toxicity of polyQ amyloid arises from its lamellar architecture.

### CONCLUSION

The etiology of polyQ pathology has been elusive. Decades-long efforts by many labs have revealed precise measurements of polyQ aggregation kinetics, atomistic details of the amyloid structure, a catalog of proteotoxic candidates, and snapshots of the conformational preferences of disordered polyQ. What they have not yet led to are treatments. We synthesized those insights to recognize that pathogenesis likely begins with a very specific conformational fluctuation. We set out to characterize the nature of that event in the cellular milieu, deploying a technique we developed to do just that, and arrived at an intramolecular four-stranded polymer crystal. Our findings rationalize key aspects of polyQ diseases, such as length thresholds, kinetics of progression, and involvement of pre-amyloid multimers. More importantly, they illuminate a new avenue for potential treatments. Current therapeutic efforts focused on lowering the levels of mutant Huntingtin have not been successful. As an admittedly radical alternative, we suggest that therapies designed to (further) oligomerize huntingtin preemptively will delay nucleation and thereby decelerate the disease.

### Example 8

### Gene Fusion Induced Oligomerization Prevents Amyloid Aggregation of TDP-43

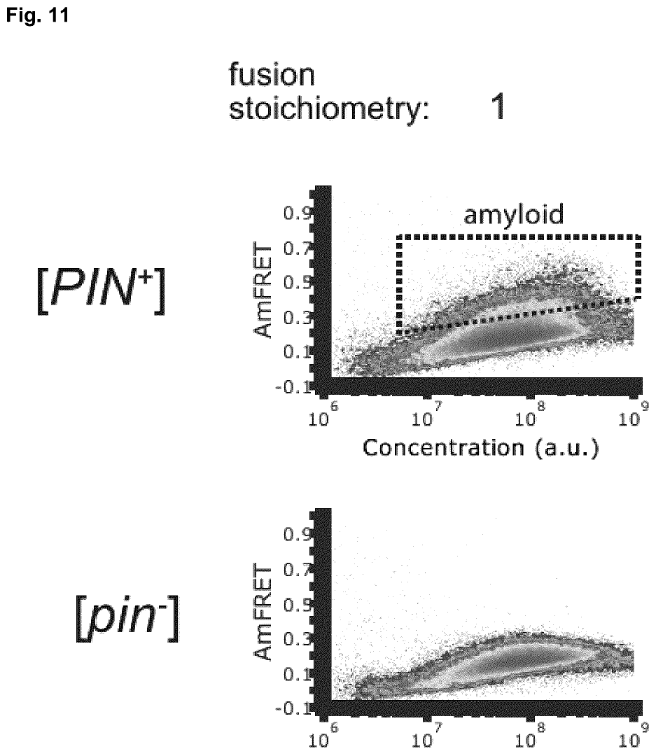

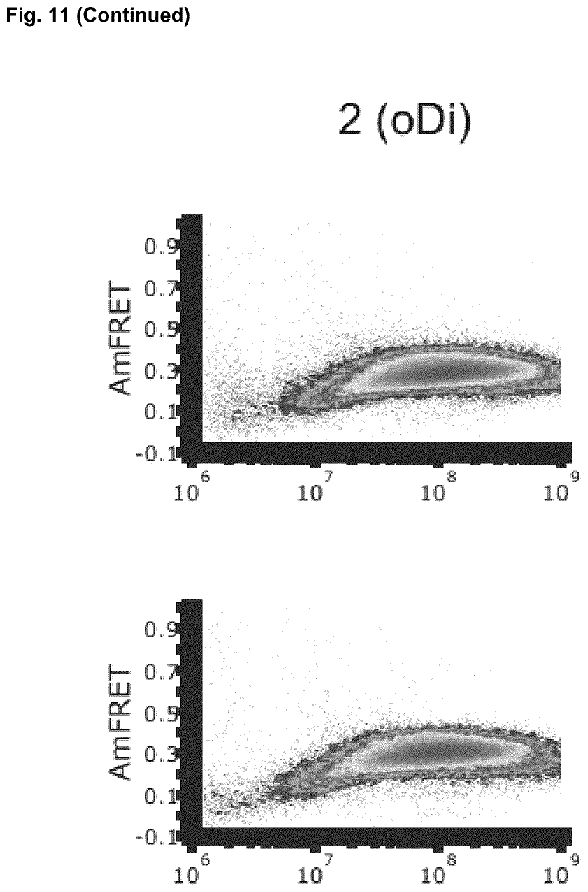

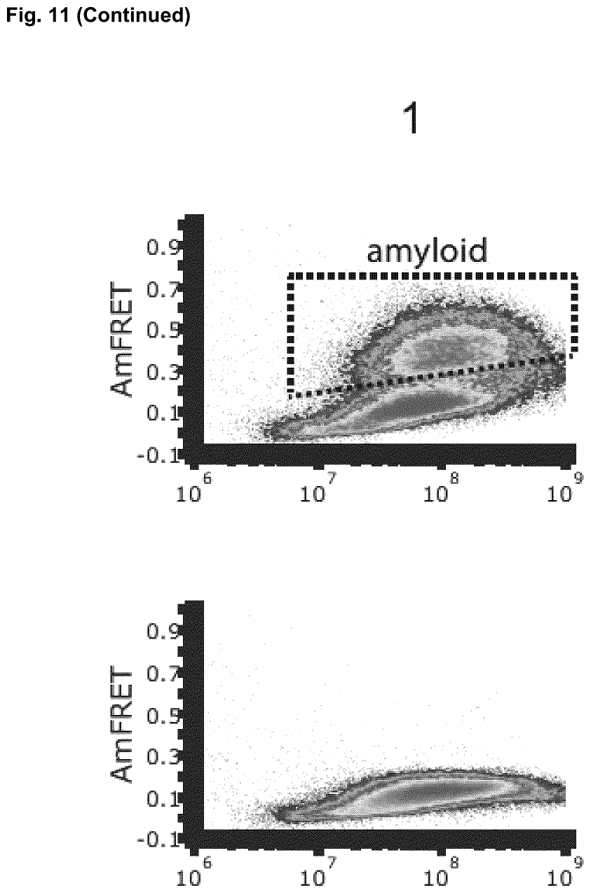

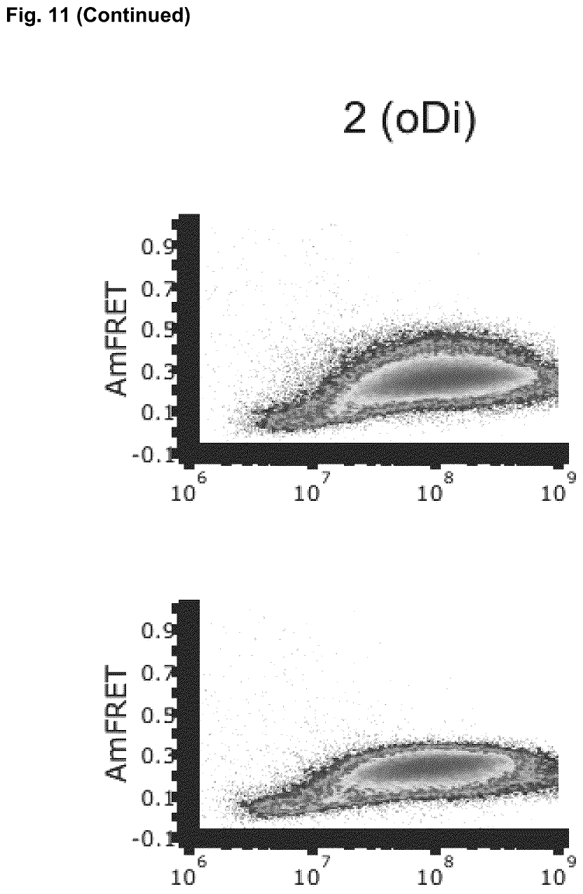

To further explore the effect of preemptive oligomerization in other amyloid-associated condition, TAR DNA-binding protein 43 (TDP-43), the aggregation of which that is responsible for amyotrophic lateral sclerosis (ALS), was also tested in the new assay. FIG. 11 shows the DAmFRET plots for the C-terminal prion-like domain of TDP-43 (“CTD”, residues 273-414) expressed in yeast with an N-terminal fusion to mEos3.1. Nucleation to the amyloid state only occurs in cells harboring a pre-existing amyloid of similar sequence composition ([PIN+]). Fusing a coiled coil dimer peptide to the extreme N-terminus (i.e. on the other side of mEos3.1 from the TDP-43 273-414 sequence) prevents cells from acquiring amyloid. This effect can be observed more clearly when the TDP-43 sequence has an amyloid-promoting mutation (M337P). The oDi fusion slightly increases the basal AmFRET for both WT and mutant, as expected for dimerization, but prevents them from forming the higher AmFRET amyloid-containing population in [PIN+] cells.

### Example 9

### DAmFRET Biosensor Construction and Validation in HEK293T Cells

Biosensor cell lines were constructed by integrating the reporter genes that encode mEos3.2-fused tau or TDP-43 protein into the genome of HEK293T cell lines with lentiviral transfection technique. Specifically, the reporter gene was first cloned into a lentiviral transfer plasmid, which was then transfected together with packaging plasmid (PAX2) and envelope plasmid (VSV-G) into HEK293T cells using the TransIT® transfection reagent. After the transfection, the cells were incubated for 48-96 hours at 37° C. to allow the production and the release of viral particles containing the reporter genes into the media which were collected, filtered, treated with HEPES, and stored at 4° C. before downstream integration. Next, the media containing the viral particles was diluted with fresh DMEM media (with 10% FBS and L-glutamine), and was added into a new plate of HEK293T cells at 60% confluence, followed by the overnight incubation at 37° C. The overnight culture media was then removed and replaced with fresh complete DMEM (with 10% FBS, L-glutamine, penicillin, and streptomycin), and the cells were incubated for an additional 72 hours. The single clones containing the integrated reporter genes were sorted for green fluorescence, and the selected single cell clone was expanded and stored as corresponding biosensor cell lines.

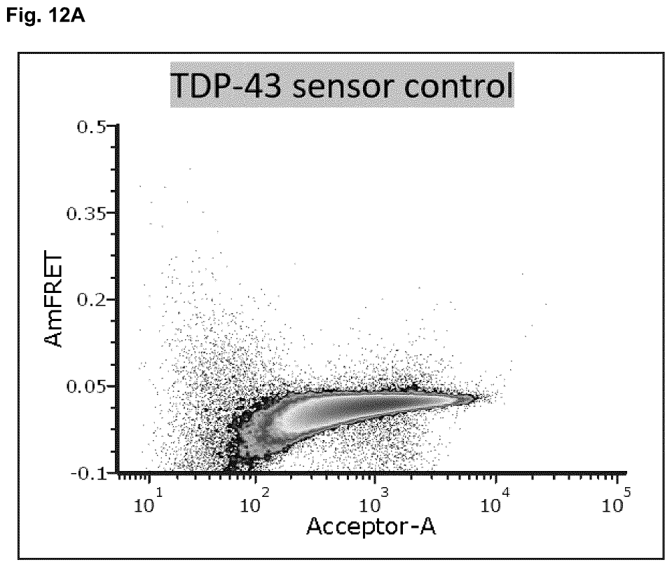

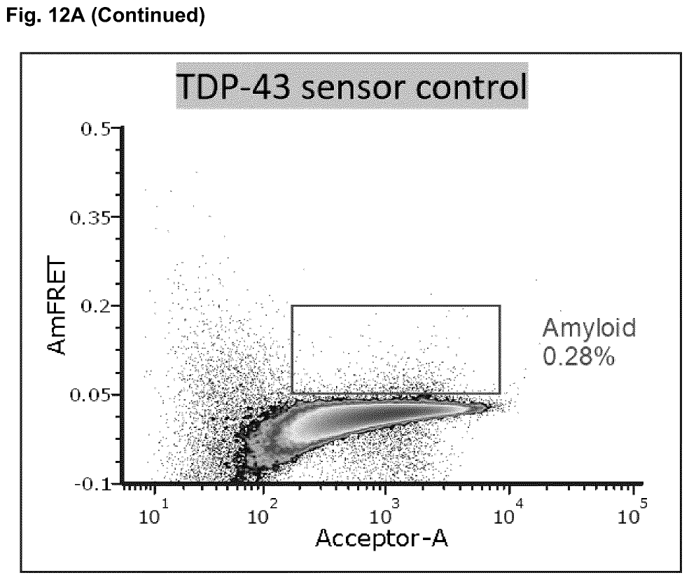

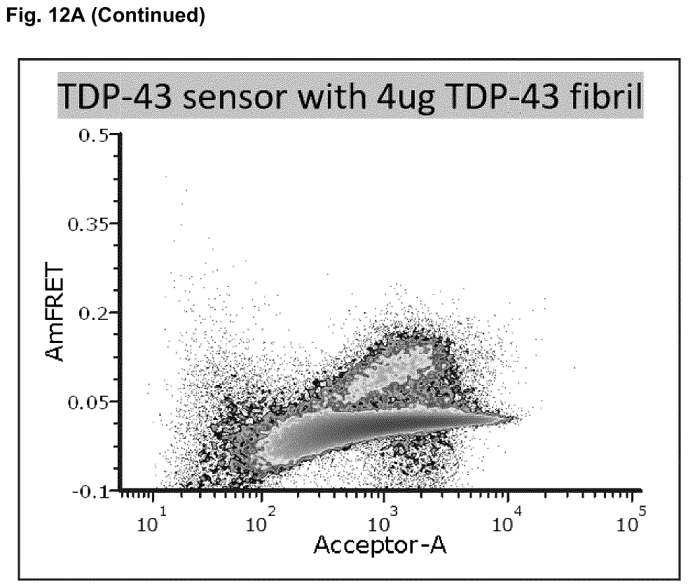

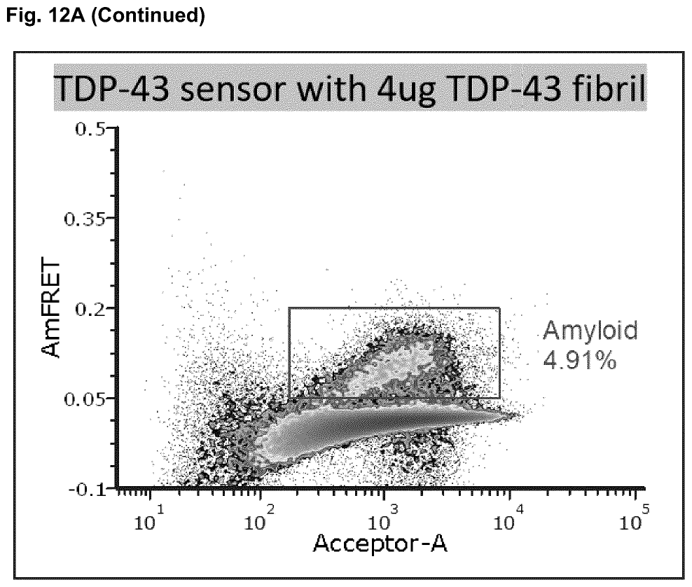

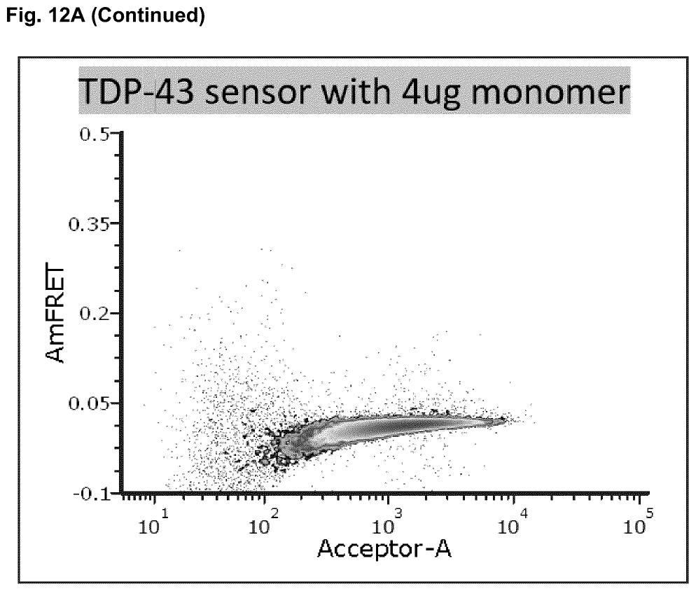

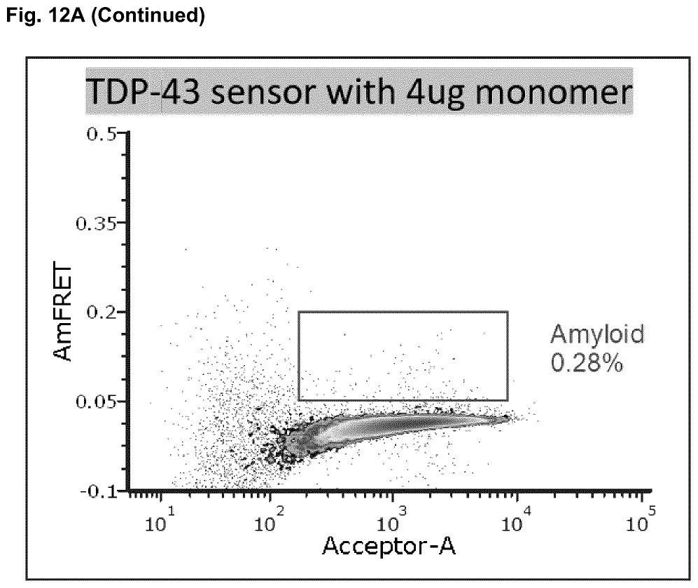

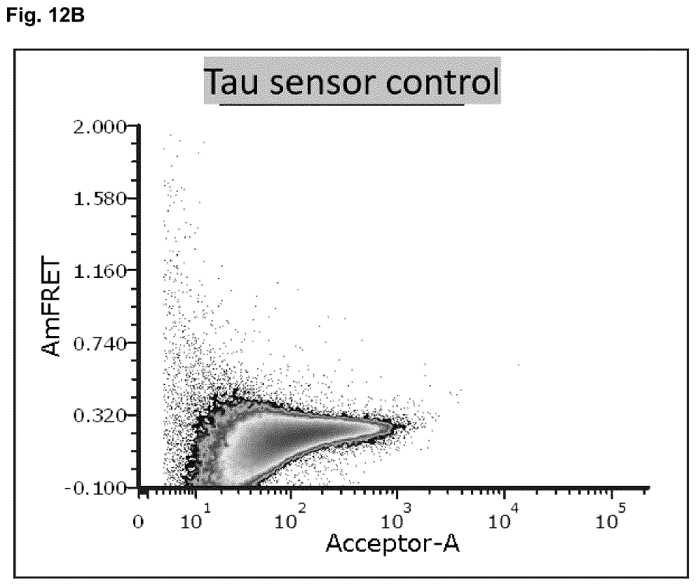

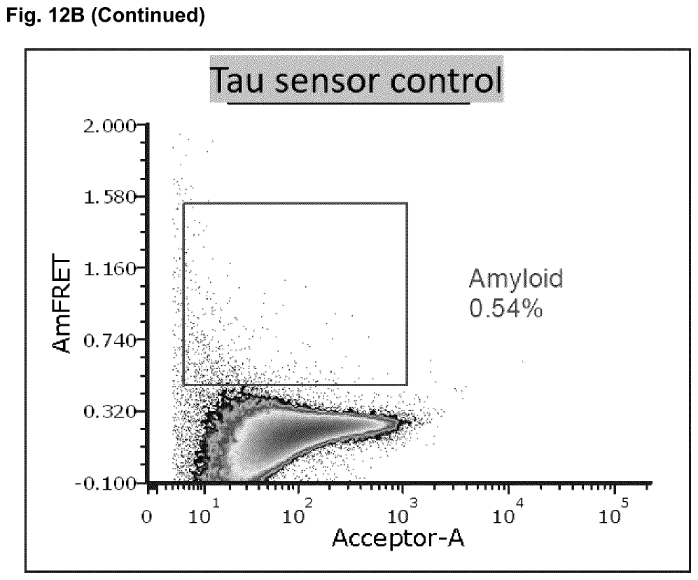

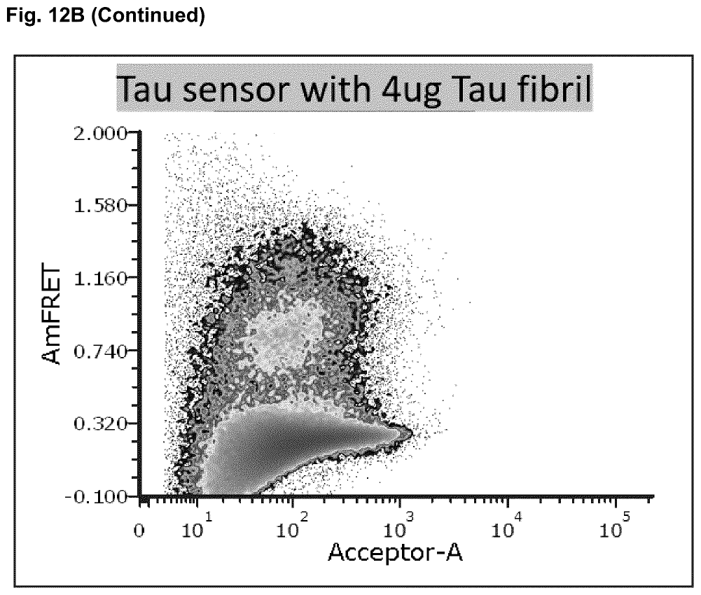

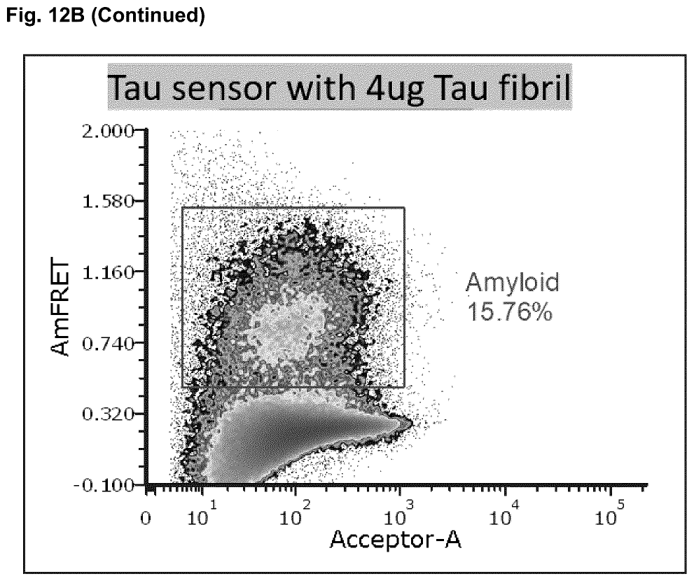

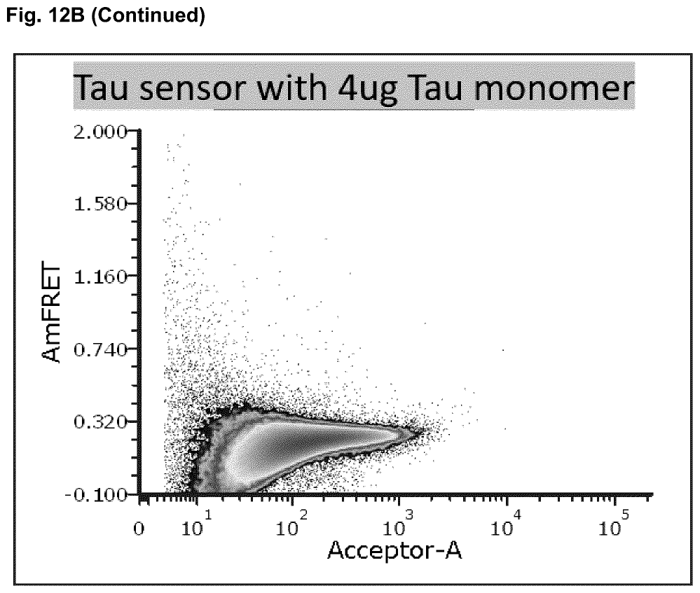

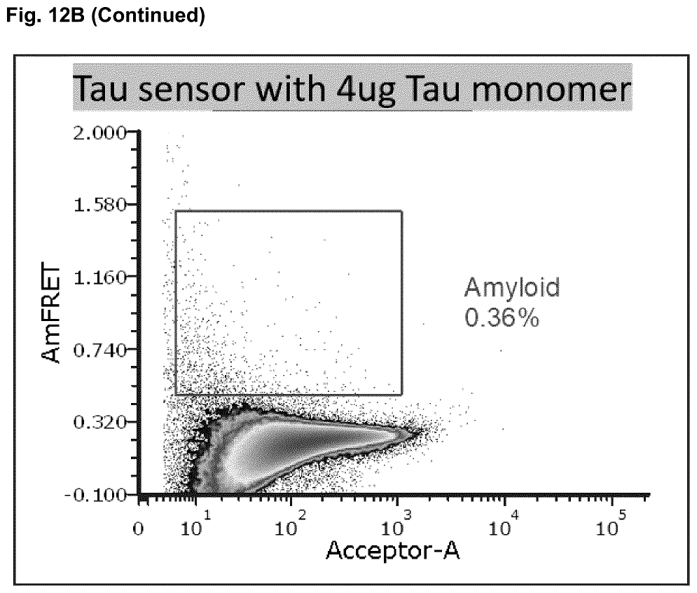

To validate the use of the biosensor cell lines to detect the presence of specific amyloids in a given sample using DAmFRET, recombinant tau or TDP-43 LCS (311-414) were assembled into amyloid using well-established protocols and then transfected into the corresponding biosensor cell line using Lipofectamine™ transfection reagent, followed by a 48-hour incubation at 37° C. The resulting cells were then fixed with 4% PFA, collected, exposed to a 5-minute illumination of 405 nm light, and analyzed by flow cytometry as in Venkatesan et al. 2019. Results are shown in FIGS. 12A (TDP-43 sensor) and 12B (tau sensor).

### Detailed Protocol:

### Packaging of Lentiviral Constructs

Day 0: Plate 293T cells in 10 cm plates in complete DMEM (10% FBS, L-glutamine, Pen/strep)—need to be ˜60% confluency at time of transfection (3e6/dish). Alternatively, use media containing NO Pen/Strep to pass cells now, otherwise change media tomorrow.

Day 1: in the morning, change media to NO Pen/Strep DMEM, if not done yesterday. In the afternoon, transfect (TransIT-LT1; Mirus): prepare DNA (7 μg lentivirus construct (construct to integrate), 7 μg PAX2 (packaging plasmid), 1 μg VSV-G (envelope plasmid)); mix 1.5 mL OMEM+45 μL TransIT-LTI; incubate 5 min; add OMEM-LTI mixture to DNA mixture and mix well; incubate at RT for 30 min; add dropwise to 10 cm plate. Be sure media does not change pH during this time (orange-ish-pink media is okay).

Day 2-3: after 48 hours, collect the viral containing media and filter through a sterile 0.45 μM syringe filter (First Harvest). This is optional, you can proceed with only one harvest on day 4 (if many cells die, spin down supernatant first, to remove debris, then sterile filter). Add 20 mM HEPES and store at 4° C. Add 10 mL fresh media (NO Pen/Strep) back to 10 cm plate. Incubate 24 hours more

Day 3-4: collect media, filter, and treat with HEPES as before (Second Harvest). Combine Harvests (20 mL total). Store at 4° C. until ready to use, up to a week. Or store at −80° C. for the long term.

### Transduction of Lentivirus

Adherent cells (293T): Plate cells to be ˜60% confluency at the time of infection in 6 well plate 24 hr prior to infection (0.65e6/well) (cells should be transduced at such a density such that they would become near confluent in 48-72 hrs). Prepare viral dilutions in a 15 mL falcon tube. Start first with 2 mL of fresh DMEM (10% FBS, L-glutamine, NO Pen/Strep). Add lentiviral particles—serial dilutions or titers may be necessary—maintain 2 mL per well volume. A good start is to add the same volume of media containing virus to the fresh DMEM (2 mL to 2 mL). Add Polybrene to each well-final concentration/well=8 μg/mL (dilute an aliquot of 10 mg/mL stock to 1 mg/mL add 16 μL to each tube). Remove media of 6-well plates and replace with media containing virus. Incubate overnight at 37° C. The next morning, replace viral media with fresh DMEM (10% FBS, L-glut, Pen/Strep). Grow for an additional 72 hours (pass if necessary). When cells reach confluency on the 6-well plate. Expand to a T-25 and start drug selection. Let grow until confluent and then expand to a T-75. At this point schedule S6 sorter to bulk sort or do single clone expansion. Single cell sorting for transfected cells (remove media add 4 ml Triple E; waiting for 3 mins, add 8 ml DMEM, transfer to 15 ml centrifuge tube, centrifuge 3 mins 800×g; resuspend in 3 ml DMEM and filter in a sorting tube).

Transfect Amyloid Fibrils into Biosensor Cell Lines
Protein solution: PBS+protein
Transfection Plan: Prepare Lipofectamine solution: 100 μL for each well (12.5 μL of Lipofectamine+87.5 μL Opti-MEM. To create master-mix for 57 wells, we need 5.7 mL total (712.5 μL Lipofectamine+4987.5 μL Opti-MEM)).

Prepare TDP43 fibril solution: 100 μL for each well (X μL fibril dilution+(100−x) μL Opti-MEM; 3 wells/variable, then 300 μL/variable total; need to prepare enough solution for three plates (Plate A1, B1, and C1)):

TDP43 Fibril solution/3 wells for One plate

| Total μg of | Volume of | Volume of |
|---|---|---|
| Fibrils/well | Fibril Dilution | Opti-MEM |
| Fiber 4 (4 μg) | (9.6*3)*3 = 86.4 μL | 271.2*3 = 813.6 μL |
| Fiber 2 (2 μg) | (4.8*3)*3 = 43.2 μL | 285.6*3 = 856.8 μL |
| Fiber 1 (1 μg) | (2.4*3)*3 = 21.6 μL | 292.8*3 = 878.4 μL |
| Fiber 0 (0 μg) | 0 μL | 300*3 = 900 μL |

Prepare TDP43 fibril working master mix (200 μL for each well): mix (with 1:1 ratio) 900 μL of Lipofectamine solution with 900 μl of each of the TDP43 fibril solution shown in the table above, and incubate at room temperature for 20 minutes.

Prepare TDP43 monomer solution: 100 μl for each well. Sonication fibrils for 5 minutes total (30 s on and 30 s off per cycle), X μl fibril dilution+(100−x) μl Opti-MEM, 3 wells/variable, then 300 μl/variable total. Need to prepare for two plates (Plate A2 and B2):

Master Mix monomer Volumes/3 wells for One plate

| Total μg of | Volume of | Volume of |
|---|---|---|
| Fibrils/well | Fibril Dilution | Opti-MEM |
| Monomer 4 (4 μg) | (9.6*3)*2 = 57.6 μL | 271.2*2 = 542.4 μL |
| Monomer 2 (2 μg) | (4.8*3)*2 = 28.8 μL | 285.6*2 = 571.2 μL |
| Monomer 1 (1 μg) | (2.4*3)*2 = 14.4 μL | 292.8*2 = 585.6 μL |

Prepare TDP43 monomer working master mix (200 μl for each well): mix (with 1:1 ratio) 600 μl of Lipofectamine solution with 600 μl of each of the TDP43 monomer solutions shown in the table above, and incubate at room temperature for 20 minutes.

Prepare tau fibril solution and tau fibril working solution for 3 wells: 12 μl of stock tau fibril solution+288 μl of Opti-MEM; mix (with 1:1 ratio) 300 μl of Lipofectamine solution with 300 μl of each of the tau fibril solutions and incubate at room temperature for 20 minutes.

Prepare Positive Control: use pCW57.1_VPGVG-mEosNB-BDFP plasmid and follow the the FuGENE transfection protocol. (Cells need to be treated with doxy for positive control).

Cell culture and transfection plan: Grow the cells to 50% confluence for each cell line indicated below. Then add 200 μl of the combined master mix to each well

### Plate A1 (293T TDP Sensor):

| | | | |
|---|---|---|---|
| 0 μg Fibril | 1 μg Fibril | 2 μg Fibril | 4 μg Fibril |
| 0 μg Fibril | 1 μg Fibril | 2 μg Fibril | 4 μg Fibril |
| 0 μg Fibril | 1 μg Fibril | 2 μg Fibril | 4 μg Fibril |

### Plate A2: (293T TDP Sensor):

| | | | |
|---|---|---|---|
| Pos. control | 1 μg Monomer | 2 μg Monomer | 4 μg Monomer |
| Pos. control | 1 μg Monomer | 2 μg Monomer | 4 μg Monomer |
| Pos. control | 1 μg Monomer | 2 μg Monomer | 4 μg Monomer |

### Plate B1 (293T TDP-NLS Sensor):

| | | | |
|---|---|---|---|
| 0 μg Fibril | 1 μg Fibril | 2 μg Fibril | 4 μg Fibril |
| 0 μg Fibril | 1 μg Fibril | 2 μg Fibril | 4 μg Fibril |
| 0 μg Fibril | 1 μg Fibril | 2 μg Fibril | 4 μg Fibril |

### Plate B2: (293T TDP-NLS Sensor):

| | | | |
|---|---|---|---|
| Pos. control | 1 μg Monomer | 2 μg Monomer | 4 μg Monomer |
| Pos. control | 1 μg Monomer | 2 μg Monomer | 4 μg Monomer |
| Pos. control | 1 μg Monomer | 2 μg Monomer | 4 μg Monomer |

### Plate C1 (293T Tau Sensor):

| | | | |
|---|---|---|---|
| 4 μg Tau Fibril | 1 μg Fibril | 2 μg Fibril | 4 μg Fibril |
| 4 μg Tau Fibril | 1 μg Fibril | 2 μg Fibril | 4 μg Fibril |
| 4 μg Tau Fibril | 1 μg Fibril | 2 μg Fibril | 4 μg Fibril |

Harvest & Fixing 293 cells for DAmFret

Thaw a tube of PFA in the 42-degree water bath to make sure it is ready when needed. In the tissue culture hood, Vacuum the old media off the cells, being careful not to touch the cells. Gently wash the cells with 1 ml of PBS per well. This step can be skipped, especially if you are worried about your cell count (pipet onto the side of the well and let it gently fall onto the cells to avoid lifting). Vacuum away the PBS, same as before. Add 500 μl of TrypLE (rock the plate gently to lift the cells faster. If desperate, you can gently hit the side of the plate with your palm to lift them even faster). Once the cells are lifted, add 500 PBS to each well and transfer to a labeled Eppendorf tube (need to make sure that you transfer as many cells as possible). Wash the well (with the liquid already in it) as many times as needed to get all the cells in suspension. Spin down the ependorf tubes at 1000 g for 2-3 minutes. Vacuum up the liquid being sure not to touch the pellet (if preferred, you can pipet the liquid away instead of vacuuming). Resuspend pellet in 500 μl of PFA. Incubate the tubes at 37° C. on the shaker for 5 minutes. Spin down the tubes at 1000 g for 2-3 minutes again. Vacuum up the PFA. Resuspend in 450 μl of PBS+EDTA (10 mM) (this amount can change based on desired concentration and amount needed). Transfer non-photo converted mEOS3.1 cells and “dark” cells to eppendorf tube (dsRED control is in the −80° C.). Transfer 150 μl cells to 96-well plate for ZE5 analysis (30 min before cytometry appt.). Photoconvert for 5 mins by exposing the 96-well plate (or 384-well plate) to 405 nm illumination using the UV lamp on B1 floor, mol bio. Keep the plate shaking at 800 RPM during photoconversion to avoid cells settling which leads to non-uniform photoconversion. If multiple 96-well plates are being used, rearray into 384-well plate (4×96-well plates). PFA is only good for one time. Any thawed PFA cannot be frozen and used again. No need to save any leftover.

### Example 10

### Isomerization of a Single Proline Governs TDP-43 Aggregation

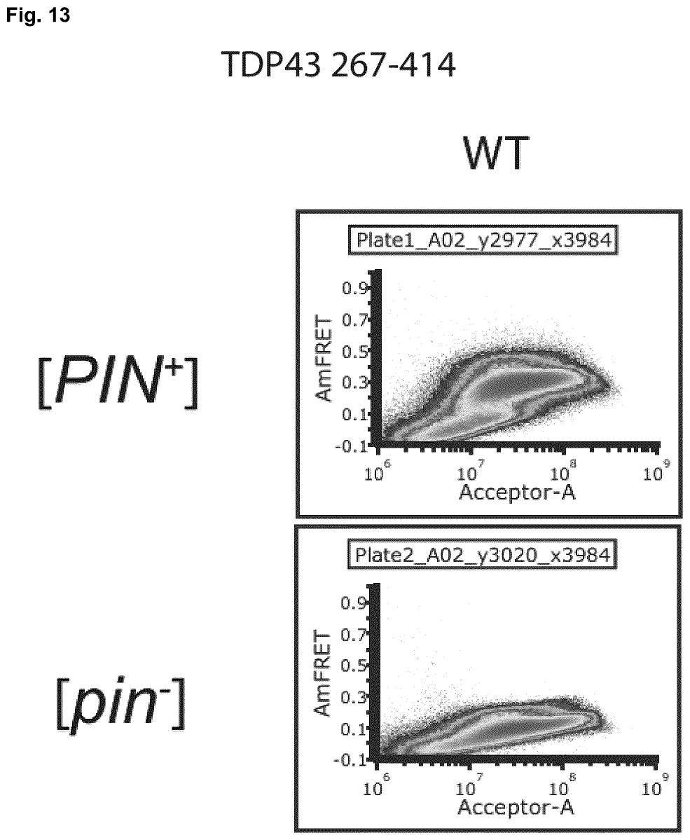

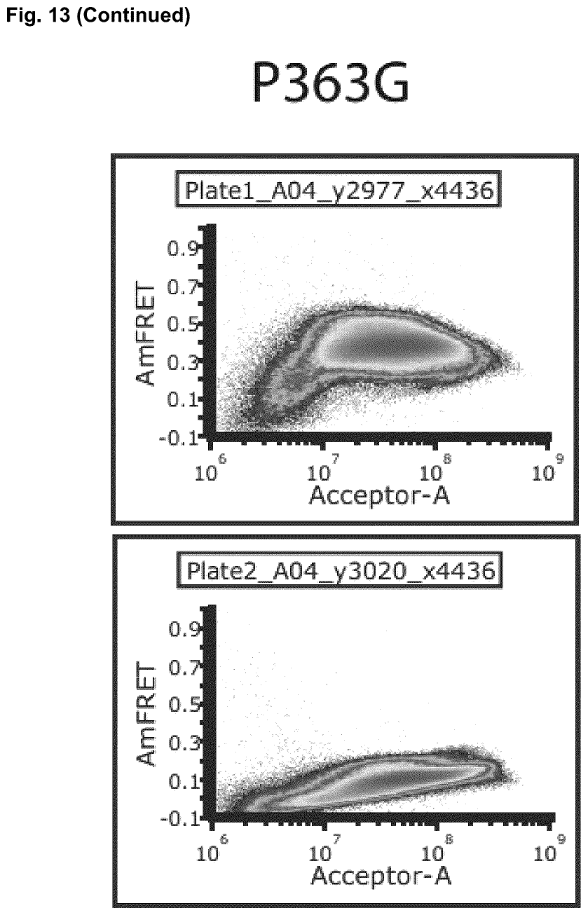

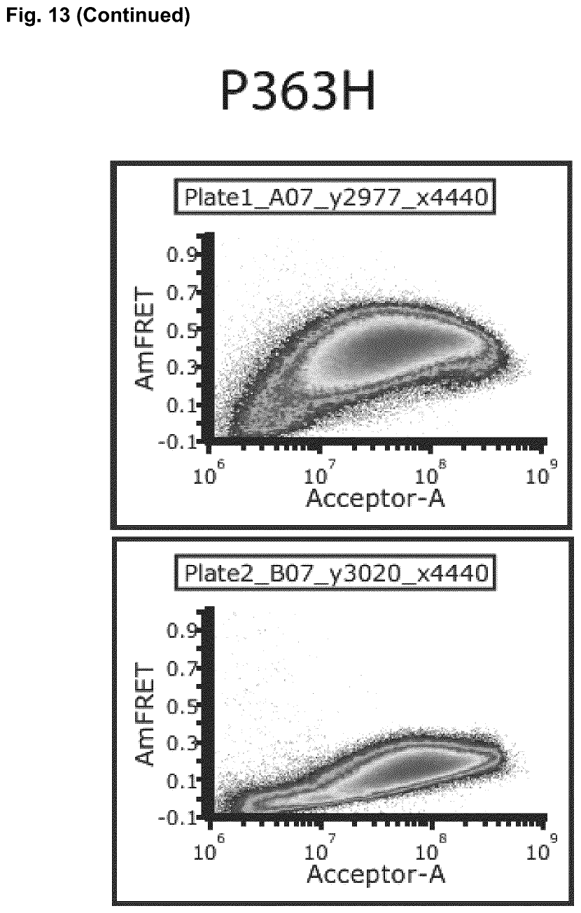

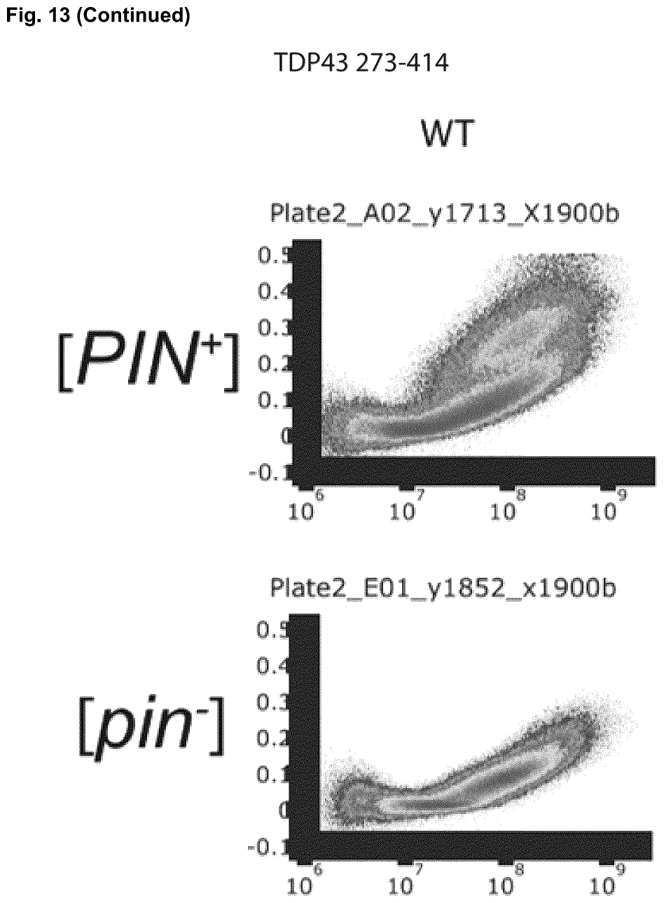

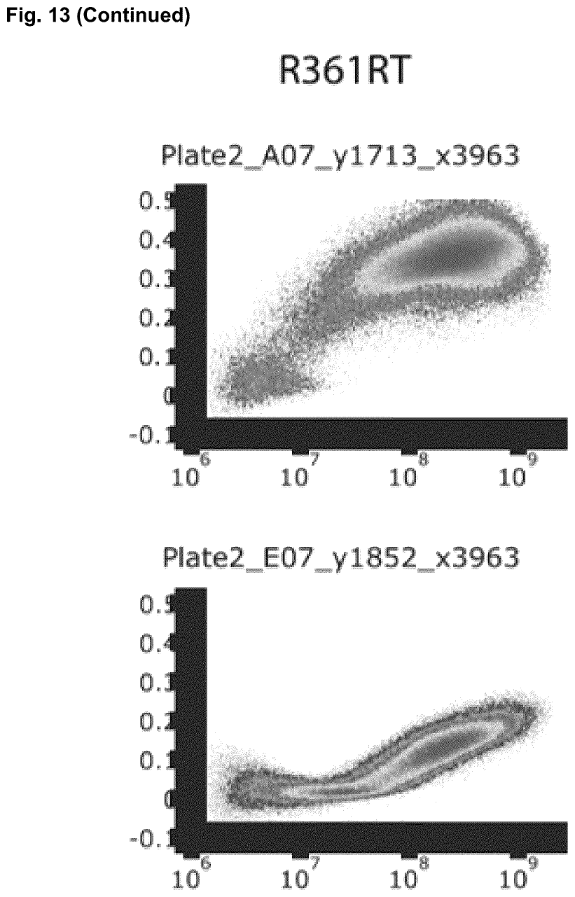

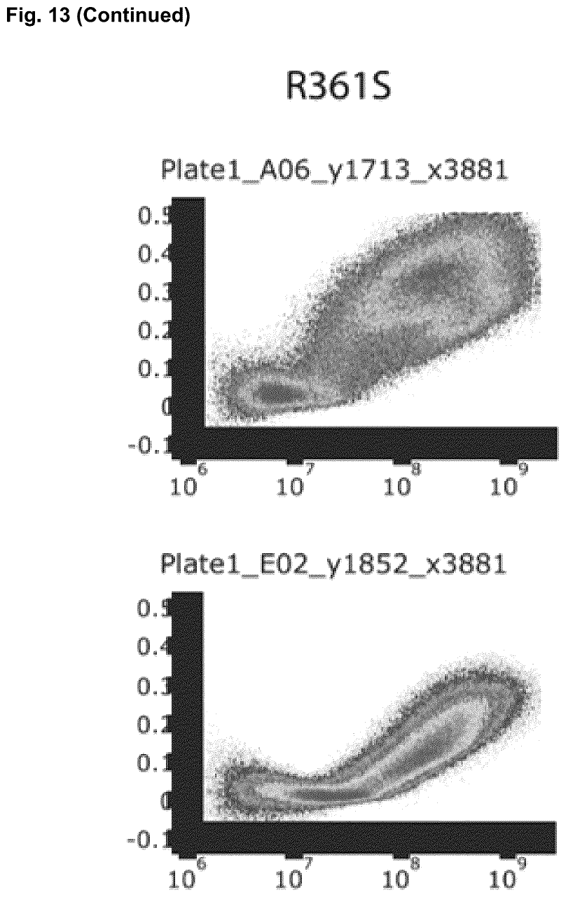

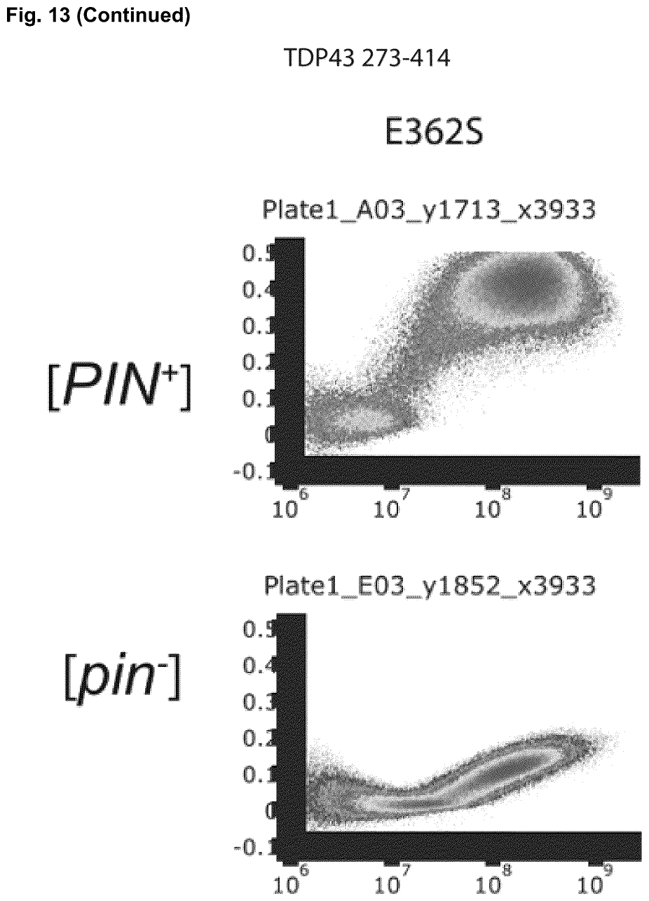

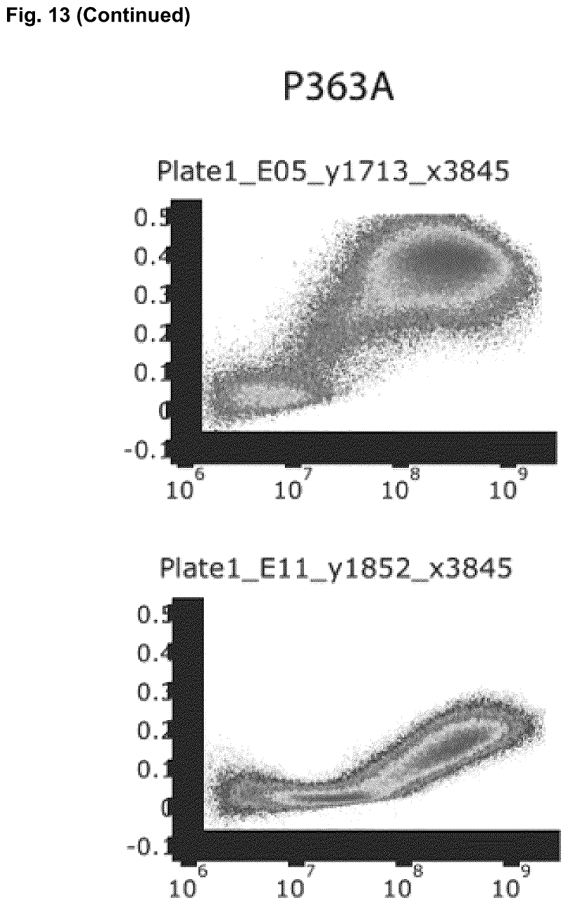

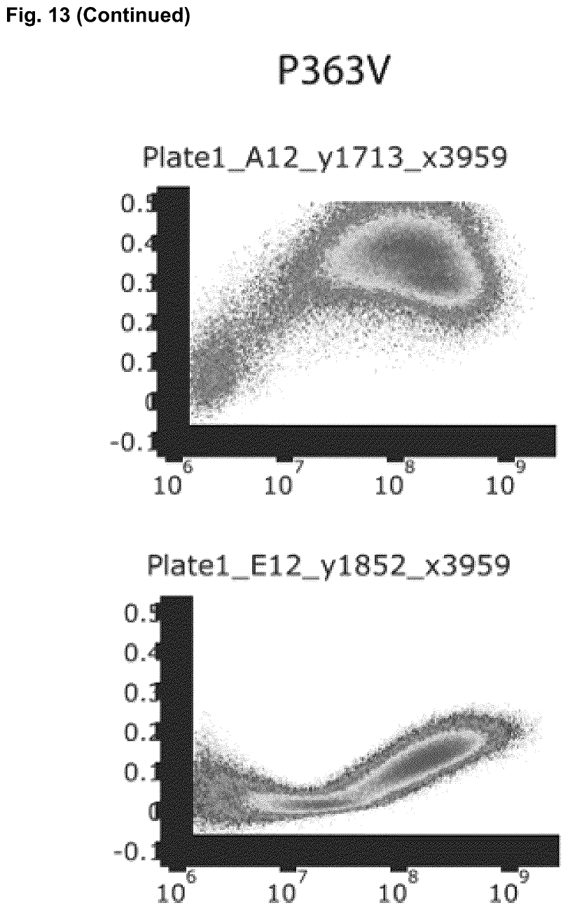

Additional DAmFRET experiments were conducted following similar protocols as described in Example 1. Yeast cells either contaning ([PIN⁺]) or lacking ([pin⁻]) amyloids were transformed with WT or mutant TDP-43 plasmids. Results are shown in FIG. 13.

We found that the conformation of a single proline at residue 363 (P363) profoundly influences amyloid nucleation by TDP-43. Specifically, deleting or introducing any mutation to this residue—regardless of its physicochemical properties—massively accelerated aggregation in the presence of other amyloids. Deleting or mutating either of the preceding charged side chains (R361S or E362S) had the same effect. This suggests that it is proline's unique ability to populate a cis isoform that prevents aggregation, and further, that that isoform does so in a dominant fashion as the great majority (˜90%) of molecules instead contain the highly amyloidogenic trans isoform. By conducting the largest atomistic simulations yet undertaken for a disordered protein, we confirmed that the cis isoform of P363 reduces helicity in the conserved hydrophobic patch and collapses the global conformational ensemble of TDP-43, while prolines at other positions had only minor effects.

This finding suggests that TDP-43 LCS proteins containing a mutation to, or in the immediate vicinity of, P363, may be used to sensitively detect ectopically introduced amyloids of compatible sequence, as in Example 9. It also provides a promising mechanistic link between TDP-43 aggregation and the greatest genetic risk factor for ALS: a hexanucleotide repeat expansion at C9ORF72. The latter causes an aberrant accumulation of proline-arginine, proline-alanine, and proline-glycine dipeptide repeat polypeptides, which have been shown to inactivate the major prolyl isomerase, PP1A (https://doi.org/10.1038/s41467-021-23691-y), which functions to accelerate trans-to-cis isomerization of proline residues. Genetic ablation of PP1A was independently shown to induce TDP-43 pathology in mice (https://doi.org/10.1038/s41467-021-23691-y). Together with the new findings, these observations allow us to propose that TDP-43 pathology is intimately linked to the cis/trans equilibrium of P363.

### Example 11

DAmFRET Biosensor Construction and Validation Using frFAST

Yeast were transformed and expression induced as for the standard (mEos3.1) DAmFRET protocol. Following induction, cells were resuspended in fresh inducing media containing tfLime and tfPoppy fluorogens each at a final concentration of 100 micromolar. Cells were then analyzed by flow cytometry using excitation and emission settings for autofluorescence, tfLime, tfPoppy, and FRET (tfLime excitation with tfPoppy emission). Data are then gated and analyzed as for the standard DAmFRET protocol.

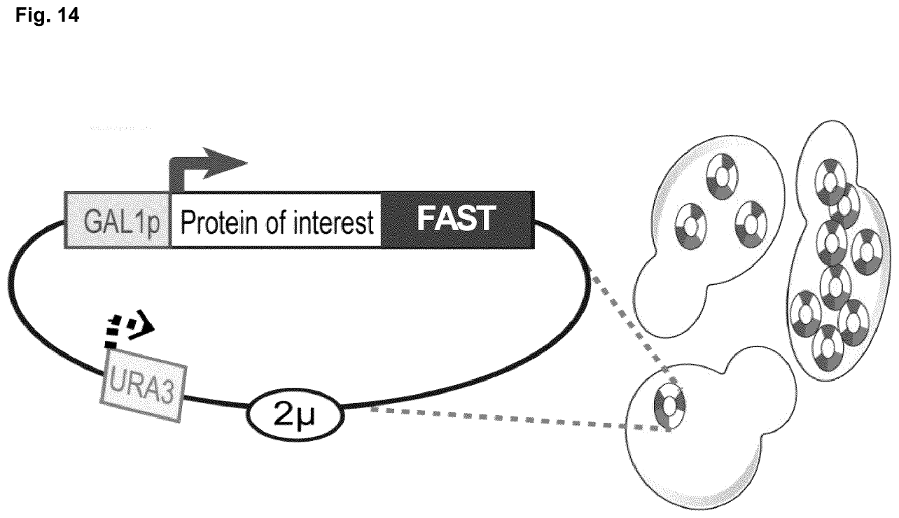

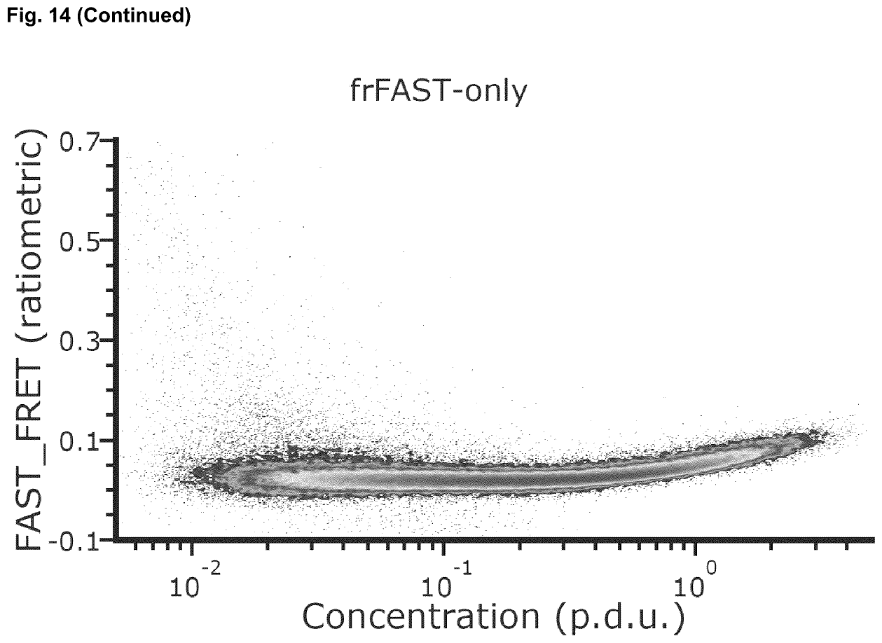

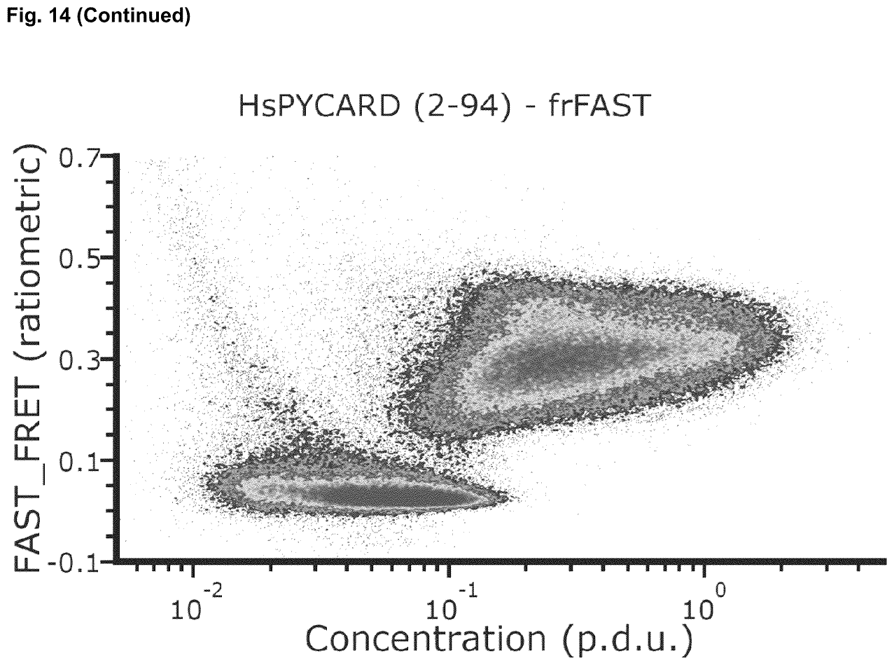

We constructed a DAmFRET plasmid expressing far red (fr)FAST in place of mEos3.1. Inclusion of the fluorogens TFLime and TFPoppy in the culture media allows for mixed labeling of the frFAST-tagged protein with a FRET donor and acceptor, respectively. As shown in FIG. 14, the DAmFRET plot of cells expressing frFAST alone, shows only a modest acquisition of FRET at high expression levels indicating the tag itself is largely monomeric. The DAmFRET plot of cells expressing the prion-like protein ASC fused to frFAST, revealed a bimodal distribution comprising both low- and high-FRET populations of cells qualitatively similar to that achieved by ASC fused to mEos3.1, confirming that self-labeling protein fusions allow for robust amyloid detection in cells.

### DOCUMENTS CITED

1. Aktar F, Burudpakdee C, Polanco M, Pei S, Swayne T C, Lipke P N, Emtage L. 2019. The huntingtin inclusion is a dynamic phase-separated compartment. Life Sci Alliance 2. doi:10.26508/Isa.201900489
2. Alexandrov A I, Polyanskaya A B, Serpionov G V, Ter-Avanesyan M D, Kushnirov V V. 2012. The effects of amino acid composition of glutamine-rich domains on amyloid formation and fragmentation. PLoS ONE 7: e46458. doi:10.1371/journal.pone.0046458
3. Alexandrov I M, Vishnevskaya A B, Ter-Avanesyan M D, Kushnirov V V. 2008. Appearance and propagation of polyglutamine-based amyloids in yeast: tyrosine residues enable polymer fragmentation. J Biol Chem 283:15185-15192. doi:10.1074/jbc.M802071200
4. Arrasate M, Mitra S, Schweitzer E S, Segal M R, Finkbeiner S. 2004. Inclusion body formation reduces levels of mutant huntingtin and the risk of neuronal death. Nature 431:805-810. doi:10.1038/nature02998
5. Auer S, Meersman F, Dobson C M, Vendruscolo M. 2008. A generic mechanism of emergence of amyloid protofilaments from disordered oligomeric aggregates. PLoS Comput Biol 4: e1000222. doi:10.1371/journal.pcbi. 1000222
6. Babinchak W M, Surewicz W K. 2020. Liquid-Liquid Phase Separation and Its Mechanistic Role in Pathological Protein Aggregation. J Mol Biol 432:1910-1925. doi:10.1016/j.jmb.2020.03.004
7. Barrera E E, Zonta F, Pantano S. 2021. Dissecting the role of glutamine in seeding peptide aggregation. Comput Struct Biotechnol J 19:1595-1602. doi:10.1016/j.csbj.2021.02.014
8. Bhattacharyya A M, Thakur A K, Wetzel R. 2005. polyglutamine aggregation nucleation: thermodynamics of a highly unfavorable protein folding reaction. Proc Natl Acad Sci USA 102:15400-15405. doi:10.1073/pnas.0501651102
9. Boatz J C, Piretra T, Lasorsa A, Matlahov I, Conway J F, van der Wel P C A. 2020. Protofilament structure and supramolecular polymorphism of aggregated mutant huntingtin exon 1. J Mol Biol 432:4722-4744. doi:10.1016/j.jmb.2020.06.021
10. Book A, Guella I, Candido T, Brice A, Hattori N, Jeon B, Farrer M J, SNCA Multiplication Investigators of the GEoPD Consortium. 2018. A Meta-Analysis of α-Synuclein Multiplication in Familial Parkinsonism. Front Neurol 9:1021. doi:10.3389/fneur.2018.01021
11. Bracha D, Walls M T, Wei M T, Zhu L, Kurian M, Avalos J L, Toettcher J E, Brangwynne C P. 2018. Mapping local and global liquid phase behavior in living cells using photo-oligomerizable seeds. Cell 175:1467-1480.e13. doi:10.1016/j.cell.2018.10.048
12. Bradley M E, Edskes H K, Hong J Y, Wickner R B, Liebman S W. 2002. Interactions among prions and prion “strains” in yeast. Proc Natl Acad Sci USA 99 Suppl 4:16392-16399. doi:10.1073/pnas. 152330699
13. Buchanan L E, Carr J K, Fluitt A M, Hoganson A J, Moran S D, de Pablo J J, Skinner J L, Zanni M T. 2014. Structural motif of polyglutamine amyloid fibrils discerned with mixed-isotope infrared spectroscopy. Proc Natl Acad Sci USA 111:5796-5801. doi:10.1073/pnas. 1401587111
14. Buell A K. 2017. The Nucleation of Protein Aggregates—From Crystals to Amyloid Fibrils. Int Rev Cell Mol Biol 329:187-226. doi:10.1016/bs.ircmb.2016.08.014
15. Camino J D, Gracia P, Cremades N. 2021. The role of water in the primary nucleation of protein amyloid aggregation. Biophys Chem 269:106520. doi:10.1016/j.bpc.2020.106520
16. Case D A, Belfon K, Ben-Shalom I, Brozell S R, Cerutti D, Cheatham T, Cruzeiro V W D, Darden T, Duke R E, Giambasu G, Gilson M, Gohlke H, Götz A, Harris R, Izadi S, [symbol] C A, Kasavajhala K, Kovalenko A, Krasny R, Kurtzman T, Kollman P A. 2020. Amber 2020.
17. Charoenkwan P, Kanthawong S, Nantasenamat C, Hasan M M, Shoombuatong W. 2021. iAMY-SCM: Improved prediction and analysis of amyloid proteins using a scoring card method with propensity scores of dipeptides. Genomics 113:689-698. doi:10.1016/j.ygeno.2020.09.065
18. Chen S, Berthelier V, Yang W, Wetzel R. 2001. Polyglutamine aggregation behavior in vitro supports a recruitment mechanism of cytotoxicity. J Mol Biol 311:173-182. doi:10.1006/jmbi.2001.4850
19. Chen S, Ferrone F A, Wetzel R. 2002. Huntington's disease age-of-onset linked to polyglutamine aggregation nucleation. Proc Natl Acad Sci USA 99:11884-11889. doi:10.1073/pnas. 182276099
20. Chiti F, Dobson C M. 2017. Protein misfolding, amyloid formation, and human disease: A summary of progress over the last decade. Annu Rev Biochem 86:27-68. doi:10.1146/annurev-biochem-061516-045115
21. Chou P Y, Fasman G D. 1977. β-turns in proteins. J Mol Biol 115:135-175. doi:10.1016/0022-2836(77) 90094-8
22. Clarke G, Collins R A, Leavitt B R, Andrews D F, Hayden M R, Lumsden C J, McInnes R R. 2000. A one-hit model of cell death in inherited neuronal degenerations. Nature 406:195-199. doi:10.1038/35018098
23. Colby D W, Cassady J P, Lin G C, Ingram V M, Wittrup K D. 2006. Stochastic kinetics of intracellular huntingtin aggregate formation. Nat Chem Biol 2:319-323. doi:10.1038/nchembio792
24. Collinge J, Clarke A R. 2007. A general model of prion strains and their pathogenicity. Science 318:930-936. doi:10.1126/science. 1138718
25. Crick S L, Jayaraman M, Frieden C, Wetzel R, Pappu R V. 2006. Fluorescence correlation spectroscopy shows that monomeric polyglutamine molecules form collapsed structures in aqueous solutions. Proc Natl Acad Sci USA 103:16764-16769. doi:10.1073/pnas.0608175103
26. Cubo E, Martinez-Horta S-I, Santalo F S, Descalls A M, Calvo S, Gil-Polo C, Muñoz I, Llano K, Mariscal N, Diaz D, Gutierrez A, Aguado L, Ramos-Arroyo M A, European H D Network. 2019. Clinical manifestations of homozygote allele carriers in Huntington disease. Neurology 92:e2101-e2108. doi:10.1212/WNL.0000000000007147
27. Derkatch I L, Bradley M E, Hong J Y, Liebman S W. 2001. Prions affect the appearance of other prions: the story of [PIN(+)]. Cell 106:171-182. doi:10.1016/s0092-8674(01)00427-5
28. de Mattos E P, Musskopf M K, Bergink S, Kampinga H H. 2022. In vivo suppression of polyglutamine aggregation via co-condensation of the molecular chaperone DNAJB6. BioRxiv. doi:10.1101/2022.08.23.504914
29. Dougan L, Li J, Badilla C L, Berne B J, Fernandez J M. 2009. Single homopolypeptide chains collapse into mechanically rigid conformations. Proc Natl Acad Sci USA 106:12605-12610. doi:10.1073/pnas.0900678106
30. Duennwald M L, Jagadish S, Giorgini F, Muchowski P J, Lindquist S. 2006a. A network of protein interactions determines polyglutamine toxicity. Proc Natl Acad Sci USA 103:11051-11056. doi:10.1073/pnas.0604548103
31. Duennwald M L, Jagadish S, Muchowski P J, Lindquist S. 2006b. Flanking sequences profoundly alter polyglutamine toxicity in yeast. Proc Natl Acad Sci USA 103:11045-11050. doi:10.1073/pnas.0604547103
32. Eisenberg D, Jucker M. 2012. The amyloid state of proteins in human diseases. Cell 148:1188-1203. doi:10.1016/j.cell.2012.02.022
33. Eisenberg D S, Sawaya M R. 2017. Structural studies of amyloid proteins at the molecular level. Annu Rev Biochem 86:69-95. doi:10.1146/annurev-biochem-061516-045104
34. Esposito L, Paladino A, Pedone C, Vitagliano L. 2008. Insights into structure, stability, and toxicity of monomeric and aggregated polyglutamine models from molecular dynamics simulations. Biophys J 94:4031-4040. doi:10.1529/biophysj. 107.118935
35. Essmann U, Perera L, Berkowitz M L, Darden T, Lee H, Pedersen L G. 1995. A smooth particle mesh Ewald method. J Chem Phys 103:8577. doi:10.1063/1.470117
36. Ferreira P C, Ness F, Edwards S R, Cox B S, Tuite M F. 2001. The elimination of the yeast [PSI+] prion by guanidine hydrochloride is the result of Hsp104 inactivation. Mol Microbiol 40:1357-1369. doi:10.1046/j. 1365-2958.2001.02478.x
37. Fletcher J M, Boyle A L, Bruning M, Bartlett G J, Vincent T L, Zaccai N R, Armstrong C T, Bromley E H C, Booth P J, Brady R L, Thomson A R, Woolfson D N. 2012. A basis set of de novo coiled-coil peptide oligomers for rational protein design and synthetic biology. ACS Synth Biol 1:240-250. doi:10.1021/sb300028q
38. Fujiwara K, Toda H, Ikeguchi M. 2012. Dependence of α-helical and β-sheet amino acid propensities on the overall protein fold type. BMC Struct Biol 12:18. doi:10.1186/1472-6807-12-18
39. Gabryelczyk B, Alag R, Philips M, Low K, Venkatraman A, Kannaian B, Shi X, Linder M, Pervushin K, Miserez A. 2022. In vivo liquid-liquid phase separation protects amyloidogenic and aggregation-prone peptides during overexpression in Escherichia coli. Protein Sci 31: e4292. doi:10.1002/pro.4292
40. Galaz-Montoya J G, Shahmoradian S H, Shen K, Frydman J, Chiu W. 2021. Cryo-electron tomography provides topological insights into mutant huntingtin exon 1 and polyQ aggregates. Commun Biol 4:849. doi:10.1038/s42003-021-02360-2
41. Gallagher-Jones M, Glynn C, Boyer D R, Martynowycz M W, Hernandez E, Miao J, Zee C-T, Novikova I V, Goldschmidt L, McFarlane H T, Helguera G F, Evans J E, Sawaya M R, Cascio D, Eisenberg D S, Gonen T, Rodriguez J A. 2018. Sub-ångström cryo-E M structure of a prion protofibril reveals a polar clasp. Nat Struct Mol Biol 25:131-134. doi:10.1038/s41594-017-0018-0
42. Goldstein A L, McCusker J H. 1999. Three new dominant drug resistance cassettes for gene disruption in Saccharomyces cerevisiae. Yeast.
43. Halfmann R, Alberti S, Krishnan R, Lyle N, O'Donnell C W, King O D, Berger B, Pappu R V, Lindquist S. 2011. Opposing effects of glutamine and asparagine govern prion formation by intrinsically disordered proteins. Mol Cell 43:72-84. doi:10.1016/j.molcel.2011.05.013
44. Halfmann R, Lindquist S. 2008. Screening for amyloid aggregation by Semi-Denaturing Detergent-Agarose Gel Electrophoresis. J Vis Exp 17: e838. doi:10.3791/838
45. Hennetin J, Jullian B, Steven A C, Kajava A V. 2006. Standard conformations of beta-arches in beta-solenoid proteins. J Mol Biol 358:1094-1105. doi:10.1016/j.jmb.2006.02.039
46. Hervas R, Rau M J, Park Y, Zhang W, Murzin A G, Fitzpatrick J A J, Scheres S H W, Si K. 2020. Cryo-E M structure of a neuronal functional amyloid implicated in memory persistence in Drosophila. Science 367:1230-1234. doi:10.1126/science.aba3526
47. Hoop C L, Lin H-K, Kar K, Magyarfalvi G, Lamley J M, Boatz J C, Mandal A, Lewandowski J R, Wetzel R, van der Wel P C A. 2016. Huntingtin exon 1 fibrils feature an interdigitated β-hairpin-based polyglutamine core. Proc Natl Acad Sci USA 113:1546-1551. doi:10.1073/pnas. 1521933113
48. Hsieh M-C, Liang C, Mehta A K, Lynn D G, Grover M A. 2017. Multistep conformation selection in amyloid assembly. J Am Chem Soc 139:17007-17010. doi:10.1021/jacs.7b09362
49. Hu W. 2018. The physics of polymer chain-folding. Physics Reports 747:1-50. doi:10.1016/j.physrep.2018.04.004
50. Huang C, Wagner-Valladolid S, Stephens A D, Jung R, Poudel C, Sinnige T, Lechler M C, Schlörit N, Lu M, Laine R F, Michel C H, Vendruscolo M, Kaminski C F, Kaminski Schierle G S, David D C. 2019. Intrinsically aggregation-prone proteins form amyloid-like aggregates and contribute to tissue aging in Caenorhabditis elegans. eLife 8. doi:10.7554/eLife.43059
51. Jiang X, Reiter G, Hu W. 2016. How Chain-Folding Crystal Growth Determines the Thermodynamic Stability of Polymer Crystals. J Phys Chem B 120:566-571. doi:10.1021/acs.jpcb.5b09324
52. Jiang Y, Di Gregorio S E, Duennwald M L, Lajoie P. 2017. Polyglutamine toxicity in yeast uncovers phenotypic variations between different fluorescent protein fusions. Traffic 18:58-70. doi:10.1111/tra. 12453
53. Kakkar V, Månsson C, de Mattos E P, Bergink S, van der Zwaag M, van Waarde M A W H, Kloosterhuis N J, Melki R, van Cruchten R T P, Al-Karadaghi S, Arosio P, Dobson C M, Knowles T P J, Bates G P, van Deursen J M, Linse S, van de Sluis B, Emanuelsson C, Kampinga H H. 2016. The S/T-Rich Motif in the DNAJB6 Chaperone Delays Polyglutamine Aggregation and the Onset of Disease in a Mouse Model. Mol Cell 62:272-283. doi:10.1016/j.molcel.2016.03.017
54. Kang H, Luan B, Zhou R. 2018. Glassy dynamics in mutant huntingtin proteins. J Chem Phys 149:072333. doi:10.1063/1.5029369
55. Kang H, Vazquez F X, Zhang L, Das P, Toledo-Sherman L, Luan B, Levitt M, Zhou R. 2017. Emerging β-Sheet Rich Conformations in Supercompact Huntingtin Exon-1 Mutant Structures. J Am Chem Soc 139:8820-8827. doi:10.1021/jacs.7b00838
56. Kar K, Jayaraman M, Sahoo B, Kodali R, Wetzel R. 2011. Critical nucleus size for disease-related polyglutamine aggregation is repeat-length dependent. Nat Struct Mol Biol 18:328-336. doi:10.1038/nsmb. 1992
57. Keefer K M, Stein K C, True H L. 2017. Heterologous prion-forming proteins interact to cross-seed aggregation in Saccharomyces cerevisiae. Sci Rep 7:5853. doi:10.1038/s41598-017-05829-5
58. Keller A, O'Connor A. 1957. Large Periods in Polyethylene: the Origin of Low-Angle X-ray Scattering. Nature 180:1289-1290. doi:10.1038/1801289a0
59. Keresztes L, Szögi E, Varga B, Farkas V, Perczel A, Grolmusz V. 2021. The budapest amyloid predictor and its applications. Biomolecules 11. doi:10.3390/biom11040500
60. Khan T, Kandola T S, Wu J, Venkatesan S, Ketter E, Lange J J, Rodríguez Gama A, Box A, Unruh J R, Cook M, Halfmann R. 2018. Quantifying nucleation in vivo reveals the physical basis of prion-like phase behavior. Mol Cell 71:155-168.e7. doi:10.1016/j.molcel.2018.06.016
61. Kim G, Gautier O, Tassoni-Tsuchida E, Ma X R, Gitler A D. 2020. ALS genetics: gains, losses, and implications for future therapies. Neuron 108:822-842. doi:10.1016/j.neuron.2020.08.022
62. Kim Y E, Hosp F, Frottin F, Ge H, Mann M, Hayer-Hartl M, Hartl F U. 2016. Soluble Oligomers of PolyQ-Expanded Huntingtin Target a Multiplicity of Key Cellular Factors. Mol Cell 63:951-964. doi:10.1016/j.molcel.2016.07.022
63. Klosin A, Oltsch F, Harmon T, Honigmann A, Jülicher F, Hyman A A, Zechner C. 2020. Phase separation provides a mechanism to reduce noise in cells. Science 367:464-468. doi:10.1126/science.aav6691
64. Knowles T P J, Waudby C A, Devlin G L, Cohen S I A, Aguzzi A, Vendruscolo M, Terentjev E M, Welland M E, Dobson C M. 2009. An analytical solution to the kinetics of breakable filament assembly. Science 326:1533-1537. doi:10.1126/science. 1178250
65. Kryndushkin D, Pripuzova N, Burnett B G, Shewmaker F. 2013. Non-targeted identification of prions and amyloid-forming proteins from yeast and mammalian cells. J Biol Chem 288:27100-27111. doi:10.1074/jbc.M113.485359
66. Kryndushkin D S, Alexandrov I M, Ter-Avanesyan M D, Kushnirov V V. 2003. Yeast [PSI+] prion aggregates are formed by small Sup35 polymers fragmented by Hsp104. J Biol Chem 278:49636-49643. doi:10.1074/jbc.M307996200
67. Kuffner A M, Linsenmeier M, Grigolato F, Prodan M, Zuccarini R, Capasso Palmiero U, Faltova L, Arosio P. 2021. Sequestration within biomolecular condensates inhibits AB-42 amyloid formation. Chem Sci 12:4373-4382. doi:10.1039/d0sc04395h
68. Kuiper E F E, de Mattos E P, Jardim L B, Kampinga H H, Bergink S. 2017. Chaperones in Polyglutamine Aggregation: Beyond the Q-Stretch. Front Neurosci 11:145. doi:10.3389/fnins.2017.00145
69. Lee J-M, Correia K, Loupe J, Kim K-H, Barker D, Hong E P, Chao M J, Long J D, Lucente D, Vonsattel J P G, Pinto R M, Abu Elneel K, Ramos E M, Mysore J S, Gillis T, Wheeler V C, MacDonald M E, Gusella J F, McAllister B, Massey T, Myers R H. 2019. CAG repeat not polyglutamine length determines timing of huntington's disease onset. Cell 178:887-900.e14. doi:10.1016/j.cell.2019.06.036
70. Legleiter J, Mitchell E, Lotz G P, Sapp E, Ng C, DiFiglia M, Thompson L M, Muchowski P J. 2010. Mutant huntingtin fragments form oligomers in a polyglutamine length-dependent manner in vitro and in vivo. J Biol Chem 285:14777-14790. doi:10.1074/jbc.M109.093708
71. Leitman J, Ulrich Hartl F, Lederkremer G Z. 2013. Soluble forms of polyQ-expanded huntingtin rather than large aggregates cause endoplasmic reticulum stress. Nat Commun 4:2753. doi:10.1038/ncomms3753
72. Levin A, Mason T O, Adler-Abramovich L, Buell A K, Meisl G, Galvagnion C, Bram Y, Stratford S A, Dobson C M, Knowles T P J, Gazit E. 2014. Ostwald's rule of stages governs structural transitions and morphology of dipeptide supramolecular polymers. Nat Commun 5:5219. doi:10.1038/ncomms6219
73. Liang C, Hsieh M-C, Li N X, Lynn D G. 2018. Conformational evolution of polymorphic amyloid assemblies. Curr Opin Struct Biol 51:135-140. doi:10.1016/j.sbi.2018.04.004
74. Lieberman A P, Shakkottai V G, Albin R L. 2019. Polyglutamine repeats in neurodegenerative diseases. Annu Rev Pathol 14:1-27. doi:10.1146/annurev-pathmechdis-012418-012857
75. Linsley J W, Tripathi A, Epstein I, Schmunk G, Mount E, Campioni M, Oza V, Barch M, Javaherian A, Nowakowski T J, Samsi S, Finkbeiner S. 2019. Automated four-dimensional long term imaging enables single cell tracking within organotypic brain slices to study neurodevelopment and degeneration. Commun Biol 2:155. doi:10.1038/s42003-019-0411-9
76. Lin H-K, Boatz J C, Krabbendam I E, Kodali R, Hou Z, Wetzel R, Dolga A M, Poirier M A, van der Wel P C A. 2017. Fibril polymorphism affects immobilized non-amyloid flanking domains of huntingtin exon1 rather than its polyglutamine core. Nat Commun 8:15462. doi:10.1038/ncomms15462
77. Li J, Browning S, Mahal S P, Oelschlegel A M, Weissmann C. 2010. Darwinian evolution of prions in cell culture. Science 327:869-872. doi:10.1126/science. 1183218
78. Lipiński W P, Visser B S, Robu I, Fakhree M A A, Lindhoud S, Claessens M M A E, Spruijt E. 2022. Biomolecular condensates can both accelerate and suppress aggregation of α-synuclein. Sci Adv 8: eabq6495. doi:10.1126/sciadv.abq6495
79. Lu B, Palacino J. 2013. A novel human embryonic stem cell-derived Huntington's disease neuronal model exhibits mutant huntingtin (mHTT) aggregates and soluble mHTT-dependent neurodegeneration. FASEB J 27:1820-1829. doi:10.1096/fj. 12-219220
80. Maier J A, Martinez C, Kasavajhala K, Wickstrom L, Hauser K E, Simmerling C. 2015. ff14SB: Improving the Accuracy of Protein Side Chain and Backbone Parameters from ff99SB. J Chem Theory Comput 11:3696-3713. doi:10.1021/acs.jctc.5b00255
81. Man V H, Roland C, Sagui C. 2015. Structural determinants of polyglutamine protofibrils and crystallites. ACS Chem Neurosci 6:632-645. doi:10.1021/cn500358g
82. Margittai M, Langen R. 2008. Fibrils with parallel in-register structure constitute a major class of amyloid fibrils: molecular insights from electron paramagnetic resonance spectroscopy. Q Rev Biophys 41:265-297. doi:10.1017/S0033583508004733
83. Matlahov I, van der Wel P C. 2019. Conformational studies of pathogenic expanded polyglutamine protein deposits from Huntington's disease. Exp Biol Med (Maywood) 244:1584-1595. doi:10.1177/1535370219856620
84. Menon R P, Nethisinghe S, Faggiano S, Vannocci T, Rezaei H, Pemble S, Sweeney M G, Wood N W, Davis M B, Pastore A, Giunti P. 2013. The role of interruptions in polyQ in the pathology of SCA1. PLoS Genet 9: e1003648. doi:10.1371/journal.pgen. 1003648
85. Meriin A B, Zhang X, He X, Newnam G P, Chernoff Y O, Sherman M Y. 2002. Huntington toxicity in yeast model depends on polyglutamine aggregation mediated by a prion-like protein Rnq1. J Cell Biol 157:997-1004. doi:10.1083/jcb.200112104
86. Michaels T C T, Liu L X, Meisl G, Knowles T P J. 2017. Physical principles of filamentous protein self-assembly kinetics. J Phys Condens Matter 29:153002. doi:10.1088/1361-648X/aa5f10
87. Mier P, Elena-Real C, Urbanek A, Bernado P, Andrade-Navarro M A. 2020. The importance of definitions in the study of polyQ regions: A tale of thresholds, impurities and sequence context. Comput Struct Biotechnol J 18:306-313. doi:10.1016/j.csbj.2020.01.012
88. Miller J, Arrasate M, Brooks E, Libeu C P, Legleiter J, Hatters D, Curtis J, Cheung K, Krishnan P, Mitra S, Widjaja K, Shaby B A, Lotz G P, Newhouse Y, Mitchell E J, Osmand A, Gray M, Thulasiramin V, Saudou F, Segal M, Finkbeiner S. 2011. Identifying polyglutamine protein species in situ that best predict neurodegeneration. Nat Chem Biol 7:925-934. doi:10.1038/nchembio.694
89. Moradi M, Babin V, Roland C, Sagui C. 2012. Are long-range structural correlations behind the aggregration phenomena of polyglutamine diseases? PLoS Comput Biol 8: e1002501. doi:10.1371/journal.pcbi.1002501
90. Nacar C. 2020. Propensities of amino acid pairings in secondary structure of globular proteins. Protein J 39:21-32. doi:10.1007/s10930-020-09880-6
91. Nazarov S, Chiki A, Boudeffa D, Lashuel H A. 2022. Structural basis of huntingtin fibril polymorphism revealed by cryogenic electron microscopy of exon 1 HTT fibrils. J Am Chem Soc. doi:10.1021/jacs.2c00509
92. Nethisinghe S, Pigazzini M L, Pemble S, Sweeney M G, Labrum R, Manso K, Moore D, Warner J, Davis M B, Giunti P. 2018. Polyq tract toxicity in SCA1 is length dependent in the absence of CAG repeat interruption. Front Cell Neurosci 12:200. doi:10.3389/fncel.2018.00200
93. Newcombe E A, Ruff K M, Sethi A, Ormsby A R, Ramdzan Y M, Fox A, Purcell A W, Gooley P R, Pappu R V, Hatters D M. 2018. Tadpole-like Conformations of Huntingtin Exon 1 Are Characterized by Conformational Heterogeneity that Persists regardless of Polyglutamine Length. J Mol Biol 430:1442-1458. doi:10.1016/j.jmb.2018.03.031
94. Nizhnikov A A, Alexandrov A I, Ryzhova T A, Mitkevich O V, Dergalev A A, Ter-Avanesyan M D, Galkin A P. 2014. Proteomic screening for amyloid proteins. PLoS ONE 9: e116003. doi:10.1371/journal.pone.0116003
95. Olshina M A, Angley L M, Ramdzan Y M, Tang J, Bailey M F, Hill A F, Hatters D M. 2010. Tracking mutant huntingtin aggregation kinetics in cells reveals three major populations that include an invariant oligomer pool. J Biol Chem 285:21807-21816. doi:10.1074/jbc.M109.084434
96. Osherovich L Z, Cox B S, Tuite M F, Weissman J S. 2004. Dissection and design of yeast prions. PLoS Biol 2: E86. doi:10.1371/journal.pbio.0020086
97. Otzen D, Riek R. 2019. Functional Amyloids. Cold Spring Harb Perspect Biol 11. doi:10.1101/cshperspect.a033860
98. Peskett T R, Rau F, O'Driscoll J, Patani R, Lowe A R, Saibil H R. 2018. A liquid to solid phase transition underlying pathological huntingtin exon1 aggregation. Mol Cell 70:588-601.e6. doi:10.1016/j.molcel.2018.04.007
99. Posey A E, Ruff K M, Lalmansingh J M, Kandola T S, Lange J J, Halfmann R, Pappu R V. 2021. Mechanistic inferences from analysis of measurements of protein phase transitions in n live cells. J Mol Biol 433:166848. doi:10.1016/j.jmb.2021.166848
100. Prabakaran R, Rawat P, Thangakani A M, Kumar S, Gromiha M M. 2021. Protein aggregation: in silico algorithms and applications. Biophys Rev 13:71-89. doi:10.1007/s12551-021-00778-w
101. Price D J, Brooks C L. 2004. A modified TIP3P water potential for simulation with Ewald summation. J Chem Phys 121:10096-10103. doi:10.1063/1.1808117
102. Punihaole D, Jakubek R S, Workman R J, Asher S A. 2018. Interaction enthalpy of side chain and backbone amides in polyglutamine solution monomers and fibrils. J Phys Chem Lett 9:1944-1950. doi:10.1021/acs.jpclett.8b00348
103. Sadler D M. 1983. Roughness of growth faces of polymer crystals: Evidence from morphology and implications for growth mechanisms and types of folding. Polymer 24:1401-1409. doi:10.1016/0032-3861(83) 90220-3
104. Sanders D W, Kaufman S K, DeVos S L, Sharma A M, Mirbaha H, Li A, Barker S J, Foley A C, Thorpe J R, Serpell L C, Miller T M, Grinberg L T, Seeley W W, Diamond M I. 2014. Distinct tau prion strains propagate in cells and mice and define different tauopathies. Neuron 82:1271-1288. doi:10.1016/j.neuron.2014.04.047
105. Sathasivam K, Lane A, Legleiter J, Warley A, Woodman B, Finkbeiner S, Paganetti P, Muchowski P J, Wilson S, Bates G P. 2010. Identical oligomeric and fibrillar structures captured from the brains of R6/2 and knock-in mouse models of Huntington's disease. Hum Mol Genet 19:65-78. doi:10.1093/hmg/ddp467
106. Sawaya M R, Hughes M P, Rodriguez J A, Riek R, Eisenberg D S. 2021. The expanding amyloid family: Structure, stability, function, and pathogenesis. Cell 184:4857-4873. doi:10.1016/j.cell.2021.08.013
107. Sawaya M R, Sambashivan S, Nelson R, Ivanova M I, Sievers S A, Apostol M I, Thompson M J, Balbirnie M, Wiltzius J J W, McFarlane H T, Madsen AØ, Riekel C, Eisenberg D. 2007. Atomic structures of amyloid cross-beta spines reveal varied steric zippers. Nature 447:453-457. doi:10.1038/nature05695
108. Schneider R, Schumacher M C, Mueller H, Nand D, Klaukien V, Heise H, Riedel D, Wolf G, Behrmann E, Raunser S, Seidel R, Engelhard M, Baldus M. 2011. Structural characterization of polyglutamine fibrils by solid-state NMR spectroscopy. J Mol Biol 412:121-136. doi:10.1016/j.jmb.2011.06.045
109. Selkoe D J, Hardy J. 2016. The amyloid hypothesis of Alzheimer's disease at 25 years. EMBO Mol Med 8:595-608. doi:10.15252/emmm.201606210
110. Sen S, Dash D, Pasha S, Brahmachari S K. 2003. Role of histidine interruption in mitigating the pathological effects of long polyglutamine stretches in SCA1: A molecular approach. Protein Sci 12:953-962. doi:10.1110/ps.0224403
111. Serio T R, Cashikar A G, Kowal A S, Sawicki G J, Moslehi J J, Serpell L, Arnsdorf M F, Lindquist S L. 2000. Nucleated conformational conversion and the replication of conformational information by a prion determinant. Science 289:1317-1321. doi:10.1126/science.289.5483.1317
112. Serio T R. 2018. [PIN+]ing down the mechanism of prion appearance. FEMS Yeast Res 18. doi:10.1093/femsyr/foy026
113. Serpionov G V, Alexandrov A I, Antonenko Y N, Ter-Avanesyan M D. 2015. A protein polymerization cascade mediates toxicity of non-pathological human huntingtin in yeast. Sci Rep 5:18407. doi:10.1038/srep18407
114. Sharma D, Shinchuk L M, Inouye H, Wetzel R, Kirschner D A. 2005. Polyglutamine homopolymers having 8-45 residues form slablike beta-crystallite assemblies. Proteins 61:398-411. doi:10.1002/prot.20602
115. Shin Y, Brangwynne C P. 2017. Liquid phase condensation in cell physiology and disease. Science 357. doi:10.1126/science.aaf4382
116. Sikorski P, Atkins E. 2005. New model for crystalline polyglutamine assemblies and their connection with amyloid fibrils. Biomacromolecules 6:425-432. doi:10.1021/bm0494388
117. Sil T B, Sahoo B, Bera S C, Garai K. 2018. Quantitative characterization of metastability and heterogeneity of amyloid aggregates. Biophys J 114:800-811. doi:10.1016/j.bpj.2017.12.023
118. Simm S, Einloft J, Mirus O, Schleiff E. 2016. 50 years of amino acid hydrophobicity scales: revisiting the capacity for peptide classification. Biol Res 49:31. doi:10.1186/s40659-016-0092-5
119. Sinnige T, Meisl G, Michaels T C T, Vendruscolo M, Knowles T P J, Morimoto R I. 2021. Kinetic analysis reveals that independent nucleation events determine the progression of polyglutamine aggregation in C. elegans. Proc Natl Acad Sci USA 118. doi:10.1073/pnas.2021888118
120. Sprunger M L, Jackrel M E. 2021. Prion-Like Proteins in Phase Separation and Their Link to Disease. Biomolecules 11. doi:10.3390/biom11071014
121. Strodel B. 2021. Amyloid aggregation simulations: challenges, advances and perspectives. Curr Opin Struct Biol 67:145-152. doi:10.1016/j.sbi.2020.10.019
122. Takahashi T, Kikuchi S, Katada S, Nagai Y, Nishizawa M, Onodera O. 2008. Soluble polyglutamine oligomers formed prior to inclusion body formation are cytotoxic. Hum Mol Genet 17:345-356. doi:10.1093/hmg/ddm311
123. Tanaka M, Collins S R, Toyama B H, Weissman J S. 2006. The physical basis of how prion conformations determine strain phenotypes. Nature 442:585-589. doi:10.1038/nature04922
124. Thakur A K, Wetzel R. 2002. Mutational analysis of the structural organization of polyglutamine aggregates. Proc Natl Acad Sci USA 99:17014-17019. doi:10.1073/pnas.252523899
125. Törnquist M, Michaels T C T, Sanagavarapu K, Yang X, Meisl G, Cohen S I A, Knowles T P J, Linse S. 2018. Secondary nucleation in amyloid formation. Chem Commun 54:8667-8684. doi:10.1039/c8cc02204f
126. Ungar G, Keller A. 1987. Inversion of the temperature dependence of crystallization rates due to onset of chain folding. Polymer 28:1899-1907. doi:10.1016/0032-3861(87) 90298-9
127. Ungar G, Putra E G R, de Silva D S M, Shcherbina M A, Waddon A J. 2005. The Effect of Self-Poisoning on Crystal Morphology and Growth Rates In: Allegra G, editor. Interphases and Mesophases in Polymer Crystallization I, Advances in Polymer Science. Berlin, Heidelberg: Springer Berlin Heidelberg. pp. 45-87. doi:10.1007/b107232
128. Vekilov P G. 2012. Phase diagrams and kinetics of phase transitions in protein solutions. J Phys Condens Matter 24:193101. doi:10.1088/0953-8984/24/19/193101
129. Venkatesan S, Kandola T S, Rodríguez-Gama A, Box A, Halfmann R. 2019. Detecting and characterizing protein self-assembly in vivo by flow cytometry. J Vis Exp. doi:10.3791/59577
130. Verges K J, Smith M H, Toyama B H, Weissman J S. 2011. Strain conformation, primary structure and the propagation of the yeast prion [PSI+]. Nat Struct Mol Biol 18:493-499. doi:10.1038/nsmb.2030
131. Vitalis A, Pappu R V. 2011. Assessing the contribution of heterogeneous distributions of oligomers to aggregation mechanisms of polyglutamine peptides. Biophys Chem 159:14-23. doi:10.1016/j.bpc.2011.04.006
132. Vitalis A, Wang X, Pappu R V. 2007. Quantitative characterization of intrinsic disorder in polyglutamine: insights from analysis based on polymer theories. Biophys J 93:1923-1937. doi:10.1529/biophysj.107.110080
133. Walters R H, Jacobson K H, Pedersen J A, Murphy R M. 2012. Elongation kinetics of polyglutamine peptide fibrils: a quartz crystal microbalance with dissipation study. J Mol Biol 421:329-347. doi:10.1016/j.jmb.2012.03.017
134. Walters R H, Murphy R M. 2009. Examining polyglutamine peptide length: a connection between collapsed conformations and increased aggregation. J Mol Biol 393:978-992. doi:10.1016/j.jmb.2009.08.034
135. Wang X, Vitalis A, Wyczalkowski M A, Pappu R V. 2006. Characterizing the conformational ensemble of monomeric polyglutamine. Proteins 63:297-311. doi:10.1002/prot.20761
136. Wan Q, Mouton S N, Veenhoff L M, Boersma A J. 2021. A precise and general FRET-based method for monitoring structural transitions in protein self-organization. BioRxiv. doi:10.1101/2021.02.25.432866
137. Wei M-T, Elbaum-Garfinkle S, Holehouse A S, Chen C C-H, Feric M, Arnold C B, Priestley R D, Pappu R V, Brangwynne C P. 2017. Phase behaviour of disordered proteins underlying low density and high permeability of liquid organelles. Nat Chem 9:1118-1125. doi:10.1038/nchem.2803
138. Wetzel R. 2020. Exploding the Repeat Length Paradigm while Exploring Amyloid Toxicity in Huntington's Disease. Acc Chem Res 53:2347-2357. doi:10.1021/acs.accounts.0c00450
139. Wetzel R. 2006. Nucleation of huntingtin aggregation in cells. Nat Chem Biol 2:297-298. doi:10.1038/nchembio0606-297
140. Wexler N S, Young A B, Tanzi R E, Travers H, Starosta-Rubinstein S, Penney J B, Snodgrass S R, Shoulson I, Gomez F, Ramos Arroyo M A. 1987. Homozygotes for Huntington's disease. Nature 326:194-197. doi:10.1038/326194a0
141. Whitelam S, Dahal Y R, Schmit J D. 2016. Minimal physical requirements for crystal growth self-poisoning. J Chem Phys 144:064903. doi:10.1063/1.4941457
142. Xu J, Reiter G, Alamo R. 2021. Concepts of nucleation in polymer crystallization. Crystals 11:304. doi:10.3390/cryst11030304
143. Yamaguchi K-I, Takahashi S, Kawai T, Naiki H, Goto Y. 2005. Seeding-dependent propagation and maturation of amyloid fibril conformation. J Mol Biol 352:952-960. doi:10.1016/j.jmb.2005.07.061
144. Zanjani A A H, Reynolds N P, Zhang A, Schilling T, Mezzenga R, Berryman J T. 2020. Amyloid evolution: antiparallel replaced by parallel. Biophys J 118:2526-2536. doi:10.1016/j.bpj.2020.03.023
145. Zhang L, Schmit J D. 2016. Pseudo-one-dimensional nucleation in dilute polymer solutions. Phys Rev E 93:060401. doi:10.1103/PhysRevE.93.060401
146. Zhang M, Guo B-H, Xu J. 2016. A review on polymer crystallization theories. Crystals 7:4. doi:10.3390/cryst7010004
147. Zhang S, Wang Z, Guo B, Xu J. 2021. Secondary nucleation in polymer crystallization: A kinetic view. Polymer Crystallization 4. doi:10.1002/pcr2.10173
148. Zhang X, Marxsen S F, Ortmann P, Mecking S, Alamo R G. 2020. Crystallization of Long-Spaced Precision Polyacetals II: Effect of Polymorphism on Isothermal Crystallization Kinetics. Macromolecules 53:7899-7913. doi:10.1021/acs.macromol.0c01443
149. Zhang X, Zhang W, Wagener K B, Boz E, Alamo R G. 2018. Effect of Self-Poisoning on Crystallization Kinetics of Dimorphic Precision Polyethylenes with Bromine. Macromolecules 51:1386-1397. doi:10.1021/acs.macromol.7b02745
150. Zhang Y, Man V H, Roland C, Sagui C. 2016. Amyloid Properties of Asparagine and Glutamine in Prion-like Proteins. ACS Chem Neurosci 7:576-587. doi:10.1021/acschemneuro.5b00337

All documents cited in this application are hereby incorporated by reference as if recited in full herein.

Although illustrative embodiments of the present disclosure have been described herein, it should be understood that the disclosure is not limited to those described, and that various other changes or modifications may be made by one skilled in the art without departing from the scope or spirit of the disclosure.

What is claimed is:

1. A method for preventing, treating or ameliorating the effects of an amyloid-associated condition in a subject, comprising:
(a) identifying a target protein whose aggregation causes the amyloid-associated condition in the subject; and
(b) treating the subject by modifying the target protein to induce preemptive oligomerization thereof.

2. The method of claim 1, wherein the amyloid-associated condition is a neurodegenerative disease selected from the group consisting of amyotrophic lateral sclerosis (ALS), frontotemporal dementia (FTD), dementia with Lewy bodies, familial British dementia, familial Danish dementia, Alzheimer's disease (AD), limbic-predominant age-related TDP-43 encephalopathy (LATE), Parkinson's disease (PD), spongiform encephalopathies, and a polyglutamine (polyQ) disease.

3. The method of claim 2, wherein the polyQ disease is selected from the group consisting of spinocerebellar ataxia type 1 (SCA1), SCA2, SCA6, SCA7, SCA17, Machado-Joseph disease (MJD/SCA3), Huntington's disease (HD), dentatorubral pallidoluysian atrophy (DRPLA), and spinal and bulbar muscular atrophy, X-linked 1 (SMAX1/SBMA).

4. The method of claim 2, wherein the target protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), TAR DNA-binding protein 43 (TDP-43), Cav2.1, ataxin-2, Huntingtin, androgen receptor, ataxin-7, ataxin-1, TATA-binding protein, atrophin-1, and ataxin-3.

5. The method of claim 1, wherein the amyloid-associated condition is a non-neurodegenerative disease selected from the group consisting of AL amyloidosis, AA amyloidosis, familial Mediterranean fever, senile systemic amyloidosis, familial amyloidotic polyneuropathy, hemodialysis-related amyloidosis, apoAI amyloidosis, apoAII amyloidosis, apoAIV amyloidosis, Finnish hereditary amyloidosis, lysozyme amyloidosis, fibrinogen amyloidosis, Icelandic hereditary cerebral amyloid angiopathy, Type II diabetes, medullary carcinoma of the thyroid, atrial amyloidosis, hereditary cerebral haemorrhage with amyloidosis, pituitary prolactinoma, injection-localized amyloidosis, aortic medial amyloidosis, hereditary lattice corneal dystrophy, corneal amylodosis associated with trichiasis, cataract, calcifying epithelial odontogenic tumors, pulmonary alveolar proteinosis, inclusion-body myositis, multisystem proteinopathy, senile seminal vesicle amyloidosis, amyloid tumor, LECT2 amyloidosis, and cutaneous lichen amyloidosis.

6. The method of claim 5, wherein the target protein is selected from the group consisting of immunoglobulin light chains or fragments, serum amyloid A protein, transthyretin, β2-microglobulin, apolipoprotein AI, apolipoprotein AII, apolipoprotein AIV, gelsolin, lysozyme, fibrinogen α-chain, cystatin C, amylin, calcitonin, atrial natriuretic factor, amyloid β peptide, prolactin, insulin, medin, kerato-epithelin, lactoferrin, γ-crystallins, lung surfactant protein C, heterogeneous nuclear ribonucleoprotein D like (hnRNPDL), receptor-interacting serine/threonine-protein kinase 1 (RIPK1), receptor-interacting serine/threonine-protein kinase 3 (RIPK3), galectin-7, S100 calcium-binding protein A8 (S100A8), S100 calcium-binding protein A9 (S100A9), semenogelin 1 (SEM1), leukocyte chemotactic factor 2 (LECT-2), and keratins.

7. The method of claim 1, wherein the treatment is carried out in vivo or ex vivo.

8. The method of claim 1, wherein the modification of the target protein is pre- or post-translational.

9. The method of claim 1, wherein the modification of the target protein is carried out by modifying its encoding gene.

10. The method of claim 9, wherein the encoding gene of the target protein is modified by mutagenesis or gene fusion.

11. The method of claim 1, wherein the modification of the target protein is carried out by introducing into the subject a transgene expressing a homo-oligomerizing moiety that is fused to a binding protein against the target protein.

12. The method of claim 11, wherein the transgene is introduced by an adeno-associated virus (AAV).

13. The method of claim 11, wherein the binding protein is an intrabody.

14. The method of claim 1, wherein the modification of the target protein is carried out by administering to the subject an effective amount of a small molecule binder against the target protein.

15. The method of claim 14, wherein the small molecule binder comprise a self-reactive moiety that makes the small molecule binder multivalent.

16. The method of claim 1, wherein the subject is a mammal selected from the group consisting of humans, primates, farm animals, and domestic animals.

17. The method of claim 1, wherein the subject is a human.

18. A method for preventing, treating or ameliorating the effects of a polyglutamine (polyQ) disease in a subject, comprising:
(a) identifying a target protein whose aggregation causes the polyQ disease in the subject;
(b) obtaining a biological sample from the subject and screening for expanded cytosine-adenine-guanine (CAG) repeats encoding a long polyQ tract in the target protein;
(c) identifying the subject as having high risk to develop the polyQ disease, if the long polyQ tract in the target protein contains a number of glutamines exceeding a pathogenic threshold for the polyQ disease; and
(d) treating the identified subject by modifying the target protein to induce preemptive oligomerization thereof.

19. The method of claim 18, wherein the polyQ disease is selected from the group consisting of spinocerebellar ataxia type 1 (SCA1), SCA2, SCA6, SCA7, SCA17, Machado-Joseph disease (MJD/SCA3), Huntington's disease (HD), dentatorubral pallidoluysian atrophy (DRPLA), and spinal and bulbar muscular atrophy, X-linked 1 (SMAX1/SBMA).

20. The method of claim 18, wherein the target protein is selected from the group consisting of Cav2.1, ataxin-2, Huntingtin, androgen receptor, ataxin-7, ataxin-1, TATA-binding protein, atrophin-1, and ataxin-3.

21. The method of claim 18, wherein the treatment is carried out in vivo or ex vivo.

22. The method of claim 18, wherein the modification of the target protein is pre- or post-translational.

23. The method of claim 18, wherein the modification of the target protein is carried out by modifying its encoding gene.

24. The method of claim 23, wherein the encoding gene of the target protein is modified by mutagenesis or gene fusion.

25. The method of claim 18, wherein the subject is a mammal selected from the group consisting of humans, primates, farm animals, and domestic animals.

26. The method of claim 18, wherein the subject is a human.

27. The method of claim 18, wherein the pathogenic threshold of the polyQ disease is in the range of 32 to 54 glutamines in the long polyQ tract of the target protein.

28. The method of claim 18, wherein the modification of the target protein is carried out by mutating its encoding gene that results in a mutant target protein having the amino acid pattern of Q_{A}X_{B} in its long polyQ tract, wherein X is any amino acid other than Q and wherein A and B are independently positive integers.

29. The method of claim 28, wherein A=2 or 4 and B=1.

30. The method of claim 21, wherein A=3 or 5 and B=1.

31. A method for preventing, treating or ameliorating the effects of Huntington's disease (HD) in a subject, comprising:
(a) obtaining a biological sample from the subject and screening for expanded cytosine-adenine-guanine (CAG) repeats encoding a long polyQ tract in the Huntingtin protein;
(b) identifying the subject as having high risk to develop HD, if the long polyQ tract in the Huntingtin protein contains 36 or more glutamines; and
(c) treating the identified subject by modifying the Huntingtin protein to induce preemptive oligomerization thereof.

32. The method of claim 31, wherein the treatment is carried out in vivo or ex vivo.

33. The method of claim 31, wherein the modification of the Huntingtin protein is pre- or post-translational.

34. The method of claim 31, wherein the modification of the Huntingtin protein is carried out by modifying the HTT gene.

35. The method of claim 34, wherein the HTT gene is modified by mutagenesis or gene fusion.

36. The method of claim 31, wherein the subject is a mammal selected from the group consisting of humans, primates, farm animals, and domestic animals.

37. The method of claim 31, wherein the subject is a human.

38. The method of claim 31, wherein the modification of the Huntingtin protein is carried out by mutating the HTT gene that results in a mutant Huntingtin protein having the amino acid pattern of Q_{A}X_{B} in its long polyQ tract, wherein X is any amino acid other than Q and wherein A and B are independently positive integers.

39. The method of claim 38, wherein A=2 or 4 and B=1.

40. The method of claim 38, wherein A=3 or 5 and B=1.

41. The method of claim 38, wherein X is an amino acid selected from the group consisting of asparagine (N), glycine (G), alanine (A), serine(S), and histidine (H).

42. The method of claim 38, wherein X is asparagine (N).

43. The method of claim 38, wherein the amino acid pattern of Q_{A}X_{B} is Q₅N₁.

44. The method of claim 31, wherein modification of the Huntingtin protein is carried out by fusing the HTT gene with a sequence encoding a homo-oligomeric protein.

45. The method of claim 44, wherein the homo-oligomeric protein is a coiled coil dimer or human FTH1.

46. A method for preventing, treating or ameliorating the effects of an amyloid-associated neurodegenerative disease in a subject, comprising:
(a) identifying an amyloid-forming protein whose aggregation causes the amyloid-associated neurodegenerative disease in the subject;
(b) obtaining a biological sample from the subject and screening for the amyloid-forming protein;
(c) identifying the subject as having high risk to develop the amyloid-associated neurodegenerative disease, if the amyloid-forming protein exists in the subject; and
(d) treating the identified subject by modifying the amyloid-forming protein to induce preemptive oligomerization thereof.

47. The method of claim 46, wherein the amyloid-forming protein is wild type.

48. The method of claim 46, wherein the amyloid-forming protein comprises at least one amyloid-promoting mutation.

49. The method of claim 46, wherein the amyloid-associated neurodegenerative disease is a selected from the group consisting of amyotrophic lateral sclerosis (ALS), frontotemporal dementia (FTD), dementia with Lewy bodies, familial British dementia, familial Danish dementia, Alzheimer's disease (AD), limbic-predominant age-related TDP-43 encephalopathy (LATE), spongiform encephalopathies, and Parkinson's disease (PD).

50. The method of claim 46, wherein the amyloid-forming protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), and TAR DNA-binding protein 43 (TDP-43).

51. The method of claim 46, wherein the amyloid-forming protein is β-amyloid precursor protein (APP) and the at least one amyloid-promoting mutation is selected from the group consisting of K670_M671delinsNL, A673T, A673V, D678N, D678H, E682K, K687Q, L688V, A692G, E693K, E693Q, E693G, E693del, D694N, L705V, T714A, T714I, V715M, V715A, I716F, V717L, V717I, V717F, V717G, T719N, M722K, L723P, and combinations thereof.

52. The method of claim 46, wherein the amyloid-forming protein is tau and the at least one amyloid-promoting mutation is selected from the group consisting of R5L, G55R, K257T, I260V, L266V, G272V, G273R, N279K, L284R, N296D, N296del, N296H, P301T, P301S, P301L, G303V, S305I, S305N, K317M, K317N, S320F, P332S, G335S, G335A, Q336R, Q336H, V337M, E342V, S352L, S356T, P364S, G366R, K369I, E372G, G389R, P397S, R406W, N410H, T427M, S320Y, S385R, and combinations thereof.

53. The method of claim 46, wherein the amyloid-forming protein is α-synuclein and the at least one amyloid-promoting mutation is selected from the group consisting of A30P, E46K, H50Q, G51D, A53E, A53T, and combinations thereof.

54. The method of claim 46, wherein the amyloid-forming protein is TDP-43 and the at least one amyloid-promoting mutation is selected from the group consisting of G294A, G294V, G295S, A315T, Q331K, M337V, M337P, Q343R, N345K, R361S, R361RT, E362S, P363A, P363V, P363G, P363H, N390D, N390S, and combinations thereof.

55. The method of claim 46, wherein the treatment is carried out in vivo or ex vivo.

56. The method of claim 46, wherein the modification of the amyloid-forming protein is pre- or post-translational.

57. The method of claim 46, wherein the modification of the amyloid-forming protein is carried out by modifying its encoding gene.

58. The method of claim 57, wherein the encoding gene of the amyloid-forming protein is modified by mutagenesis or gene fusion.

59. The method of claim 58, wherein the gene fusion is carried out by fusing the encoding gene of the amyloid-forming protein with a sequence encoding a homo-oligomeric protein.

60. The method of claim 59, wherein the homo-oligomeric protein is a coiled coil dimer or human FTH1.

61. The method of claim 46, wherein the subject is a mammal selected from the group consisting of humans, primates, farm animals, and domestic animals.

62. The method of claim 46, wherein the subject is a human.

63. A method for preventing, treating or ameliorating the effects of a condition associated with TAR DNA-binding protein 43 (TDP-43) in a subject, comprising:
(a) obtaining a biological sample from the subject and screening for TDP-43;
(b) identifying the subject as having high risk to develop the condition, if TDP-43 exists in the subject; and
(c) treating the identified subject by modifying TDP-43 to induce preemptive oligomerization thereof.

64. The method of claim 63, wherein the condition is selected from the group consisting of amyotrophic lateral sclerosis (ALS), ALS related cognitive/behavioural impairment (ALS-ci/bi), multiple system proteinopathy-A familial disorder (MSP), frontotemporal lobar degeneration (FTLD), limbic-predominant agerelated TDP-43 encephalopathy (LATE), cerebral age-related TDP-43 with sclerosis (CARTS), Perry disease, facial onset sensory and motor neuronopathy (FOSMN), sporadic inclusion body myositis (sIBM), Alzheimer's disease (AD), dementia with Lewy bodies, Parkinson's disease (PD), Huntington's disease (HD), chronic traumatic encephalopathy (CTE), primary progressive aphasia (PSP), corticobasal degeneration (CBD), argyrophilic grain disease (AGD), and combinations thereof.

65. The method of claim 63, wherein the treatment is carried out in vivo or ex vivo.

66. The method of claim 63, wherein the modification of TDP-43 is pre- or post-translational.

67. The method of claim 63, wherein the modification of TDP-43 is carried out by modifying the TARDBP gene.

68. The method of claim 67, wherein the TARDBP gene is modified by gene fusion.

69. The method of claim 63, wherein the modification of TDP-43 is carried out by fusing the TARDBP gene with a sequence encoding a homo-oligomeric protein.

70. The method of claim 69, wherein the homo-oligomeric protein is a coiled coil dimer or human FTH1.

71. The method of claim 63, wherein TDP-43 is wild type.

72. The method of claim 63, wherein TDP-43 comprises at least one amyloid-promoting mutation.

73. The method of claim 72, wherein the at least one amyloid-promoting mutation is selected from the group consisting of G294A, G294V, G295S, A315T, Q331K, M337V, M337P, Q343R, N345K, R361S, R361RT, E362S, P363A, P363V, P363G, P363H, N390D, N390S, and combinations thereof.

74. The method of claim 63, wherein the subject is a mammal selected from the group consisting of humans, primates, farm animals, and domestic animals.

75. The method of claim 63, wherein the subject is a human.

76. A method for detecting amyloid nucleation of a protein of interest, comprising:
(a) generating a first yeast strain by (i) deleting PDR5 and ATG8 genes from a yeast strain rhy1713, and then (ii) integrating BDFP1.6:1.6 prior to the stop codon of chromosomal PGK1;
(b) generating a second yeast strain by eliminating the amyloid form of Rnq1 from the first yeast strain;
(c) transforming the first and second yeast strains with a plasmid encoding the protein of interest as a fusion to a fluorescent tag;
(d) incubating the transformed strains under suitable conditions and inducing them for a suitable time;
(e) conducting high-throughput flow cytometry on these induced cells and collecting fluorescence signals; and
(f) analyzing the collected signals to determine amyloid nucleation of the protein of interest.

77. A method for constructing a biosensor cell that is used to detect aggregates of a specific protein in a biological sample, comprising:
(a) constructing a reporter gene that encodes the specific protein fused with a fluorescent tag;
(b) cloning the reporter gene onto a lentiviral transfer plasmid;
(c) transfecting the lentviral transfer plasmid together with a packaging plasmid and an envelope plasmid into HEK293T cells;
(d) incubating the transfected cells and collecting viral particles containing the reporter gene;
(e) incubating the viral particles collected in step (d) with fresh HE293T cells; and
(f) sorting for single cell clone containing the integrated reporter gene and expanding it as the biosensor cell.

78. The method of claim 77, wherein the specific protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), and TAR DNA-binding protein 43 (TDP-43).

79. A biosensor cell prepared by the method of claim 77.

80. A method for detecting aggregates of a specific protein in a biological sample, comprising:
(a) incudating the sample with a biosensor cell according to claim 79; and
(b) detecting and quantifying biosensor cells that have acquired increased FRET by flow cytometry to determine aggregates of the specific protein.

81. The method of claim 80, wherein the specific protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), and TAR DNA-binding protein 43 (TDP-43).

82. A biosensor cell effective to detect aggregates of a specific protein in a biological sample, the biosensor cell comprising a reporter gene that encodes the specific protein fused with a fluorescent tag.

83. The biosensor cell of claim 82, wherein the biosensor cell is a mammalian cell.

84. The biosensor cell of claim 82, wherein the biosensor cell is a HEK293T cell.

85. The biosensor cell of claim 82, wherein the fluorescent tag is one or more of a photoconvertible fluorescent tag and a self labeling fluorescent tag.

86. The biosensor cell of claim 85, wherein the fluorescent tag is one or more of mEos3.1, mEos3.2, HaloTag, SNAP-tag, TMP-tag, and fluorescence-activating and absorption shifting tag (FAST).

87. The biosensor cell of claim 82, wherein the specific protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), and TAR DNA-binding protein 43 (TDP-43).

88. A method for detecting aggregates of a specific protein in a biological sample comprising: contacting the biological sample with a biosensor cell according to claim 82; and detecting and quantifying biosensor cells that have acquired increased FRET by flow cytometry to determine aggregates of the specific protein.

89. The method of claim 88, wherein the specific protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), and TAR DNA-binding protein 43 (TDP-43).

90. A method for constructing a biosensor cell that is used to detect aggregates of a specific protein in a biological sample, comprising:
constructing a reporter gene that encodes the specific protein fused with a fluorescent tag;
transporting the reporter gene inside a cell; and
sorting and/or expanding cells containing the reporter gene as the biosensor cell.

91. The method of claim 90, wherein the fluorescent tag is one or more of a photoconvertible fluorescent tag and a self labeling fluorescent tag.

92. The method of claim 91, wherein the fluorescent tag is one or more of mEos3.1, mEos3.2, HaloTag, SNAP-tag, TMP-tag, and fluorescence-activating and absorption shifting tag (FAST).

93. The method of claim 90, wherein the cell is a mammalian cell.

94. The method of claim 90, wherein the cell is a HEK293T cell.

95. The method of claim 90, wherein the transporting of the reporter gene comprises one or more of transfection, transduction, infection, or combinations thereof.

96. The method of claim 90, wherein the transporting of the reporter gene comprises the use of a lentiviral vector.

97. The method of claim 90, wherein the specific protein is selected from the group consisting of β-amyloid precursor protein (APP), tau, α-synuclein, prion protein or fragments thereof, superoxide dismutase 1, Abri, Adan, transmembrane protein 106B (TMEM106B), and TATA-binding protein-associated factor 15 (TAF15), heterogeneous nuclear ribonucleoprotein A1 (hnRNPA1), heterogeneous nuclear ribonucleoprotein A2 (hnRNPA2), and TAR DNA-binding protein 43 (TDP-43).
